SEQUENCE LISTING (52 sequences)

SEQ ID NO: 1 - protein, 5 aa, synthetic construct
NYGMN

SEQ ID NO: 2 - protein, 17 aa, synthetic construct
WINTYTGEPTYTDDFKG

SEQ ID NO: 3 - protein, 12 aa, synthetic construct
GGFGSSYWYFDV

SEQ ID NO: 4 - protein, 11 aa, synthetic construct
KASQDVSIAVA

SEQ ID NO: 5 - protein, 7 aa, synthetic construct
SASYRYT

SEQ ID NO: 6 - protein, 9 aa, synthetic construct
QQHYITPLT

SEQ ID NO: 7 - protein, 5 aa, synthetic construct
TAGMQ

SEQ ID NO: 8 - protein, 17 aa, synthetic construct
WINTHSGVPKYAEDFKG

SEQ ID NO: 9 - protein, 12 aa, synthetic construct
SGFGSSYWYFDV

SEQ ID NO: 10 - protein, 11 aa, synthetic construct
KASQDVSTAVA

SEQ ID NO: 11 - protein, 7 aa, synthetic construct
SASYRYT

SEQ ID NO: 12 - protein, 9 aa, synthetic construct
QQHYITPLT

SEQ ID NO: 13 - protein, 8 aa, synthetic construct
GYTFTNYG

SEQ ID NO: 14 - protein, 8 aa, synthetic construct
INTYTGEP

SEQ ID NO: 15 - protein, 14 aa, synthetic construct
ARGGFGSSYWYFDV

SEQ ID NO: 16 - protein, 6 aa, synthetic construct
QDVSIA

SEQ ID NO: 17 - skipped

SEQ ID NO: 18 - protein, 9 aa, synthetic construct
QQHYITPLT

SEQ ID NO: 19 - protein, 8 aa, synthetic construct
GYTFTTAG

SEQ ID NO: 20 - protein, 8 aa, synthetic construct
INTHSGVP

SEQ ID NO: 21 - protein, 14 aa, synthetic construct
ARSGFGSSYWYFDV

SEQ ID NO: 22 - protein, 6 aa, synthetic construct
QDVSTA

SEQ ID NO: 23 - skipped

SEQ ID NO: 24 - protein, 9 aa, synthetic construct
QQHYITPLT

SEQ ID NO: 25 - protein, 7 aa, synthetic construct
GYTFTNY

SEQ ID NO: 26 - protein, 4 aa, synthetic construct
TYTG

SEQ ID NO: 27 - protein, 10 aa, synthetic construct
GFGSSYWYFD

SEQ ID NO: 28 - protein, 7 aa, synthetic construct
SQDVSIA

SEQ ID NO: 29 - skipped

SEQ ID NO: 30 - protein, 6 aa, synthetic construct
HYITPL

SEQ ID NO: 31 - protein, 7 aa, synthetic construct
GYTFTTA

SEQ ID NO: 32 - protein, 4 aa, synthetic construct
THSG

SEQ ID NO: 33 - protein, 10 aa, synthetic construct
GFGSSYWYFD

SEQ ID NO: 34 - protein, 7 aa, synthetic construct
SQDVSTA

SEQ ID NO: 35 - skipped

SEQ ID NO: 36 - protein, 6 aa, synthetic construct
HYITPL

SEQ ID NO: 37 - protein, 10 aa, synthetic construct
ASGYTFTNYG

SEQ ID NO: 38 - protein, 17 aa, synthetic construct
INTYTGEPTYTDDFKGR

SEQ ID NO: 39 - protein, 11 aa, synthetic construct
GGFGSSYWYFD

SEQ ID NO: 40 - protein, 8 aa, synthetic construct
ASQDVSIA

SEQ ID NO: 41 - protein, 12 aa, synthetic construct
SASYRYTGVPDR

SEQ ID NO: 42 - protein, 6 aa, synthetic construct
HYITPL

SEQ ID NO: 43 - protein, 10 aa, synthetic construct
ASGYTFTTAG

SEQ ID NO: 44 - protein, 17 aa, synthetic construct
INTHSGVPKYAEDFKGR

SEQ ID NO: 45 - protein, 11 aa, synthetic construct
SGFGSSYWYFD

SEQ ID NO: 46 - protein, 8 aa, synthetic construct
ASQDVSTA

SEQ ID NO: 47 - protein, 12 aa, synthetic construct
SASYRYTGVPSR

SEQ ID NO: 48 - protein, 6 aa, synthetic construct
HYITPL

SEQ ID NO: 49 - protein, 120 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVS

SEQ ID NO: 50 - protein, 107 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 51 - protein, 121 aa, synthetic construct
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 52 - protein, 107 aa, synthetic construct
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIK

(19) United States
(12) Patent Application Publication
(10) Pub. No.: US 2025/0249115 A1
(43) Pub. Date: Aug. 7, 2025

(54) COMBINATION THERAPY FOR TREATING TUMOR ANTIGEN EXPRESSING CANCERS

(71) Applicants: Gilead Sciences, Inc., Foster City, CA (US); Arcus Biosciences, Inc., Hayward, CA (US)

(72) Inventors: Oyewale Abidoye, Atherton, CA (US); Chih-Chien Chou, Redwood City, CA (US); William J. Grossman, Danville, CA (US); Jessica N. Orf, San Francisco, CA (US); Joseph Kwang Park, Newbury Park, CA (US); Nathalie Scholler, Pacific Grove, CA (US); Mitchell R. Sierecki, Cold Spring Harbor, NY (US)

(21) Appl. No.: 18/856,032

(22) PCT Filed: Apr. 12, 2023
(86) PCT No.: PCT/US2023/065683

§ 371 Date: Oct. 10, 2024

Related U.S. Application Data
(60) Provisional application No. 63/377,990, filed on Sep. 30, 2022.
(60) Provisional application No. 63/359,016, filed on Jul. 7, 2022.
(60) Provisional application No. 63/330,708, filed on Apr. 13, 2022.

(51) Int. Cl. A61K 47/68; A61K 9/00; A61K 39/00; A61P 35/00; C07K 16/28; C07K 16/30

(52) U.S. Cl. CPC A61K 47/68037; A61K 9/0019; A61K 47/6851; A61P 35/00; C07K 16/2818; C07K 16/30; A61K 2039/507; A61K 2039/54; A61K 2039/545; C07K 2317/92

(57) ABSTRACT

The present disclosure relates to methods of treating, mitigating, or preventing or delaying the recurrence or metastasis of a cancer in a subject by administering an effective amount of: (a) an antibody-drug conjugate (ADC) comprising (i) an anti-TROP-2 antibody; and/or (ii) a topoisomerase I inhibitor; (b) an anti-PD-(L)1 antibody; and, optionally, (c) an anti-TIGIT antibody.

### CROSS-REFERENCE TO RELATED APPLICATIONS

This application claims the benefit under 35 U.S.C. § 119(e) of U.S. Provisional Application No. 63/330,708, filed Apr. 13, 2022, U.S. Provisional Application No. 63/359,016, filed Jul. 7, 2022, and U.S. Provisional Application No. 63/377,990, filed Sep. 30, 2022, each of which is hereby incorporated by reference in its entirety.

### FIELD

The present disclosure relates to methods of treating, mitigating, or preventing or delaying the recurrence or metastasis of, a Trop-2 expressing cancer (e.g., metastatic urothelial cancer or non-small cell lung cancer) in a subject by administering an effective amount of: (a) a TROP-2 targeted antibody drug conjugate (ADC) comprising an anti-TROP-2 antibody; (b) an anti-PD-(L)1 antibody; and, optionally, (c) an anti-TIGIT antibody to the subject. The present disclosure further relates to methods of treating, mitigating, or preventing or delaying the recurrence or metastasis of a tumor antigen (TA) positive (TA⁺) cancer in a subject by administering an effective amount of: (a) a tumor antigen targeted antibody-drug conjugate (ADC) comprising a topoisomerase I inhibitor (TopI ADC); (b) an anti-PD-(L)1 antibody; and, optionally, (c) an anti-TIGIT antibody to the subject.

### BACKGROUND

Antibody drug conjugates, such as sacituzumab govitecan and datopotamab deruxtecan, are under clinical investigation for the treatment of a variety of Trop-2 expressing or tumor antigen positive cancers. While evidence of clinical efficacy has been obtained in a monotherapy setting. Further therapeutic benefits for patients are desired.

Trop-2 expression has been reported for a variety of epithelial cancers, including breast, bladder, lung, colorectal and prostate cancers. It is estimated that over 430,000 men and women are diagnosed worldwide with bladder cancer. In addition, bladder cancer accounts for nearly 170,000 deaths worldwide annually. Urothelial cancer (UC) is the predominant histologic type in the United States and Europe. For over forty years, platinum-based chemotherapy was the standard of care for treating bladder cancer. However, recent advances in the genomic characterization of bladder cancer has led to investigations into the use of immune checkpoint inhibitors for the treatment of bladder cancer. In fact, from 2016 to 2019, the US Food and Drug Administration approved nine new therapies for the treatment of advanced urothelial carcinoma, seven of which involved immune checkpoint inhibitors (Patel, et L., Treatment of Muscle-Invasive and Advanced Bladder Cancer in 2020, CA Cancer J CLIN, 70:404-423, 2020).

Despite these advances, there is still a need for the development of novel therapies and combinations for the treatment of cancers, such as Trop-2 expressing or tumor antigen positive cancers.

### SUMMARY

In one aspect, provided herein are methods of treating, mitigating, reducing, preventing or delaying the recurrence or metastasis of a Trop-2 positive cancer, comprising co-administering to a subject an effective amount of: a) a TROP-2-targeted antibody-drug conjugate (ADC) comprising an anti-TROP-2 antibody (anti-TROP-2 ADC); b) an anti-PD-(L)1 antibody; and, optionally, c) an anti-TIGIT antibody.

In some embodiments, the methods provided herein are for treating a Trop-2 positive cancer comprising co-administering to a subject an effective amount of: a) a TROP-2-targeted antibody-drug conjugate (ADC) comprising an anti-TROP-2 antibody (anti-TROP-2 ADC); b) an anti-PD-(L)1 antibody; and, optionally, c) an anti-TIGIT antibody.

Further provided herein are methods of treating, mitigating, reducing, preventing or delaying the recurrence of urothelial cancer (UC), comprising co-administering to a subject an effective amount of: a) a TROP-2-targeted antibody-drug conjugate (ADC) comprising an anti-TROP-2 antibody (anti-TROP-2 ADC); b) an anti-PD-(L)1 antibody; and, optionally, c) an anti-TIGIT antibody.

In some embodiments, the methods provided herein are for treating urothelial cancer comprising co-administering to a subject an effective amount of: a) a TROP-2-targeted antibody-drug conjugate (ADC) comprising an anti-TROP-2 antibody (anti-TROP-2 ADC); b) an anti-PD-(L)1 antibody; and, optionally, c) an anti-TIGIT antibody.

Further disclosed herein are methods of treating, mitigating, reducing, preventing or delaying the recurrence or metastasis of a Trop-2 positive cancer, comprising co-administering to a subject an effective amount of: (a) a TROP-2-targeted antibody-drug conjugate (ADC) comprising an anti-TROP-2 antibody (anti-TROP-2 ADC); (b) zimberelimab; and, optionally, (c) an anti-TIGIT antibody.

Further disclosed herein are methods of treating a Trop-2 positive cancer comprising co-administering to a subject an effective amount of: (a) a TROP-2-targeted antibody-drug conjugate (ADC) comprising an anti-TROP-2 antibody (anti-TROP-2 ADC); (b) zimberelimab; and, optionally, (c) an anti-TIGIT antibody.

Further disclosed herein are methods of treating, mitigating, reducing, preventing or delaying the recurrence or metastasis of a Trop-2 positive cancer, co-administering to a subject an effective amount of: (a) a TROP-2-targeted antibody-drug conjugate (ADC) comprising an anti-TROP-2 antibody (anti-TROP-2 ADC); (b) zimberelimab; and, optionally, (c) domvanalimab.

Further disclosed herein are methods of treating a Trop-2 positive cancer comprising co-administering to a subject an effective amount of: (a) a TROP-2-targeted antibody-drug conjugate (ADC) comprising an anti-TROP-2 antibody (anti-TROP-2 ADC); (b) zimberelimab; and, optionally, (c) domvanalimab.

Further disclosed herein are methods of treating, mitigating, reducing, preventing or delaying the recurrence or metastasis of a Trop-2 positive cancer, comprising co-administering to a subject an effective amount of: (a) a TROP-2-targeted antibody-drug conjugate (ADC) comprising an anti-TROP-2 antibody (anti-TROP-2 ADC); (b) an anti-PD-(L)1 antibody; and, optionally, (c) domvanalimab.

Further disclosed herein are methods of treating a Trop-2 positive cancer comprising co-administering to a subject an effective amount of: (a) a TROP-2-targeted antibody-drug conjugate (ADC) comprising an anti-TROP-2 antibody (anti-TROP-2 ADC); (b) an anti-PD-(L)1 antibody; and, optionally, (c) domvanalimab.

Further disclosed herein are methods of treating, mitigating, reducing, preventing or delaying the recurrence or metastasis of urothelial cancer, comprising co-administering to a subject an effective amount of: (a) a TROP-2-targeted antibody-drug conjugate (ADC) comprising an anti-TROP-2 antibody (anti-TROP-2 ADC); (b) zimberelimab; and, optionally, (c) an anti-TIGIT antibody.

Further disclosed herein are methods of treating urothelial cancer comprising co-administering to a subject an effective amount of: (a) a TROP-2-targeted antibody-drug conjugate (ADC) comprising an anti-TROP-2 antibody (anti-TROP-2 ADC); (b) zimberelimab; and, optionally, (c) an anti-TIGIT antibody.

Further disclosed herein are methods of treating, mitigating, reducing, preventing or delaying the recurrence or metastasis of urothelial cancer, co-administering to a subject an effective amount of: (a) a TROP-2-targeted antibody-drug conjugate (ADC) comprising an anti-TROP-2 antibody (anti-TROP-2 ADC); (b) zimberelimab; and, optionally, (c) domvanalimab.

Further disclosed herein are methods of treating urothelial cancer comprising co-administering to a subject an effective amount of: (a) a TROP-2-targeted antibody-drug conjugate (ADC) comprising an anti-TROP-2 antibody (anti-TROP-2 ADC); (b) zimberelimab; and, optionally, (c) domvanalimab.

Further disclosed herein are methods of treating, mitigating, reducing, preventing or delaying the recurrence or metastasis of urothelial cancer, comprising co-administering to a subject an effective amount of: (a) a TROP-2-targeted antibody-drug conjugate (ADC) comprising an anti-TROP-2 antibody (anti-TROP-2 ADC); (b) an anti-PD-(L)1 antibody; and, optionally, (c) domvanalimab.

Further disclosed herein are methods of treating urothelial cancer comprising co-administering to a subject an effective amount of: (a) a TROP-2-targeted antibody-drug conjugate (ADC) comprising an anti-TROP-2 antibody (anti-TROP-2 ADC); (b) an anti-PD-(L)1 antibody; and, optionally, (c) domvanalimab.

In some embodiments, the anti-TROP-2 ADC comprises an anticancer agent payload.

In some embodiments, the anticancer agent payload is selected from a microtubule inhibitor, DNA cleavage agent, and topoisomerase I inhibitor.

In some embodiments, the anti-TROP-2 ADC comprises a topoisomerase I inhibitor.

In some embodiments, the topoisomerase I inhibitor is selected from irinotecan, topetecan, and SN-38.

In some embodiments, the anti-TROP-2 ADC has a structural formula of mAb-CL2A-SN-38, with a structure represented by:

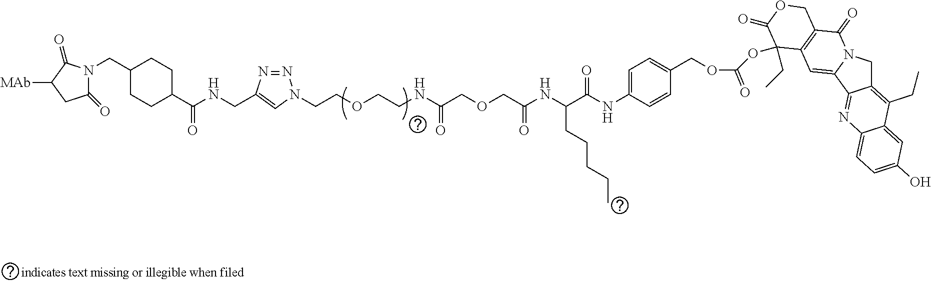

(described, e.g., in U.S. Pat. No. 7,999,083).

In some embodiments, the anti-TROP-2 ADC comprises sacituzumab (hRS7; described, e.g., in WO2003074566, FIGS. 3 and 4).

In some embodiments, the anti-TROP-2 ADC is selected from sacituzumab govitecan, datopotamab deruxtecan (DS-1062), ESG-401, SKB-264, DAC-02 and BAT-8003.

In some embodiments, the anti-TROP-2 ADC is sacituzumab govitecan.

In some embodiments, the anti-TROP-2 ADC comprises a microtuble inhibitor.

In some embodiments, the microtubule inhibitor is selected from is an auristatin, a taxane, a vinca alkaloid, an epothilone, and maytansinoid.

In some embodiments, the auristatin is selected from monomethyl auristatin E (MMAE) and monomethyl auristatin F (MMAF).

In some embodiments, the maytansinoid is selected from mertansine (DM1) and ravtansine (DM4).

In some embodiments, the anticancer agent payload is selected from anthracyline (e.g., doxorubicin, daunorubicin, epirubicin, idarubicin), pyrrolobenzodiazepine (PBD), or dimer thereof, DNA cross-linking agent SC-DR002 (D6.5), duocarmycin, a duocarmycin (A, B1, B2, C1, C2, D, SA, CC-1065), tubulysin B and analogs thereof.

In another aspect, provided herein are methods of treating, mitigating, reducing, preventing or delaying the recurrence or metastasis of a tumor antigen positive (TA⁺) cancer comprising co-administering to a subject an effective amount of: a) a tumor antigen-targeted antibody-drug conjugate (ADC) comprising a topoisomerase I inhibitor (TopI ADC); b) an anti-PD-(L)1 antibody; and, optionally, c) an anti-TIGIT antibody.

In some embodiments, the methods provided herein are for treating a tumor antigen positive (TA⁺) cancer comprising co-administering to a subject an effective amount of: a) a tumor antigen-targeted antibody-drug conjugate (ADC) comprising a topoisomerase I inhibitor (TopI ADC); b) an anti-PD-(L)1 antibody; and, optionally, c) an anti-TIGIT antibody.

Further provided herein are methods of treating, mitigating, reducing, preventing or delaying the recurrence of urothelial cancer (UC), comprising co-administering to a subject an effective amount of: a) a tumor antigen-targeted antibody-drug conjugate (ADC) comprising a topoisomerase I inhibitor (TopI ADC); b) an anti-PD-(L)1 antibody; and, optionally, c) an anti-TIGIT antibody.

In some embodiments, the methods provided herein are for treating urothelial cancer comprising co-administering to a subject an effective amount of: a) a tumor antigen-targeted antibody-drug conjugate (ADC) comprising a topoisomerase I inhibitor (TopI ADC); b) an anti-PD-(L)1 antibody; and, optionally, c) an anti-TIGIT antibody.

Further disclosed herein are methods of treating, mitigating, reducing, preventing or delaying the recurrence or metastasis of a tumor antigen positive (TA⁺) cancer, comprising co-administering to a subject an effective amount of: (a) a tumor antigen-targeted antibody-drug conjugate (ADC) comprising a topoisomerase I inhibitor (TopI ADC); (b) zimberelimab; and, optionally, (c) an anti-TIGIT antibody.

Further disclosed herein are methods of treating a tumor antigen positive (TA⁺) cancer comprising co-administering to a subject an effective amount of: (a) a tumor antigen-targeted antibody-drug conjugate (ADC) comprising a topoisomerase I inhibitor (TopI ADC); (b) zimberelimab; and, optionally, (c) an anti-TIGIT antibody.

Further disclosed herein are methods of treating, mitigating, reducing, preventing or delaying the recurrence or metastasis of a tumor antigen positive (TA⁺) cancer, co-administering to a subject an effective amount of: (a) a tumor antigen-targeted antibody-drug conjugate (ADC) comprising a topoisomerase I inhibitor (TopI ADC); (b) zimberelimab; and, optionally, (c) domvanalimab.

Further disclosed herein are methods of treating a tumor antigen positive (TA⁴) cancer comprising co-administering to a subject an effective amount of: (a) a tumor antigen-targeted antibody-drug conjugate (ADC) comprising a topoisomerase I inhibitor (TopI ADC); (b) zimberelimab; and, optionally, (c) domvanalimab.

Further disclosed herein are methods of treating, mitigating, reducing, preventing or delaying the recurrence or metastasis of a tumor antigen positive (TA⁺) cancer, comprising co-administering to a subject an effective amount of: (a) a tumor antigen-targeted antibody-drug conjugate (ADC) comprising a topoisomerase I inhibitor (TopI ADC); (b) an anti-PD-(L)1 antibody; and, optionally, (c) domvanalimab.

Further disclosed herein are methods of treating a tumor antigen positive (TA⁺) cancer comprising co-administering to a subject an effective amount of: (a) a tumor antigen-targeted antibody-drug conjugate (ADC) comprising a topoisomerase I inhibitor (TopI ADC); (b) an anti-PD-(L)1 antibody; and, optionally, (c) domvanalimab.

Further disclosed herein are methods of treating, mitigating, reducing, preventing or delaying the recurrence or metastasis of urothelial cancer, comprising co-administering to a subject an effective amount of: (a) a tumor antigen-targeted antibody-drug conjugate (ADC) comprising a topoisomerase I inhibitor (TopI ADC); (b) zimberelimab; and, optionally, (c) an anti-TIGIT antibody.

Further disclosed herein are methods of treating urothelial cancer comprising co-administering to a subject an effective amount of: (a) a tumor antigen-targeted antibody-drug conjugate (ADC) comprising a topoisomerase I inhibitor (TopI ADC); (b) zimberelimab; and, optionally, (c) an anti-TIGIT antibody.

Further disclosed herein are methods of treating, mitigating, reducing, preventing or delaying the recurrence or metastasis of urothelial cancer, co-administering to a subject an effective amount of: (a) a tumor antigen-targeted antibody-drug conjugate (ADC) comprising a topoisomerase I inhibitor (TopI ADC); (b) zimberelimab; and, optionally, (c) domvanalimab.

Further disclosed herein are methods of treating urothelial cancer comprising co-administering to a subject an effective amount of: (a) a tumor antigen-targeted antibody-drug conjugate (ADC) comprising a topoisomerase I inhibitor (TopI ADC); (b) zimberelimab; and, optionally, (c) domvanalimab.

Further disclosed herein are methods of treating, mitigating, reducing, preventing or delaying the recurrence or metastasis of urothelial cancer, comprising co-administering to a subject an effective amount of: (a) a tumor antigen-targeted antibody-drug conjugate (ADC) comprising a topoisomerase I inhibitor (TopI ADC); (b) an anti-PD-(L)1 antibody; and, optionally, (c) domvanalimab.

Further disclosed herein are methods of treating urothelial cancer comprising co-administering to a subject an effective amount of: (a) a tumor antigen-targeted antibody-drug conjugate (ADC) comprising a topoisomerase I inhibitor (TopI ADC); (b) an anti-PD-(L)1 antibody; and, optionally, (c) domvanalimab.

In some embodiments, the topoisomerase I inhibitor is a camptothecin.

In some embodiments, the camptothecin is selected from irinotecan, topotecan, belotecan, and exatecan derivative.

In some embodiments, the exatecan derivative is selected from Dxd or SN38.

In some embodiments, the topoisomerase I inhibitor is selected from irinotecan, topetecan, and SN-38.

In some embodiments, the TopI ADC has a structural formula of mAb-CL2A-SN-38, with a structure represented by:

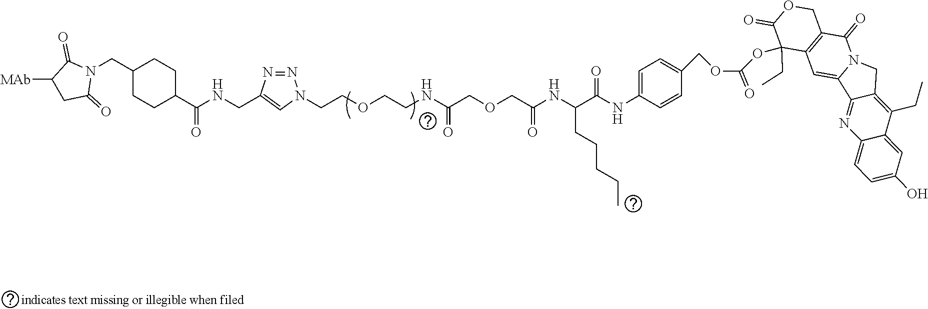

(described, e.g., in U.S. Pat. No. 7,999,083).

In some embodiments, the TopI ADC comprises an antibody that binds a tumor antigen.

In some embodiments, the tumor antigen is selected from carbonic anhydrase IX, B7, CCCL19, CCCL21, CSAp, HER-2/neu, BrE3, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD80, CD83, CD95, CD126, CD133, CD138, CD147, CD154, CEACAM5, CEACAM-6, alpha-fetoprotein (AFP), VEGF, ED-B fibronectin, EGP-1, EGP-2, EGF receptor (ErbB1), ErbB2, ErbB3, Factor H, FHL-1, Flt-3, folate receptor, Ga 733, GROB, HMGB-1, hypoxia inducible factor (HIF), HM1.24, insulin-like growth factor (ILGF), IFN-α, IFN-b, IL-g, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, HLA-DR, HLA-G, CD66a-d, MAGE, mCRP, MCP-1, MIP-1A, MIP-1B, macrophage migration-inhibitory factor (MIF), MUC1, MUC2, MUC3, MUC4, MUC5, placental growth factor (P1GF), PSA (prostate-specific antigen), PSMA, PSMA dimer, PAM4 antigen, NCA-95, NCA-90, A3, A33, Ep-CAM, KS-1, Le(y), mesothelin, S100, tenascin, TAC, Tn antigen, Thomas-Friedenreich antigens, tumor necrosis antigens, tumor angiogenesis antigens, TNF-a, TRAIL receptor (R1 and R2), VEGFR, RANTES, T101, cancer stem cell antigens, complement factors C3, C3a, C3b, C5a, C5, and an oncogene product.

In some embodiments, the TopI ADC comprises an antibody selected from gemtuzumab, brentuximab, belantamab, camidanlumab, trastuzumab, inotuzumab, glembatumumab, anetumab, mirvetuximab, depatuxizumab, vadastuximab, labetuzumab, ladiratuzumab, loncastuximab, patritumab, lifastuzumab, indusatumab, polatuzumab, pinatuzumab, coltuximab, upifitamab, indatuximab, milatuzumab, rovalpituzumab, enfortumab, tisotumab, tusamitamab, disitamab, telisotuzumab, and antigen-binding fragments thereof.

In some embodiments, the TopI ADC comprises an antibody selected from hLL1 (anti-CD74), hLL2 (anti-CD22), hRFB4 (anti-CD22), h PAM4 (anti-MUC5ac), hMN-3 (anti-NOTCH3), hMN-14 (labetuzumab; anti-CEACAM5); hMN15 (anti-CEACAM6) hA19 (anti-CD19), hA20 (anti-CD22), hMu-9 (anti-CSAp), hL243 (anti-HLA-DR), hImmu-31 (anti-AFP), and antigen-binding fragments thereof.

In some embodiments, the anti-PD-(L)1 antibody is pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, or zimberelimab.

In some embodiments, the anti-PD-(L)1 antibody is zimberelimab.

In some embodiments, the anti-PD-(L)1 antibody is zimberelimab.

In some embodiments, the anti-PD-(L)1 antibody is an Fc-silent antibody.

In some embodiments, the anti-PD-(L)1 antibody is an Fc-enabled antibody.

In some embodiments, any of the methods disclosed herein further comprise administering an anti-TIGIT antibody.

In some embodiments, the anti-TIGIT antibody is AB308, AGEN-1307 (AGEN-1327), AGEN-1777, AK127, BMS-986207, domvanalimab, EOS-448, etigilimab, JS006, M6223, ociperlimab, ralzapastotug, SEA-TGT (SGN-TGT), tiragolumab, or vibostolimab.

In some embodiments, the anti-TIGIT antibody is domvanalimab. In some embodiments, the anti-TIGIT antibody is M6223.

In some embodiments, the anti-PD-(L)1 antibody and the anti-TIGIT antibody are a) zimberelimab and domvanalimab, b) zimberelimab and AB308, c) atezolizumab and tiragolumab, d) pembrolizumab and vibostolimab, e) pembrolizumab and domvanalimab, f) pembrolizumab and AB308, g) MK-7684A (pembrolizumab/vibostolimab coformulation), h) durvalumab and domvanalimab, i) zimberelimab and ralzapastotug, or j) pembrolizumab and ralzapastotug.

In some embodiments, the anti-PD-(L)1 antibody and the anti-TIGIT antibody are zimberelimab and domvanalimab.

In some embodiments, the method further comprises co-administering an additional therapeutic agent or therapeutic modality.

In some embodiments, the additional therapeutic agent or therapeutic modality comprises one, two, three, or four additional therapeutic agents and/or therapeutic modalities.

In some embodiments, the Trop-2 positive cancer is a solid epithelial cancer.

In some embodiments, the TA⁺ cancer is a solid epithelial cancer.

In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer.

In some embodiments, the bladder cancer is urothelial cancer (UC).

In some embodiments, the bladder cancer is (i) unresectable, locally advanced bladder cancer, (ii) metastatic bladder cancer, or (iii) muscle-invasive bladder cancer.

In some embodiments, the urothelial cancer is (i) unresectable, locally advanced urothelial cancer, (ii) metastatic urothelial cancer, or (iii) muscle-invasive urothelial cancer.

In some embodiments, the lung cancer is non-small cell lung cancer (NSCLC).

In some embodiments, the lung cancer is (i) advanced lung cancer or (ii) metastatic lung cancer.

In some embodiments, the lung cancer is (i) advanced NSCLC or (ii) metastatic NSCLC.

In some embodiments, the NSCLC is squamous NSCLC.

In some embodiments, the NSCLC is non-squamous NSCLC.

In some embodiments, the metastatic NSCLC is metastatic squamous NSCLC.

In some embodiments, the metastatic NSCLC is metastatic non-squamous NSCLC.

In some embodiments, the NSCLC is NSCLC without EGFR, ALK, or other actionable genomic alterations.

In some embodiments, the cancer is (i) unresectable, locally advanced, (ii) metastatic, or (iii) muscle invasive.

In some embodiments, the cancer is (i) advanced or (ii) metastatic.

In some embodiments, the Trop-2 positive cancer is (i) unresectable, locally advanced, (ii) metastatic, or (iii) muscle invasive.

In some embodiments, the TA⁺ cancer is (i) unresectable, locally advanced, (ii) metastatic, or (iii) muscle invasive.

In some embodiments, the cancer has progressed following at least one prior anti-cancer therapy.

In some embodiments, the subject is treatment naïve.

In some embodiments, the treatment naïve subject has not received prior anti-cancer therapy for (i) unresectable, locally advanced cancer, (ii) metastatic cancer, or (iii) muscle invasive cancer.

In some embodiments, the subject has received one or more anti-cancer therapies before administration of the combination of the ADC, anti-PD-(L)1 antibody, and optionally anti-TIGIT antibody.

In some embodiments, the subject is not treatment naïve.

In some embodiments, the subject has received one or more prior anti-cancer therapies for (i) unresectable, locally advanced cancer, (ii) metastatic cancer, (iii) muscle invasive cancer.

In some embodiments, the subject has received one or more anti-cancer therapies before administration of the combination of the ADC, anti-PD-(L)1 antibody, and optionally anti-TIGIT antibody.

In some embodiments, the cancer is resistant or refractory to one or more anti-cancer therapies.

In some embodiments, the anti-cancer therapy is selected from surgery, radiation therapy, chemotherapy, and checkpoint inhibitor therapy.

In some embodiments, the combination of the ADC, anti-PD-(L)1 antibody, and optionally anti-TIGIT antibody is administered in a neoadjuvant setting (e.g., in preparation for surgery or radiation therapy).

In some embodiments, the combination of the ADC, anti-PD-(L)1 antibody, and optionally anti-TIGIT antibody is administered in an adjuvant setting (e.g., following a primary treatment such as surgery or radiation therapy).

In some embodiments, the combination of the ADC, anti-PD-(L)1 antibody, and optionally anti-TIGIT antibody is administered in a therapeutic setting (e.g., as the primary therapy).

In some embodiments, the combination of the ADC, anti-PD-(L)1 antibody, and optionally anti-TIGIT antibody is administered in a maintenance setting.

In some embodiments, the subject receives one or more doses of the ADC.

In some embodiments, the subject receives one or more doses of the anti-PD-(L)1 antibody.

In some embodiments, the subject receives one or more doses of the anti-TIGIT antibody.

In some embodiments, the ADC and the anti-PD-(L)1 antibody are administered concurrently.

In some embodiments, the ADC and the anti-PD-(L)1 antibody are administered sequentially.

In some embodiments, the ADC and the anti-TIGIT antibody are administered concurrently.

In some embodiments, the ADC and the anti-TIGIT antibody are administered sequentially.

In some embodiments, the anti-PD-(L)1 antibody and the anti-TIGIT antibody are administered concurrently.

In some embodiments, the anti-PD-(L)1 antibody and the anti-TIGIT antibody are administered sequentially.

In some embodiments, the subject is human.

In some embodiments, the subject is cisplatin-ineligible.

In some embodiments, the anti-TROP-2 ADC is administered at one or more doses in the range of 8 mg/kg to 10 mg/kg.

In some embodiments, the anti-TROP-2 ADC is administered at one or more doses of 8 mg/kg or 10 mg/kg.

In some embodiments, the anti-TROP-2 ADC is administered at one or more doses of 10 mg/kg.

In some embodiments, the anti-TROP-2 ADC is administered intravenously.

In some embodiments, the anti-TROP-2 ADC is administered on days 1 and 8 of a 21-day cycle.

In some embodiments, the anti-PD-(L)1 antibody is administered at one or more doses in the range of 300 mg to 400 mg.

In some embodiments, the anti-PD-(L)1 antibody is administered at dose of 360 mg.

In some embodiments, the anti-PD-(L)1 antibody is administered intravenously.

In some embodiments, the anti-PD-(L)1 antibody is administered on day 1 of a 21-day cycle.

In some embodiments, the anti-TIGIT antibody is administered at one or more doses in the range of 800 mg to 1600 mg.

In some embodiments, the anti-TIGIT antibody is administered at a dose of 1200 mg.

In some embodiments, the anti-TIGIT antibody is administered intravenously.

In some embodiments, the anti-TIGIT antibody is administered on day 1 of a 21-day cycle.

In some embodiments, provided herein are methods of treating, mitigating, reducing, preventing or delaying the recurrence or metastasis of a Trop-2 positive cancer comprising co-administering to a human patient an effective amount of a) sacituzumab govitecan; b) zimberelimab; and, optionally, c) domvanalimab.

In some embodiments, provided herein are methods of treating a Trop-2 positive cancer comprising co-administering to a human patient an effective amount of a) sacituzumab govitecan; b) zimberelimab; and, optionally, c) domvanalimab.

In some embodiments, provided herein are methods of treating, mitigating, reducing, preventing or delaying the recurrence or metastasis of a tumor antigen positive (TA⁺) cancer comprising co-administering to a human patient an effective amount of a) sacituzumab govitecan; b) zimberelimab; and, optionally, c) domvanalimab.

In some embodiments, provided herein are methods of treating a tumor antigen positive (TA⁺) cancer comprising co-administering to a human patient an effective amount of a) sacituzumab govitecan; b) zimberelimab; and, optionally, c) domvanalimab.

In some embodiments, provided herein are methods of treating, mitigating, reducing, preventing or delaying the recurrence or metastasis of urothelial cancer (UC) comprising co-administering to a human patient an effective amount of a) sacituzumab govitecan; b) zimberelimab; and, optionally, c) domvanalimab.

In some embodiments, provided herein are methods of treating urothelial cancer (UC) comprising co-administering to a human patient an effective amount of a) sacituzumab govitecan; b) zimberelimab; and, optionally, c) domvanalimab.

In some embodiments, provided herein are methods of treating, mitigating, reducing, preventing or delaying the recurrence of metastatic urothelial cancer (mUC) comprising co-administering to a human patient an effective amount of a) sacituzumab govitecan; b) zimberelimab; and, optionally, c) domvanalimab.

In some embodiments, provided herein are methods of treating metastatic urothelial cancer (mUC) comprising co-administering to a human patient an effective amount of a) sacituzumab govitecan; b) zimberelimab; and, optionally, c) domvanalimab.

In some embodiments, sacituzumab govitecan is administered at one or more doses in the range of 8 mg/kg to 10 mg/kg.

In some embodiments, sacituzumab govitecan is administered at one or more doses of 10 mg/kg.

In some embodiments, sacituzumab govitecan is administered intravenously.

In some embodiments, sacituzumab govitecan is administered on days 1 and 8 of a 21-day cycle.

In some embodiments, zimberelimab is administered at one or more doses in the range of 300 mg to 400 mg.

In some embodiments, zimberelimab is administered at dose of 360 mg.

In some embodiments, zimberelimab is administered intravenously.

In some embodiments, zimberelimab is administered on day 1 of a 21-day cycle.

In some embodiments, domvanalimab is administered at one or more doses in the range of 800 mg to 1600 mg.

In some embodiments, domvanalimab is administered at a dose of 1200 mg.

In some embodiments, domvanalimab is administered intravenously.

In some embodiments, domvanalimab is administered on day 1 of a 21-day cycle.

In some embodiments, sacituzumab govitecan is administered at a dose of 10 mg/kg on days 1 and 8 of a 21-day cycle; zimberelimab is administered at a dose of 360 mg on day 1 of the 21-day cycle.

In some embodiments, sacituzumab govitecan is administered at a dose of 10 mg/kg on days 1 and 8 of a 21-day cycle; zimberelimab is administered at a dose of 360 mg on day 1 of the 21-day cycle, and domvanalimab is administered at a dose of 1200 mg on day 1 of the 21-day cycle.

In some embodiments, the combination of sacituzumab govitecan zimberelimab, and, optionally, domvanalimab is administered in a neoadjuvant setting (e.g., in preparation for surgery or radiation therapy).

In some embodiments, the combination of sacituzumab govitecan zimberelimab, and, optionally, domvanalimab is administered in an adjuvant setting (e.g., following a primary treatment such as surgery or radiation therapy).

In some embodiments, the sacituzumab govitecan zimberelimab, and, optionally, domvanalimab is administered in a therapeutic setting (e.g., as the primary therapy).

In some embodiments, the combination of sacituzumab govitecan zimberelimab, and, optionally, domvanalimab is administered in a maintenance setting.

In some embodiments of the methods provided herein, an anti-CD47 antibody is not co-administered to the subject or human patient.

In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643.

In some embodiments, the anti-CD47 antibody is magrolimab.

In some embodiments of the methods provided herein, an MCL1 inhibitor is not co-administered to the subject or human patient.

In some embodiments, the MCL1 inhibitor is selected from GS-9716, AMG-176, AMG-397, S-64315, AZD-5991, 483-LM, A-1210477, UMI-77 and JKY-5-037.

In some embodiments, the MCL1 inhibitor is GS-9716.

In some embodiments of the methods provided herein, a FLT3 agonist is not administered to the subject or human patient.

In some embodiments, the FLT3 agonist is selected from GS-3583, CDX-301, TAK-605, ONCR-177, Alb-Ftl3L, and SYM-027.

In some embodiments, the FLT3 agonist is GS-3583.

### BRIEF DESCRIPTION OF THE DRAWINGS

FIG. 1 shows a schematic of the treatment arms for the study described in Example 1.

FIG. 2 shows a schedule of assessments/study calendar for the study described in Example 1.

FIG. 3 shows a heatmap of the percentage of tumor infiltrating immune cell types, per live cells for CD45+ cells or per CD45+ cells for T cells, CD8 T cells, CD4 T cells, DC, NK and macrophages, correlated to treatment type (Y axis, as indicated); two-way ANOVA ordinary; row factor p<0.001.

FIG. 4 shows survival curves of hTrop-2 transgenic mice bearing orthotopic hTrop-2E0771 syngeneic breast cancer.

FIGS. 5A-F show growth curves of E0771 hTrop-2 in hTrop-2 KI mice, 26 days after tumor implant. FIG. 5A shows the mean tumor volume in mice treated with PBS (Group 1), 500 μg of control ADC (Group 2), 200 μg of control ADC (Group 3), 500 μg of murinized SG (Group 4), or 200 μg murinized SG (Group 5). FIG. 5B shows growth curves of E0771 hTrop-2 in hTrop-2 KI mice treated with PBS (Group 1). FIG. 5C shows growth curves of E0771 hTrop-2 in hTrop-2 KI mice treated with 500 μg of control ADC (Group 2). FIG. 5D shows growth curves of E0771 hTrop-2 in hTrop-2 KI mice treated with 200 μg of control ADC (Group 3). FIG. 5E shows growth curves of E0771 hTrop-2 in hTrop-2 KI mice treated with 500 μg of murinized SG (Group 4). FIG. 5F shows growth curves of curves E0771 hTrop-2 in hTrop-2 KI mice treated with 200 μg of murinized SG (Group 5).

### DETAILED DESCRIPTION

Provided herein are combination therapies for treating, mitigating, reducing, preventing, or delaying the recurrence or metastasis of a TROP-2 expressing cancer by administering effective amounts of (a) an anti-TROP-2 ADC; (b) an anti-PD-(L)1 antibody; and, optionally, (c) an anti-TIGIT antibody to a subject.

Further provided herein are combination therapies for treating, mitigating, reducing, preventing, or delaying the recurrence or metastasis of a tumor antigen positive (TA⁺) cancer by administering effective amounts of (a) a tumor antigen-targeted antibody-drug conjugate (ADC) comprising a topoisomerase I inhibitor (TopI ADC); (b) an anti-PD-(L)1 antibody; and, optionally, (c) an anti-TIGIT antibody to a subject.

In some embodiments, the treatment methods disclosed herein mitigate the occurrence or recurrence of a cancer (e.g., Trop-2 positive, TA⁺ cancer, bladder cancer, urothelial cancer, or metastatic urothelial cancer) by administering the combinations described herein. In some embodiments, the treatment methods disclosed herein reduce the occurrence or recurrence of a cancer (e.g., Trop-2 positive, TA⁺ cancer, bladder cancer, urothelial cancer, or metastatic urothelial cancer) by administering the combinations described herein. In some embodiments, the treatment methods disclosed herein prevent the occurrence or recurrence of a cancer (e.g., Trop-2 positive, TA⁺ cancer, bladder cancer, urothelial cancer, or metastatic urothelial cancer) by administering the combinations described herein. In some embodiments, the treatment methods disclosed herein delay the occurrence or recurrence of a cancer (e.g., Trop-2 positive, TA⁺ cancer, bladder cancer, urothelial cancer, or metastatic urothelial cancer) by administering the combinations described herein.

In some embodiments, the method further comprises co-administering an additional therapeutic agent or therapeutic modality.

In some embodiments, the additional therapeutic agent or therapeutic modality comprises one, two, three, or four additional therapeutic agents and/or therapeutic modalities.

In some embodiments, the Trop-2 positive cancer is a solid epithelial cancer.

In some embodiments, the TA⁺ cancer is a solid epithelial cancer.

In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer. In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations.

In some embodiments, the cancer is (i) unresectable, locally advanced, (ii) metastatic, or (iii) muscle invasive. In some embodiments, the cancer is (i) advanced or (ii) metastatic.

In some embodiments, the Trop-2 positive cancer is (i) unresectable, locally advanced or (ii) metastatic (e.g., mUC).

In some embodiments, the TA⁺ cancer is (i) unresectable, locally advanced or (ii) metastatic (e.g., mUC).

In some embodiments, the cancer has progressed following at least one prior anti-cancer therapy.

In some embodiments, the subject is treatment naïve. In some embodiments, the treatment naïve subject has not received prior anti-cancer therapy for (i) unresectable, locally advanced cancer, (ii) metastatic cancer, or (iii) muscle invasive cancer.

In some embodiments, the subject has received one or more anti-cancer therapies before administration of the combination of the ADC, anti-PD-(L)1 antibody, and optionally anti-TIGIT antibody.

In some embodiments, the subject is not treatment naïve.

In some embodiments, the subject has received one or more prior anti-cancer therapies for (i) unresectable, locally advanced cancer, (ii) metastatic cancer, or (iii) muscle invasive cancer.

In some embodiments, the subject has received one or more anti-cancer therapies before administration of the combination of the ADC, anti-PD-(L)1 antibody, and optionally anti-TIGIT antibody.

In some embodiments, the cancer is resistant or refractory to one or more anti-cancer therapies.

In some embodiments, the anti-cancer therapy is selected from surgery, radiation therapy, chemotherapy, and checkpoint inhibitor therapy.

In some embodiments, the combination of the ADC, anti-PD-(L)1 antibody, and optionally anti-TIGIT antibody is administered in a neoadjuvant setting (e.g., in preparation for surgery or radiation therapy).

In some embodiments, the combination of the ADC, anti-PD-(L)1 antibody, and optionally anti-TIGIT antibody is administered in an adjuvant setting (e.g., following a primary treatment such as surgery or radiation therapy).

In some embodiments, the combination of the ADC, anti-PD-(L)1 antibody, and optionally anti-TIGIT antibody is administered in a therapeutic setting (e.g., as the primary therapy).

In some embodiments, the combination of the ADC, anti-PD-(L)1 antibody, and optionally anti-TIGIT antibody is administered in a maintenance setting.

In some embodiments, the subject receives one or more doses of the ADC.

In some embodiments, the subject receives one or more doses of the anti-PD-(L)1 antibody.

In some embodiments, the subject receives one or more doses of the anti-TIGIT antibody.

In some embodiments, the anti-TROP-2 ADC, the PD-(L)1 antibody, and, optionally, the anti-TIGIT antibody are administered concurrently.

In some embodiments, the anti-TROP-2 ADC the PD-(L)1 antibody, and, optionally, the anti-TIGIT antibody are administered sequentially.

In some embodiments, the TOPI ADC, the PD-(L)1 antibody, and, optionally, the anti-TIGIT antibody are administered concurrently.

In some embodiments, the TOPI ADC the PD-(L)1 antibody, and, optionally, the anti-TIGIT antibody are administered sequentially.

In some embodiments, the subject is human.

In some embodiments, the subject is cisplatin-ineligible.

In some embodiments, the anti-TROP-2 ADC is administered at one or more doses in the range of 8 mg/kg to 10 mg/kg. In some embodiments, the anti-TROP-2 ADC is administered at one or more doses of 8 mg/kg or 10 mg/kg. In some embodiments, the anti-TROP-2 ADC is administered at one or more doses of 10 mg/kg. In some embodiments, the anti-TROP-2 ADC is administered intravenously. In some embodiments, the anti-TROP-2 ADC is administered on days 1 and 8 of a 21-day cycle.

In some embodiments of the methods provided herein, an anti-CD47 antibody is not co-administered to the subject or human patient (CD47; integrin associated protein; IAP; NCBI Gene ID: 961). In some embodiments, the subject or human patient is not administered an anti-CD47 antibody selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the subject or human patient is not administered magrolimab.

In some embodiments of the methods provided herein, an MCL1 inhibitor is not co-administered to the subject or human patient (MCL1; myeloid leukemia cell differentiation protein; NCBI Gene ID: 4170). In some embodiments, the subject or human patient is not administered an MCL1 inhibitor selected from GS-9716, AMG-397, AMG-176, PRT-1419, and S6431. In some embodiments the subject or human patient is not administered GS-9716.

In some embodiments of the methods provided herein, a FLT3 agonist is not administered to the subject or human patient. In some embodiments, the subject or human patient is not administered a FLT3 agonist selected from GS-3583, CDX-301, TAK-605, ONCR-177, Alb-Ftl3L, and SYM-027. In some embodiments, the subject or human patient is not administered GS-3583.

### Definitions

As used herein, the term “antibody” refers to a polypeptide that includes canonical immunoglobulin sequence elements sufficient to confer specific binding to a particular target antigen (e.g., a heavy chain variable domain, a light chain variable domain, and/or one or more CDRs sufficient to confer specific binding to a particular target antigen). Thus, the term antibody includes, for example, and without limitation, human antibodies, non-human antibodies, antibody fragments, and antigen-binding agents that include antibody fragments, inclusive of synthetic, engineered, and modified forms thereof. The term antibody includes, by way of example, both naturally occurring and non-naturally occurring antibodies. In general, an antibody may comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system. Naturally-produced antibodies are glycosylated, typically on the CH2 domain. Examples of antibodies include monoclonal antibodies, monospecific antibodies, polyclonal antibodies, multispecific antibodies (including bispecific antibodies), engineered antibodies, recombinantly produced antibodies, wholly synthetic antibodies, humanized antibodies, chimeric antibodies, immunoglobulins, tetrameric antibodies comprising two heavy chain and two light chain molecules, antibody light chain monomers, antibody heavy chain monomers, antibody light chain dimers, antibody heavy chain dimers, antibody light chain-antibody heavy chain pairs, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affybodies, Fab fragments, Fab′ fragments, F(ab′)2 fragments, Fd′ fragments, Fd fragments, isolated CDRs, single chain Fvs, polypeptide-Fc fusions, single domain antibodies (e.g., shark single domain antibodies such as IgNAR or fragments thereof); cameloid antibodies; disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”), single chain or Tandem diabodies (TandAb®), Anticalins®, Nanobodies®, minibodies, BiTE®s, ankyrin repeat proteins or DARPINs®, Avimers®, DARTs, TCR-like antibodies, Adnectins®, Affilins®, Trans-bodies®, Affibodies®, TrimerX®, MicroProteins,m Fynomers®, Centyrins®, KALBITOR®s, and antigen-binding fragments of any of the above.

As used herein, the term “Fc-silent antibody” refers to an antibody comprising one or more mutations in the Fc domain that reduce, prevent, or eliminate binding of the Fe region of the antibody to Fc receptors, such as FcγR or FcR, which may result in decreased antibody-dependent cellular cytotoxicity (ADCC), antibody-dependent cellular phagocytosis (ADCP), and/or complement-dependent cytotoxicity (CDC). Exemplary mutations that may reduce, prevent, or eliminate antibody binding to an Fe receptor include, but are not limited to, S228P, E233P, L234A, L235A, L235E, L235F, G236R, G237A, D265A, N297A, L328R, P331S, and any combination thereof (Saunders, Conceptual Approaches to Modulate Antibody Effector Functions and Circulation Half-Life, Front. Immunol., 2019, doi.org/10.3389/fimmu.2019.01296). In some embodiments, substitution of any or all of positions 234, 235, 236 and/or 237 reduces affinity for Fey receptors, particularly FcγRI receptor (see, e.g., U.S. Pat. No. 6,624,821). In some embodiments, alanine is a preferred residue for substitution and L234A/L235A is a preferred dual mutation to reduce effector function. In some embodiments, other combinations of mutations with reduced effector functions include, but are not limited to, L234A/L235A/G237A, E233P/L234V/L235A/G236, A327G/A330S/P331S, K322A, L234A and L235A, L234F/L235E/P331S. Optionally, positions 234, 236 and/or 237 in human IgG2 are substituted with alanine and position 235 with glutamine. (see, e.g., U.S. Pat. No. 5,624,821.) Two amino acid substitutions in the complement C1q binding site at EU index positions 330 and 331 reduce complement fixation (see Tao et al., J. Exp. Med. 178:661 (1993) and Canfield and Morrison, J. Exp. Med. 173:1483 (1991)). Substitution into human IgG1 of IgG2 residues at positions 233-236 and IgG4 residues at positions 327, 330 and 331 greatly reduces ADCC and CDC (see, for example, Armour K L. et al., 1999 Eur J Immunol. 29(8):2613-24; and Shields R L. et al., 2001. J Biol Chem. 276(9):6591-604). N297A, N297Q, or N297G (Eu numbering) mutations reduce glycosylation and thereby effector functions. In some embodiments, any of the antibodies disclosed herein comprise one or more mutations in the Fc region to reduce, prevent, or eliminate binding to Fc receptors. In some embodiments, any of the antibodies disclosed herein comprise one or more mutations in the Fc region to reduce, prevent, or eliminate binding to FcγR. In some embodiments, any of the antibodies disclosed herein comprise one or more mutations in the Fc region to reduce, prevent, or eliminate binding to FcγRIIIA. In some embodiments, any of the antibodies disclosed herein comprise one or more mutations in the Fc region to reduce, prevent, or eliminate binding to FcγRIV. In some embodiments, any of the antibodies disclosed herein comprise one or more mutations in the Fc region to reduce, eliminate or prevent ADCC, ADCP, and/or CDC. In some embodiments, mutations in the Fc region to reduce, prevent, or eliminate binding to Fc receptors occur at EU index positions 228, 233, 234, 235, 235, 235, 236, 237, 265, 297, 322, 327, 328, 330, 331, and any combination thereof. In some embodiments, mutations in the Fe region to reduce, prevent, or eliminate binding to Fc receptors include, but are not limited to, S228P, E233P, L234A, L235A, L235E, L235F, G236R, G237A, D265A, N297A, K322A, A327G, L328R, A330S, P331S, and any combination thereof. Additional mutations in the Fe region that reduce, prevent, or eliminate binding to Fe receptors and alternative strategies for reducing, preventing, or eliminating binding to Fc receptors are described in, e.g., Saunders, 2019, Tao, 1993, Canfield and Morrison, 1991, Armour, 1999, Shields, 2001, and U.S. Pat. No. 6,624,821.

As used herein, the terms “Fc-enabled antibody,” “Fc-enhanced antibody,” and “Fc-competent antibody” are used interchangeably and refer to an antibody comprising an FC domain that is capable of binding to Fc receptors, such as FcγR or FcR. These antibodies may further comprise one or more mutations to enhance or increase binding to Fc receptors, which may result in enhanced antibody-dependent cellular cytotoxicity (ADCC), antibody-dependent cellular phagocytosis (ADCP), and/or complement-dependent cytotoxicity (CDC). Exemplary mutations that may enhance ADCC include, but are not limited to, S298A, E333A, K334A, S239D, 1332E, P2471, A339Q, and any combination thereof (van der Horst, et at., Fe-Engineered Antibodies with Enhanced Fc-Effector Function for the Treatment of B-Cell Malignancies, Cancers (Basel), 12(10):3041, 2020). Exemplary mutations that may enhance ADCP include, but are not limited to, F234L, R292P, Y300L, V305I, P396L, A330L, G236A, and any combination thereof (van der Horst, et al., 2020). Exemplary mutations that may enhance CDC include, but are not limited to, E345G, E430G, K326W, E333S, S267E, H268E, S324T, and any combination thereof (van der Horst, et al., 2020). In some embodiments, any of the antibodies disclosed herein comprise one or more mutations in the Fc region to enhance or enable binding to Fe receptors. In some embodiments, any of the antibodies disclosed herein comprise one or more mutations in the Fc region to enhance or enable binding to FcγR. In some embodiments, any of the antibodies disclosed herein comprise one or more mutations in the Fe region to enhance or enable binding to FcγRIIIA. In some embodiments, any of the antibodies disclosed herein comprise one or more mutations in the Fc region to enhance or enable binding to FcγRIV. In some embodiments, any of the antibodies disclosed herein comprise one or more mutations in the Fc region to enable or enhance ADCC, ADCP, and/or CDC. In some embodiments, one or more substitutions in the Fc region to enhance or enable binding to Fc receptors occur at EU index positions 234, 235, 236, 239, 243, 247, 267, 268, 292, 298, 300, 305, 324, 326, 330, 332, 333, 334, 339, 345, 396, 430, and any combination thereof. In some embodiments, mutations in the Fc region to enhance or enable binding to Fc receptors include, but are not limited to, F234L, L235V, G236A, S239D, F243L, P2471, S267E, H268E, R292P, S298A, Y300L, V305I, S324T, K326W, A330L, I332E, E333A, E333S, K334A, A339Q, E345G, P396L, E430G, and any combination thereof. In some embodiments, the Fc-enabled antibody comprises a modified IgG1 domain characterized by substitutions at S239D, A330L, and I1332E (Eu numbering). Alternatively, glycoform perturbation can be used to enhance Fc-mediated therapeutic antibody function. The N-linked Fc glycosylations on IgG1 antibodies are important for effector function. Sialylation, galactosylation, bisecting sugars, and fucosylation can all affect binding and activity of IgG molecules. Controlling the glycosylation patterns on therapeutic antibodies can be done a number of different ways. The type of cell producing the recombinant antibody and its culture conditions can affect glycosylation and activity of therapeutic antibodies. Furthermore, bioreactor conditions and downstream processing can also affect the glycan microheterogenity. Low or afucosylated antibodies have been shown to enhance Fc-mediating properties. Numerous ways to achieve this reduction of fucose levels by glycoengineering are well known in the art. One way is to manipulate the enzymes involved in the post-translational modification of antibodies. This can involve overexpression of glucosidases, such as β-1-4-N-acetylglucosaminyltransferase III, knocking out fucoslytransferases, or using cell lines that are naturally fucose-deficient or have been mutated to express low fucosylation levels. In addition, inhibitors of N-linked glucosidases, such as castanospermine, can also be used to obtain low fucose bearing IgG molecules. In some embodiments amino acid engineered variants can have more broadly enhanced affinity for multiple FcγR, whereas glycoform engineered antibody can generally have more specific affinity for enhanced FcγRIIIa binding. Glycoforms interact with proximal amino acids on the Fc portion and replacement of the amino acid that come in contact with Ig oligosaccharides can result in different glycoform structures. Additional mutations in the Fc region that enhance or enable binding to Fc receptors and alternative strategies for enhancing or enabling binding to Fc receptors are described in Saunders, 2019.

As used herein, the term “antibody-drug conjugate” generally refers to a compound comprising an antibody targeting a tumor antigen and an anticancer agent payload, optionally connected by a linker. In some embodiments the tumor antigen is tumor-associated calcium signal transducer 2 (Trop-2; NCBI Gene ID: 4070). In some embodiments the tumor antigen targeted antibody is an anti-Trop-2 antibody (e.g., sacituzumab or datopotamab) In some embodiments the payload is a topoisomerase I inhibitor (e.g., SN38 or Dxd). Many ADC linker chemistries are known to a skilled artisan and referenced herein (e.g., CL2A).

As used herein, the terms “effective amount” or “therapeutically effective amount” refer to that amount of a therapeutic agent administered in the methods provided herein (e.g., ADC, adenosine pathway inhibitor, checkpoint inhibitor) that, when administered alone or in combination with another therapeutic agent to a cell, tissue, or subject is sufficient to effect treatment or a beneficial result in the subject. The therapeutically effective amount may vary depending on the subject, and disease or condition being treated, the weight and age of the subject, the severity of the disease or condition, and the manner of administering, which can readily be determined by one of ordinary skill in the art. An effective amount further refers to that amount of the therapeutic agent, which when used in the context of the combination therapies provided herein, is sufficient to treat, prevent, alleviate, ameliorate or mitigate a disease condition, or delay or slow the progression of a disease, and that amount sufficient to effect an increase in rate of treatment, healing, prevention or amelioration of such conditions. When applied to an individual therapeutic agent administered alone, an effective amount refers to that active ingredient alone. When applied to a combination, a therapeutically effective amount refers to combined amounts of the active ingredients that result in the therapeutic effect, whether administered in combination, sequentially or simultaneously. In some embodiments an effective amount or therapeutically effective amount of a therapeutic agent (e.g., ADC, adenosine pathway inhibitor, checkpoint inhibitor) administered to a subject in the methods provided herein with one or more additional therapeutic agents, as described herein, can (i) reduce the number of diseased cells; (ii) reduce tumor size; (iii) inhibit, retard, slow to some extent, and preferably stop the diseased cell infiltration into peripheral organs; (iv) inhibit (e.g., slow to some extent and preferably stop) tumor metastasis; (v) inhibit tumor growth; (vi) prevent or delay occurrence and/or recurrence of a tumor; and/or (vii) relieve to some extent one or more of the symptoms associated with cancer. In various embodiments, the amount is sufficient to ameliorate, palliate, lessen, and/or delay one or more of symptoms of cancer.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. In some embodiments the methods provided herein refer to the treatment of a subject having cancer (e.g., a human cancer patient). In some embodiments treating a subject having cancer (e.g., a human cancer patient) comprises inhibiting cancer or cancer cell proliferation in the treated subject. In some embodiments treating a human cancer patient using the methods provided herein results in the observation of anti-tumor effects or anti-cancer effects in the treated patient.

As used herein, the terms “inhibition of cancer” and “inhibition of cancer cell proliferation” refer to the inhibition of the growth, division, maturation or viability of cancer cells, and/or causing the death of cancer cells, individually or in aggregate with other cancer cells, by cytotoxicity, nutrient depletion, or the induction of apoptosis.

An “anti-tumor effect” or “anti-cancer effect” as used herein, refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. In some embodiments anti-cancer effects are measured using one or more of the endpoint criteria applied in the clinical studies described herein (e.g., primary, secondary, or exploratory endpoints). Exemplary clinical endpoint criteria that can be used to measure anti-cancer effects in connection with the methods provided herein include objective response rate (ORR), complete response (CR) rate, partial response (PR) rate, disease control rate (DCR), progression-free survival (PFS), overall survival (OS), biomarker-based signals, e.g., of intratumoral immune activation or induction of cancer cell death (e.g., tumor tissue or blood based biomarkers), patient quality of life (QoL) indicators (e.g., based on patient surveys), and others.

As used herein, an “increased” or “enhanced” amount is typically a “statistically significant” amount (e.g., with respect to tumor size, cancer cell proliferation or growth), and may include an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein. It may also include an increase of at least 10%, at least 20%, at least 30%, at least 40%, at least 50%, at least 60%, at least 70%, at least 80%, at least 90%, at least 100%, at least 150%, at least 200%, at least 500%, or at least 1000% of an amount or level described herein.

As used herein, a “decreased” or “reduced” or “lesser” amount (e.g., with respect to tumor size, cancer cell proliferation or growth) refers to a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7, 1.8, etc.) an amount or level described herein. It may also include a decrease of at least 10%, at least 20%, at least 30%, at least 40%, at least 50%, at least 60%, at least 70%, at least 80%, or at least 90%, at least 100%, at least 150%, at least 200%, at least 500%, or at least 1000% of an amount or level described herein.

As used herein, the term “adverse event” (AE) refers to any untoward medical occurrence in a clinical study participant administered a study drug that does not necessarily have a causal relationship with the treatment. An AE can therefore be any unfavorable and/or unintended sign (including clinically significant abnormal laboratory findings), symptom, or disease temporally associated with the use of a study drug, whether or not the AE is considered related to the study drug. Adverse events may also include pretreatment or posttreatment complications that occur as a result of protocol-specified procedures or special situations. In some embodiments, preexisting events that increase in severity or change in nature after study drug initiation or during or as a consequence of participation in the clinical study are also considered AEs.

As used herein, the term “serious adverse event” (SAE) refers to a) death; b) a life-threatening situation; c) in-participant hospitalization or prolongation of existing hospitalization; d) persistent or significant disability or incapacity; e) a congenital anomaly or birth defect; or f) a medically important event or reaction as determined by an attending physician. Examples of medically important events include intensive treatment in an emergency room or at home for allergic bronchospasm; blood dyscrasias or convulsions that do not result in hospitalization; and development of drug dependency or drug abuse.

As used herein, the terms “tumor antigen expressing cancer” or “tumor antigen positive cancer” are used interchangeably to refer to cancers having detectable levels of tumor antigen (TA) expression. In some embodiments the tumor antigen Trop-2. Tumor antigen expression in a cancer tissue or cancer cell can be detected in a sample from a subject having cancer (e.g., a human cancer patient) by any method known to a skilled artisan, e.g., as a protein, mRNA, or cell-surface expression level. For example, tumor antigen expression can be determined by methods such as immunohistochemistry (IHC), western blot, fluorescence in-situ hybridization (FISH), polymerase chain reaction (PCR), next-generation exome sequencing, or fluorescence associated cell sorting (FACS). As used herein, to be considered TA positive (e.g., Trop-2 positive) it is not required that every cell in a tumor or tumor sample have detectable levels of tumor antigen expression. In some embodiments, TA expressing cancer or TA positive cancer (e.g., Trop-2 positive cancer) refers to a cancer for which treatment with a certain anti-Trop-2 ADC or TopI ADC is indicated either as a single-agent therapy or in a combination. TA positive cancer indications of anti-Trop-2 ADCs or TopI ADCs that have received a marketing authorization from a regulatory health agency (e.g., FDA, EMA) are listed, for example, on agency approved drug product labels. In some embodiments a TA positive (e.g., Trop-2⁺) cancer, as used herein, is a cancer in which an anti-Trop-2 ADC or TopI ADC has demonstrated an anti-cancer effect that is attributable to the anti-Trop-2 ADC or TopI ADC. Such anti-cancer effects can be demonstrated in a preclinical model (e.g., a mouse xenograft or syngeneic cancer model) or in a clinical trial conducted with human cancer patients.

### Antibody-Drug-Conjugates (ADCs)

The treatment methods provided herein comprise administering an antibody-drug-conjugate (ADC) to a subject, such as a human cancer patient. In some embodiments the ADC comprises an anti-Trop-2 antibody, an anticancer agent payload, and an optional linker connecting the anti-Trop-2 antibody and payload (Anti-Trop-2 ADC). In some embodiments, the ADC comprises a tumor antigen (TA) targeted antibody, a topoisomerase I inhibitor payload, and an optional linker connecting the TA targeted antibody and payload (TopI ADC).

ADCs that can be used in the methods provided herein can comprise antibodies or antigen-binding fragments thereof of any format. For example, without limitation, the ADC can include a monospecific or multispecific (e.g., bispecific, trispecific) antibody, or an antigen-binding fragment thereof, in any format, such as DART®, Duobody®, BiTE®, BiKE, TriKE, XmAb®, TandAb®, scFv, Fab, or Fab derivative. In some embodiments, the ADC comprises a non-immunoglobulin antibody mimetic (e.g., including adnectin, affibody, affilin, affimer, affitin, alphabody, anticalin, peptide aptamer, armadillo repeat protein (ARM), atrimer, avimer, designed ankyrin repeat protein (DARPin®), fynomer, knottin, Kunitz domain peptide, monobody, and nanoCLAMPs).

In some embodiments the ADC antibody is a blocking antibody. In some embodiments the ADC antibody is a neutralizing antibody. In some embodiments the ADC antibody is an agonistic or activating antibody. In some embodiments the ADC antibody is an antagonistic or inhibitory antibody.

In some embodiments the ADC comprises an IgG antibody or antigen-binding fragment thereof. The IgG antibody or antigen-binding fragment thereof can be of various isotypes, such as IgG1, IgG2, IgG3 or IgG4. In some embodiments the ADC antibody comprises human IgG1 hinge and constant region sequences. The ADC antibody can be a chimeric human-mouse, a chimeric human-primate, a humanized (human framework and murine hypervariable (CDR) regions), or a fully human antibody, as well as a variation thereof. In some embodiments the ADC antibody is a half-IgG4 antibody (referred to as “unibody”), as described, e.g., by van der Neut Kolfschoten et al. (Science 2007; 317:1554-1557). In some embodiments the ADC antibody or antigen-binding fragment thereof is designed or selected to comprise human constant region sequences that belong to specific allotypes, which may result in reduced immunogenicity when the antibody or ADC is administered to a human subject. In some embodiments, the ADC antibody or antigen-binding fragment thereof is of a non-Glm1 allotype (nGlm1), such as Glm3, Glm3,1, Glm3,2 or Glm3,1,2. In some embodiments, the allotype is selected from the group consisting of the nGlm1, Glm3, nGlm1, 2 and Km3 allotypes.

### Anti-TROP-2 ADCs

In some embodiments the ADCs that can be used in the methods provided herein comprise an anti-Trop-2 antibody, an anticancer agent payload, and an optional linker connecting the anti-Trop-2 antibody and payload (Anti-Trop-2 ADC).

Examples of anti-TROP-2 antibodies that can be used in anti-Trop-2 ADCs to perform the methods provided herein include, but are not limited to, those described in WO2020016662 (Abmart), WO2020249063 (Bio-Thera Solutions), US20190048095 (Bio-Thera Solutions), WO2013077458 (LivTech/Chiome), EP20110783675 (Chiome), WO2015098099 (Daiichi Sankyo), WO2017002776 (Daiichi Sankyo), WO2020130125 (Daiichi Sankyo), WO2020240467 (Daiichi Sankyo), US2021093730 (Daiichi Sankyo), U.S. Pat. No. 9,850,312 (Daiichi Sankyo), CN112321715 (Biosion), US2006193865 (Immunomedics/Gilead), WO2011068845 (Immunomedics/Gilead), US2016296633 (Immunomedics/Gilead), US2017021017 (Immunomedics/Gilead), US2017209594 (Immunomedics/Gilead), US2017274093 (Immunomedics/Gilead), US2018110772 (Immunomedics/Gilead), US2018185351 (Immunomedics/Gilead), US2018271992 (Immunomedics/Gilead), WO2018217227 (Immunomedics/Gilead), US2019248917 (Immunomedics/Gilead), CN111534585 (Immunomedics/Gilead), US2021093730 (Immunomedics/Gilead), US2021069343 (Immunomedics/Gilead), U.S. Pat. No. 8,435,539 (Immunomedics/Gilead), U.S. Pat. No. 8,435,529 (Immunomedics/Gilead), U.S. Pat. No. 9,492,566 (Immunomedics/Gilead), WO2003074566 (Gilead), WO2020257648 (Gilead), US2013039861 (Gilead), WO2014163684 (Gilead), U.S. Pat. No. 9,427,464 (LivTech/Chiome), U.S. Ser. No. 10/501,555 (Abruzzo Theranostic/Oncoxx), WO2018036428 (Sichuan Kelun Pharma), WO2013068946 (Pfizer), WO2007095749 (Roche), and WO2020094670 (SynAffix).

In some embodiments of the methods provided herein, the anti-Trop-2 ADC comprises an antibody is selected from sacituzumab (hRS7), datopotamab (hTINA H1L1), Trop-2-XPAT, and BAT-8003. In some embodiments the anti-Trop-2 ADC comprises sacituzumab (hRS7). In some embodiments the anti-Trop-2 antibody comprises datopotamab (hTINA H1L1).

In some embodiments of the methods provided herein, the anti-Trop-2 ADC comprises a VH-CDR1, a VH-CDR2, a VH-CDR3, a VL-CDR1, a VL-CDR2 and a VL-CDR3 selected from one of Tables 1 to 4. In some embodiments the anti-Trop-2 ADC comprises the following VH-CDR1, a VH-CDR2, a VH-CDR3, a VL-CDR1, a VL-CDR2 and a VL-CDR3 amino acid sequences (according to Kabat), respectively:
SEQ ID NOs: 1, 2, 3, 4, 5, and 6, or
SEQ ID NOs: 37, 8, 9, 10, 11, and 12.

In some embodiments of the methods provided herein, the anti-Trop-2 ADC comprise variable domains (VH and VL) selected from Table 5. In some embodiments the anti-Trop-2 ADC comprises the following VH and VL amino acid sequences, respectively:
SEQ ID NOs: 49 and 50, or
N SEQ ID NOs: 51 and 52.

TABLE 1
CDRs for illustrative anti-Trop-2 binding antibodies (Kabat)

| Ab | VH- | | | | VL- | VL- |
|---|---|---|---|---|---|---|
| Name | CDR1 | VH-CDR2 | VH-CDR3 | VL-CDR1 | CDR2 | CDR3 |
| 1 | NYGMN | WINTYTGEPT | GGFGSSYW | KASQDVSIA | SASYRYT | QQHYITP |
| | SEQ ID | YTDDFKG | YFDV | VA | SEQ ID | LT |
| | NO: 1 | SEQ ID NO: 2 | SEQ ID NO: 3 | SEQ ID | NO: 5 | SEQ ID |
| | | | | NO: 4 | | NO: 6 |
| 2 | TAGMQ | WINTHSGVPK | SGFGSSYW | KASQDVST | SASYRYT | QQHYITP |
| | SEQ ID | YAEDFKG | YFDV | AVA | SEQ ID | LT |
| | NO: 7 | SEQ ID NO: 8 | SEQ ID NO: 9 | SEQ ID | NO: 11 | SEQ ID |
| | | | | NO: 10 | | NO: 12 |

TABLE 2
CDRs for illustrative anti-Trop-2 binding antibodies (IMGT)

| Ab | | VH- | | VL- | VL- | |
|---|---|---|---|---|---|---|
| Name | VH-CDR1 | CDR2 | VH-CDR3 | CDR1 | CDR2 | VL-CDR3 |
| 3 | GYTFTNYG | INTYTGEP | ARGGFGSSYWYFDV | QDVSIA | SAS | QQHYITPLT |
| | SEQ ID | SEQ ID | SEQ ID NO: 15 | SEQ ID | SEQ ID | SEQ ID |
| | NO: 13 | NO: 14 | | NO: 16 | NO: 17 | NO: 18 |
| 4 | GYTFTTAG | INTHSGVP | ARSGFGSSYWYFDV | QDVSTA | SAS | QQHYITPLT |
| | SEQ ID | SEQ ID | SEQ ID NO: 21 | SEQ ID | SEQ ID | SEQ ID |
| | NO: 19 | NO: 20 | | NO: 22 | NO: 23 | NO: 24 |

TABLE 3
CDRs for illustrative anti-Trop-2 binding antibodies (Chothia)

| Ab | | VH- | | | VL- | |
|---|---|---|---|---|---|---|
| Name | VH-CDR1 | CDR2 | VH-CDR3 | VL-CDR1 | CDR2 | VL-CDR3 |
| 5 | GYTFTNY | TYTG | GFGSSYWYFD | SQDVSIA | SAS | HYITPL |
| | SEQ ID | SEQ ID | SEQ ID NO: 27 | SEQ ID | SEQ ID | SEQ ID |
| | NO: 25 | NO: 26 | | NO: 28 | NO: 29 | NO: 30 |
| 6 | GYTFTTA | THSG | GFGSSYWYFD | SQDVSTA | SAS | HYITPL |
| | SEQ ID | SEQ ID | SEQ ID NO: 33 | SEQ ID | SEQ ID | SEQ ID |
| | NO: 31 | NO: 32 | | NO: 34 | NO: 35 | NO: 36 |

TABLE 4
CDRs for illustrative anti-Trop-2 binding antibodies (Honegger)

| Ab | | | | VL- | | VL- |
|---|---|---|---|---|---|---|
| Name | VH-CDR1 | VH-CDR2 | VH-CDR3 | CDR1 | VL-CDR2 | CDR3 |
| 7 | ASGYTFTNY | INTYTGEPTY | GGFGSSYW | ASQDVSI | SASYRYTG | HYITPL |
| | G | TDDFKGR | YFD | A | VPDR | SEQ ID |
| | SEQ ID | SEQ ID NO: 38 | SEQ ID | SEQ ID | SEQ ID | NO: 42 |
| | NO: 37 | | NO: 39 | NO: 40 | NO: 41 | |
| 8 | ASGYTFTTA | INTHSGVPK | SGFGSSYW | ASQDVST | SASYRYTG | HYITPL |
| | G | YAEDFKGR | YFD | A | VPSR | SEQ ID |
| | SEQ ID | SEQ ID NO: 44 | SEQ ID | SEQ ID | SEQ ID | NO: 48 |
| | NO: 43 | | NO: 45 | NO: 46 | NO: 47 | |

TABLE 5
VH/VL for illustrative anti-Trop-2 binding antibodies

| Ab | | |
|---|---|---|
| Name | VH | VL |
| 9 | SEQ ID NO: 49 | SEQ ID NO: 50 |
| | QVQLQQSGSELKKPGASVKVSCKASG | DIQLTQSPSSLSASVGDRVSITCKASQD |
| | YTFTNYGMNWVKQAPGQGLKWMGW | VSIAVAWYQQKPGKAPKLLIYSASYR |
| | INTYTGEPTYTDDFKGRFAFSLDTSVST | YTGVPDRFSGSGSGTDFTLTISSLQPED |
| | AYLQISSLKADDTAVYFCARGGFGSSY | FAVYYCQQHYITPLTFGAGTKVEIK |
| | WYFDVWGQGSLVTVS | |
| 10 | SEQ ID NO: 51 | SEQ ID NO: 52 |
| | QVQLVQSGAEVKKPGASVKVSCKASG | DIQMTQSPSSLSASVGDRVTITCKASQ |
| | YTFTTAGMQWVRQAPGQGLEWMGWI | DVSTAVAWYQQKPGKAPKLLIYSASY |
| | NTHSGVPKYAEDFKGRVTISADTSTST | RYTGVPSRFSGSGSGTDFTLTISSLQPE |
| | AYLQLSSLKSEDTAVYYCARSGFGSSY | DFAVYYCQQHYITPLTFGQGTKLEIK |
| | WYFDVWGQGTLVTVSS | |

In some embodiments of the methods provided herein the anti-Trop-2 ADC comprises an anti-Trop-2 antibody, an anticancer agent payload, and an optional linker connecting antibody and payload. In some embodiments the linker is non-cleavable (e.g., a maleimidocaproyl or maleimidomethyl cyclohexane-1-carboxylate linker). In some embodiments the linker is cleavable. In some embodiments the linker is acid cleavable (e.g., a hydrazone linker). In some embodiments the cleavable linker is reducible (e.g., a disulphide linker). In some embodiments the linker is protease cleavable (e.g., a dipeptide or tetrapeptide linker). In some embodiments, the linker is selected from linkers disclosed in U.S. Pat. No. 7,999,083 (e.g., CL2A, CL6, CL7, CLX, or CLY). In some embodiments, the linker is CL2A. Additional linker chemistries useful for anti-Trop-2 ADCs are described, for example in WO21225892 (Shanghai Escugen Biotechnology; ESG-401, STI-3258), WO22010797 (BiOneCure Therapeutics; BIO-106), CN112237634 (Shanghai Fudan-Zhangjiang Biopharmaceutical; FDA018-ADC), WO19114666 (Sichuan Kelun Pharmaceutical; KLA264), WO22078524 (Hangzhou DAC Biotech; DAC-002), WO15098099 (Daiichi Sankyo; datopotamab deruxtecan), WO21147993 (Jiangsu Hengrui Medicine; SHR-A1921), and WO21052402 (Sichuan Baili Pharmaceutical; BL-M02D1).

Exemplary anticancer agent payloads that can be used in anti-Trop-2 ADCs in the methods provided herein include, for example microtubule inhibitors, DNA cleavage agents, and topoisomerase I inhibitors. In some embodiments the microtubule inhibitor is an auristatin (e.g., monomethyl auristatin E (MMAE) or monomethyl auristatin F (MMAF), a taxane, a vinca alkaloid, an epothilone) or maytansinoid (e.g., mertansine (DM1) or ravtansine (DM4)). In some embodiments the DNA cleavage agent is a calicheamicin (e.g., ozogamicin). In some embodiments the topoisomerase I inhibitor is a camptothecin (e.g., an irinotecan, topotecan, belotecan, or exatecan derivative, such as SN38 or Dxd). In some embodiment the anticancer agent payload is SN38. In some embodiments the anticancer agent payload is Dxd.

Additional illustrative anticancer agents that can be conjugated to the anti-Trop-2 ADCs include without limitation anthracyline (e.g., doxorubicin, daunorubicin, epirubicin, idarubicin), pyrrolobenzodiazepine (PBD), or dimer thereof, DNA cross-linking agent SC-DR002 (D6.5), duocarmycin, a duocarmycin (A, B1, B2, C1, C2, D, SA, CC-1065), tubulysin B and analogs thereof (e.g., Tub196), and other anticancer agents described herein.

Exemplary anti-Trop-2 ADCs that can be used in the methods provided herein are described in WO21225892 (Shanghai Escugen Biotechnology; ESG-401, STI-3258), WO22010797 (BiOneCure Therapeutics; BIO-106), CN112237634 (Shanghai Fudan-Zhangjiang Biopharmaceutical; FDA018-ADC), WO19114666 (Sichuan Kelun Pharmaceutical; KLA264), WO22078524 (Hangzhou DAC Biotech; DAC-002), WO15098099 (Daiichi Sankyo; datopotamab deruxtecan), WO21147993 (Jiangsu Hengrui Medicine; SHR-A1921), and WO21052402 (Sichuan Baili Pharmaceutical; BL-M02D1).

In some embodiments of the methods provided herein the anti-Trop-2 ADC is selected from sacituzumab govitecan (Immunomedics/Gilead), datopotamab deruxtecan (DS-1062, Dato-Dxd; Daiichi Snakyo/AstraZeneca), SKB-264 (KL-A264; Klus Pharma, Sichuan Kelun Pharma), ESG-401 (Shanghai Escugen Biotechnology/Levena Biophanma), JS-108 (DAC-002; Junshi Bio/Hangzhou DAC), FDA018-ADC (Shanghai Fudan Zhangjiang Bio Pharma), STI-3258 (Sorrento), OXG-64 (Oncoxx), BDI-4702 (OBI Pharma), BL-M02D1 (Systimmune), Anti-Trop-2 Ab (Mediterrania Theranostic/Legochem), KD-065 (Nanjing KAEDI Biotech), Anti-Trop2 sdAb (Kisoli Biotech), Anti-Trop-2 ADC (Shandong Fontacea). LIV-2008 (LivTech/Yakult Honsha), TROP2-TRACTr (BiTE; Janux), TROP-2-IR700 (Chiome, photosensitizer), TROP2-XPAT (Amunix), BAT-8003 (Bio-Thera Solutions), GQ-1003 (Genequantum Healthcare, Samsung BioLogics), DAC-002 (Hangzhou DAC Biotech, Shanghai Junshi Biosciences), E1-3s (Immunomedics/Gilead, IBC Pharmaceuticals), s BioTech), humanized anti-Trop2-SN38 antibody conjugate (Shanghai Escugen Biotechnology, TOT Biopharma), anti-Trop2 antibody-CLB-SN-38 conjugate (Shanghai Fudan-Zhangjiang Bio-Pharmaceutical), TROP2-Ab8 (Abmart), Trop2-IgG (Nanjing Medical University (NMU)), 90Y-DTPA-AF650 (Peking University First Hospital), and hRS7-CM (SynAffix), 89Zr-DFO-AF650 (University of Wisconsin-Madison). In some embodiments the anti-Trop-2 ADC is sacituzumab govitecan (Immunomedics/Gilead). In some embodiments, the anti-Trop-2 ADC is selected from sacituzumab govitecan, datopotamab deruxtecan (DS-1062), ESG-401, SKB-264, DAC-02 and BAT-8003. In some embodiments, the anti-Trop-2 ADC is sacituzumab govitecan. In some embodiments the anti-Trop-2 ADC is datopotamab deruxtecan (DS-1062, Dato-Dxd; Daiichi Snakyo/AstraZeneca). Further examples of anti-TROP-2 therapeutics include, but are not limited to, those described in WO2016201300 (Gilead), and CN108440674 (Hangzhou Lonzyme Biological Technology).

Exemplary anti-Trop-2 ADCs that can be used in the methods provided herein are described, for example, in U.S. Pat. Nos. 7,999,083 and 9,028,833, which are hereby incorporated herein by reference in their entireties. In some embodiments, the anti-Trop-2 ADC comprises a topoisomerase I inhibitor. In some embodiments, the topoisomerase I inhibitor is selected from irinotecan, topetecan, and SN-38. In some embodiments, the anti-Trop-2 ADC has a structural formula of mAb-CL2A-SN-38, with a structure represented by:

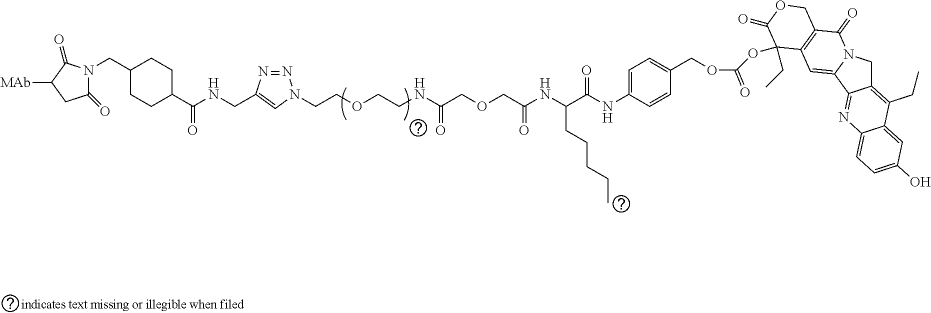

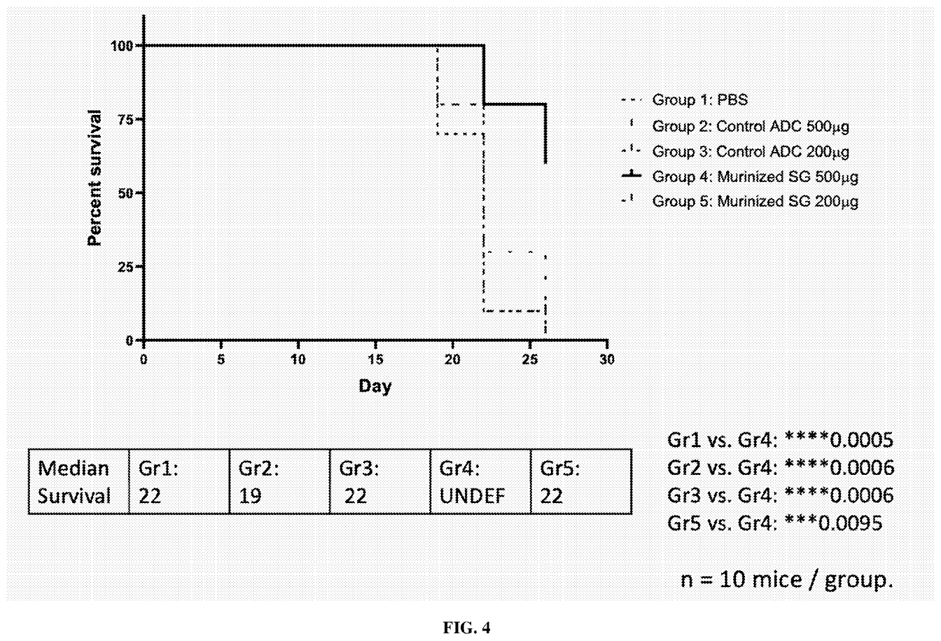

(described, e.g., in U.S. Pat. No. 7,999,083). In some embodiments, the drug-antibody ratio (DAR) of CL2A-SN38 to anti-Trop-2 antibody in the anti-Trop2 ADC is >7.0 (e.g., DAR=7.6). In some embodiments, the anti-Trop-2 ADC comprises sacituzumab (hRS7; described, e.g., in WO2003074566, FIGS. 3 and 4). In some embodiments the anti-Trop-2 ADC is sacituzumab govitecan (IMMU-132). Sacituzumab govitecan (SG) is an antibody-drug conjugate (ADC) composed of the following 3 components:
The humanized monoclonal antibody hRS7 IgG1κ, which binds to trophoblast cell-surface antigen 2 (Trop-2; TACSTD2; EGP-1; NCBI Gene ID: 4070), a transmembrane calcium signal transducer that is overexpressed in many epithelial cancers, including triple-negative breast cancer (TNBC).
The camptothecin-derived agent SN-38, a topoisomerase I inhibitor.
A hydrolyzable linker (CL2A) that links the humanized monoclonal antibody to SN-38.

Additional exemplary anti-Trop-2 ADCs that can be used in the methods provided herein are described in WO21225892 (Shanghai Escugen Biotechnology). In some embodiments the anti-Trop-2 ADC comprises a linker-payload conjugate having a structure:

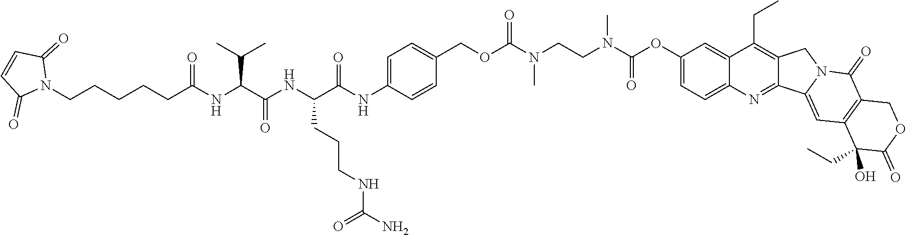 [SMILES: CCc1c2c(nc3ccc(OC(=O)N(C)CCN(C)C(=O)OCc4ccc(NC(=O)[C@H](CCCNC(N)=O)NC(=O)[C@@H](NC(=O)CCCCCN5C(=O)C=CC5=O)C(C)C)cc4)cc13)-c1cc3c(c(=O)n1C2)COC(=O)[C@]3(O)CC]

attached to an anti-Trop-2 antibody (e.g., hRS7). In some embodiments the anti-Trop-2 ADC has a DAR of 1 to 8. In some embodiments the anti-Trop-2 ADC has a DAR of >7.0. In some embodiments the anti-Trop-2 ADC is ESG-401 (STI-3258).

Additional exemplary anti-Trop-2 ADCs that can be used in the methods provided herein are described in US20210101906 (Sichuan Kelun Pharmaceutical). In some embodiments the anti-Trop-2 ADC comprises a linker-payload conjugate (TL035) having a structure:

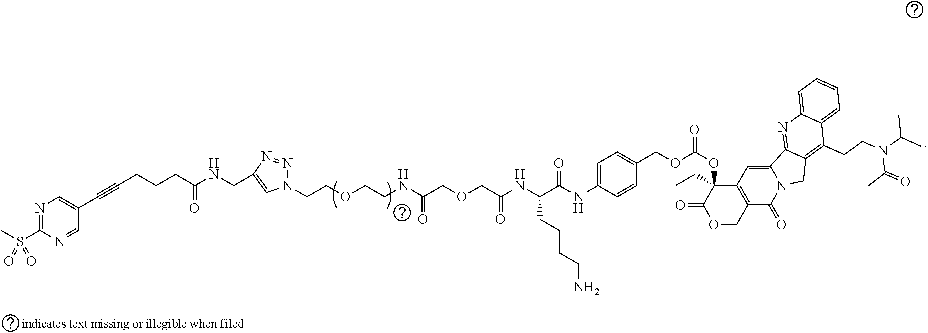

attached to an anti-Trop-2 antibody (e.g., hRS7). In some embodiments the anti-Trop-2 ADC has a DAR of 1 to 8. In some embodiments the anti-Trop-2 ADC has a DAR of about 7.0. In some embodiments the anti-Trop-2 ADC is KL-A264.

Additional exemplary anti-Trop-2 ADCs that can be used in the methods provided herein are described in US2016297890 (Daiichi Sankyo). In some embodiments the anti-Trop-2 ADC comprises a linker-payload conjugate having a structure:

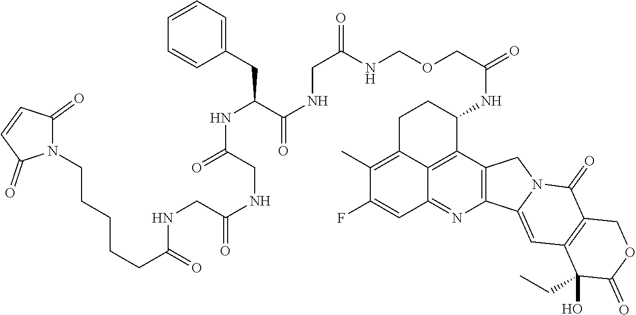 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3]

attached to an anti-Trop-2 antibody (e.g., hTINA1-H1L1). In some embodiments the anti-Trop-2 ADC has a DAR of about 4. In some embodiments the anti-Trop-2 ADC is datopotamab deruxtecan.

### TopI ADC

In some embodiments the ADCs that can be used in the methods provided herein comprise a tumor antigen (TA) targeted antibody, a topoisomerase I inhibitor payload, and an optional linker connecting the TA targeted antibody and payload (TopI ADC).

In some embodiments the TopI ADCs that can be used to in the methods provided herein comprise an antibody that binds a tumor antigen selected from carbonic anhydrase IX, B7, CCCL19, CCCL21, CSAp, HER-2/neu, BrE3, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD80, CD83, CD95, CD126, CD133, CD138, CD147, CD154, CEACAM5, CEACAM-6, alpha-fetoprotein (AFP), VEGF, ED-B fibronectin, EGP-1, EGP-2, EGF receptor (ErbB1), ErbB2, ErbB3, Factor H, FHL-1, Flt-3, folate receptor, Ga 733, GROB, HMGB-1, hypoxia inducible factor (HIF), HM1.24, insulin-like growth factor (ILGF), IFN-α, IFN-b, IL-g, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, HLA-DR, HLA-G, CD66a-d, MAGE, mCRP, MCP-1, MIP-1A, MIP-1B, macrophage migration-inhibitory factor (MIF), MUC1, MUC2, MUC3, MUC4, MUC5, placental growth factor (P1GF), PSA (prostate-specific antigen), PSMA, PSMA dimer, PAM4 antigen, NCA-95, NCA-90, A3, A33, Ep-CAM, KS-1, Le(y), mesothelin, 5100, tenascin, TAC, Tn antigen, Thomas-Friedenreich antigens, tumor necrosis antigens, tumor angiogenesis antigens, TNF-a, TRAIL receptor (R1 and R2), VEGFR, RANTES, T101, cancer stem cell antigens, complement factors C3, C3a, C3b, C5a, C5, and an oncogene product.

In some embodiments the TopI ADCs that can be used to in the methods provided herein comprise an antibody that binds a tumor antigen selected from CEACAM5, CEACAM6, CD74, CD19, CD20, CD22, CSAp, HLA-DR, HLA-G, MUC5ac, and AFP.

In some embodiments the TopI ADCs that can be used to perform the methods provided herein comprise an antibody selected from gemtuzumab, brentuximab, belantamab, camidanlumab, trastuzumab, inotuzumab, glembatumumab, anetumab, mirvetuximab, depatuxizumab, vadastuximab, labetuzumab, ladiratuzumab, loncastuximab, patritumab, lifastuzumab, indusatumab, polatuzumab, pinatuzumab, coltuximab, upifitamab, indatuximab, milatuzumab, rovalpituzumab, enfortumab, tisotumab, tusamitamab, disitamab, telisotuzumab, and antigen-binding fragments thereof.

In some embodiments the TopI ADC that can be used in the methods provided herein comprise an antibody selected from hLL1 (anti-CD74), hLL2 (anti-CD22), hRFB4 (anti-CD22), h PAM4 (anti-MUC5ac), hMN-3 (anti-NOTCH3), hMN-14 (labetuzumab; anti-CEACAM5); hMN15 (anti-CEACAM6) hA19 (anti-CD19), hA20 (anti-CD22), hMu-9 (anti-CSAp), hL243 (anti-HLA-DR), and hImmu-31 (anti-AFP) (e.g., each as further described in U.S. Pat. No. 7,999,083).

In some embodiments of the methods provided herein the TopI ADC comprises a linker connecting a topoisomerase I inhibitor payload with a tumor antigen targeted antibody. In some embodiments the linker is non-cleavable (e.g., a maleimidocaproyl or maleimidomethyl cyclohexane-1-carboxylate linker). In some embodiments the linker is cleavable. In some embodiments the linker is acid cleavable (e.g., a hydrazone linker). In some embodiments the cleavable linker is reducible (e.g., a disulphide linker). In some embodiments the linker is protease cleavable (e.g., a dipeptide or tetrapeptide linker). In some embodiments, the linker is selected from linkers disclosed in U.S. Pat. No. 7,999,083 (e.g., CL2A, CL6, CL7, CLX, or CLY). In some embodiments, the linker is CL2A. Additional linker chemistries useful for anti-TopI ADCs are described, for example in WO21225892 (Shanghai Escugen Biotechnology; ESG-401, STI-3258), WO22010797 (BiOneCure Therapeutics; BIO-106), CN112237634 (Shanghai Fudan-Zhangjiang Biopharmaceutical; FDA018-ADC), WO19114666 (Sichuan Kelun Pharmaceutical; KLA264), WO22078524 (Hangzhou DAC Biotech; DAC-002), WO15098099 (Daiichi Sankyo; datopotamab deruxtecan), WO21147993 (Jiangsu Hengrui Medicine; SHR-A1921), and WO21052402 (Sichuan Baili Pharmaceutical; BL-M02D1).

In some embodiments of the methods provided herein the TopI ADC comprises a topoisomerase I inhibitor that is a camptothecin (e.g., an irinotecan, topotecan, belotecan, or exatecan derivative, such as Dxd or SN38). In some embodiments the topoisomerase I inhibitor in the TopI ADC is Dxd. In some embodiment the topoisomerase I inhibitor in the TopI ADC is SN38.

In some embodiments, the TopI ADC has a structural formula of mAb-CL2A-SN-38, with a structure represented by:

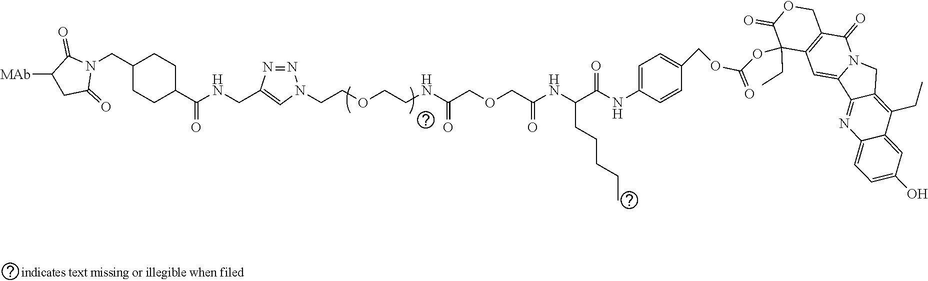

(described, e.g., in U.S. Pat. No. 7,999,083).

In some embodiments the TopI ADC that can be used in a method provided herein includes an antibody targeting carcinoembryonic antigen-related cell adhesion molecule 5 (CEACAM5; CD66e; NCBI Gene ID: 1048). In some embodiments the CEACAM5 antibody is hMN-14 (e.g., as described in WO1996011013). In some embodiments the anti-CEACAM5 ADC is as described in WO2010093395 (anti-CEACAM5-CL2A-SN38). In some embodiments the TopI ADC is labetuzumab govitecan (IMMU-130).

In some embodiments the TopI ADC that can be used in a method provided herein comprises an antibody targeting MHC class II cell surface receptor encoded by the human leukocyte antigen complex (HLA-DR). In some embodiments the HLA-DR antibody is hL243 (e.g., as described in WO2006094192). In some embodiments the HLA-DR-ADC is as described in WO2010093395 (anti-HLA-DR-CL2A-SN38). In some embodiments the antibody and/or fusion protein provided herein is administered with the HLA-DR-ADC IMMU-140.

Additional exemplary TopI ADCs that can be used in the methods provided herein are described in WO21225892 (Shanghai Escugen Biotechnology). In some embodiments the TopI ADC comprises a linker-payload conjugate having a structure:

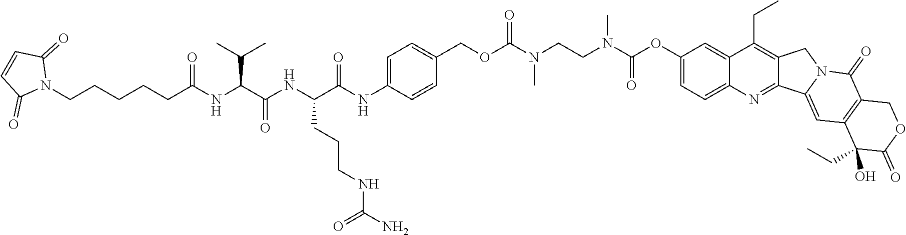 [SMILES: CCc1c2c(nc3ccc(OC(=O)N(C)CCN(C)C(=O)OCc4ccc(NC(=O)[C@H](CCCNC(N)=O)NC(=O)[C@@H](NC(=O)CCCCCN5C(=O)C=CC5=O)C(C)C)cc4)cc13)-c1cc3c(c(=O)n1C2)COC(=O)[C@]3(O)CC]

attached to a tumor antigen targeted antibody.

Additional exemplary TopI ADCs that can be used in the methods provided herein are described in US20210101906 (Sichuan Kelun Pharmaceutical). In some embodiments the TopI ADC comprises a linker-payload conjugate (TL035) having a structure:

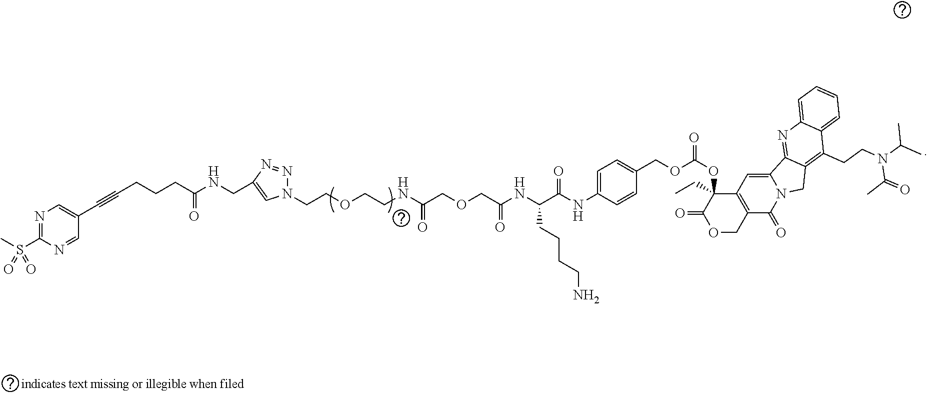

attached to a tumor antigen targeting antibody.

Additional exemplary TopI ADCs that can be used in the methods provided herein are described in US2016297890 (Daiichi Sankyo). In some embodiments the TopI ADC comprises a linker-payload conjugate having a structure:

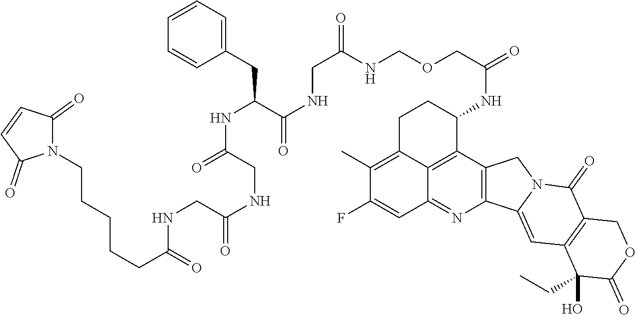 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3]

attached to a tumor antigen targeting antibody (e.g., trastuzumab). In some embodiments the TopI ADC has a DAR of about 4. In some embodiments the TopI ADC is trastuzumab-deruxtecan (T-DXd).

### Anti-PD-(L)1 Antibodies

In some embodiments, the methods disclosed herein comprise administering an anti-PD-(L)1 antibody. As used herein, the terms “anti-PD-(L)1 antibody” or “anti-PD-(L)1 antibodies” refer collectively to both (a) an anti-PD-1 antibody or antibodies or fragment thereof; and (b) anti-PD-L1 antibody or antibodies or fragment thereof.

Exemplary anti-PD-(L)1 antibodies that can be used in the any of the methods provided herein include, for example, pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, and zimberelimab. In some embodiments, the anti-PD-(L)1 antibody is zimberelimab. In some embodiments, the anti-PD-(L)1 antibody is pembrolizumab. In some embodiments, the anti-PD-(L)1 antibody is durvalumab. In some embodiments, the anti-PD-(L)1 antibody is atezolizumab.

In some embodiments, the anti-PD-(L)1 antibody is an Fc-silent antibody. In some embodiments, the anti-PD-(L)1 antibody comprises one or more mutations in the Fc region to reduce, prevent, or eliminate binding to an Fc receptor. In some embodiments, the anti-PD-(L)1 antibody comprises one or more mutations in the Fe region to reduce, prevent, or eliminate binding to FcγR. In some embodiments, any of the antibodies disclosed herein comprise one or more mutations in the Fc region to reduce, prevent, or eliminate binding to FcγRIIIA. In some embodiments, any of the antibodies disclosed herein comprise one or more mutations in the Fc region to reduce, prevent, or eliminate binding to FcγRIV. In some embodiments, the anti-PD-(L)1 antibody comprises one or more mutations in the Fc region to reduce, prevent, or eliminate ADCC, ADCP, and/or CDC. In some embodiments, the anti-PD-(L)1 antibody comprises one or more substitutions in the Fe region to reduce, prevent, or eliminate binding to Fe receptors, wherein the one or more substitutions occur at EU index positions 228, 233, 234, 235, 235, 235, 236, 237, 265, 297, 322, 327, 328, 330, 331, and any combination thereof. In some embodiments, the anti-PD-(L)1 antibody comprises one or more substitutions in the Fc region to reduce, prevent, or eliminate binding to Fc receptors, wherein the one or more substitutions comprise S228P, E233P, L234A, L235A, L235E, L235F, G236R, G237A, D265A, N297A, K322A, A327G, L328R, A330S, P331S, and any combination thereof. Additional mutations in the Fe region that reduce, prevent, or eliminate binding to Fc receptors and alternative strategies for reducing, preventing, or eliminating binding to Fe receptors are described in, e.g., Saunders, 2019, Tao, 1993, Canfield and Morrison, 1991, Armour, 1999, Shields, 2001, and U.S. Pat. No. 6,624,821.

In some embodiments, the anti-PD-(L)1 antibody is an Fc-enabled antibody. In some embodiments, the anti-PD-(L)1 antibody comprises one or more mutations in the Fc region to enable or enhance binding to an Fc receptor. In some embodiments, the anti-PD-(L)1 antibody comprises one or more mutations in the Fe region to enable or enhance binding to FcγR. In some embodiments, any of the antibodies disclosed herein comprise one or more mutations in the Fe region to enable or enhance binding to FcγRIIIA. In some embodiments, any of the antibodies disclosed herein comprise one or more mutations in the Fe region to enable or enhance binding to FcγRIV. In some embodiments, the anti-PD-(L)1 antibody comprises one or more mutations in the Fe region to enable or enhance ADCC, ADCP, and/or CDC. In some embodiments, the anti-PD-(L)1 antibody comprises one or more substitutions in Fc region to enhance or enable binding to Fe receptors, wherein the one or more substitutions occur at EU index positions 234, 235, 236, 239, 243, 247, 267, 268, 292, 298, 300, 305, 324, 326, 330, 332, 333, 334, 339, 345, 396, 430, and any combination thereof. In some embodiments, the anti-PD-(L)1 antibody comprises one or more substitutions in the Fe region to enhance or enable binding to Fe receptors, wherein the one or more substitutions comprise F234L, L235V, G236A, S239D, F243L, P2471, S267E, H268E, R292P, S298A, Y300L, V305I, S324T, K326W. A330L, 1332E. E333A, E333S, K334A, A339Q, E345G, P396L, E430G, and any combination thereof. In some embodiments, the Fc-enabled antibody comprises a modified IgG1 domain characterized by substitutions at S239D, A330L, and T332E (Eu numbering). Alternatively, the anti-PD-(L)1 antibody contains or has a glycoform perturbation. In some embodiments, the anti-PD-(L)1 antibody contains or has an N-linked Fe glycosylation. In some embodiments, the anti-PD-(L)1 antibody contains or has sialylation, galactosylation, bisecting sugars, fucosylation, or any combination thereof. Additional mutations in the Fc region that enhance or enable binding to Fc receptors and alternative strategies for enhancing or enabling binding to Fc receptors are described in Saunders, 2019.

In some embodiments, the anti-PD-(L)1 antibody is an anti-PD-1 antibody. Exemplary anti-PD-1 antibodies that can be used in any of the methods provided herein include, for example, balstilimab, budigalimab, camrelizumab, cemiplimab, cetrelimab, dostarlimab, genolimzumab, nivolumab, pembrolizumab, pidilizumab, prolgolimab, retifanlimab, sasanlimab, sintilimab, spartalizumab, tislelizumab, toripalimab, and zimberelimab. In some embodiments, the anti-PD-1 antibody is zimberelimab. In some embodiments, the anti-PD-1 antibody is pembrolizumab. In some embodiments, the anti-PD-1 antibody is an Fc-silent antibody. In some embodiments, the anti-PD-1 antibody is an Fc-enabled antibody.

In some embodiments, the anti-PD-(L)1 antibody is an anti-PD-L1 antibody. Exemplary anti-PD-L1 antibodies that can be used in any of the methods provided herein include, for example, atezolizumab, avelumab, cosibelimab, durvalumab, envafolimab, and lodapolimab. In some embodiments, the anti-PD-L1 antibody is atezolizumab. In some embodiments, the anti-PD-L1 antibody is durvalumab. In some embodiments, the anti-PD-L1 antibody is an Fc-silent antibody. In some embodiments, the anti-PD-L1 antibody is an Fc-enabled antibody.

### Anti-TIGIT Antibodies

In some embodiments, the methods disclosed herein comprise administering an anti-TIGIT antibody. Exemplary anti-TIGIT antibodies that can be used in any of the methods provided herein include, for example, AB308, AGEN-1307 (AGEN-1327), AGEN-1777, AK127, BMS-986207, domvanalimab, EOS-448, etigilimab, JS006, M6223, ociperlimab (BGB-A1217), ralzapastotug, SEA-TGT (SGN-TGT), tiragolumab, and vibostolimab. In some embodiments, the anti-TIGIT antibody is domvanalimab. In some embodiments, the anti-TIGIT antibody is AB308. In some embodiments, the anti-TIGIT antibody is ralzapastotug. In some embodiments, the anti-TIGIT antibody is tiragolumab. In some embodiments, the anti-TIGIT antibody is vibostolimab. In some embodiments, the anti-TIGIT antibody is M6223.

In some embodiments, the anti-TIGIT antibody is an Fc-silent antibody. In some embodiments, the anti-TIGIT antibody comprises one or more mutations in the Fc region to reduce, prevent, or eliminate binding to an Fc receptor. In some embodiments, the anti-TIGIT antibody comprises one or more mutations in the Fc region to reduce, prevent, or eliminate binding to FcγR. In some embodiments, any of the antibodies disclosed herein comprise one or more mutations in the Fc region to reduce, prevent, or eliminate binding to FcγRIIIA. In some embodiments, any of the antibodies disclosed herein comprise one or more mutations in the Fc region to reduce, prevent, or eliminate binding to FcγRIV. In some embodiments, the anti-TIGIT antibody comprises one or more mutations in the Fc region to reduce, prevent, or eliminate ADCC, ADCP, and/or CDC. In some embodiments, the anti-TIGIT antibody comprises one or more substitutions in the Fe region to reduce, prevent, or eliminate binding to Fc receptors, wherein the one or more substitutions occur at EU index positions 228, 233, 234, 235, 235, 235, 236, 237, 265, 297, 322, 327, 328, 330, 331, and any combination thereof. In some embodiments, the anti-TIGIT antibody comprises one or more substitutions in the Fe region to reduce, prevent, or eliminate binding to Fc receptors, wherein the one or more substitutions comprise S228P, E233P, L234A, L235A, L235E, L235F, G236R, G237A, D265A, N297A, K322A, A327G, L328R, A330S, P331S, and any combination thereof. Additional mutations in the Fc region that reduce, prevent, or eliminate binding to Fc receptors and alternative strategies for reducing, preventing, or eliminating binding to Fc receptors are described in, e.g., Saunders, 2019, Tao, 1993, Canfield and Morrison, 1991, Armour, 1999, Shields, 2001, and U.S. Pat. No. 6,624,821. In some embodiments, the Fc-silent anti-TIGIT antibody is domvanalimab. In some embodiments, the Fc-silent anti-TIGIT antibody is BGB-A1217MF. In some embodiments, the Fc-silent anti-TIGIT antibody is BMS-9862.07. In some embodiments, the anti-TIGIT antibody is an Fc-enabled antibody. In some embodiments, the anti-TIGIT antibody comprises one or more mutations in the Fc region to enable or enhance binding to an Fc receptor. In some embodiments, the anti-TIGIT antibody comprises one or more mutations in the Fc region to enable or enhance binding to FcγR. In some embodiments, any of the antibodies disclosed herein comprise one or more mutations in the Fc region to enable or enhance binding to FcγRIIIA. In some embodiments, any of the antibodies disclosed herein comprise one or more mutations in the Fc region to enable or enhance binding to FcγRIV. In some embodiments, the anti-TIGIT antibody comprises one or more mutations in the Fe region to enable or enhance ADCC, ADCP, and/or CDC. In some embodiments, the anti-PD-(L)1 antibody comprises one or more substitutions in Fc region to enhance or enable binding to Fc receptors, wherein the one or more substitutions occur at EU index positions 234, 235, 236, 239, 243, 247, 267, 268, 292, 298, 300, 305, 324, 326, 330, 332, 333, 334, 339, 345, 396, 430, and any combination thereof. In some embodiments, the anti-TIGIT antibody comprises one or more substitutions in the Fe region to enhance or enable binding to Fc receptors, wherein the one or more substitutions comprise F234L, L235V, G236A, S239D, F243L, P2471, S267E, H268E, R292P, S298A, Y300L, V305I, S324T, K326W, A330L, 1332E, E333A, E333S, K334A, A339Q, E345G, P396L, E430G, and any combination thereof. In some embodiments, the Fc-enabled antibody comprises a modified IgG1 domain characterized by substitutions at S239D, A330L, and 1332E (Eu numbering). Alternatively, the anti-TIGIT antibody contains or has a glycoform perturbation. In some embodiments, the anti-TIGIT antibody contains or has an N-linked Fc glycosylation. In some embodiments, the anti-TIGIT antibody contains or has sialylation, galactosylation, bisecting sugars, fucosylation, or any combination thereof. Additional mutations in the Fc region that enhance or enable binding to Fc receptors and alternative strategies for enhancing or enabling binding to Fc receptors are described in Saunders, 2019. In some embodiments, the Fc-enabled anti-TIGIT antibody is AB308. In some embodiments, the Fc-enabled anti-TIGIT antibody is ociperlimab. In some embodiments, the Fc-enabled anti-TIGIT antibody is ralzapastotug. In some embodiments, the Fc-enabled anti-TIGIT antibody is tiragolumab. In some embodiments, the Fc-enabled anti-TIGIT antibody is vibostolimab. In some embodiments, the Fc-enabled anti-TIGIT antibody is EOS-448. In some embodiments, the Fc-enabled anti-TIGIT antibody is SEA-TGT.

### Kits

Further disclosed herein are kits comprising any of the antibody drug conjugates (ADCs) (e.g., anti-TROP-2 ADC or TOP-1 ADC), anti-PD-(L)1 antibodies, and/or anti-TIGIT antibodies disclosed herein.

Provided herein is a kit for use as a medicament, wherein the kit comprises: a) a TROP-2-targeted antibody-drug conjugate (ADC) comprising an anti-TROP-2 antibody (anti-TROP-2 ADC); and b) an anti-PD-(L)1 antibody. In some embodiments, the kit comprises any of the anti-TROP-2 ADCs disclosed herein. In some embodiments, the kit comprises any of the anti-PD-(L)1 antibodies disclosed herein. In some embodiments, the kit further comprises any of the anti-TIGIT antibodies disclosed herein.

Provided herein is a kit for use in the treatment, mitigation, reduction, prevention, or delay of the recurrence or metastasis of a Trop-2 positive cancer, wherein the kit comprises: a) a TROP-2-targeted antibody-drug conjugate (ADC) comprising an anti-TROP-2 antibody (anti-TROP-2 ADC); and b) an anti-PD-(L)1 antibody. In some embodiments, the kit comprises any of the anti-TROP-2 ADCs disclosed herein. In some embodiments, the kit comprises any of the anti-PD-(L)1 antibodies disclosed herein. In some embodiments, the kit further comprises any of the anti-TIGIT antibodies disclosed herein.

Provided herein is a kit for use as a medicament, wherein the kit comprises a) a tumor antigen-targeted antibody-drug conjugate (ADC) comprising a topoisomerase I inhibitor (TopI ADC); and b) an anti-PD-(L)1 antibody. In some embodiments, the kit comprises any of the TopI ADCs disclosed herein. In some embodiments, the kit comprises any of the anti-PD-(L)1 antibodies disclosed herein. In some embodiments, the kit further comprises any of the anti-TIGIT antibodies disclosed herein.

Provided herein is a kit for use in the treatment, mitigation, reduction, prevention, or delay of the recurrence or metastasis of a tumor antigen positive (TA⁺) cancer, wherein the kit comprises: a) a tumor antigen-targeted antibody-drug conjugate (ADC) comprising a topoisomerase I inhibitor (TopI ADC); and b) an anti-PD-(L)1 antibody. In some embodiments, the kit comprises any of the TopI ADCs disclosed herein. In some embodiments, the kit comprises any of the anti-PD-(L)1 antibodies disclosed herein. In some embodiments, the kit further comprises any of the anti-TIGIT antibodies disclosed herein.

Further provided herein is a kit for use as a medicament, wherein the kit comprises a) sacituzumab govitecan; and b) zimberelimab. In some embodiments, the kit further comprises any of the anti-TIGIT antibodies disclosed herein.

Further provided herein is a kit for use in the treatment, mitigation, reduction, prevention, or delay of the recurrence or metastasis of urothelial cancer, wherein the kit comprises a) sacituzumab govitecan; and b) zimberelimab. In some embodiments, the kit further comprises any of the anti-TIGIT antibodies disclosed herein.

Further provided herein is a kit for use as a medicament, wherein the kit comprises a) sacituzumab govitecan; b) zimberelimab; and c) domvanalimab.

Further provided herein is a kit for use in the treatment, mitigation, reduction, prevention, or delay of the recurrence or metastasis of urothelial cancer, wherein the kit comprises: a) sacituzumab govitecan; b) zimberelimab; and c) domvanalimab.

### EXEMPLARY EMBODIMENTS

In some embodiments, provided herein are methods of treating, mitigating, reducing, preventing or delaying the recurrence or metastasis of a Trop-2 positive cancer, comprising co-administering to a subject an effective amount of: a) a TROP-2-targeted antibody-drug conjugate (ADC) comprising an anti-TROP-2 antibody (anti-TROP-2 ADC); b) an anti-PD-(L)1 antibody; and, optionally, c) an anti-TIGIT antibody. In some embodiments, the anti-TROP-2 ADC comprises an anticancer agent payload. In some embodiments, the anticancer agent payload is selected from a microtubule inhibitor, DNA cleavage agent, and topoisomerase I inhibitor. In some embodiments, the anti-TROP-2 ADC comprises a topoisomerase I inhibitor. In some embodiments, the topoisomerase I inhibitor is selected from irinotecan, topetecan, and SN-38. In some embodiments, the anti-TROP-2 ADC has a structural formula of mAb-CL2A-SN-38, with a structure represented by:

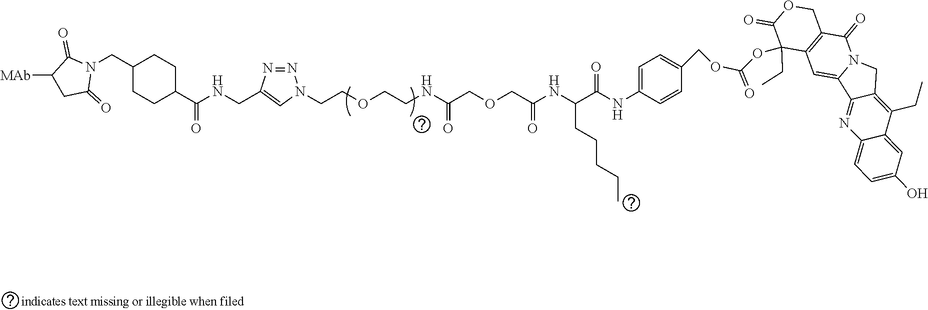

(described, e.g., in U.S. Pat. No. 7,999,083). In some embodiments, the anti-TROP-2 ADC comprises sacituzumab (hRS7; described, e.g., in WO2003074566, FIGS. 3 and 4). In some embodiments, the anti-TROP-2 ADC is selected from sacituzumab govitecan, datopotamab deruxtecan (DS-1062), ESG-401, SKB-264, DAC-02 and BAT-8003. In some embodiments, the anti-TROP-2 ADC is sacituzumab govitecan. In some embodiments, the anti-TROP-2 ADC comprises a microtuble inhibitor. In some embodiments, the microtubule inhibitor is selected from is an auristatin, a taxane, a vinca alkaloid, an epothilone, and maytansinoid. In some embodiments, the auristatin is selected from monomethyl auristatin E (MMAE) and monomethyl auristatin F (MMAF). In some embodiments, the maytansinoid is selected from mertansine (DM1) and ravtansine (DM4). In some embodiments, the anticancer agent payload is selected from anthracyline (e.g., doxorubicin, daunorubicin, epirubicin, idarubicin), pyrrolobenzodiazepine (PBD), or dimer thereof, DNA cross-linking agent SC-DR002 (D6.5), duocarmycin, a duocarmycin (A, B1, B2, C1, C2, D, SA, CC-1065), tubulysin B and analogs thereof. In some embodiments, the anti-PD-(L)1 antibody is pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, or zimberelimab. In some embodiments, the anti-PD-(L)1 antibody is zimberelimab. In some embodiments, the anti-PD-(L)1 antibody is durvalumab. In some embodiments, the anti-PD-(L)1 antibody is an Fc-silent antibody. In some embodiments, the anti-PD-(L)1 antibody is an Fc-enabled antibody. In some embodiments, any of the methods disclosed herein further comprise administering an anti-TIGIT antibody. In some embodiments, the anti-TIGIT antibody is AB308, AGEN-1307 (AGEN-1327), AGEN-1777, AK127, BMS-986207, domvanalimab, EOS-448, etigilimab, JS006, M6223, ociperlimab, ralzapastotug, SEA-TGT (SGN-TGT), tiragolumab, or vibostolimab. In some embodiments, the anti-TIGIT antibody is domvanalimab. In some embodiments, the anti-TIGIT antibody is M6223. In some embodiments, the anti-PD-(L)1 antibody and the anti-TIGIT antibody are a) zimberelimab and domvanalimab, b) zimberelimab and AB308, c) atezolizumab and tiragolumab, d) pembrolizumab and vibostolimab, e) pembrolizumab and domvanalimab, f) pembrolizumab and AB308, g) MK-7684A (pembrolizumab/vibostolimab coformulation), h) durvalumab and domvanalimab, i) zimberelimab and ralzapastotug, or j) pembrolizumab and ralzapastotug. In some embodiments, the anti-PD-(L)1 antibody and the anti-TIGIT antibody are zimberelimab and domvanalimab. In some embodiments, the anti-PD-(L)1 antibody and the anti-TIGIT antibody are zimberelimab and AB308. In some embodiments, the anti-PD-(L)1 antibody and the anti-TIGIT antibody are zimberelimab and ralzapastotug. In some embodiments, the anti-PD-(L)1 antibody and the anti-TIGIT antibody are durvalumab and domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer.

In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or unresectable locally advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of the ADC, anti-PD-(L)1 antibody, and optionally anti-TIGIT antibody. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more anti-cancer therapies before administration of the combination of the ADC, anti-PD-(L)1 antibody, and optionally anti-TIGIT antibody. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, anti-PD-(L)1 antibody, and optionally anti-TIGIT antibody is administered in a neoadjuvant setting (e.g., in preparation for surgery or radiation therapy). In some embodiments, the combination of the ADC, anti-PD-(L)1 antibody, and optionally anti-TIGIT antibody is administered in an adjuvant setting (e.g., following a primary treatment such as surgery or radiation therapy). In some embodiments, the combination of the ADC, anti-PD-(L)1 antibody, and optionally anti-TIGIT antibody is administered in a therapeutic setting (e.g., as the primary therapy). In some embodiments, the combination of the ADC, anti-PD-(L)1 antibody, and optionally anti-TIGIT antibody is administered in a maintenance setting. In some embodiments, the subject receives one or more doses of the ADC. In some embodiments, the subject receives one or more doses of the anti-PD-(L)1 antibody. In some embodiments, the subject receives one or more doses of the anti-TIGIT antibody. In some embodiments, the ADC and the anti-PD-(L)1 antibody are administered concurrently. In some embodiments, the ADC and the anti-PD-(L)1 antibody are administered sequentially. In some embodiments, the ADC and the anti-TIGIT antibody are administered concurrently. In some embodiments, the ADC and the anti-TIGIT antibody are administered sequentially. In some embodiments, the anti-PD-(L)1 antibody and the anti-TIGIT antibody are administered concurrently. In some embodiments, the anti-PD-(L)1 antibody and the anti-TIGIT antibody are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, the anti-PD-(L)1 antibody is administered at one or more doses in the range of 300 mg to 400 mg. In some embodiments, the anti-PD-(L)1 antibody is administered at dose of 360 mg. In some embodiments, the anti-PD-(L)1 antibody is administered intravenously. In some embodiments, the anti-PD-(L)1 antibody is administered on day 1 of a 21-day cycle. In some embodiments, the anti-TIGIT antibody is administered at one or more doses in the range of 800 mg to 1600 mg. In some embodiments, the anti-TIGIT antibody is administered at a dose of 1200 mg. In some embodiments, the anti-TIGIT antibody is administered intravenously. In some embodiments, the anti-TIGIT antibody is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods provided herein are for treating a Trop-2 positive cancer comprising co-administering to a subject an effective amount of: a) a TROP-2-targeted antibody-drug conjugate (ADC) comprising an anti-TROP-2 antibody (anti-TROP-2 ADC); b) an anti-PD-(L)1 antibody; and, optionally, c) an anti-TIGIT antibody. In some embodiments, the anti-TROP-2 ADC comprises an anticancer agent payload. In some embodiments, the anticancer agent payload is selected from a microtubule inhibitor, DNA cleavage agent, and topoisomerase I inhibitor. In some embodiments, the anti-TROP-2 ADC comprises a topoisomerase I inhibitor. In some embodiments, the topoisomerase I inhibitor is selected from irinotecan, topetecan, and SN-38. In some embodiments, the anti-TROP-2 ADC has a structural formula of mAb-CL2A-SN-38, with a structure represented by:

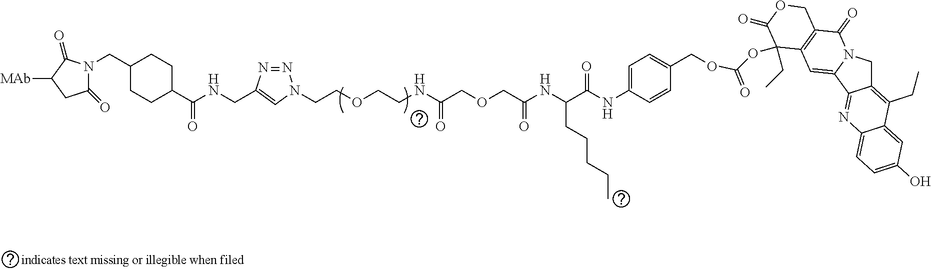

described, e.g., in U.S. Pat. No. 7,999,083). In some embodiments, the anti-TROP-2 ADC comprises sacituzumab (hRS7; described, e.g., in WO2003074566, FIGS. 3 and 4). In some embodiments, the anti-TROP-2 ADC is selected from sacituzumab govitecan, datopotamab deruxtecan (DS-1062), ESG-401, SKB-264, DAC-02 and BAT-8003. In some embodiments, the anti-TROP-2 ADC is sacituzumab govitecan. In some embodiments, the anti-TROP-2 ADC comprises a microtuble inhibitor. In some embodiments, the microtubule inhibitor is selected from is an auristatin, a taxane, a vinca alkaloid, an epothilone, and maytansinoid. In some embodiments, the auristatin is selected from monomethyl auristatin E (MMAE) and monomethyl auristatin F (MMAF). In some embodiments, the maytansinoid is selected from mertansine (DM1) and ravtansine (DM4). In some embodiments, the anticancer agent payload is selected from anthracyline (e.g., doxorubicin, daunorubicin, epirubicin, idarubicin), pyrrolobenzodiazepine (PBD), or dimer thereof, DNA cross-linking agent SC-DR002 (D6.5), duocarmycin, a duocarmycin (A, B1, B2, C1, C2, D, SA, CC-1065), tubulysin B and analogs thereof. In some embodiments, the anti-PD-(L)1 antibody is pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, or zimberelimab. In some embodiments, the anti-PD-(L)1 antibody is zimberelimab. In some embodiments, the anti-PD-(L)1 antibody is durvalumab. In some embodiments, the anti-PD-(L)1 antibody is an Fc-silent antibody. In some embodiments, the anti-PD-(L)1 antibody is an Fc-enabled antibody. In some embodiments, any of the methods disclosed herein further comprise administering an anti-TIGIT antibody. In some embodiments, the anti-TIGIT antibody is AB308, AGEN-1307 (AGEN-1327), AGEN-1777, AK127, BMS-986207, domvanalimab, EOS-448, etigilimab, JS006, M6223, ociperlimab, ralzapastotug, SEA-TGT (SGN-TGT), tiragolumab, or vibostolimab. In some embodiments, the anti-TIGIT antibody is domvanalimab. In some embodiments, the anti-TIGIT antibody is M6223. In some embodiments, the anti-PD-(L)1 antibody and the anti-TIGIT antibody are a) zimberelimab and domvanalimab, b) zimberelimab and AB308, c) atezolizumab and tiragolumab, d) pembrolizumab and vibostolimab, e) pembrolizumab and domvanalimab, f) pembrolizumab and AB308, g) MK-7684A (pembrolizumab/vibostolimab coformulation), h) durvalumab and domvanalimab, i) zimberelimab and ralzapastotug, or j) pembrolizumab and ralzapastotug. In some embodiments, the anti-PD-(L)1 antibody and the anti-TIGIT antibody are zimberelimab and domvanalimab. In some embodiments, the anti-PD-(L)1 antibody and the anti-TIGIT antibody are zimberelimab and AB308. In some embodiments, the anti-PD-(L)1 antibody and the anti-TIGIT antibody are zimberelimab and ralzapastotug. In some embodiments, the anti-PD-(L)1 antibody and the anti-TIGIT antibody are durvalumab and domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer.

In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or unresectable locally advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of the ADC, anti-PD-(L)1 antibody, and optionally anti-TIGIT antibody. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more anti-cancer therapies before administration of the combination of the ADC, anti-PD-(L)1 antibody, and optionally anti-TIGIT antibody. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, anti-PD-(L)1 antibody, and optionally anti-TIGIT antibody is administered in a neoadjuvant setting (e.g., in preparation for surgery or radiation therapy). In some embodiments, the combination of the ADC, anti-PD-(L)1 antibody, and optionally anti-TIGIT antibody is administered in an adjuvant setting (e.g., following a primary treatment such as surgery or radiation therapy). In some embodiments, the combination of the ADC, anti-PD-(L)1 antibody, and optionally anti-TIGIT antibody is administered in a therapeutic setting (e.g., as the primary therapy). In some embodiments, the combination of the ADC, anti-PD-(L)1 antibody, and optionally anti-TIGIT antibody is administered in a maintenance setting. In some embodiments, the subject receives one or more doses of the ADC. In some embodiments, the subject receives one or more doses of the anti-PD-(L)1 antibody. In some embodiments, the subject receives one or more doses of the anti-TIGIT antibody. In some embodiments, the ADC and the anti-PD-(L)1 antibody are administered concurrently. In some embodiments, the ADC and the anti-PD-(L)1 antibody are administered sequentially. In some embodiments, the ADC and the anti-TIGIT antibody are administered concurrently. In some embodiments, the ADC and the anti-TIGIT antibody are administered sequentially. In some embodiments, the anti-PD-(L)1 antibody and the anti-TIGIT antibody are administered concurrently. In some embodiments, the anti-PD-(L)1 antibody and the anti-TIGIT antibody are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, the anti-PD-(L)1 antibody is administered at one or more doses in the range of 300 mg to 400 mg. In some embodiments, the anti-PD-(L)1 antibody is administered at dose of 360 mg. In some embodiments, the anti-PD-(L)1 antibody is administered intravenously. In some embodiments, the anti-PD-(L)1 antibody is administered on day 1 of a 21-day cycle. In some embodiments, the anti-TIGIT antibody is administered at one or more doses in the range of 800 mg to 1600 mg. In some embodiments, the anti-TIGIT antibody is administered at a dose of 1200 mg. In some embodiments, the anti-TIGIT antibody is administered intravenously. In some embodiments, the anti-TIGIT antibody is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating, mitigating, reducing, preventing or delaying the recurrence or metastasis of urothelial cancer (UC), comprising co-administering to a subject an effective amount of: a) a TROP-2-targeted antibody-drug conjugate (ADC) comprising an anti-TROP-2 antibody (anti-TROP-2 ADC); b) an anti-PD-(L)1 antibody; and, optionally, c) an anti-TIGIT antibody. In some embodiments, the anti-TROP-2 ADC comprises an anticancer agent payload. In some embodiments, the anticancer agent payload is selected from a microtubule inhibitor, DNA cleavage agent, and topoisomerase I inhibitor. In some embodiments, the anti-TROP-2 ADC comprises a topoisomerase I inhibitor. In some embodiments, the topoisomerase I inhibitor is selected from irinotecan, topetecan, and SN-38. In some embodiments, the anti-TROP-2 ADC has a structural formula of mAb-CL2A-SN-38, with a structure represented by:

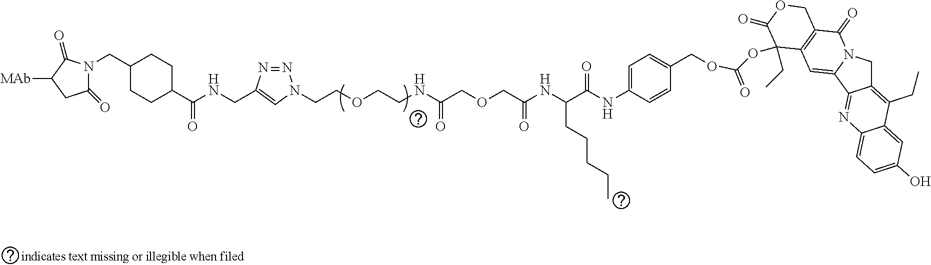

(described, e.g., in U.S. Pat. No. 7,999,083). In some embodiments, the anti-TROP-2 ADC comprises sacituzumab (hRS7; described, e.g., in WO2003074566, FIGS. 3 and 4). In some embodiments, the anti-TROP-2 ADC is selected from sacituzumab govitecan, datopotamab deruxtecan (DS-1062), ESG-401, SKB-264, DAC-02 and BAT-8003. In some embodiments, the anti-TROP-2 ADC is sacituzumab govitecan. In some embodiments, the anti-TROP-2 ADC comprises a microtuble inhibitor. In some embodiments, the microtubule inhibitor is selected from is an auristatin, a taxane, a vinca alkaloid, an epothilone, and maytansinoid. In some embodiments, the auristatin is selected from monomethyl auristatin E (MMAE) and monomethyl auristatin F (MMAF). In some embodiments, the maytansinoid is selected from mertansine (DM1) and ravtansine (DM4). In some embodiments, the anticancer agent payload is selected from anthracyline (e.g., doxorubicin, daunorubicin, epirubicin, idarubicin), pyrrolobenzodiazepine (PBD), or dimer thereof, DNA cross-linking agent SC-DR002 (D6.5), duocarmycin, a duocarmycin (A, B1, B2, C1, C2, D, SA, CC-1065), tubulysin B and analogs thereof. In some embodiments, the anti-PD-(L)1 antibody is pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, or zimberelimab. In some embodiments, the anti-PD-(L)1 antibody is zimberelimab. In some embodiments, the anti-PD-(L)1 antibody is durvalumab. In some embodiments, the anti-PD-(L)1 antibody is an Fc-silent antibody. In some embodiments, the anti-PD-(L)1 antibody is an Fc-enabled antibody. In some embodiments, any of the methods disclosed herein further comprise administering an anti-TIGIT antibody. In some embodiments, the anti-TIGIT antibody is AB308, AGEN-1307 (AGEN-1327), AGEN-1777, AK127, BMS-986207, domvanalimab, EOS-448, etigilimab, JS006, M6223, ociperlimab, ralzapastotug, SEA-TGT (SGN-TGT), tiragolumab, or vibostolimab. In some embodiments, the anti-TIGIT antibody is domvanalimab. In some embodiments, the anti-TIGIT antibody is M6223. In some embodiments, the anti-PD-(L)1 antibody and the anti-TIGIT antibody are a) zimberelimab and domvanalimab, b) zimberelimab and AB308, c) atezolizumab and tiragolumab, d) pembrolizumab and vibostolimab, e) pembrolizumab and domvanalimab, f) pembrolizumab and AB308, g) MK-7684A (pembrolizumab/vibostolimab coformulation), h) durvalumab and domvanalimab, i) zimberelimab and ralzapastotug, or j) pembrolizumab and ralzapastotug. In some embodiments, the anti-PD-(L)1 antibody and the anti-TIGIT antibody are zimberelimab and domvanalimab. In some embodiments, the anti-PD-(L)1 antibody and the anti-TIGIT antibody are zimberelimab and AB308. In some embodiments, the anti-PD-(L)1 antibody and the anti-TIGIT antibody are zimberelimab and ralzapastotug. In some embodiments, the anti-PD-(L)1 antibody and the anti-TIGIT antibody are durvalumab and domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer.

In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or unresectable locally advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of the ADC, anti-PD-(L)1 antibody, and optionally anti-TIGIT antibody. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more anti-cancer therapies before administration of the combination of the ADC, anti-PD-(L)1 antibody, and optionally anti-TIGIT antibody. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, anti-PD-(L)1 antibody, and optionally anti-TIGIT antibody is administered in a neoadjuvant setting (e.g., in preparation for surgery or radiation therapy). In some embodiments, the combination of the ADC, anti-PD-(L)1 antibody, and optionally anti-TIGIT antibody is administered in an adjuvant setting (e.g., following a primary treatment such as surgery or radiation therapy). In some embodiments, the combination of the ADC, anti-PD-(L)1 antibody, and optionally anti-TIGIT antibody is administered in a therapeutic setting (e.g., as the primary therapy). In some embodiments, the combination of the ADC, anti-PD-(L)1 antibody, and optionally anti-TIGIT antibody is administered in a maintenance setting. In some embodiments, the subject receives one or more doses of the ADC. In some embodiments, the subject receives one or more doses of the anti-PD-(L)1 antibody. In some embodiments, the subject receives one or more doses of the anti-TIGIT antibody. In some embodiments, the ADC and the anti-PD-(L)1 antibody are administered concurrently. In some embodiments, the ADC and the anti-PD-(L)1 antibody are administered sequentially. In some embodiments, the ADC and the anti-TIGIT antibody are administered concurrently. In some embodiments, the ADC and the anti-TIGIT antibody are administered sequentially. In some embodiments, the anti-PD-(L)1 antibody and the anti-TIGIT antibody are administered concurrently. In some embodiments, the anti-PD-(L)1 antibody and the anti-TIGIT antibody are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, the anti-PD-(L)1 antibody is administered at one or more doses in the range of 300 mg to 400 mg. In some embodiments, the anti-PD-(L)1 antibody is administered at dose of 360 mg. In some embodiments, the anti-PD-(L)1 antibody is administered intravenously. In some embodiments, the anti-PD-(L)1 antibody is administered on day 1 of a 21-day cycle. In some embodiments, the anti-TIGIT antibody is administered at one or more doses in the range of 800 mg to 1600 mg. In some embodiments, the anti-TIGIT antibody is administered at a dose of 1200 mg. In some embodiments, the anti-TIGIT antibody is administered intravenously. In some embodiments, the anti-TIGIT antibody is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027.

In some embodiments, the FLT3 agonist is GS-3583.

In some embodiments, provided herein are methods provided herein are for treating urothelial cancer comprising co-administering to a subject an effective amount of: a) a TROP-2-targeted antibody-drug conjugate (ADC) comprising an anti-TROP-2 antibody (anti-TROP-2 ADC); b) an anti-PD-(L)1 antibody; and, optionally, c) an anti-TIGIT antibody. In some embodiments, the anti-TROP-2 ADC comprises an anticancer agent payload. In some embodiments, the anticancer agent payload is selected from a microtubule inhibitor, DNA cleavage agent, and topoisomerase I inhibitor. In some embodiments, the anti-TROP-2 ADC comprises a topoisomerase I inhibitor. In some embodiments, the topoisomerase I inhibitor is selected from irinotecan, topetecan, and SN-38. In some embodiments, the anti-TROP-2 ADC has a structural formula of mAb-CL2A-SN-38, with a structure represented by:

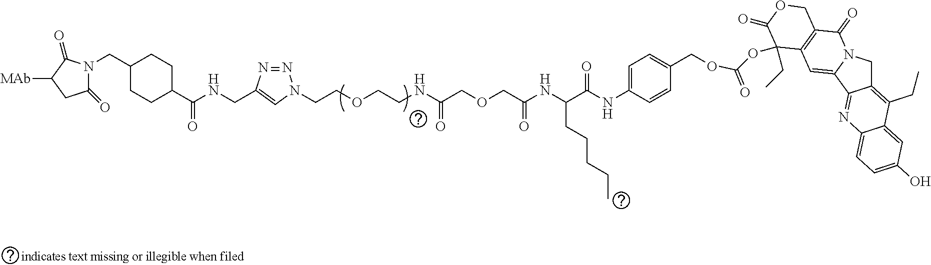

(described, e.g., in U.S. Pat. No. 7,999,083). In some embodiments, the anti-TROP-2 ADC comprises sacituzumab (hRS7; described, e.g., in WO2003074566, FIGS. 3 and 4). In some embodiments, the anti-TROP-2 ADC is selected from sacituzumab govitecan, datopotamab deruxtecan (DS-1062), ESG-401, SKB-264, DAC-02 and BAT-8003. In some embodiments, the anti-TROP-2 ADC is sacituzumab govitecan. In some embodiments, the anti-TROP-2 ADC comprises a microtuble inhibitor. In some embodiments, the microtubule inhibitor is selected from is an auristatin, a taxane, a vinca alkaloid, an epothilone, and maytansinoid. In some embodiments, the auristatin is selected from monomethyl auristatin E (MMAE) and monomethyl auristatin F (MMAF). In some embodiments, the maytansinoid is selected from mertansine (DM1) and ravtansine (DM4). In some embodiments, the anticancer agent payload is selected from anthracyline (e.g., doxorubicin, daunorubicin, epirubicin, idarubicin), pyrrolobenzodiazepine (PBD), or dimer thereof, DNA cross-linking agent SC-DR002 (D6.5), duocarmycin, a duocarmycin (A, B1, B2, C1, C2, D, SA, CC-1065), tubulysin B and analogs thereof. In some embodiments, the anti-PD-(L)1 antibody is pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, or zimberelimab. In some embodiments, the anti-PD-(L)1 antibody is zimberelimab. In some embodiments, the anti-PD-(L)1 antibody is durvalumab. In some embodiments, the anti-PD-(L)1 antibody is an Fc-silent antibody. In some embodiments, the anti-PD-(L)1 antibody is an Fc-enabled antibody. In some embodiments, any of the methods disclosed herein further comprise administering an anti-TIGIT antibody. In some embodiments, the anti-TIGIT antibody is AB308, AGEN-1307 (AGEN-1327), AGEN-1777, AK127, BMS-986207, domvanalimab, EOS-448, etigilimab, JS006, M6223, ociperlimab, ralzapastotug, SEA-TGT (SGN-TGT), tiragolumab, or vibostolimab. In some embodiments, the anti-TIGIT antibody is domvanalimab. In some embodiments, the anti-TIGIT antibody is M6223. In some embodiments, the anti-PD-(L)1 antibody and the anti-TIGIT antibody are a) zimberelimab and domvanalimab, b) zimberelimab and AB308, c) atezolizumab and tiragolumab, d) pembrolizumab and vibostolimab, e) pembrolizumab and domvanalimab, f) pembrolizumab and AB308, g) MK-7684A (pembrolizumab/vibostolimab coformulation), h) durvalumab and domvanalimab, i) zimberelimab and ralzapastotug, or j) pembrolizumab and ralzapastotug. In some embodiments, the anti-PD-(L)1 antibody and the anti-TIGIT antibody are zimberelimab and domvanalimab. In some embodiments, the anti-PD-(L)1 antibody and the anti-TIGIT antibody are zimberelimab and AB308. In some embodiments, the anti-PD-(L)1 antibody and the anti-TIGIT antibody are zimberelimab and ralzapastotug. In some embodiments, the anti-PD-(L)1 antibody and the anti-TIGIT antibody are durvalumab and domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer.

In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or unresectable locally advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of the ADC, anti-PD-(L)1 antibody, and optionally anti-TIGIT antibody. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more anti-cancer therapies before administration of the combination of the ADC, anti-PD-(L)1 antibody, and optionally anti-TIGIT antibody. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, anti-PD-(L)1 antibody, and optionally anti-TIGIT antibody is administered in a neoadjuvant setting (e.g., in preparation for surgery or radiation therapy). In some embodiments, the combination of the ADC, anti-PD-(L)1 antibody, and optionally anti-TIGIT antibody is administered in an adjuvant setting (e.g., following a primary treatment such as surgery or radiation therapy). In some embodiments, the combination of the ADC, anti-PD-(L)1 antibody, and optionally anti-TIGIT antibody is administered in a therapeutic setting (e.g., as the primary therapy). In some embodiments, the combination of the ADC, anti-PD-(L)1 antibody, and optionally anti-TIGIT antibody is administered in a maintenance setting. In some embodiments, the subject receives one or more doses of the ADC. In some embodiments, the subject receives one or more doses of the anti-PD-(L)1 antibody. In some embodiments, the subject receives one or more doses of the anti-TIGIT antibody. In some embodiments, the ADC and the anti-PD-(L)1 antibody are administered concurrently. In some embodiments, the ADC and the anti-PD-(L)1 antibody are administered sequentially. In some embodiments, the ADC and the anti-TIGIT antibody are administered concurrently. In some embodiments, the ADC and the anti-TIGIT antibody are administered sequentially. In some embodiments, the anti-PD-(L)1 antibody and the anti-TIGIT antibody are administered concurrently. In some embodiments, the anti-PD-(L)1 antibody and the anti-TIGIT antibody are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, the anti-PD-(L)1 antibody is administered at one or more doses in the range of 300 mg to 400 mg. In some embodiments, the anti-PD-(L)1 antibody is administered at dose of 360 mg. In some embodiments, the anti-PD-(L)1 antibody is administered intravenously. In some embodiments, the anti-PD-(L)1 antibody is administered on day 1 of a 21-day cycle. In some embodiments, the anti-TIGIT antibody is administered at one or more doses in the range of 800 mg to 1600 mg. In some embodiments, the anti-TIGIT antibody is administered at a dose of 1200 mg. In some embodiments, the anti-TIGIT antibody is administered intravenously. In some embodiments, the anti-TIGIT antibody is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating, mitigating, reducing, preventing or delaying the recurrence or metastasis of a Trop-2 positive cancer, comprising co-administering to a subject an effective amount of: (a) a TROP-2-targeted antibody-drug conjugate (ADC) comprising an anti-TROP-2 antibody (anti-TROP-2 ADC); (b) zimberelimab; and, optionally, (c) an anti-TIGIT antibody. In some embodiments, the anti-TROP-2 ADC comprises an anticancer agent payload. In some embodiments, the anticancer agent payload is selected from a microtubule inhibitor, DNA cleavage agent, and topoisomerase I inhibitor. In some embodiments, the anti-TROP-2 ADC comprises a topoisomerase I inhibitor. In some embodiments, the topoisomerase I inhibitor is selected from irinotecan, topetecan, and SN-38. In some embodiments, the anti-TROP-2 ADC has a structural formula of mAb-CL2A-SN-38, with a structure represented by:

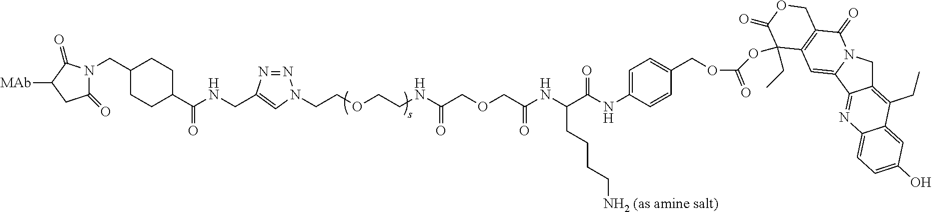

(described, e.g., in U.S. Pat. No. 7,999,083). In some embodiments, the anti-TROP-2 ADC comprises sacituzumab (hRS7; described, e.g., in WO2003074566, FIGS. 3 and 4). In some embodiments, the anti-TROP-2 ADC is selected from sacituzumab govitecan, datopotamab deruxtecan (DS-1062), ESG-401, SKB-264, DAC-02 and BAT-8003. In some embodiments, the anti-TROP-2 ADC is sacituzumab govitecan. In some embodiments, the anti-TROP-2 ADC comprises a microtuble inhibitor. In some embodiments, the microtubule inhibitor is selected from is an auristatin, a taxane, a vinca alkaloid, an epothilone, and maytansinoid. In some embodiments, the auristatin is selected from monomethyl auristatin E (MMAE) and monomethyl auristatin F (MMAF). In some embodiments, the maytansinoid is selected from mertansine (DM1) and ravtansine (DM4). In some embodiments, the anticancer agent payload is selected from anthracyline (e.g., doxorubicin, daunorubicin, epirubicin, idarubicin), pyrrolobenzodiazepine (PBD), or dimer thereof, DNA cross-linking agent SC-DR002 (D6.5), duocarmycin, a duocarmycin (A, B1, B2, C1, C2, D, SA, CC-1065), tubulysin B and analogs thereof. In some embodiments, the method further comprises administering an anti-TIGIT antibody. In some embodiments, the anti-TIGIT antibody is AB308, AGEN-1307 (AGEN-1327), AGEN-1777, AK127, BMS-986207, domvanalimab, EOS-448, etigilimab, JS006, M6223, ociperlimab, ralzapastotug, SEA-TGT (SGN-TGT), tiragolumab, or vibostolimab. In some embodiments, the anti-TIGIT antibody is domvanalimab. In some embodiments, the anti-TIGIT antibody is M6223. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer. In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or unresectable locally advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally anti-TIGIT antibody. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally anti-TIGIT antibody. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, zimberelimab, and optionally anti-TIGIT antibody is administered in a neoadjuvant setting (e.g., in preparation for surgery or radiation therapy). In some embodiments, the combination of the ADC, zimberelimab, and optionally anti-TIGIT antibody is administered in an adjuvant setting (e.g., following a primary treatment such as surgery or radiation therapy). In some embodiments, the combination of the ADC, zimberelimab, and optionally anti-TIGIT antibody is administered in a therapeutic setting (e.g., as the primary therapy). In some embodiments, the combination of the ADC, zimberelimab, and optionally anti-TIGIT antibody is administered in a maintenance setting. In some embodiments, the subject receives one or more doses of the ADC. In some embodiments, the subject receives one or more doses of zimberelimab. In some embodiments, the subject receives one or more doses of the anti-TIGIT antibody. In some embodiments, the ADC and zimberelimab are administered concurrently. In some embodiments, the ADC and zimberelimab are administered sequentially. In some embodiments, the ADC and the anti-TIGIT antibody are administered concurrently. In some embodiments, the ADC and the anti-TIGIT antibody are administered sequentially. In some embodiments, zimberelimab and the anti-TIGIT antibody are administered concurrently. In some embodiments, zimberelimab and the anti-TIGIT antibody are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, the anti-TIGIT antibody is administered at one or more doses in the range of 800 mg to 1600 mg. In some embodiments, the anti-TIGIT antibody is administered at a dose of 1200 mg. In some embodiments, the anti-TIGIT antibody is administered intravenously. In some embodiments, the anti-TIGIT antibody is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating a Trop-2 positive cancer comprising co-administering to a subject an effective amount of: (a) a TROP-2-targeted antibody-drug conjugate (ADC) comprising an anti-TROP-2 antibody (anti-TROP-2 ADC); (b) zimberelimab; and, optionally, (c) an anti-TIGIT antibody. In some embodiments, the anti-TROP-2 ADC comprises an anticancer agent payload. In some embodiments, the anticancer agent payload is selected from a microtubule inhibitor, DNA cleavage agent, and topoisomerase I inhibitor. In some embodiments, the anti-TROP-2 ADC comprises a topoisomerase I inhibitor. In some embodiments, the topoisomerase I inhibitor is selected from irinotecan, topetecan, and SN-38. In some embodiments, the anti-TROP-2 ADC has a structural formula of mAb-CL2A-SN-38, with a structure represented by:

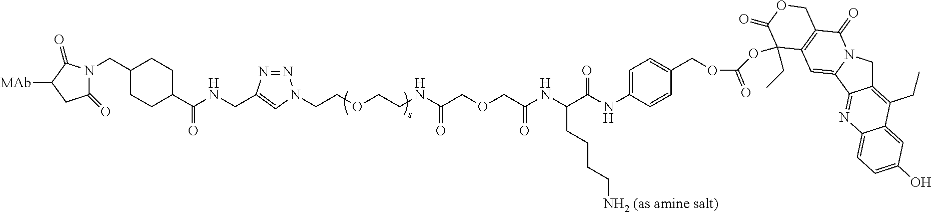

(described, e.g., in U.S. Pat. No. 7,999,083). In some embodiments, the anti-TROP-2 ADC comprises sacituzumab (hRS7; described, e.g., in WO2003074566, FIGS. 3 and 4). In some embodiments, the anti-TROP-2 ADC is selected from sacituzumab govitecan, datopotamab deruxtecan (DS-1062), ESG-401, SKB-264, DAC-02 and BAT-8003. In some embodiments, the anti-TROP-2 ADC is sacituzumab govitecan. In some embodiments, the anti-TROP-2 ADC comprises a microtuble inhibitor. In some embodiments, the microtubule inhibitor is selected from is an auristatin, a taxane, a vinca alkaloid, an epothilone, and maytansinoid. In some embodiments, the auristatin is selected from monomethyl auristatin E (MMAE) and monomethyl auristatin F (MMAF). In some embodiments, the maytansinoid is selected from mertansine (DM1) and ravtansine (DM4). In some embodiments, the anticancer agent payload is selected from anthracyline (e.g., doxorubicin, daunorubicin, epirubicin, idarubicin), pyrrolobenzodiazepine (PBD), or dimer thereof, DNA cross-linking agent SC-DR002 (D6.5), duocarmycin, a duocarmycin (A, B1, B2, C1, C2, D, SA, CC-1065), tubulysin B and analogs thereof. In some embodiments, the method further comprises administering an anti-TIGIT antibody. In some embodiments, the anti-TIGIT antibody is AB308, AGEN-1307 (AGEN-1327), AGEN-1777, AK127, BMS-986207, domvanalimab, EOS-448, etigilimab, JS006, M6223, ociperlimab, ralzapastotug, SEA-TGT (SGN-TGT), tiragolumab, or vibostolimab. In some embodiments, the anti-TIGIT antibody is domvanalimab. In some embodiments, the anti-TIGIT antibody is M6223. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer. In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally anti-TIGIT antibody. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally anti-TIGIT antibody. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, zimberelimab, and optionally anti-TIGIT antibody is administered in a neoadjuvant setting (e.g., in preparation for surgery or radiation therapy). In some embodiments, the combination of the ADC, zimberelimab, and optionally anti-TIGIT antibody is administered in an adjuvant setting (e.g., following a primary treatment such as surgery or radiation therapy). In some embodiments, the combination of the ADC, zimberelimab, and optionally anti-TIGIT antibody is administered in a therapeutic setting (e.g., as the primary therapy). In some embodiments, the combination of the ADC, zimberelimab, and optionally anti-TIGIT antibody is administered in a maintenance setting. In some embodiments, the subject receives one or more doses of the ADC. In some embodiments, the subject receives one or more doses of zimberelimab. In some embodiments, the subject receives one or more doses of the anti-TIGIT antibody. In some embodiments, the ADC and zimberelimab are administered concurrently. In some embodiments, the ADC and zimberelimab are administered sequentially.

In some embodiments, the ADC and the anti-TIGIT antibody are administered concurrently. In some embodiments, the ADC and the anti-TIGIT antibody are administered sequentially. In some embodiments, zimberelimab and the anti-TIGIT antibody are administered concurrently. In some embodiments, zimberelimab and the anti-TIGIT antibody are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, the anti-TIGIT antibody is administered at one or more doses in the range of 800 mg to 1600 mg. In some embodiments, the anti-TIGIT antibody is administered at a dose of 1200 mg. In some embodiments, the anti-TIGIT antibody is administered intravenously. In some embodiments, the anti-TIGIT antibody is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027.

In some embodiments, the FLT3 agonist is GS-3583.

In some embodiments, provided herein are methods of treating, mitigating, reducing, preventing or delaying the recurrence or metastasis of a Trop-2 positive cancer, co-administering to a subject an effective amount of: (a) a TROP-2-targeted antibody-drug conjugate (ADC) comprising an anti-TROP-2 antibody (anti-TROP-2 ADC); (b) zimberelimab; and, optionally, (c) domvanalimab. In some embodiments, the anti-TROP-2 ADC comprises an anticancer agent payload. In some embodiments, the anticancer agent payload is selected from a microtubule inhibitor, DNA cleavage agent, and topoisomerase I inhibitor. In some embodiments, the anti-TROP-2 ADC comprises a topoisomerase I inhibitor. In some embodiments, the topoisomerase I inhibitor is selected from irinotecan, topetecan, and SN-38. In some embodiments, the anti-TROP-2 ADC has a structural formula of mAb-CL2A-SN-38, with a structure represented by:

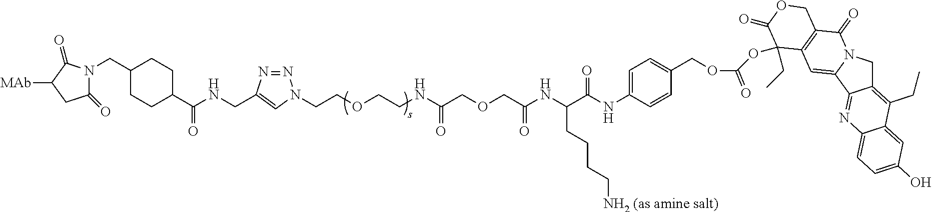

(described, e.g., in U.S. Pat. No. 7,999,083). In some embodiments, the anti-TROP-2 ADC comprises sacituzumab (hRS7; described, e.g., in WO2003074566, FIGS. 3 and 4). In some embodiments, the anti-TROP-2 ADC is selected from sacituzumab govitecan, datopotamab deruxtecan (DS-1062), ESG-401, SKB-264, DAC-02 and BAT-8003. In some embodiments, the anti-TROP-2 ADC is sacituzumab govitecan. In some embodiments, the anti-TROP-2 ADC comprises a microtuble inhibitor. In some embodiments, the microtubule inhibitor is selected from is an auristatin, a taxane, a vinca alkaloid, an epothilone, and maytansinoid. In some embodiments, the auristatin is selected from monomethyl auristatin E (MMAE) and monomethyl auristatin F (MMAF). In some embodiments, the maytansinoid is selected from mertansine (DM1) and ravtansine (DM4). In some embodiments, the anticancer agent payload is selected from anthracyline (e.g., doxorubicin, daunorubicin, epirubicin, idarubicin), pyrrolobenzodiazepine (PBD), or dimer thereof, DNA cross-linking agent SC-DR002 (D6.5), duocarmycin, a duocarmycin (A, B1, B2, C1, C2, D, SA, CC-1065), tubulysin B and analogs thereof. In some embodiments, the method further comprises administering domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer. In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally domvanalimab. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, zimberelimab, and optionally domvanalimab is administered in a neoadjuvant setting (e.g., in preparation for surgery or radiation therapy). In some embodiments, the combination of the ADC, zimberelimab, and optionally domvanalimab is administered in an adjuvant setting (e.g., following a primary treatment such as surgery or radiation therapy). In some embodiments, the combination of the ADC, zimberelimab, and optionally domvanalimab is administered in a therapeutic setting (e.g., as the primary therapy). In some embodiments, the combination of the ADC, zimberelimab, and optionally domvanalimab is administered in a maintenance setting. In some embodiments, the subject receives one or more doses of the ADC. In some embodiments, the subject receives one or more doses of zimberelimab. In some embodiments, the subject receives one or more doses of domvanalimab. In some embodiments, the ADC and zimberelimab are administered concurrently. In some embodiments, the ADC and zimberelimab are administered sequentially. In some embodiments, the ADC and domvanalimab are administered concurrently. In some embodiments, the ADC and domvanalimab are administered sequentially. In some embodiments, zimberelimab and domvanalimab are administered concurrently. In some embodiments, zimberelimab and domvanalimab are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating a Trop-2 positive cancer comprising co-administering to a subject an effective amount of: (a) a TROP-2-targeted antibody-drug conjugate (ADC) comprising an anti-TROP-2 antibody (anti-TROP-2 ADC); (b) zimberelimab; and, optionally, (c) domvanalimab. In some embodiments, the anti-TROP-2 ADC comprises an anticancer agent payload. In some embodiments, the anticancer agent payload is selected from a microtubule inhibitor, DNA cleavage agent, and topoisomerase I inhibitor. In some embodiments, the anti-TROP-2 ADC comprises a topoisomerase I inhibitor. In some embodiments, the topoisomerase I inhibitor is selected from irinotecan, topetecan, and SN-38. In some embodiments, the anti-TROP-2 ADC has a structural formula of mAb-CL2A-SN-38, with a structure represented by:

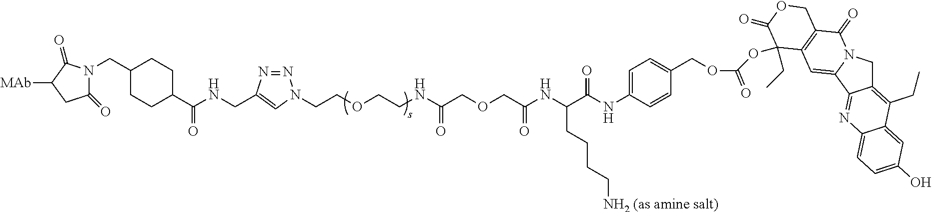

(described, e.g., in U.S. Pat. No. 7,999,083). In some embodiments, the anti-TROP-2 ADC comprises sacituzumab (hRS7; described, e.g., in WO2003074566, FIGS. 3 and 4). In some embodiments, the anti-TROP-2 ADC is selected from sacituzumab govitecan, datopotamab deruxtecan (DS-1062), ESG-401, SKB-264, DAC-02 and BAT-8003. In some embodiments, the anti-TROP-2 ADC is sacituzumab govitecan. In some embodiments, the anti-TROP-2 ADC comprises a microtuble inhibitor. In some embodiments, the microtubule inhibitor is selected from is an auristatin, a taxane, a vinca alkaloid, an epothilone, and maytansinoid. In some embodiments, the auristatin is selected from monomethyl auristatin E (MMAE) and monomethyl auristatin F (MMAF). In some embodiments, the maytansinoid is selected from mertansine (DM1) and ravtansine (DM4). In some embodiments, the anticancer agent payload is selected from anthracyline (e.g., doxorubicin, daunorubicin, epirubicin, idarubicin), pyrrolobenzodiazepine (PBD), or dimer thereof, DNA cross-linking agent SC-DR002 (D6.5), duocarmycin, a duocarmycin (A, B1, B2, C1, C2, D, SA, CC-1065), tubulysin B and analogs thereof. In some embodiments, the method further comprises administering domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer. In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally domvanalimab. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, zimberelimab, and optionally domvanalimab is administered in a neoadjuvant setting (e.g., in preparation for surgery or radiation therapy). In some embodiments, the combination of the ADC, zimberelimab, and optionally domvanalimab is administered in an adjuvant setting (e.g., following a primary treatment such as surgery or radiation therapy). In some embodiments, the combination of the ADC, zimberelimab, and optionally domvanalimab is administered in a therapeutic setting (e.g., as the primary therapy). In some embodiments, the combination of the ADC, zimberelimab, and optionally domvanalimab is administered in a maintenance setting. In some embodiments, the subject receives one or more doses of the ADC. In some embodiments, the subject receives one or more doses of zimberelimab. In some embodiments, the subject receives one or more doses of domvanalimab. In some embodiments, the ADC and zimberelimab are administered concurrently. In some embodiments, the ADC and zimberelimab are administered sequentially. In some embodiments, the ADC and domvanalimab are administered concurrently. In some embodiments, the ADC and domvanalimab are administered sequentially. In some embodiments, zimberelimab and domvanalimab are administered concurrently. In some embodiments, zimberelimab and domvanalimab are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating, mitigating, reducing, preventing or delaying the recurrence or metastasis of a Trop-2 positive cancer, comprising co-administering to a subject an effective amount of: (a) a TROP-2-targeted antibody-drug conjugate (ADC) comprising an anti-TROP-2 antibody (anti-TROP-2 ADC); (b) an anti-PD-(L)1 antibody; and, optionally, (c) domvanalimab. In some embodiments, the anti-TROP-2 ADC comprises an anticancer agent payload. In some embodiments, the anticancer agent payload is selected from a microtubule inhibitor, DNA cleavage agent, and topoisomerase I inhibitor. In some embodiments, the anti-TROP-2 ADC comprises a topoisomerase I inhibitor. In some embodiments, the topoisomerase I inhibitor is selected from irinotecan, topetecan, and SN-38. In some embodiments, the anti-TROP-2 ADC has a structural formula of mAb-CL2A-SN-38, with a structure represented by:

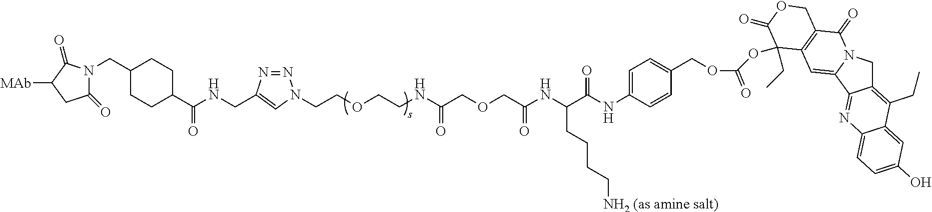

(described, e.g., in U.S. Pat. No. 7,999,083). In some embodiments, the anti-TROP-2 ADC comprises sacituzumab (hRS7; described, e.g., in WO2003074566, FIGS. 3 and 4). In some embodiments, the anti-TROP-2 ADC is selected from sacituzumab govitecan, datopotamab deruxtecan (DS-1062), ESG-401, SKB-264, DAC-02 and BAT-8003. In some embodiments, the anti-TROP-2 ADC is sacituzumab govitecan. In some embodiments, the anti-TROP-2 ADC comprises a microtuble inhibitor. In some embodiments, the microtubule inhibitor is selected from is an auristatin, a taxane, a vinca alkaloid, an epothilone, and maytansinoid. In some embodiments, the auristatin is selected from monomethyl auristatin E (MMAE) and monomethyl auristatin F (MMAF). In some embodiments, the maytansinoid is selected from mertansine (DM1) and ravtansine (DM4). In some embodiments, the anticancer agent payload is selected from anthracyline (e.g., doxorubicin, daunorubicin, epirubicin, idarubicin), pyrrolobenzodiazepine (PBD), or dimer thereof, DNA cross-linking agent SC-DR002 (D6.5), duocarmycin, a duocarmycin (A, B1, B2, C1, C2, D, SA, CC-1065), tubulysin B and analogs thereof. In some embodiments, the anti-PD-(L)1 antibody is pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, or zimberelimab. In some embodiments, the anti-PD-(L)1 antibody is zimberelimab. In some embodiments, the anti-PD-(L)1 antibody is durvalumab. In some embodiments, the anti-PD-(L)1 antibody is an Fc-silent antibody. In some embodiments, the anti-PD-(L)1 antibody is an Fc-enabled antibody. In some embodiments, the method further comprises administering domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer. In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or unresectable locally advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the treatment naïve subject has not received prior anti-cancer therapy for (i) advanced cancer or (ii) metastatic cancer. In some embodiments, the subject has received one or more anti-cancer therapies before administration of the combination of the ADC, anti-PD-(L)1 antibody, and optionally domvanalimab. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more prior anti-cancer therapies for (i) advanced cancer or (ii) metastatic cancer. In some embodiments, the subject has received one or more anti-cancer therapies before administration of the combination of the ADC, anti-PD-(L)1 antibody, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, anti-PD-(L)1 antibody, and optionally domvanalimab is administered in a neoadjuvant setting (e.g., in preparation for surgery or radiation therapy). In some embodiments, the combination of the ADC, anti-PD-(L)1 antibody, and optionally domvanalimab is administered in an adjuvant setting (e.g., following a primary treatment such as surgery or radiation therapy). In some embodiments, the combination of the ADC, anti-PD-(L)1 antibody, and optionally domvanalimab is administered in a therapeutic setting (e.g., as the primary therapy). In some embodiments, the combination of the ADC, anti-PD-(L)1 antibody, and optionally domvanalimab is administered in a maintenance setting. In some embodiments, the subject receives one or more doses of the ADC. In some embodiments, the subject receives one or more doses of the anti-PD-(L)1 antibody. In some embodiments, the subject receives one or more doses of domvanalimab. In some embodiments, the ADC and the anti-PD-(L)1 antibody are administered concurrently. In some embodiments, the ADC and the anti-PD-(L)1 antibody are administered sequentially. In some embodiments, the ADC and domvanalimab are administered concurrently. In some embodiments, the ADC and domvanalimab are administered sequentially. In some embodiments, the anti-PD-(L)1 antibody and domvanalimab are administered concurrently. In some embodiments, the anti-PD-(L)1 antibody and domvanalimab are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, the anti-PD-(L)1 antibody is administered at one or more doses in the range of 300 mg to 400 mg. In some embodiments, the anti-PD-(L)1 antibody is administered at dose of 360 mg. In some embodiments, the anti-PD-(L)1 antibody is administered intravenously. In some embodiments, the anti-PD-(L)1 antibody is administered on day 1 of a 21-day cycle. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating a Trop-2 positive cancer comprising co-administering to a subject an effective amount of: (a) a TROP-2-targeted antibody-drug conjugate (ADC) comprising an anti-TROP-2 antibody (anti-TROP-2 ADC); (b) an anti-PD-(L)1 antibody; and, optionally, (c) domvanalimab. In some embodiments, the anti-TROP-2 ADC comprises an anticancer agent payload. In some embodiments, the anticancer agent payload is selected from a microtubule inhibitor, DNA cleavage agent, and topoisomerase I inhibitor. In some embodiments, the anti-TROP-2 ADC comprises a topoisomerase I inhibitor. In some embodiments, the topoisomerase I inhibitor is selected from irinotecan, topetecan, and SN-38. In some embodiments, the anti-TROP-2 ADC has a structural formula of mAb-CL2A-SN-38, with a structure represented by:

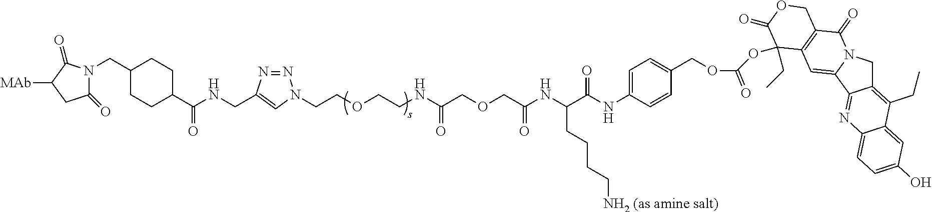

(described, e.g., in U.S. Pat. No. 7,999,083). In some embodiments, the anti-TROP-2 ADC comprises sacituzumab (hRS7; described, e.g., in WO2003074566, FIGS. 3 and 4). In some embodiments, the anti-TROP-2 ADC is selected from sacituzumab govitecan, datopotamab deruxtecan (DS-1062), ESG-401, SKB-264, DAC-02 and BAT-8003. In some embodiments, the anti-TROP-2 ADC is sacituzumab govitecan. In some embodiments, the anti-TROP-2 ADC comprises a microtuble inhibitor. In some embodiments, the microtubule inhibitor is selected from is an auristatin, a taxane, a vinca alkaloid, an epothilone, and maytansinoid. In some embodiments, the auristatin is selected from monomethyl auristatin E (MMAE) and monomethyl auristatin F (MMAF). In some embodiments, the maytansinoid is selected from mertansine (DM1) and ravtansine (DM4). In some embodiments, the anticancer agent payload is selected from anthracyline (e.g., doxorubicin, daunorubicin, epirubicin, idarubicin), pyrrolobenzodiazepine (PBD), or dimer thereof, DNA cross-linking agent SC-DR002 (D6.5), duocarmycin, a duocarmycin (A, B1, B2, C1, C2, D, SA, CC-1065), tubulysin B and analogs thereof. In some embodiments, the anti-PD-(L)1 antibody is pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, or zimberelimab. In some embodiments, the anti-PD-(L)1 antibody is zimberelimab. In some embodiments, the anti-PD-(L)1 antibody is durvalumab. In some embodiments, the anti-PD-(L)1 antibody is an Fc-silent antibody. In some embodiments, the anti-PD-(L)1 antibody is an Fc-enabled antibody. In some embodiments, the method further comprises administering domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer. In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or unresectable locally advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the treatment naïve subject has not received prior anti-cancer therapy for (i) advanced cancer or (ii) metastatic cancer. In some embodiments, the subject has received one or more anti-cancer therapies before administration of the combination of the ADC, anti-PD-(L)1 antibody, and optionally domvanalimab. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more prior anti-cancer therapies for (i) advanced cancer or (ii) metastatic cancer. In some embodiments, the subject has received one or more anti-cancer therapies before administration of the combination of the ADC, anti-PD-(L)1 antibody, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, anti-PD-(L)1 antibody, and optionally domvanalimab is administered in a neoadjuvant setting (e.g., in preparation for surgery or radiation therapy). In some embodiments, the combination of the ADC, anti-PD-(L)1 antibody, and optionally domvanalimab is administered in an adjuvant setting (e.g., following a primary treatment such as surgery or radiation therapy). In some embodiments, the combination of the ADC, anti-PD-(L)1 antibody, and optionally domvanalimab is administered in a therapeutic setting (e.g., as the primary therapy). In some embodiments, the combination of the ADC, anti-PD-(L)1 antibody, and optionally domvanalimab is administered in a maintenance setting. In some embodiments, the subject receives one or more doses of the ADC. In some embodiments, the subject receives one or more doses of the anti-PD-(L)1 antibody. In some embodiments, the subject receives one or more doses of domvanalimab. In some embodiments, the ADC and the anti-PD-(L)1 antibody are administered concurrently. In some embodiments, the ADC and the anti-PD-(L)1 antibody are administered sequentially. In some embodiments, the ADC and domvanalimab are administered concurrently. In some embodiments, the ADC and domvanalimab are administered sequentially. In some embodiments, the anti-PD-(L)1 antibody and domvanalimab are administered concurrently. In some embodiments, the anti-PD-(L)1 antibody and domvanalimab are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, the anti-PD-(L)1 antibody is administered at one or more doses in the range of 300 mg to 400 mg. In some embodiments, the anti-PD-(L)1 antibody is administered at dose of 360 mg. In some embodiments, the anti-PD-(L)1 antibody is administered intravenously. In some embodiments, the anti-PD-(L)1 antibody is administered on day 1 of a 21-day cycle. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating, mitigating, reducing, preventing or delaying the recurrence or metastasis of urothelial cancer, comprising co-administering to a subject an effective amount of: (a) a TROP-2-targeted antibody-drug conjugate (ADC) comprising an anti-TROP-2 antibody (anti-TROP-2 ADC); (b) zimberelimab; and, optionally, (c) an anti-TIGIT antibody. In some embodiments, the anti-TROP-2 ADC comprises an anticancer agent payload. In some embodiments, the anticancer agent payload is selected from a microtubule inhibitor, DNA cleavage agent, and topoisomerase I inhibitor. In some embodiments, the anti-TROP-2 ADC comprises a topoisomerase I inhibitor. In some embodiments, the topoisomerase I inhibitor is selected from irinotecan, topetecan, and SN-38. In some embodiments, the anti-TROP-2 ADC has a structural formula of mAb-CL2A-SN-38, with a structure represented by:

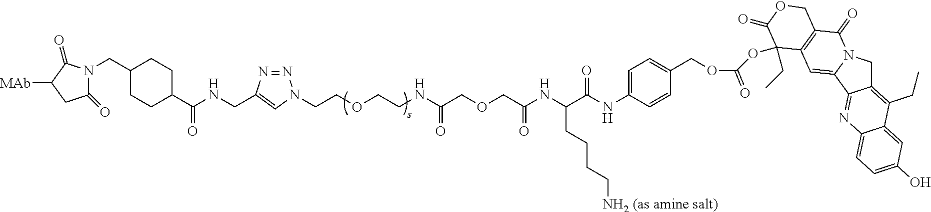

(described, e.g., in U.S. Pat. No. 7,999,083). In some embodiments, the anti-TROP-2 ADC comprises sacituzumab (hRS7; described, e.g., in WO2003074566, FIGS. 3 and 4). In some embodiments, the anti-TROP-2 ADC is selected from sacituzumab govitecan, datopotamab deruxtecan (DS-1062), ESG-401, SKB-264, DAC-02 and BAT-8003. In some embodiments, the anti-TROP-2 ADC is sacituzumab govitecan. In some embodiments, the anti-TROP-2 ADC comprises a microtuble inhibitor. In some embodiments, the microtubule inhibitor is selected from is an auristatin, a taxane, a vinca alkaloid, an epothilone, and maytansinoid. In some embodiments, the auristatin is selected from monomethyl auristatin E (MMAE) and monomethyl auristatin F (MMAF). In some embodiments, the maytansinoid is selected from mertansine (DM1) and ravtansine (DM4). In some embodiments, the anticancer agent payload is selected from anthracyline (e.g., doxorubicin, daunorubicin, epirubicin, idarubicin), pyrrolobenzodiazepine (PBD), or dimer thereof, DNA cross-linking agent SC-DR002 (D6.5), duocarmycin, a duocarmycin (A, B1, B2, C1, C2, D, SA, CC-1065), tubulysin B and analogs thereof. In some embodiments, the method further comprises administering an anti-TIGIT antibody. In some embodiments, the anti-TIGIT antibody is AB308, AGEN-1307 (AGEN-1327), AGEN-1777, AK127, BMS-986207, domvanalimab, EOS-448, etigilimab, JS006, M6223, ociperlimab, ralzapastotug, SEA-TGT (SGN-TGT), tiragolumab, or vibostolimab. In some embodiments, the anti-TIGIT antibody is domvanalimab. In some embodiments, the anti-TIGIT antibody is M6223. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is (i) unresectable, locally advanced, (ii) metastatic, or (iii) muscle invasive. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally anti-TIGIT antibody. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally anti-TIGIT antibody. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, zimberelimab, and optionally anti-TIGIT antibody is administered in a neoadjuvant setting (e.g., in preparation for surgery or radiation therapy). In some embodiments, the combination of the ADC, zimberelimab, and optionally anti-TIGIT antibody is administered in an adjuvant setting (e.g., following a primary treatment such as surgery or radiation therapy). In some embodiments, the combination of the ADC, zimberelimab, and optionally anti-TIGIT antibody is administered in a therapeutic setting (e.g., as the primary therapy). In some embodiments, the combination of the ADC, zimberelimab, and optionally anti-TIGIT antibody is administered in a maintenance setting. In some embodiments, the subject receives one or more doses of the ADC. In some embodiments, the subject receives one or more doses of zimberelimab. In some embodiments, the subject receives one or more doses of the anti-TIGIT antibody. In some embodiments, the ADC and zimberelimab are administered concurrently. In some embodiments, the ADC and zimberelimab are administered sequentially. In some embodiments, the ADC and the anti-TIGIT antibody are administered concurrently. In some embodiments, the ADC and the anti-TIGIT antibody are administered sequentially. In some embodiments, zimberelimab and the anti-TIGIT antibody are administered concurrently. In some embodiments, zimberelimab and the anti-TIGIT antibody are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, the anti-TIGIT antibody is administered at one or more doses in the range of 800 mg to 1600 mg. In some embodiments, the anti-TIGIT antibody is administered at a dose of 1200 mg. In some embodiments, the anti-TIGIT antibody is administered intravenously. In some embodiments, the anti-TIGIT antibody is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177. Alb-Ftl3L, and SYM-027. In some embodiments, the FLT3 agonist is GS-3583.

In some embodiments, provided herein are methods of treating urothelial cancer comprising co-administering to a subject an effective amount of: (a) a TROP-2-targeted antibody-drug conjugate (ADC) comprising an anti-TROP-2 antibody (anti-TROP-2 ADC); (b) zimberelimab; and, optionally, (c) an anti-TIGIT antibody. In some embodiments, the anti-TROP-2 ADC comprises an anticancer agent payload. In some embodiments, the anticancer agent payload is selected from a microtubule inhibitor, DNA cleavage agent, and topoisomerase I inhibitor. In some embodiments, the anti-TROP-2 ADC comprises a topoisomerase I inhibitor. In some embodiments, the topoisomerase I inhibitor is selected from irinotecan, topetecan, and SN-38. In some embodiments, the anti-TROP-2 ADC has a structural formula of mAb-CL2A-SN-38, with a structure represented by:

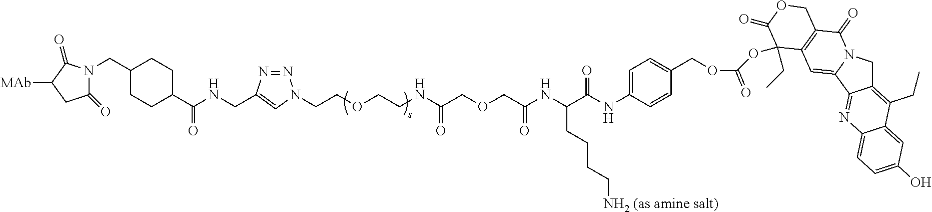

(described, e.g., in U.S. Pat. No. 7,999,083). In some embodiments, the anti-TROP-2 ADC comprises sacituzumab (hRS7; described, e.g., in WO2003074566, FIGS. 3 and 4). In some embodiments, the anti-TROP-2 ADC is selected from sacituzumab govitecan, datopotamab deruxtecan (DS-1062), ESG-401, SKB-264, DAC-02 and BAT-8003. In some embodiments, the anti-TROP-2 ADC is sacituzumab govitecan. In some embodiments, the anti-TROP-2 ADC comprises a microtuble inhibitor. In some embodiments, the microtubule inhibitor is selected from is an auristatin, a taxane, a vinca alkaloid, an epothilone, and maytansinoid. In some embodiments, the auristatin is selected from monomethyl auristatin E (MMAE) and monomethyl auristatin F (MMAF). In some embodiments, the maytansinoid is selected from mertansine (DM1) and ravtansine (DM4). In some embodiments, the anticancer agent payload is selected from anthracyline (e.g., doxorubicin, daunorubicin, epirubicin, idarubicin), pyrrolobenzodiazepine (PBD), or dimer thereof, DNA cross-linking agent SC-DR002 (D6.5), duocarmycin, a duocarmycin (A, B1, B2, C1, C2, D, SA, CC-1065), tubulysin B and analogs thereof. In some embodiments, the method further comprises administering an anti-TIGIT antibody. In some embodiments, the anti-TIGIT antibody is AB3308, AGEN-1307 (AGEN-1327), AGEN-1777, AK127, BMS-986207, domvanalimab, EOS-448, etigilimab, JS006, M6223, ociperlimab, ralzapastotug, SEA-TGT (SGN-TGT), tiragolumab, or vibostolimab. In some embodiments, the anti-TIGIT antibody is domvanalimab. In some embodiments, the anti-TIGIT antibody is M6223. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is (i) unresectable, locally advanced, (ii) metastatic, or (iii) muscle invasive. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally anti-TIGIT antibody. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally anti-TIGIT antibody. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, zimberelimab, and optionally anti-TIGIT antibody is administered in a neoadjuvant setting (e.g., in preparation for surgery or radiation therapy). In some embodiments, the combination of the ADC, zimberelimab, and optionally anti-TIGIT antibody is administered in an adjuvant setting (e.g., following a primary treatment such as surgery or radiation therapy). In some embodiments, the combination of the ADC, zimberelimab, and optionally anti-TIGIT antibody is administered in a therapeutic setting (e.g., as the primary therapy). In some embodiments, the combination of the ADC, zimberelimab, and optionally anti-TIGIT antibody is administered in a maintenance setting. In some embodiments, the subject receives one or more doses of the ADC. In some embodiments, the subject receives one or more doses of zimberelimab. In some embodiments, the subject receives one or more doses of the anti-TIGIT antibody. In some embodiments, the ADC and zimberelimab are administered concurrently. In some embodiments, the ADC and zimberelimab are administered sequentially. In some embodiments, the ADC and the anti-TIGIT antibody are administered concurrently. In some embodiments, the ADC and the anti-TIGIT antibody are administered sequentially. In some embodiments, zimberelimab and the anti-TIGIT antibody are administered concurrently. In some embodiments, zimberelimab and the anti-TIGIT antibody are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, the anti-TIGIT antibody is administered at one or more doses in the range of 800 mg to 1600 mg. In some embodiments, the anti-TIGIT antibody is administered at a dose of 1200 mg. In some embodiments, the anti-TIGIT antibody is administered intravenously. In some embodiments, the anti-TIGIT antibody is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating, mitigating, reducing, preventing or delaying the recurrence or metastasis of urothelial cancer, co-administering to a subject an effective amount of: (a) a TROP-2-targeted antibody-drug conjugate (ADC) comprising an anti-TROP-2 antibody (anti-TROP-2 ADC); (b) zimberelimab; and, optionally, (c) domvanalimab. In some embodiments, the anti-TROP-2 ADC comprises an anticancer agent payload. In some embodiments, the anticancer agent payload is selected from a microtubule inhibitor, DNA cleavage agent, and topoisomerase I inhibitor. In some embodiments, the anti-TROP-2 ADC comprises a topoisomerase I inhibitor. In some embodiments, the topoisomerase I inhibitor is selected from irinotecan, topetecan, and SN-38. In some embodiments, the anti-TROP-2 ADC has a structural formula of mAb-CL2A-SN-38, with a structure represented by:

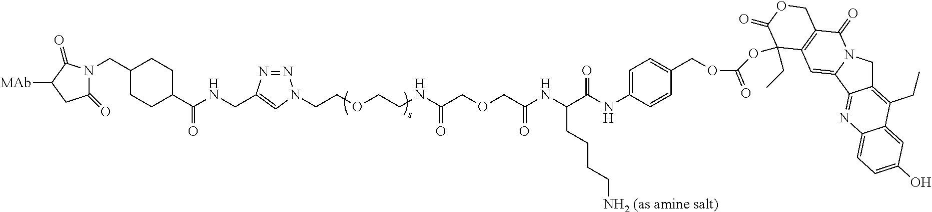

(described, e.g., in U.S. Pat. No. 7,999,083). In some embodiments, the anti-TROP-2 ADC comprises sacituzumab (hRS7; described, e.g., in WO2003074566, FIGS. 3 and 4). In some embodiments, the anti-TROP-2 ADC is selected from sacituzumab govitecan, datopotamab deruxtecan (DS-1062), ESG-401, SKB-264, DAC-02 and BAT-8003. In some embodiments, the anti-TROP-2 ADC is sacituzumab govitecan. In some embodiments, the anti-TROP-2 ADC comprises a microtuble inhibitor. In some embodiments, the microtubule inhibitor is selected from is an auristatin, a taxane, a vinca alkaloid, an epothilone, and maytansinoid. In some embodiments, the auristatin is selected from monomethyl auristatin E (MMAE) and monomethyl auristatin F (MMAF). In some embodiments, the maytansinoid is selected from mertansine (DM1) and ravtansine (DM4). In some embodiments, the anticancer agent payload is selected from anthracyline (e.g., doxorubicin, daunorubicin, epirubicin, idarubicin), pyrrolobenzodiazepine (PBD), or dimer thereof, DNA cross-linking agent SC-DR002 (D6.5), duocarmycin, a duocarmycin (A, B1, B2, C1, C2, D, SA, CC-1065), tubulysin B and analogs thereof. In some embodiments, the method further comprises administering domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is (i) unresectable, locally advanced, (ii) metastatic, or (iii) muscle invasive. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally domvanalimab. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, zimberelimab, and optionally domvanalimab is administered in a neoadjuvant setting (e.g., in preparation for surgery or radiation therapy). In some embodiments, the combination of the ADC, zimberelimab, and optionally domvanalimab is administered in an adjuvant setting (e.g., following a primary treatment such as surgery or radiation therapy). In some embodiments, the combination of the ADC, zimberelimab, and optionally domvanalimab is administered in a therapeutic setting (e.g., as the primary therapy). In some embodiments, the combination of the ADC, zimberelimab, and optionally domvanalimab is administered in a maintenance setting. In some embodiments, the subject receives one or more doses of the ADC. In some embodiments, the subject receives one or more doses of zimberelimab. In some embodiments, the subject receives one or more doses of domvanalimab. In some embodiments, the ADC and zimberelimab are administered concurrently. In some embodiments, the ADC and zimberelimab are administered sequentially. In some embodiments, the ADC and domvanalimab are administered concurrently. In some embodiments, the ADC and domvanalimab are administered sequentially. In some embodiments, zimberelimab and domvanalimab are administered concurrently. In some embodiments, zimberelimab and domvanalimab are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating urothelial cancer comprising co-administering to a subject an effective amount of: (a) a TROP-2-targeted antibody-drug conjugate (ADC) comprising an anti-TROP-2 antibody (anti-TROP-2 ADC); (b) zimberelimab; and, optionally, (c) domvanalimab. In some embodiments, the anti-TROP-2 ADC comprises an anticancer agent payload. In some embodiments, the anticancer agent payload is selected from a microtubule inhibitor, DNA cleavage agent, and topoisomerase I inhibitor. In some embodiments, the anti-TROP-2 ADC comprises a topoisomerase I inhibitor. In some embodiments, the topoisomerase I inhibitor is selected from irinotecan, topetecan, and SN-38. In some embodiments, the anti-TROP-2 ADC has a structural formula of mAb-CL2A-SN-38, with a structure represented by:

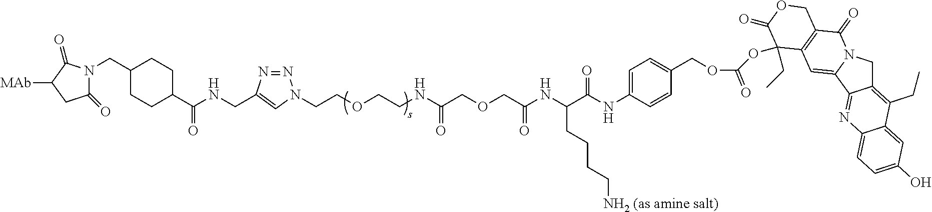

(described, e.g., in U.S. Pat. No. 7,999,083). In some embodiments, the anti-TROP-2 ADC comprises sacituzumab (hRS7; described, e.g., in WO2003074566, FIGS. 3 and 4). In some embodiments, the anti-TROP-2 ADC is selected from sacituzumab govitecan, datopotamab deruxtecan (DS-1062), ESG-401, SKB-264, DAC-02 and BAT-8003. In some embodiments, the anti-TROP-2 ADC is sacituzumab govitecan. In some embodiments, the anti-TROP-2 ADC comprises a microtuble inhibitor. In some embodiments, the microtubule inhibitor is selected from is an auristatin, a taxane, a vinca alkaloid, an epothilone, and maytansinoid. In some embodiments, the auristatin is selected from monomethyl auristatin E (MMAE) and monomethyl auristatin F (MMAF). In some embodiments, the maytansinoid is selected from mertansine (DM1) and ravtansine (DM4). In some embodiments, the anticancer agent payload is selected from anthracyline (e.g., doxorubicin, daunorubicin, epirubicin, idarubicin), pyrrolobenzodiazepine (PBD), or dimer thereof, DNA cross-linking agent SC-DR002 (D6.5), duocarmycin, a duocarmycin (A, B1, B2, C1, C2, D, SA, CC-1065), tubulysin B and analogs thereof. In some embodiments, the method further comprises administering domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is (i) unresectable, locally advanced, (ii) metastatic, or (iii) muscle invasive. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally domvanalimab. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, zimberelimab, and optionally domvanalimab is administered in a neoadjuvant setting (e.g., in preparation for surgery or radiation therapy). In some embodiments, the combination of the ADC, zimberelimab, and optionally domvanalimab is administered in an adjuvant setting (e.g., following a primary treatment such as surgery or radiation therapy). In some embodiments, the combination of the ADC, zimberelimab, and optionally domvanalimab is administered in a therapeutic setting (e.g., as the primary therapy). In some embodiments, the combination of the ADC, zimberelimab, and optionally domvanalimab is administered in a maintenance setting. In some embodiments, the subject receives one or more doses of the ADC. In some embodiments, the subject receives one or more doses of zimberelimab. In some embodiments, the subject receives one or more doses of domvanalimab. In some embodiments, the ADC and zimberelimab are administered concurrently.

In some embodiments, the ADC and zimberelimab are administered sequentially. In some embodiments, the ADC and domvanalimab are administered concurrently. In some embodiments, the ADC and domvanalimab are administered sequentially. In some embodiments, zimberelimab and domvanalimab are administered concurrently. In some embodiments, zimberelimab and domvanalimab are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating, mitigating, reducing, preventing or delaying the recurrence or metastasis of urothelial cancer, comprising co-administering to a subject an effective amount of: (a) a TROP-2-targeted antibody-drug conjugate (ADC) comprising an anti-TROP-2 antibody (anti-TROP-2 ADC); (b) an anti-PD-(L)1 antibody; and, optionally, (c) domvanalimab. In some embodiments, the anti-TROP-2 ADC comprises an anticancer agent payload. In some embodiments, the anticancer agent payload is selected from a microtubule inhibitor, DNA cleavage agent, and topoisomerase I inhibitor. In some embodiments, the anti-TROP-2 ADC comprises a topoisomerase I inhibitor. In some embodiments, the topoisomerase I inhibitor is selected from irinotecan, topetecan, and SN-38. In some embodiments, the anti-TROP-2 ADC has a structural formula of mAb-CL2A-SN-38, with a structure represented by:

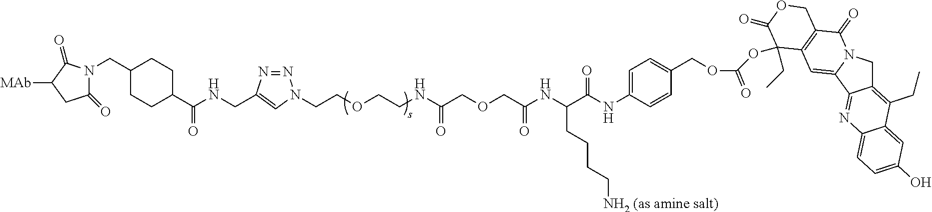

(described, e.g., in U.S. Pat. No. 7,999,083). In some embodiments, the anti-TROP-2 ADC comprises sacituzumab (hRS7; described, e.g., in WO2003074566, FIGS. 3 and 4). In some embodiments, the anti-TROP-2 ADC is selected from sacituzumab govitecan, datopotamab deruxtecan (DS-1062), ESG-401, SKB-264, DAC-02 and BAT-8003. In some embodiments, the anti-TROP-2 ADC is sacituzumab govitecan. In some embodiments, the anti-TROP-2 ADC comprises a microtuble inhibitor. In some embodiments, the microtubule inhibitor is selected from is an auristatin, a taxane, a vinca alkaloid, an epothilone, and maytansinoid. In some embodiments, the auristatin is selected from monomethyl auristatin E (MMAE) and monomethyl auristatin F (MMAF). In some embodiments, the maytansinoid is selected from mertansine (DM1) and ravtansine (DM4). In some embodiments, the anticancer agent payload is selected from anthracyline (e.g., doxorubicin, daunorubicin, epirubicin, idarubicin), pyrrolobenzodiazepine (PBD), or dimer thereof, DNA cross-linking agent SC-DR002 (D6.5), duocarmycin, a duocarmycin (A, B1, B2, C1, C2, D, SA, CC-1065), tubulysin B and analogs thereof. In some embodiments, the anti-PD-(L)1 antibody is pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, or zimberelimab. In some embodiments, the anti-PD-(L)1 antibody is zimberelimab. In some embodiments, the anti-PD-(L)1 antibody is durvalumab. In some embodiments, the anti-PD-(L)1 antibody is an Fc-silent antibody. In some embodiments, the anti-PD-(L)1l antibody is an Fc-enabled antibody. In some embodiments, the method further comprises administering domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is (i) unresectable, locally advanced, (ii) metastatic, or (iii) muscle invasive. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of the ADC, anti-PD-(L)1 antibody, and optionally domvanalimab. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more anti-cancer therapies before administration of the combination of the ADC, anti-PD-(L)1 antibody, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, anti-PD-(L)1 antibody, and optionally domvanalimab is administered in a neoadjuvant setting (e.g., in preparation for surgery or radiation therapy). In some embodiments, the combination of the ADC, anti-PD-(L)1 antibody, and optionally domvanalimab is administered in an adjuvant setting (e.g., following a primary treatment such as surgery or radiation therapy). In some embodiments, the combination of the ADC, anti-PD-(L)1 antibody, and optionally domvanalimab is administered in a therapeutic setting (e.g., as the primary therapy). In some embodiments, the combination of the ADC, anti-PD-(L)1 antibody, and optionally domvanalimab is administered in a maintenance setting. In some embodiments, the subject receives one or more doses of the ADC. In some embodiments, the subject receives one or more doses of the anti-PD-(L)1 antibody. In some embodiments, the subject receives one or more doses of domvanalimab. In some embodiments, the ADC and the anti-PD-(L)1 antibody are administered concurrently. In some embodiments, the ADC and the anti-PD-(L)1 antibody are administered sequentially. In some embodiments, the ADC and domvanalimab are administered concurrently. In some embodiments, the ADC and domvanalimab are administered sequentially. In some embodiments, the anti-PD-(L)1 antibody and domvanalimab are administered concurrently. In some embodiments, the anti-PD-(L)1 antibody and domvanalimab are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, the anti-PD-(L)1 antibody is administered at one or more doses in the range of 300 mg to 400 mg. In some embodiments, the anti-PD-(L)1 antibody is administered at dose of 360 mg. In some embodiments, the anti-PD-(L)1 antibody is administered intravenously. In some embodiments, the anti-PD-(L)1 antibody is administered on day 1 of a 21-day cycle. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating urothelial cancer comprising co-administering to a subject an effective amount of: (a) a TROP-2-targeted antibody-drug conjugate (ADC) comprising an anti-TROP-2 antibody (anti-TROP-2 ADC); (b) an anti-PD-(L)1 antibody; and, optionally, (c) domvanalimab. In some embodiments, the anti-TROP-2 ADC comprises an anticancer agent payload. In some embodiments, the anticancer agent payload is selected from a microtubule inhibitor, DNA cleavage agent, and topoisomerase I inhibitor. In some embodiments, the anti-TROP-2 ADC comprises a topoisomerase I inhibitor. In some embodiments, the topoisomerase I inhibitor is selected from irinotecan, topetecan, and SN-38. In some embodiments, the anti-TROP-2 ADC has a structural formula of mAb-CL2A-SN-38, with a structure represented by:

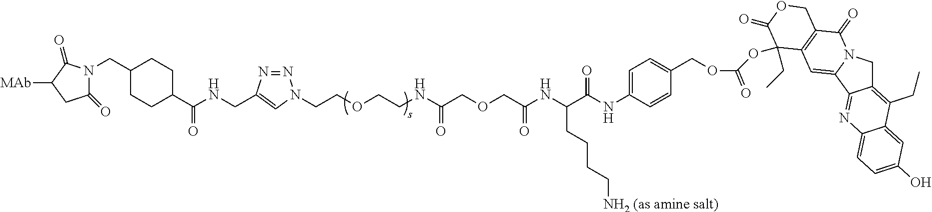

(described, e.g., in U.S. Pat. No. 7,999,083). In some embodiments, the anti-TROP-2 ADC comprises sacituzumab (hRS7; described, e.g., in WO2003074566, FIGS. 3 and 4). In some embodiments, the anti-TROP-2 ADC is selected from sacituzumab govitecan, datopotamab deruxtecan (DS-1062), ESG-401, SKB-264, DAC-02 and BAT-8003. In some embodiments, the anti-TROP-2 ADC is sacituzumab govitecan. In some embodiments, the anti-TROP-2 ADC comprises a microtuble inhibitor. In some embodiments, the microtubule inhibitor is selected from is an auristatin, a taxane, a vinca alkaloid, an epothilone, and maytansinoid. In some embodiments, the auristatin is selected from monomethyl auristatin E (MMAE) and monomethyl auristatin F (MMAF). In some embodiments, the maytansinoid is selected from mertansine (DM1) and ravtansine (DM4). In some embodiments, the anticancer agent payload is selected from anthracyline (e.g., doxorubicin, daunorubicin, epirubicin, idarubicin), pyrrolobenzodiazepine (PBD), or dimer thereof, DNA cross-linking agent SC-DR002 (D6.5), duocarmycin, a duocarmycin (A, B1, B2, C1, C2, D, SA, CC-1065), tubulysin B and analogs thereof. In some embodiments, the anti-PD-(L)1 antibody is pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, or zimberelimab. In some embodiments, the anti-PD-(L)1 antibody is zimberelimab. In some embodiments, the anti-PD-(L)1 antibody is durvalumab. In some embodiments, the anti-PD-(L)1 antibody is an Fc-silent antibody. In some embodiments, the anti-PD-(L)1l antibody is an Fc-enabled antibody. In some embodiments, the method further comprises administering domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is (i) unresectable, locally advanced, (ii) metastatic, or (iii) muscle invasive. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of the ADC, anti-PD-(L)1 antibody, and optionally domvanalimab. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more anti-cancer therapies before administration of the combination of the ADC, anti-PD-(L)1 antibody, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, anti-PD-(L)1 antibody, and optionally domvanalimab is administered in a neoadjuvant setting (e.g., in preparation for surgery or radiation therapy). In some embodiments, the combination of the ADC, anti-PD-(L)1 antibody, and optionally domvanalimab is administered in an adjuvant setting (e.g., following a primary treatment such as surgery or radiation therapy). In some embodiments, the combination of the ADC, anti-PD-(L)1 antibody, and optionally domvanalimab is administered in a therapeutic setting (e.g., as the primary therapy). In some embodiments, the combination of the ADC, anti-PD-(L)1 antibody, and optionally domvanalimab is administered in a maintenance setting. In some embodiments, the subject receives one or more doses of the ADC. In some embodiments, the subject receives one or more doses of the anti-PD-(L)1 antibody. In some embodiments, the subject receives one or more doses of domvanalimab. In some embodiments, the ADC and the anti-PD-(L)1 antibody are administered concurrently. In some embodiments, the ADC and the anti-PD-(L)1 antibody are administered sequentially. In some embodiments, the ADC and domvanalimab are administered concurrently. In some embodiments, the ADC and domvanalimab are administered sequentially. In some embodiments, the anti-PD-(L)1 antibody and domvanalimab are administered concurrently. In some embodiments, the anti-PD-(L)1 antibody and domvanalimab are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, the anti-PD-(L)1 antibody is administered at one or more doses in the range of 300 mg to 400 mg. In some embodiments, the anti-PD-(L)1 antibody is administered at dose of 360 mg. In some embodiments, the anti-PD-(L)1 antibody is administered intravenously. In some embodiments, the anti-PD-(L)1 antibody is administered on day 1 of a 21-day cycle. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating, mitigating, reducing, preventing or delaying the recurrence or metastasis of a tumor antigen positive (TA⁺) cancer, comprising co-administering to a subject an effective amount of: a) a tumor antigen-targeted antibody-drug conjugate (ADC) comprising a topoisomerase I inhibitor (TopI ADC); b) an anti-PD-(L)1 antibody; and, optionally, c) an anti-TIGIT antibody. In some embodiments, the topoisomerase I inhibitor is a camptothecin. In some embodiments, the camptothecin is selected from irinotecan, topotecan, belotecan, and exatecan derivative. In some embodiments, the exatecan derivative is selected from Dxd or SN38. In some embodiments, the topoisomerase I inhibitor is selected from irinotecan, topetecan, and SN-38. In some embodiments, the TopI ADC has a structural formula of mAb-CL2A-SN-38, with a structure represented by:

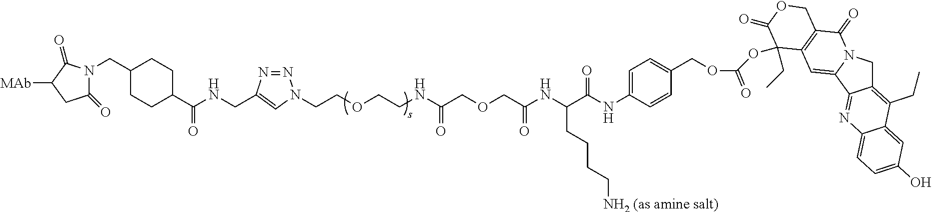

(described, e.g., in U.S. Pat. No. 7,999,083). In some embodiments, the TopI ADC comprises an antibody that binds a tumor antigen. In some embodiments, the tumor antigen is selected from carbonic anhydrase IX, B7, CCCL19, CCCL21, CSAp, HER-2/neu, BrE3, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD80, CD83, CD95, CD126, CD133, CD138, CD147, CD154, CEACAM5, CEACAM-6, alpha-fetoprotein (AFP), VEGF, ED-B fibronectin, EGP-1, EGP-2, EGF receptor (ErbB1), ErbB2, ErbB3, Factor H, FHL-1, Flt-3, folate receptor, Ga 733, GROB, HMGB-1, hypoxia inducible factor (HIF), HM1.24, insulin-like growth factor (ILGF), IFN-a, IFN-b, IL-g, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, HLA-DR, HLA-G, CD66a-d, MAGE, mCRP, MCP-1, MIP-1A, MIP-1B, macrophage migration-inhibitory factor (MIF), MUC1, MUC2, MUC3, MUC4, MUC5, placental growth factor (P1GF), PSA (prostate-specific antigen), PSMA, PSMA dimer, PAM4 antigen, NCA-95, NCA-90, A3, A33, Ep-CAM, KS-1, Le(y), mesothelin, S100, tenascin, TAC, Tn antigen, Thomas-Friedenreich antigens, tumor necrosis antigens, tumor angiogenesis antigens, TNF-a, TRAIL receptor (R1 and R2), VEGFR, RANTES, T101, cancer stem cell antigens, complement factors C3, C3a, C3b, C5a, C5, and an oncogene product. In some embodiments, the TopI ADC comprises an antibody selected from gemtuzumab, brentuximab, belantamab, camidanlumab, trastuzumab, inotuzumab, glembatumumab, anetumab, mirvetuximab, depatuxizumab, vadastuximab, labetuzumab, ladiratuzumab, loncastuximab, patritumab, lifastuzumab, indusatumab, polatuzumab, pinatuzumab, coltuximab, upifitamab, indatuximab, milatuzumab, rovalpituzumab, enfortumab, tisotumab, tusamitamab, disitamab, telisotuzumab, and antigen-binding fragments thereof. In some embodiments, the TopI ADC comprises an antibody selected from hLL1 (anti-CD74), hLL2 (anti-CD22), hRFB4 (anti-CD22), h PAM4 (anti-MUC5ac), hMN-3 (anti-NOTCH3), hMN-14 (labetuzumab; anti-CEACAM5); hMN15 (anti-CEACAM6) hA19 (anti-CD19), hA20 (anti-CD22), hMu-9 (anti-CSAp), hL243 (anti-HLA-DR), hImmu-31 (anti-AFP), and antigen-binding fragments thereof. In some embodiments, the anti-PD-(L)1 antibody is pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, or zimberelimab. In some embodiments, the anti-PD-(L)1 antibody is zimberelimab. In some embodiments, the anti-PD-(L)1 antibody is durvalumab. In some embodiments, the anti-PD-(L)1 antibody is an Fe-silent antibody. In some embodiments, the anti-PD-(L)1 antibody is an Fc-enabled antibody. In some embodiments, any of the methods disclosed herein further comprise administering an anti-TIGIT antibody. In some embodiments, the anti-TIGIT antibody is AB308, AGEN-1307 (AGEN-1327), AGEN-1777, AK127, BMS-986207, domvanalimab, EOS-448, etigilimab, JS006, M6223, ociperlimab, ralzapastotug, SEA-TGT (SGN-TGT), tiragolumab, or vibostolimab. In some embodiments, the anti-TIGIT antibody is domvanalimab. In some embodiments, the anti-TIGIT antibody is M6223. In some embodiments, the anti-PD-(L)1 antibody and the anti-TIGIT antibody are a) zimberelimab and domvanalimab, b) zimberelimab and AB308, c) atezolizumab and tiragolumab, d) pembrolizumab and vibostolimab, e) pembrolizumab and domvanalimab, f) pembrolizumab and AB308, g) MK-7684A (pembrolizumab/vibostolimab coformulation), h) durvalumab and domvanalimab, i) zimberelimab and ralzapastotug, or j) pembrolizumab and ralzapastotug. In some embodiments, the anti-PD-(L)1 antibody and the anti-TIGIT antibody are zimberelimab and domvanalimab. In some embodiments, the anti-PD-(L)1 antibody and the anti-TIGIT antibody are zimberelimab and AB308. In some embodiments, the anti-PD-(L)1 antibody and the anti-TIGIT antibody are zimberelimab and ralzapastotug. In some embodiments, the anti-PD-(L)1 antibody and the anti-TIGIT antibody are durvalumab and domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer. In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the treatment naïve subject has not received prior anti-cancer therapy for (i) advanced cancer or (ii) metastatic cancer. In some embodiments, the subject has received one or more anti-cancer therapies before administration of the combination of the ADC, anti-PD-(L)1 antibody, and optionally anti-TIGIT antibody. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more prior anti-cancer therapies for (i) advanced cancer or (ii) metastatic cancer. In some embodiments, the subject has received one or more anti-cancer therapies before administration of the combination of the ADC, anti-PD-(L)1 antibody, and optionally anti-TIGIT antibody. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, anti-PD-(L)1 antibody, and optionally anti-TIGIT antibody is administered in a neoadjuvant setting (e.g., in preparation for surgery or radiation therapy). In some embodiments, the combination of the ADC, anti-PD-(L)1 antibody, and optionally anti-TIGIT antibody is administered in an adjuvant setting (e.g., following a primary treatment such as surgery or radiation therapy). In some embodiments, the combination of the ADC, anti-PD-(L)1 antibody, and optionally anti-TIGIT antibody is administered in a therapeutic setting (e.g., as the primary therapy). In some embodiments, the combination of the ADC, anti-PD-(L)1 antibody, and optionally anti-TIGIT antibody is administered in a maintenance setting. In some embodiments, the subject receives one or more doses of the ADC. In some embodiments, the subject receives one or more doses of the anti-PD-(L)1 antibody. In some embodiments, the subject receives one or more doses of the anti-TIGIT antibody. In some embodiments, the ADC and the anti-PD-(L)1 antibody are administered concurrently. In some embodiments, the ADC and the anti-PD-(L)1 antibody are administered sequentially. In some embodiments, the ADC and the anti-TIGIT antibody are administered concurrently. In some embodiments, the ADC and the anti-TIGIT antibody are administered sequentially. In some embodiments, the anti-PD-(L)1 antibody and the anti-TIGIT antibody are administered concurrently. In some embodiments, the anti-PD-(L)1 antibody and the anti-TIGIT antibody are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, the anti-PD-(L)1 antibody is administered at one or more doses in the range of 300 mg to 400 mg. In some embodiments, the anti-PD-(L)1 antibody is administered at dose of 360 mg. In some embodiments, the anti-PD-(L)1 antibody is administered intravenously. In some embodiments, the anti-PD-(L)1 antibody is administered on day 1 of a 21-day cycle. In some embodiments, the anti-TIGIT antibody is administered at one or more doses in the range of 800 mg to 1600 mg. In some embodiments, the anti-TIGIT antibody is administered at a dose of 1200 mg. In some embodiments, the anti-TIGIT antibody is administered intravenously. In some embodiments, the anti-TIGIT antibody is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods provided herein are for treating a tumor antigen positive (TA⁺) cancer comprising co-administering to a subject an effective amount of: a) a tumor antigen-targeted antibody-drug conjugate (ADC) comprising a topoisomerase I inhibitor (TopI ADC); b) an anti-PD-(L)1 antibody; and, optionally, c) an anti-TIGIT antibody. In some embodiments, the topoisomerase I inhibitor is a camptothecin. In some embodiments, the camptothecin is selected from irinotecan, topotecan, belotecan, and exatecan derivative. In some embodiments, the exatecan derivative is selected from Dxd or SN38. In some embodiments, the topoisomerase I inhibitor is selected from irinotecan, topetecan, and SN-38. In some embodiments, the TopI ADC has a structural formula of mAb-CL2A-SN-38, with a structure represented by:

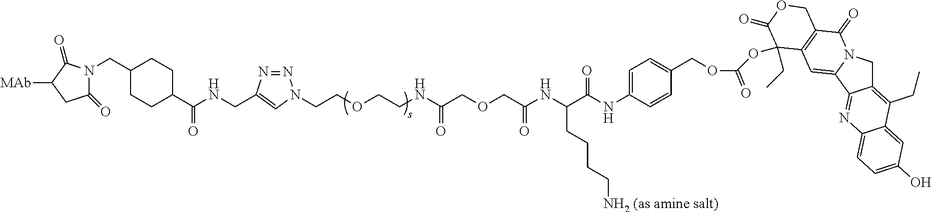

(described, e.g., in U.S. Pat. No. 7,999,083). In some embodiments, the TopI ADC comprises an antibody that binds a tumor antigen. In some embodiments, the tumor antigen is selected from carbonic anhydrase IX, B7, CCCL19, CCCL21, CSAp, HER-2/neu, BrE3, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD80, CD83, CD95, CD126, CD133, CD138, CD147, CD154, CEACAM5, CEACAM-6, alpha-fetoprotein (AFP), VEGF, ED-B fibronectin, EGP-1, EGP-2, EGF receptor (ErbB1), ErbB2, ErbB3, Factor H, FHL-1, Flt-3, folate receptor, Ga 733, GROB, HMGB-1, hypoxia inducible factor (HIF), HM1.24, insulin-like growth factor (ILGF), IFN-a, IFN-b, IL-g, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, HLA-DR, HLA-G, CD66a-d, MAGE, mCRP, MCP-1, MIP-1A, MIP-1B, macrophage migration-inhibitory factor (MIF), MUC1, MUC2, MUC3, MUC4, MUC5, placental growth factor (P1GF), PSA (prostate-specific antigen), PSMA, PSMA dimer, PAM4 antigen, NCA-95, NCA-90, A3, A33, Ep-CAM, KS-1, Le(y), mesothelin, S100, tenascin, TAC, Tn antigen, Thomas-Friedenreich antigens, tumor necrosis antigens, tumor angiogenesis antigens, TNF-a, TRAIL receptor (R1 and R2), VEGFR, RANTES, T101, cancer stem cell antigens, complement factors C3, C3a, C3b, C5a, C5, and an oncogene product. In some embodiments, the TopI ADC comprises an antibody selected from gemtuzumab, brentuximab, belantamab, camidanlumab, trastuzumab, inotuzumab, glembatumumab, anetumab, mirvetuximab, depatuxizumab, vadastuximab, labetuzumab, ladiratuzumab, loncastuximab, patritumab, lifastuzumab, indusatumab, polatuzumab, pinatuzumab, coltuximab, upifitamab, indatuximab, milatuzumab, rovalpituzumab, enfortumab, tisotumab, tusamitamab, disitamab, telisotuzumab, and antigen-binding fragments thereof. In some embodiments, the TopI ADC comprises an antibody selected from hLL1 (anti-CD74), hLL2 (anti-CD22), hRFB4 (anti-CD22), h PAM4 (anti-MUC5ac), hMN-3 (anti-NOTCH3), hMN-14 (labetuzumab; anti-CEACAM5); hMN15 (anti-CEACAM6) hA19 (anti-CD19), hA20 (anti-CD22), hMu-9 (anti-CSAp), hL243 (anti-HLA-DR), hImmu-31 (anti-AFP), and antigen-binding fragments thereof. In some embodiments, the anti-PD-(L)1 antibody is pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, or zimberelimab. In some embodiments, the anti-PD-(L)1 antibody is zimberelimab. In some embodiments, the anti-PD-(L)1 antibody is durvalumab. In some embodiments, the anti-PD-(L)1 antibody is an Fc-silent antibody. In some embodiments, the anti-PD-(L)1 antibody is an Fc-enabled antibody. In some embodiments, any of the methods disclosed herein further comprise administering an anti-TIGIT antibody. In some embodiments, the anti-TIGIT antibody is AB308, AGEN-1307 (AGEN-1327), AGEN-1777, AK127, BMS-986207, domvanalimab, EOS-448, etigilimab, JS006, M6223, ociperlimab, ralzapastotug, SEA-TGT (SGN-TGT), tiragolumab, or vibostolimab. In some embodiments, the anti-TIGIT antibody is domvanalimab. In some embodiments, the anti-TIGIT antibody is M6223. In some embodiments, the anti-PD-(L)1 antibody and the anti-TIGIT antibody are a) zimberelimab and domvanalimab, b) zimberelimab and AB308, c) atezolizumab and tiragolumab, d) pembrolizumab and vibostolimab, e) pembrolizumab and domvanalimab, f) pembrolizumab and AB308, g) MK-7684A (pembrolizumab/vibostolimab coformulation), h) durvalumab and domvanalimab, i) zimberelimab and ralzapastotug, or j) pembrolizumab and ralzapastotug. In some embodiments, the anti-PD-(L)1 antibody and the anti-TIGIT antibody are zimberelimab and domvanalimab. In some embodiments, the anti-PD-(L)1 antibody and the anti-TIGIT antibody are zimberelimab and AB308. In some embodiments, the anti-PD-(L)1 antibody and the anti-TIGIT antibody are zimberelimab and ralzapastotug. In some embodiments, the anti-PD-(L)1 antibody and the anti-TIGIT antibody are durvalumab and domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer. In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or unresectable locally advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the treatment naïve subject has not received prior anti-cancer therapy for (i) advanced cancer or (ii) metastatic cancer. In some embodiments, the subject has received one or more anti-cancer therapies before administration of the combination of the ADC, anti-PD-(L)1 antibody, and optionally anti-TIGIT antibody. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more prior anti-cancer therapies for (i) advanced cancer or (ii) metastatic cancer. In some embodiments, the subject has received one or more anti-cancer therapies before administration of the combination of the ADC, anti-PD-(L)1 antibody, and optionally anti-TIGIT antibody. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, anti-PD-(L)1 antibody, and optionally anti-TIGIT antibody is administered in a neoadjuvant setting (e.g., in preparation for surgery or radiation therapy). In some embodiments, the combination of the ADC, anti-PD-(L)1 antibody, and optionally anti-TIGIT antibody is administered in an adjuvant setting (e.g., following a primary treatment such as surgery or radiation therapy). In some embodiments, the combination of the ADC, anti-PD-(L)1 antibody, and optionally anti-TIGIT antibody is administered in a therapeutic setting (e.g., as the primary therapy). In some embodiments, the combination of the ADC, anti-PD-(L)1 antibody, and optionally anti-TIGIT antibody is administered in a maintenance setting. In some embodiments, the subject receives one or more doses of the ADC. In some embodiments, the subject receives one or more doses of the anti-PD-(L)1 antibody. In some embodiments, the subject receives one or more doses of the anti-TIGIT antibody. In some embodiments, the ADC and the anti-PD-(L)1 antibody are administered concurrently. In some embodiments, the ADC and the anti-PD-(L)1 antibody are administered sequentially. In some embodiments, the ADC and the anti-TIGIT antibody are administered concurrently. In some embodiments, the ADC and the anti-TIGIT antibody are administered sequentially. In some embodiments, the anti-PD-(L)1 antibody and the anti-TIGIT antibody are administered concurrently. In some embodiments, the anti-PD-(L)1 antibody and the anti-TIGIT antibody are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, the anti-PD-(L)1 antibody is administered at one or more doses in the range of 300 mg to 400 mg. In some embodiments, the anti-PD-(L)1 antibody is administered at dose of 360 mg. In some embodiments, the anti-PD-(L)1 antibody is administered intravenously. In some embodiments, the anti-PD-(L)1 antibody is administered on day 1 of a 21-day cycle. In some embodiments, the anti-TIGIT antibody is administered at one or more doses in the range of 800 mg to 1600 mg. In some embodiments, the anti-TIGIT antibody is administered at a dose of 1200 mg. In some embodiments, the anti-TIGIT antibody is administered intravenously. In some embodiments, the anti-TIGIT antibody is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating, mitigating, reducing, preventing or delaying the recurrence or metastasis of urothelial cancer (UC), comprising co-administering to a subject an effective amount of: a) a tumor antigen-targeted antibody-drug conjugate (ADC) comprising a topoisomerase I inhibitor (TopI ADC); b) an anti-PD-(L)1 antibody; and, optionally, c) an anti-TIGIT antibody. In some embodiments, the topoisomerase I inhibitor is a camptothecin. In some embodiments, the camptothecin is selected from irinotecan, topotecan, belotecan, and exatecan derivative. In some embodiments, the exatecan derivative is selected from Dxd or SN38. In some embodiments, the topoisomerase I inhibitor is selected from irinotecan, topetecan, and SN-38. In some embodiments, the TopI ADC has a structural formula of mAb-CL2A-SN-38, with a structure represented by:

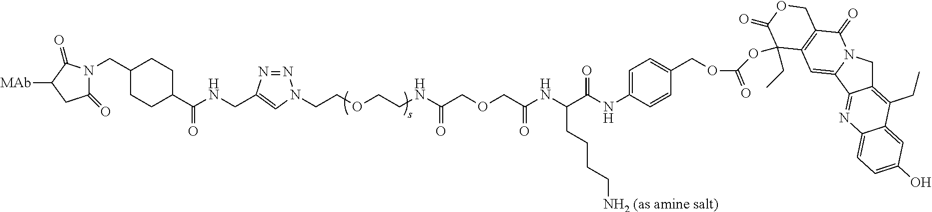

(described, e.g., in U.S. Pat. No. 7,999,083). In some embodiments, the TopI ADC comprises an antibody that binds a tumor antigen. In some embodiments, the tumor antigen is selected from carbonic anhydrase IX, B7, CCCL19, CCCL21, CSAp, HER-2/neu, BrE3, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD80, CD83, CD95, CD126, CD133, CD138, CD147, CD154, CEACAM5, CEACAM-6, alpha-fetoprotein (AFP), VEGF, ED-B fibronectin, EGP-1, EGP-2, EGF receptor (ErbB1), ErbB2, ErbB3, Factor H, FHL-1, Flt-3, folate receptor, Ga 733, GROB, HMGB-1, hypoxia inducible factor (HIF), HM1.24, insulin-like growth factor (ILGF), IFN-a, IFN-b, IL-g, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, HLA-DR, HLA-G, CD66a-d, MAGE, mCRP, MCP-1, MIP-1A, MIP-1B, macrophage migration-inhibitory factor (MIF), MUC1, MUC2, MUC3, MUC4, MUC5, placental growth factor (P1GF), PSA (prostate-specific antigen), PSMA, PSMA dimer, PAM4 antigen, NCA-95, NCA-90, A3, A33, Ep-CAM, KS-1, Le(y), mesothelin, S100, tenascin, TAC, Tn antigen, Thomas-Friedenreich antigens, tumor necrosis antigens, tumor angiogenesis antigens, TNF-a, TRAIL receptor (R1 and R2), VEGFR, RANTES, T101, cancer stem cell antigens, complement factors C3, C3a, C3b, C5a, C5, and an oncogene product. In some embodiments, the TopI ADC comprises an antibody selected from gemtuzumab, brentuximab, belantamab, camidanlumab, trastuzumab, inotuzumab, glembatumumab, anetumab, mirvetuximab, depatuxizumab, vadastuximab, labetuzumab, ladiratuzumab, loncastuximab, patritumab, lifastuzumab, indusatumab, polatuzumab, pinatuzumab, coltuximab, upifitamab, indatuximab, milatuzumab, rovalpituzumab, enfortumab, tisotumab, tusamitamab, disitamab, telisotuzumab, and antigen-binding fragments thereof. In some embodiments, the TopI ADC comprises an antibody selected from hLL1 (anti-CD74), hLL2 (anti-CD22), hRFB4 (anti-CD22), h PAM4 (anti-MUC5ac), hMN-3 (anti-NOTCH3), hMN-14 (labetuzumab; anti-CEACAM5); hMN15 (anti-CEACAM6) hA19 (anti-CD19), hA20 (anti-CD22), hMu-9 (anti-CSAp), hL243 (anti-HLA-DR), hImmu-31 (anti-AFP), and antigen-binding fragments thereof. In some embodiments, the anti-PD-(L)1 antibody is pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, or zimberelimab. In some embodiments, the anti-PD-(L)1 antibody is zimberelimab. In some embodiments, the anti-PD-(L)1 antibody is durvalumab. In some embodiments, the anti-PD-(L)1 antibody is an Fc-silent antibody. In some embodiments, the anti-PD-(L)1 antibody is an Fc-enabled antibody. In some embodiments, any of the methods disclosed herein further comprise administering an anti-TIGIT antibody. In some embodiments, the anti-TIGIT antibody is AB308, AGEN-1307 (AGEN-1327), AGEN-1777, AK127, BMS-986207, domvanalimab, EOS-448, etigilimab, JS006, M6223, ociperlimab, ralzapastotug, SEA-TGT (SGN-TGT), tiragolumab, or vibostolimab. In some embodiments, the anti-TIGIT antibody is domvanalimab. In some embodiments, the anti-TIGIT antibody is M6223. In some embodiments, the anti-PD-(L)1 antibody and the anti-TIGIT antibody are a) zimberelimab and domvanalimab, b) zimberelimab and AB308, c) atezolizumab and tiragolumab, d) pembrolizumab and vibostolimab, e) pembrolizumab and domvanalimab, f) pembrolizumab and AB308, g) MK-7684A (pembrolizumab/vibostolimab coformulation), h) durvalumab and domvanalimab, i) zimberelimab and ralzapastotug, or j) pembrolizumab and ralzapastotug. In some embodiments, the anti-PD-(L)1 antibody and the anti-TIGIT antibody are zimberelimab and domvanalimab. In some embodiments, the anti-PD-(L)1 antibody and the anti-TIGIT antibody are zimberelimab and AB308. In some embodiments, the anti-PD-(L)1 antibody and the anti-TIGIT antibody are zimberelimab and ralzapastotug. In some embodiments, the anti-PD-(L)1 antibody and the anti-TIGIT antibody are durvalumab and domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer. In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or unresectable locally advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the treatment naïve subject has not received prior anti-cancer therapy for (i) advanced cancer or (ii) metastatic cancer. In some embodiments, the subject has received one or more anti-cancer therapies before administration of the combination of the ADC, anti-PD-(L)1 antibody, and optionally anti-TIGIT antibody. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more prior anti-cancer therapies for (i) advanced cancer or (ii) metastatic cancer. In some embodiments, the subject has received one or more anti-cancer therapies before administration of the combination of the ADC, anti-PD-(L)1 antibody, and optionally anti-TIGIT antibody. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, anti-PD-(L)1 antibody, and optionally anti-TIGIT antibody is administered in a neoadjuvant setting (e.g., in preparation for surgery or radiation therapy). In some embodiments, the combination of the ADC, anti-PD-(L)1 antibody, and optionally anti-TIGIT antibody is administered in an adjuvant setting (e.g., following a primary treatment such as surgery or radiation therapy). In some embodiments, the combination of the ADC, anti-PD-(L)1 antibody, and optionally anti-TIGIT antibody is administered in a therapeutic setting (e.g., as the primary therapy). In some embodiments, the combination of the ADC, anti-PD-(L)1 antibody, and optionally anti-TIGIT antibody is administered in a maintenance setting. In some embodiments, the subject receives one or more doses of the ADC. In some embodiments, the subject receives one or more doses of the anti-PD-(L)1 antibody. In some embodiments, the subject receives one or more doses of the anti-TIGIT antibody. In some embodiments, the ADC and the anti-PD-(L)1 antibody are administered concurrently. In some embodiments, the ADC and the anti-PD-(L)1 antibody are administered sequentially. In some embodiments, the ADC and the anti-TIGIT antibody are administered concurrently. In some embodiments, the ADC and the anti-TIGIT antibody are administered sequentially. In some embodiments, the anti-PD-(L)1 antibody and the anti-TIGIT antibody are administered concurrently. In some embodiments, the anti-PD-(L)1 antibody and the anti-TIGIT antibody are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, the anti-PD-(L)1 antibody is administered at one or more doses in the range of 300 mg to 400 mg. In some embodiments, the anti-PD-(L)1 antibody is administered at dose of 360 mg. In some embodiments, the anti-PD-(L)1 antibody is administered intravenously. In some embodiments, the anti-PD-(L)1 antibody is administered on day 1 of a 21-day cycle. In some embodiments, the anti-TIGIT antibody is administered at one or more doses in the range of 800 mg to 1600 mg. In some embodiments, the anti-TIGIT antibody is administered at a dose of 1200 mg. In some embodiments, the anti-TIGIT antibody is administered intravenously. In some embodiments, the anti-TIGIT antibody is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods provided herein are for treating urothelial cancer comprising co-administering to a subject an effective amount of: a) a tumor antigen-targeted antibody-drug conjugate (ADC) comprising a topoisomerase I inhibitor (TopI ADC); b) an anti-PD-(L)1 antibody; and, optionally, c) an anti-TIGIT antibody. In some embodiments, the topoisomerase I inhibitor is a camptothecin. In some embodiments, the camptothecin is selected from irinotecan, topotecan, belotecan, and exatecan derivative. In some embodiments, the exatecan derivative is selected from Dxd or SN38. In some embodiments, the topoisomerase I inhibitor is selected from irinotecan, topetecan, and SN-38. In some embodiments, the TopI ADC has a structural formula of mAb-CL2A-SN-38, with a structure represented by:

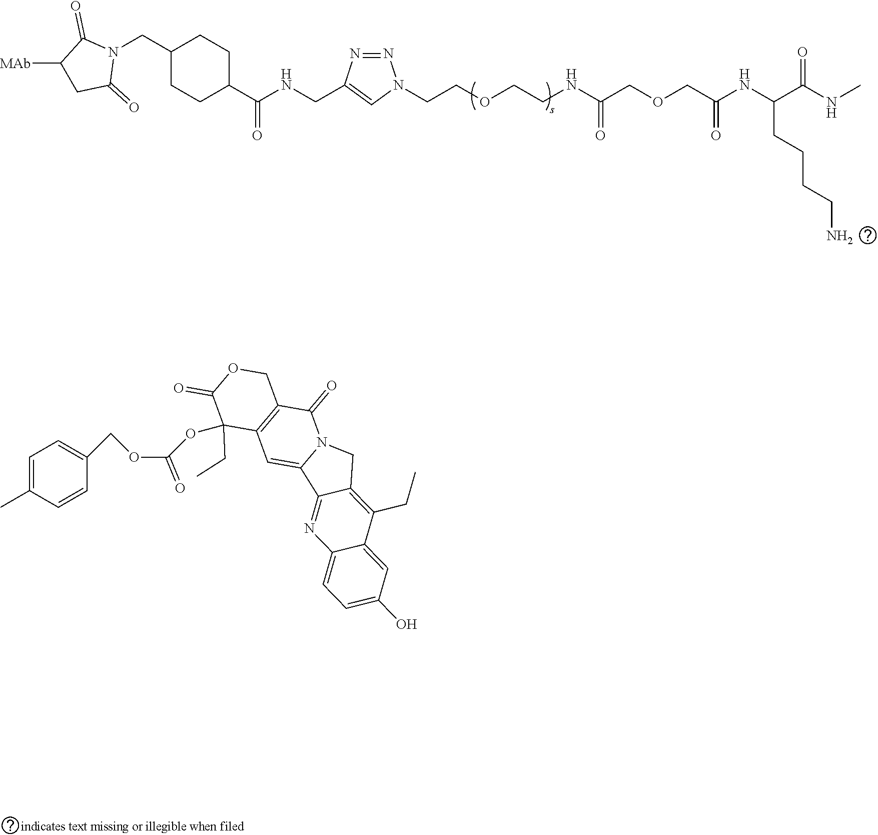

(described, e.g., in U.S. Pat. No. 7,999,083). In some embodiments, the TopI ADC comprises an antibody that binds a tumor antigen. In some embodiments, the tumor antigen is selected from carbonic anhydrase IX, B7, CCCL19, CCCL21, CSAp, HER-2/neu, BrE3, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD80, CD83, CD95, CD126, CD133, CD138, CD147, CD154, CEACAM5, CEACAM-6, alpha-fetoprotein (AFP), VEGF, ED-B fibronectin, EGP-1, EGP-2, EGF receptor (ErbB1), ErbB2, ErbB3, Factor H, FHL-1, Flt-3, folate receptor, Ga 733, GROB, HMGB-1, hypoxia inducible factor (HIF), HM1.24, insulin-like growth factor (ILGF), IFN-a, IFN-b, IL-g, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, HLA-DR, HLA-G, CD66a-d, MAGE, mCRP, MCP-1, MIP-1A, MIP-1B, macrophage migration-inhibitory factor (MIF), MUC1, MUC2, MUC3, MUC4, MUC5, placental growth factor (P1GF), PSA (prostate-specific antigen), PSMA, PSMA dimer, PAM4 antigen, NCA-95, NCA-90, A3, A33, Ep-CAM, KS-1, Le(y), mesothelin, 5100, tenascin, TAC, Tn antigen, Thomas-Friedenreich antigens, tumor necrosis antigens, tumor angiogenesis antigens, TNF-a, TRAIL receptor (R1 and R2), VEGFR, RANTES, T101, cancer stem cell antigens, complement factors C3, C3a, C3b, C5a, C5, and an oncogene product. In some embodiments, the TopI ADC comprises an antibody selected from gemtuzumab, brentuximab, belantamab, camidanlumab, trastuzumab, inotuzumab, glembatumumab, anetumab, mirvetuximab, depatuxizumab, vadastuximab, labetuzumab, ladiratuzumab, loncastuximab, patritumab, lifastuzumab, indusatumab, polatuzumab, pinatuzumab, coltuximab, upifitamab, indatuximab, milatuzumab, rovalpituzumab, enfortumab, tisotumab, tusamitamab, disitamab, telisotuzumab, and antigen-binding fragments thereof. In some embodiments, the TopI ADC comprises an antibody selected from hLL1 (anti-CD74), hLL2 (anti-CD22), hRFB4 (anti-CD22), h PAM4 (anti-MUC5ac), hMN-3 (anti-NOTCH3), hMN-14 (labetuzumab; anti-CEACAM5); hMN15 (anti-CEACAM6) hA19 (anti-CD19), hA20 (anti-CD22), hMu-9 (anti-CSAp), hL243 (anti-HLA-DR), hImmu-31 (anti-AFP), and antigen-binding fragments thereof. In some embodiments, the anti-PD-(L)1 antibody is pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, or zimberelimab. In some embodiments, the anti-PD-(L)1 antibody is zimberelimab. In some embodiments, the anti-PD-(L)1 antibody is durvalumab. In some embodiments, the anti-PD-(L)1 antibody is an Fc-silent antibody. In some embodiments, the anti-PD-(L)1 antibody is an Fc-enabled antibody. In some embodiments, any of the methods disclosed herein further comprise administering an anti-TIGIT antibody. In some embodiments, the anti-TIGIT antibody is AB308, AGEN-1307 (AGEN-1327), AGEN-1777, AK127, BMS-986207, domvanalimab, EOS-448, etigilimab, JS006, M6223, ociperlimab, ralzapastotug, SEA-TGT (SGN-TGT), tiragolumab, or vibostolimab. In some embodiments, the anti-TIGIT antibody is domvanalimab. In some embodiments, the anti-TIGIT antibody is M6223. In some embodiments, the anti-PD-(L)1 antibody and the anti-TIGIT antibody are a) zimberelimab and domvanalimab, b) zimberelimab and AB308, c) atezolizumab and tiragolumab, d) pembrolizumab and vibostolimab, e) pembrolizumab and domvanalimab, f) pembrolizumab and AB308, g) MK-7684A (pembrolizumab/vibostolimab coformulation), h) durvalumab and domvanalimab, i) zimberelimab and ralzapastotug, or j) pembrolizumab and ralzapastotug. In some embodiments, the anti-PD-(L)1 antibody and the anti-TIGIT antibody are zimberelimab and domvanalimab. In some embodiments, the anti-PD-(L)1 antibody and the anti-TIGIT antibody are zimberelimab and AB308. In some embodiments, the anti-PD-(L)1 antibody and the anti-TIGIT antibody are zimberelimab and ralzapastotug. In some embodiments, the anti-PD-(L)1 antibody and the anti-TIGIT antibody are durvalumab and domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer. In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or unresectable locally advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the treatment naïve subject has not received prior anti-cancer therapy for (i) advanced cancer or (ii) metastatic cancer. In some embodiments, the subject has received one or more anti-cancer therapies before administration of the combination of the ADC, anti-PD-(L)1 antibody, and optionally anti-TIGIT antibody. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more prior anti-cancer therapies for (i) advanced cancer or (ii) metastatic cancer. In some embodiments, the subject has received one or more anti-cancer therapies before administration of the combination of the ADC, anti-PD-(L)1 antibody, and optionally anti-TIGIT antibody. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, anti-PD-(L)1 antibody, and optionally anti-TIGIT antibody is administered in a neoadjuvant setting (e.g., in preparation for surgery or radiation therapy). In some embodiments, the combination of the ADC, anti-PD-(L)1 antibody, and optionally anti-TIGIT antibody is administered in an adjuvant setting (e.g., following a primary treatment such as surgery or radiation therapy). In some embodiments, the combination of the ADC, anti-PD-(L)1 antibody, and optionally anti-TIGIT antibody is administered in a therapeutic setting (e.g., as the primary therapy). In some embodiments, the combination of the ADC, anti-PD-(L)1 antibody, and optionally anti-TIGIT antibody is administered in a maintenance setting. In some embodiments, the subject receives one or more doses of the ADC. In some embodiments, the subject receives one or more doses of the anti-PD-(L)1 antibody. In some embodiments, the subject receives one or more doses of the anti-TIGIT antibody. In some embodiments, the ADC and the anti-PD-(L)1 antibody are administered concurrently. In some embodiments, the ADC and the anti-PD-(L)1 antibody are administered sequentially. In some embodiments, the ADC and the anti-TIGIT antibody are administered concurrently. In some embodiments, the ADC and the anti-TIGIT antibody are administered sequentially. In some embodiments, the anti-PD-(L)1 antibody and the anti-TIGIT antibody are administered concurrently. In some embodiments, the anti-PD-(L)1 antibody and the anti-TIGIT antibody are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, the anti-PD-(L)1 antibody is administered at one or more doses in the range of 300 mg to 400 mg. In some embodiments, the anti-PD-(L)1 antibody is administered at dose of 360 mg. In some embodiments, the anti-PD-(L)1 antibody is administered intravenously. In some embodiments, the anti-PD-(L)1 antibody is administered on day 1 of a 21-day cycle. In some embodiments, the anti-TIGIT antibody is administered at one or more doses in the range of 800 mg to 1600 mg. In some embodiments, the anti-TIGIT antibody is administered at a dose of 1200 mg. In some embodiments, the anti-TIGIT antibody is administered intravenously. In some embodiments, the anti-TIGIT antibody is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, 5-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating, mitigating, reducing, preventing or delaying the recurrence or metastasis of a tumor antigen positive (TA⁺) cancer, comprising co-administering to a subject an effective amount of: (a) a tumor antigen-targeted antibody-drug conjugate (ADC) comprising a topoisomerase I inhibitor (TopI ADC); (b) zimberelimab; and, optionally, (c) an anti-TIGIT antibody. In some embodiments, the topoisomerase I inhibitor is a camptothecin. In some embodiments, the camptothecin is selected from irinotecan, topotecan, belotecan, and exatecan derivative. In some embodiments, the exatecan derivative is selected from Dxd or SN38. In some embodiments, the topoisomerase I inhibitor is selected from irinotecan, topetecan, and SN-38. In some embodiments, the TopI ADC has a structural formula of mAb-CL2A-SN-38, with a structure represented by:

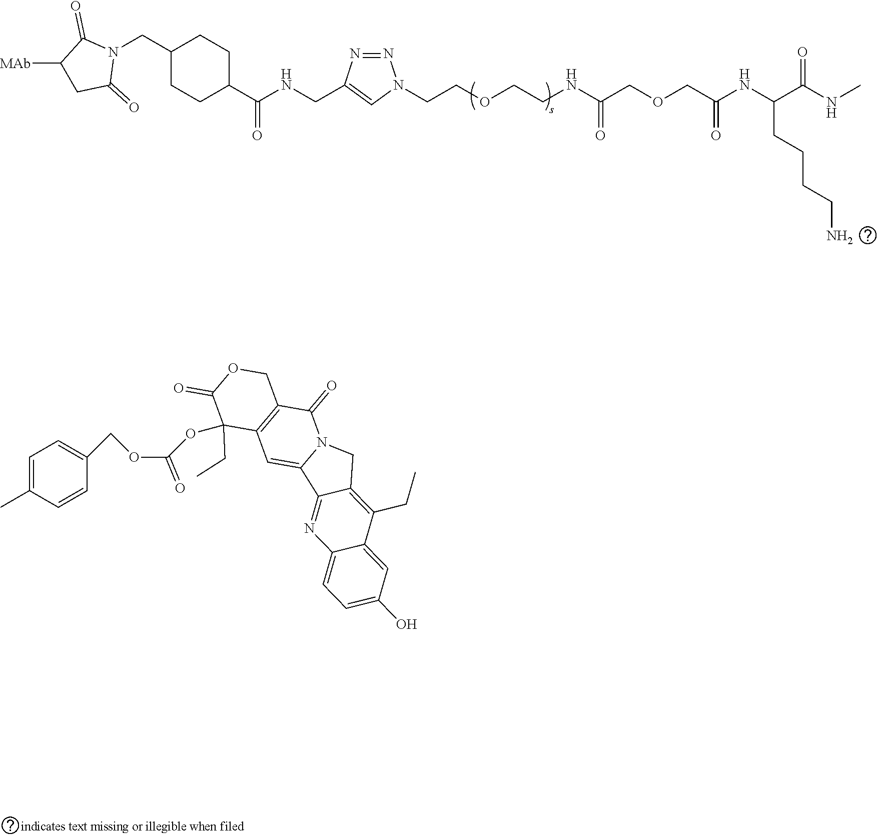

(described, e.g., in U.S. Pat. No. 7,999,083). In some embodiments, the TopI ADC comprises an antibody that binds a tumor antigen. In some embodiments, the tumor antigen is selected from carbonic anhydrase IX, B7, CCCL19, CCCL21, CSAp, HER-2/neu, BrE3, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD80, CD83, CD95, CD126, CD133, CD138, CD147, CD154, CEACAM5, CEACAM-6, alpha-fetoprotein (AFP), VEGF, ED-B fibronectin, EGP-1, EGP-2, EGF receptor (ErbB1), ErbB2, ErbB3, Factor H, FHL-1, Flt-3, folate receptor, Ga 733, GROB, HMGB-1, hypoxia inducible factor (HIF), HM1.24, insulin-like growth factor (ILGF), IFN-a, IFN-b, IL-g, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, HLA-DR, HLA-G, CD66a-d, MAGE, mCRP, MCP-1, MIP-1A, MIP-1B, macrophage migration-inhibitory factor (MIF), MUC1, MUC2, MUC3, MUC4, MUC5, placental growth factor (P1GF), PSA (prostate-specific antigen), PSMA, PSMA dimer, PAM4 antigen, NCA-95, NCA-90, A3, A33, Ep-CAM, KS-1, Le(y), mesothelin, S100, tenascin, TAC, Tn antigen, Thomas-Friedenreich antigens, tumor necrosis antigens, tumor angiogenesis antigens, TNF-a, TRAIL receptor (R1 and R2), VEGFR, RANTES, T101, cancer stem cell antigens, complement factors C3, C3a, C3b, C5a, C5, and an oncogene product. In some embodiments, the TopI ADC comprises an antibody selected from gemtuzumab, brentuximab, belantamab, camidanlumab, trastuzumab, inotuzumab, glembatumumab, anetumab, mirvetuximab, depatuxizumab, vadastuximab, labetuzumab, ladiratuzumab, loncastuximab, patritumab, lifastuzumab, indusatumab, polatuzumab, pinatuzumab, coltuximab, upifitamab, indatuximab, milatuzumab, rovalpituzumab, enfortumab, tisotumab, tusamitamab, disitamab, telisotuzumab, and antigen-binding fragments thereof. In some embodiments, the TopI ADC comprises an antibody selected from hLL1 (anti-CD74), hLL2 (anti-CD22), hRFB4 (anti-CD22), h PAM4 (anti-MUC5ac), hMN-3 (anti-NOTCH3), hMN-14 (labetuzumab; anti-CEACAM5); hMN15 (anti-CEACAM6) hA19 (anti-CD19), hA20 (anti-CD22), hMu-9 (anti-CSAp), hL243 (anti-HLA-DR), hImmu-31 (anti-AFP), and antigen-binding fragments thereof. In some embodiments, the method further comprises administering an anti-TIGIT antibody. In some embodiments, the anti-TIGIT antibody is AB308, AGEN-1307 (AGEN-1327), AGEN-1777, AK127, BMS-986207, domvanalimab, EOS-448, etigilimab, JS006, M6223, ociperlimab, ralzapastotug, SEA-TGT (SGN-TGT), tiragolumab, or vibostolimab. In some embodiments, the anti-TIGIT antibody is domvanalimab. In some embodiments, the anti-TIGIT antibody is M6223. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer. In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or unresectable locally advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the treatment naïve subject has not received prior anti-cancer therapy for (i) advanced cancer or (ii) metastatic cancer. In some embodiments, the subject has received one or more anti-cancer therapies before administration of the combination of the ADC, zimberelimab, and optionally anti-TIGIT antibody. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more prior anti-cancer therapies for (i) advanced cancer or (ii) metastatic cancer. In some embodiments, the subject has received one or more anti-cancer therapies before administration of the combination of the ADC, zimberelimab, and optionally anti-TIGIT antibody. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, zimberelimab, and optionally anti-TIGIT antibody is administered in a neoadjuvant setting (e.g., in preparation for surgery or radiation therapy). In some embodiments, the combination of the ADC, zimberelimab, and optionally anti-TIGIT antibody is administered in an adjuvant setting (e.g., following a primary treatment such as surgery or radiation therapy). In some embodiments, the combination of the ADC, zimberelimab, and optionally anti-TIGIT antibody is administered in a therapeutic setting (e.g., as the primary therapy). In some embodiments, the combination of the ADC, zimberelimab, and optionally anti-TIGIT antibody is administered in a maintenance setting. In some embodiments, the subject receives one or more doses of the ADC. In some embodiments, the subject receives one or more doses of zimberelimab. In some embodiments, the subject receives one or more doses of the anti-TIGIT antibody. In some embodiments, the ADC and zimberelimab are administered concurrently. In some embodiments, the ADC and zimberelimab are administered sequentially. In some embodiments, the ADC and the anti-TIGIT antibody are administered concurrently. In some embodiments, the ADC and the anti-TIGIT antibody are administered sequentially. In some embodiments, zimberelimab and the anti-TIGIT antibody are administered concurrently. In some embodiments, zimberelimab and the anti-TIGIT antibody are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, the anti-TIGIT antibody is administered at one or more doses in the range of 800 mg to 1600 mg. In some embodiments, the anti-TIGIT antibody is administered at a dose of 1200 mg. In some embodiments, the anti-TIGIT antibody is administered intravenously. In some embodiments, the anti-TIGIT antibody is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating a tumor antigen positive (TA⁺) cancer comprising co-administering to a subject an effective amount of: (a) a tumor antigen-targeted antibody-drug conjugate (ADC) comprising a topoisomerase I inhibitor (TopI ADC); (b) zimberelimab; and, optionally, (c) an anti-TIGIT antibody. In some embodiments, the topoisomerase I inhibitor is a camptothecin. In some embodiments, the camptothecin is selected from irinotecan, topotecan, belotecan, and exatecan derivative. In some embodiments, the exatecan derivative is selected from Dxd or SN38. In some embodiments, the topoisomerase I inhibitor is selected from irinotecan, topetecan, and SN-38. In some embodiments, the TopI ADC has a structural formula of mAb-CL2A-SN-38, with a structure represented by:

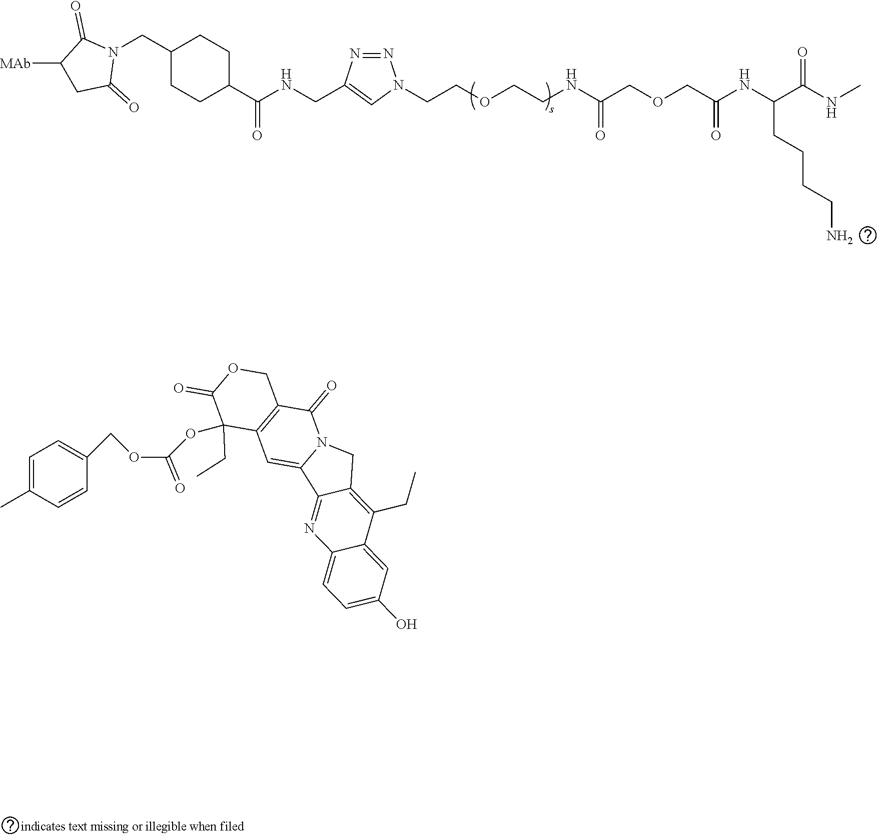

(described, e.g., in U.S. Pat. No. 7,999,083). In some embodiments, the TopI ADC comprises an antibody that binds a tumor antigen. In some embodiments, the tumor antigen is selected from carbonic anhydrase IX, B7, CCCL19, CCCL21, CSAp, HER-2/neu, BrE3, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD80, CD83, CD95, CD126, CD133, CD138, CD147, CD154, CEACAM5, CEACAM-6, alpha-fetoprotein (AFP), VEGF, ED-B fibronectin, EGP-1, EGP-2, EGF receptor (ErbB1), ErbB2, ErbB3, Factor H, FHL-1, Flt-3, folate receptor, Ga 733, GROB, HMGB-1, hypoxia inducible factor (HIF), HM1.24, insulin-like growth factor (ILGF), IFN-a, IFN-b, IL-g, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, HLA-DR, HLA-G, CD66a-d, MAGE, mCRP, MCP-1, MIP-1A, MIP-1B, macrophage migration-inhibitory factor (MIF), MUC1, MUC2, MUC3, MUC4, MUC5, placental growth factor (P1GF), PSA (prostate-specific antigen), PSMA, PSMA dimer, PAM4 antigen, NCA-95, NCA-90, A3, A33, Ep-CAM, KS-1, Le(y), mesothelin, S100, tenascin, TAC, Tn antigen, Thomas-Friedenreich antigens, tumor necrosis antigens, tumor angiogenesis antigens, TNF-a, TRAIL receptor (R1 and R2), VEGFR, RANTES, T101, cancer stem cell antigens, complement factors C3, C3a, C3b, C5a, C5, and an oncogene product. In some embodiments, the TopI ADC comprises an antibody selected from gemtuzumab, brentuximab, belantamab, camidanlumab, trastuzumab, inotuzumab, glembatumumab, anetumab, mirvetuximab, depatuxizumab, vadastuximab, labetuzumab, ladiratuzumab, loncastuximab, patritumab, lifastuzumab, indusatumab, polatuzumab, pinatuzumab, coltuximab, upifitamab, indatuximab, milatuzumab, rovalpituzumab, enfortumab, tisotumab, tusamitamab, disitamab, telisotuzumab, and antigen-binding fragments thereof. In some embodiments, the TopI ADC comprises an antibody selected from hLL1 (anti-CD74), hLL2 (anti-CD22), hRFB4 (anti-CD22), h PAM4 (anti-MUC5ac), hMN-3 (anti-NOTCH3), hMN-14 (labetuzumab; anti-CEACAM5); hMN15 (anti-CEACAM6) hA19 (anti-CD19), hA20 (anti-CD22), hMu-9 (anti-CSAp), hL243 (anti-HLA-DR), hImmu-31 (anti-AFP), and antigen-binding fragments thereof. In some embodiments, the method further comprises administering an anti-TIGIT antibody. In some embodiments, the anti-TIGIT antibody is AB308, AGEN-1307 (AGEN-1327), AGEN-1777, AK127, BMS-986207, domvanalimab, EOS-448, etigilimab, JS006, M6223, ociperlimab, ralzapastotug, SEA-TGT (SGN-TGT), tiragolumab, or vibostolimab. In some embodiments, the anti-TIGIT antibody is domvanalimab. In some embodiments, the anti-TIGIT antibody is M6223. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer. In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or unresectable locally advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the treatment naïve subject has not received prior anti-cancer therapy for (i) advanced cancer or (ii) metastatic cancer. In some embodiments, the subject has received one or more anti-cancer therapies before administration of the combination of the ADC, zimberelimab, and optionally anti-TIGIT antibody. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more prior anti-cancer therapies for (i) advanced cancer or (ii) metastatic cancer. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more prior anti-cancer therapies for (i) advanced cancer or (ii) metastatic cancer. some embodiments, the subject has received one or more anti-cancer therapies before administration of the combination of the ADC, zimberelimab, and optionally anti-TIGIT antibody. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, zimberelimab, and optionally anti-TIGIT antibody is administered in a neoadjuvant setting (e.g., in preparation for surgery or radiation therapy). In some embodiments, the combination of the ADC, zimberelimab, and optionally anti-TIGIT antibody is administered in an adjuvant setting (e.g., following a primary treatment such as surgery or radiation therapy). In some embodiments, the combination of the ADC, zimberelimab, and optionally anti-TIGIT antibody is administered in a therapeutic setting (e.g., as the primary therapy). In some embodiments, the combination of the ADC, zimberelimab, and optionally anti-TIGIT antibody is administered in a maintenance setting. In some embodiments, the subject receives one or more doses of the ADC. In some embodiments, the subject receives one or more doses of zimberelimab. In some embodiments, the subject receives one or more doses of the anti-TIGIT antibody. In some embodiments, the ADC and zimberelimab are administered concurrently. In some embodiments, the ADC and zimberelimab are administered sequentially. In some embodiments, the ADC and the anti-TIGIT antibody are administered concurrently. In some embodiments, the ADC and the anti-TIGIT antibody are administered sequentially. In some embodiments, zimberelimab and the anti-TIGIT antibody are administered concurrently. In some embodiments, zimberelimab and the anti-TIGIT antibody are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, the anti-TIGIT antibody is administered at one or more doses in the range of 800 mg to 1600 mg. In some embodiments, the anti-TIGIT antibody is administered at a dose of 1200 mg. In some embodiments, the anti-TIGIT antibody is administered intravenously. In some embodiments, the anti-TIGIT antibody is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating, mitigating, reducing, preventing or delaying the recurrence or metastasis of a tumor antigen positive (TA⁺) cancer, co-administering to a subject an effective amount of: (a) a tumor antigen-targeted antibody-drug conjugate (ADC) comprising a topoisomerase I inhibitor (TopI ADC); (b) zimberelimab; and, optionally, (c) domvanalimab. In some embodiments, the topoisomerase I inhibitor is a camptothecin. In some embodiments, the camptothecin is selected from irinotecan, topotecan, belotecan, and exatecan derivative. In some embodiments, the exatecan derivative is selected from Dxd or SN38. In some embodiments, the topoisomerase I inhibitor is selected from irinotecan, topetecan, and SN-38. In some embodiments, the TopI ADC has a structural formula of mAb-CL2A-SN-38, with a structure represented by:

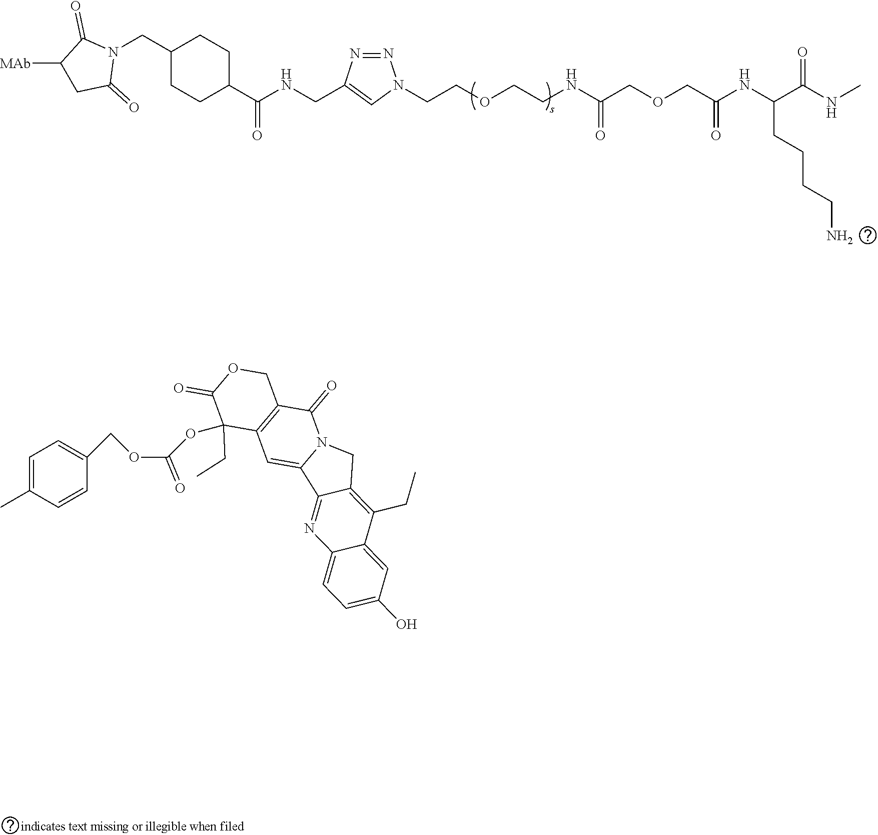

(described, e.g., in U.S. Pat. No. 7,999,083). In some embodiments, the TopI ADC comprises an antibody that binds a tumor antigen. In some embodiments, the tumor antigen is selected from carbonic anhydrase IX, B7, CCCL19, CCCL21, CSAp, HER-2/neu, BrE3, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD80, CD83, CD95, CD126, CD133, CD138, CD147, CD154, CEACAM5, CEACAM-6, alpha-fetoprotein (AFP), VEGF, ED-B fibronectin, EGP-1, EGP-2, EGF receptor (ErbB1), ErbB2, ErbB3, Factor H, FHL-1, Flt-3, folate receptor, Ga 733, GROB, HMGB-1, hypoxia inducible factor (HIF), HM1.24, insulin-like growth factor (ILGF), IFN-a, IFN-b, IL-g, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, HLA-DR, HLA-G, CD66a-d, MAGE, mCRP, MCP-1, MIP-1A, MIP-1B, macrophage migration-inhibitory factor (MIF), MUC1, MUC2, MUC3, MUC4, MUC5, placental growth factor (P1GF), PSA (prostate-specific antigen), PSMA, PSMA dimer, PAM4 antigen, NCA-95, NCA-90, A3, A33, Ep-CAM, KS-1, Le(y), mesothelin, S100, tenascin, TAC, Tn antigen, Thomas-Friedenreich antigens, tumor necrosis antigens, tumor angiogenesis antigens, TNF-a, TRAIL receptor (R1 and R2), VEGFR, RANTES, T101, cancer stem cell antigens, complement factors C3, C3a, C3b, C5a, C5, and an oncogene product. In some embodiments, the TopI ADC comprises an antibody selected from gemtuzumab, brentuximab, belantamab, camidanlumab, trastuzumab, inotuzumab, glembatumumab, anetumab, mirvetuximab, depatuxizumab, vadastuximab, labetuzumab, ladiratuzumab, loncastuximab, patritumab, lifastuzumab, indusatumab, polatuzumab, pinatuzumab, coltuximab, upifitamab, indatuximab, milatuzumab, rovalpituzumab, enfortumab, tisotumab, tusamitamab, disitamab, telisotuzumab, and antigen-binding fragments thereof. In some embodiments, the TopI ADC comprises an antibody selected from hLL1 (anti-CD74), hLL2 (anti-CD22), hRFB4 (anti-CD22), h PAM4 (anti-MUC5ac), hMN-3 (anti-NOTCH3), hMN-14 (labetuzumab; anti-CEACAM5); hMN15 (anti-CEACAM6) hA19 (anti-CD19), hA20 (anti-CD22), hMu-9 (anti-CSAp), hL243 (anti-HLA-DR), hImmu-31 (anti-AFP), and antigen-binding fragments thereof. In some embodiments, the method further comprises administering domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer. In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or unresectable locally advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the treatment naïve subject has not received prior anti-cancer therapy for (i) advanced cancer or (ii) metastatic cancer. In some embodiments, the subject has received one or more anti-cancer therapies before administration of the combination of the ADC, zimberelimab, and optionally domvanalimab. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more prior anti-cancer therapies for (i) advanced cancer or (ii) metastatic cancer. In some embodiments, the subject has received one or more anti-cancer therapies before administration of the combination of the ADC, zimberelimab, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, zimberelimab, and optionally domvanalimab is administered in a neoadjuvant setting (e.g., in preparation for surgery or radiation therapy). In some embodiments, the combination of the ADC, zimberelimab, and optionally domvanalimab is administered in an adjuvant setting (e.g., following a primary treatment such as surgery or radiation therapy). In some embodiments, the combination of the ADC, zimberelimab, and optionally domvanalimab is administered in a therapeutic setting (e.g., as the primary therapy). In some embodiments, the combination of the ADC, zimberelimab, and optionally domvanalimab is administered in a maintenance setting. In some embodiments, the subject receives one or more doses of the ADC. In some embodiments, the subject receives one or more doses of zimberelimab. In some embodiments, the subject receives one or more doses of domvanalimab. In some embodiments, the ADC and zimberelimab are administered concurrently. In some embodiments, the ADC and zimberelimab are administered sequentially. In some embodiments, the ADC and domvanalimab are administered concurrently. In some embodiments, the ADC and domvanalimab are administered sequentially. In some embodiments, zimberelimab and domvanalimab are administered concurrently. In some embodiments, zimberelimab and domvanalimab are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating a tumor antigen positive (TA⁺) cancer comprising co-administering to a subject an effective amount of: (a) a tumor antigen-targeted antibody-drug conjugate (ADC) comprising a topoisomerase I inhibitor (TopI ADC); (b) zimberelimab; and, optionally, (c) domvanalimab. In some embodiments, the topoisomerase I inhibitor is a camptothecin. In some embodiments, the camptothecin is selected from irinotecan, topotecan, belotecan, and exatecan derivative. In some embodiments, the exatecan derivative is selected from Dxd or SN38. In some embodiments, the topoisomerase I inhibitor is selected from irinotecan, topetecan, and SN-38. In some embodiments, the TopI ADC has a structural formula of mAb-CL2A-SN-38, with a structure represented by:

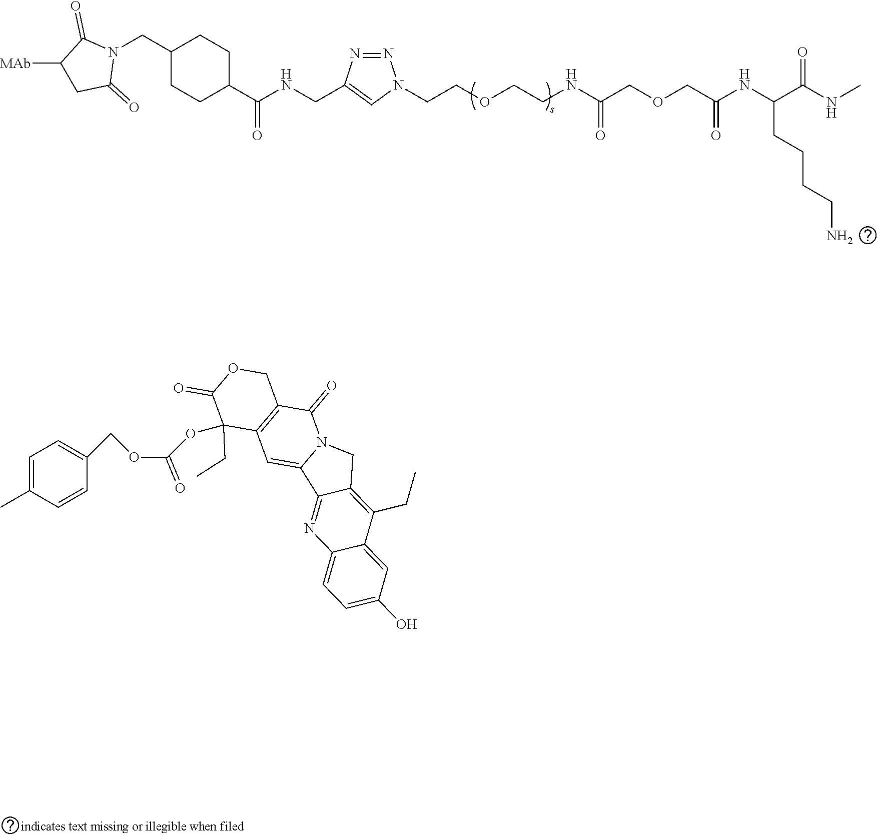

(described, e.g., in U.S. Pat. No. 7,999,083). In some embodiments, the TopI ADC comprises an antibody that binds a tumor antigen. In some embodiments, the tumor antigen is selected from carbonic anhydrase IX, B7, CCCL19, CCCL21, CSAp, HER-2/neu, BrE3, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD80, CD83, CD95, CD126, CD133, CD138, CD147, CD154, CEACAM5, CEACAM-6, alpha-fetoprotein (AFP), VEGF, ED-B fibronectin, EGP-1, EGP-2, EGF receptor (ErbB1), ErbB2, ErbB3, Factor H, FHL-1, Flt-3, folate receptor, Ga 733, GROB, HMGB-1, hypoxia inducible factor (HIF), HM1.24, insulin-like growth factor (ILGF), IFN-a, IFN-b, IL-g, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, HLA-DR, HLA-G, CD66a-d, MAGE, mCRP, MCP-1, MIP-1A, MIP-1B, macrophage migration-inhibitory factor (MIF), MUC1, MUC2, MUC3, MUC4, MUC5, placental growth factor (P1GF), PSA (prostate-specific antigen), PSMA, PSMA dimer, PAM4 antigen, NCA-95, NCA-90, A3, A33, Ep-CAM, KS-1, Le(y), mesothelin, S100, tenascin, TAC, Tn antigen, Thomas-Friedenreich antigens, tumor necrosis antigens, tumor angiogenesis antigens, TNF-a, TRAIL receptor (R1 and R2), VEGFR, RANTES, T101, cancer stem cell antigens, complement factors C3, C3a, C3b, C5a, C5, and an oncogene product. In some embodiments, the TopI ADC comprises an antibody selected from gemtuzumab, brentuximab, belantamab, camidanlumab, trastuzumab, inotuzumab, glembatumumab, anetumab, mirvetuximab, depatuxizumab, vadastuximab, labetuzumab, ladiratuzumab, loncastuximab, patritumab, lifastuzumab, indusatumab, polatuzumab, pinatuzumab, coltuximab, upifitamab, indatuximab, milatuzumab, rovalpituzumab, enfortumab, tisotumab, tusamitamab, disitamab, telisotuzumab, and antigen-binding fragments thereof. In some embodiments, the TopI ADC comprises an antibody selected from hLL1 (anti-CD74), hLL2 (anti-CD22), hRFB4 (anti-CD22), h PAM4 (anti-MUC5ac), hMN-3 (anti-NOTCH3), hMN-14 (labetuzumab; anti-CEACAM5); hMN15 (anti-CEACAM6) hA19 (anti-CD19), hA20 (anti-CD22), hMu-9 (anti-CSAp), hL243 (anti-HLA-DR), hImmu-31 (anti-AFP), and antigen-binding fragments thereof. In some embodiments, the method further comprises administering domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer. In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or unresectable locally advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the treatment naïve subject has not received prior anti-cancer therapy for (i) advanced cancer or (ii) metastatic cancer. In some embodiments, the subject has received one or more anti-cancer therapies before administration of the combination of the ADC, zimberelimab, and optionally domvanalimab. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more prior anti-cancer therapies for (i) advanced cancer or (ii) metastatic cancer. In some embodiments, the subject has received one or more anti-cancer therapies before administration of the combination of the ADC, zimberelimab, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, zimberelimab, and optionally domvanalimab is administered in a neoadjuvant setting (e.g., in preparation for surgery or radiation therapy). In some embodiments, the combination of the ADC, zimberelimab, and optionally domvanalimab is administered in an adjuvant setting (e.g., following a primary treatment such as surgery or radiation therapy). In some embodiments, the combination of the ADC, zimberelimab, and optionally domvanalimab is administered in a therapeutic setting (e.g., as the primary therapy). In some embodiments, the combination of the ADC, zimberelimab, and optionally domvanalimab is administered in a maintenance setting. In some embodiments, the subject receives one or more doses of the ADC. In some embodiments, the subject receives one or more doses of zimberelimab. In some embodiments, the subject receives one or more doses of domvanalimab. In some embodiments, the ADC and zimberelimab are administered concurrently. In some embodiments, the ADC and zimberelimab are administered sequentially. In some embodiments, the ADC and domvanalimab are administered concurrently. In some embodiments, the ADC and domvanalimab are administered sequentially. In some embodiments, zimberelimab and domvanalimab are administered concurrently. In some embodiments, zimberelimab and domvanalimab are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating, mitigating, reducing, preventing or delaying the recurrence or metastasis of a tumor antigen positive (TA⁺) cancer, comprising co-administering to a subject an effective amount of: (a) a tumor antigen-targeted antibody-drug conjugate (ADC) comprising a topoisomerase I inhibitor (TopI ADC); (b) an anti-PD-(L)1 antibody; and, optionally, (c) domvanalimab. In some embodiments, the topoisomerase I inhibitor is a camptothecin. In some embodiments, the camptothecin is selected from irinotecan, topotecan, belotecan, and exatecan derivative. In some embodiments, the exatecan derivative is selected from Dxd or SN38. In some embodiments, the topoisomerase I inhibitor is selected from irinotecan, topetecan, and SN-38. In some embodiments, the TopI ADC has a structural formula of mAb-CL2A-SN-38, with a structure represented by:

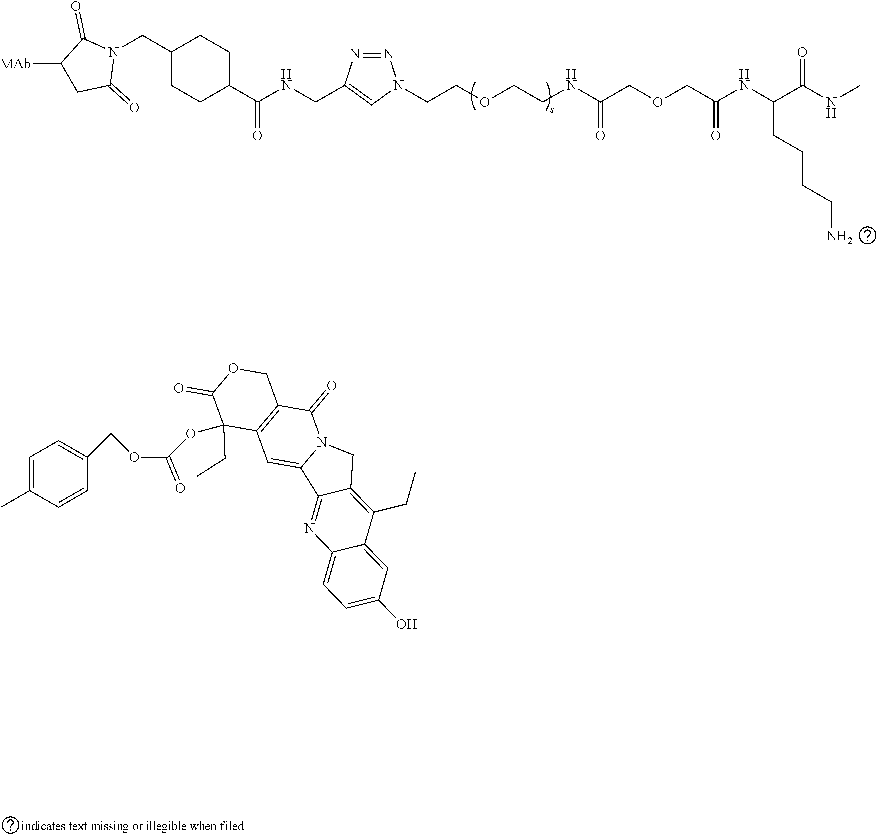

(described, e.g., in U.S. Pat. No. 7,999,083). In some embodiments, the TopI ADC comprises an antibody that binds a tumor antigen. In some embodiments, the tumor antigen is selected from carbonic anhydrase IX, B7, CCCL19, CCCL21, CSAp, HER-2/neu, BrE3, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD80, CD83, CD95, CD126, CD133, CD138, CD147, CD154, CEACAM5, CEACAM-6, alpha-fetoprotein (AFP), VEGF, ED-B fibronectin, EGP-1, EGP-2, EGF receptor (ErbB1), ErbB2, ErbB3, Factor H, FHL-1, Flt-3, folate receptor, Ga 733, GROB, HMGB-1, hypoxia inducible factor (HIF), HM1.24, insulin-like growth factor (ILGF), IFN-a, IFN-b, IL-g, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, HLA-DR, HLA-G, CD66a-d, MAGE, mCRP, MCP-1, MIP-1A, MIP-1B, macrophage migration-inhibitory factor (MIF), MUC1, MUC2, MUC3, MUC4, MUC5, placental growth factor (P1GF), PSA (prostate-specific antigen), PSMA, PSMA dimer, PAM4 antigen, NCA-95, NCA-90, A3, A33, Ep-CAM, KS-1, Le(y), mesothelin, S100, tenascin, TAC, Tn antigen, Thomas-Friedenreich antigens, tumor necrosis antigens, tumor angiogenesis antigens, TNF-a, TRAIL receptor (R1 and R2), VEGFR, RANTES, T101, cancer stem cell antigens, complement factors C3, C3a, C3b, C5a, C5, and an oncogene product. In some embodiments, the TopI ADC comprises an antibody selected from gemtuzumab, brentuximab, belantamab, camidanlumab, trastuzumab, inotuzumab, glembatumumab, anetumab, mirvetuximab, depatuxizumab, vadastuximab, labetuzumab, ladiratuzumab, loncastuximab, patritumab, lifastuzumab, indusatumab, polatuzumab, pinatuzumab, coltuximab, upifitamab, indatuximab, milatuzumab, rovalpituzumab, enfortumab, tisotumab, tusamitamab, disitamab, telisotuzumab, and antigen-binding fragments thereof. In some embodiments, the TopI ADC comprises an antibody selected from hLL1 (anti-CD74), hLL2 (anti-CD22), hRFB4 (anti-CD22), h PAM4 (anti-MUC5ac), hMN-3 (anti-NOTCH3), hMN-14 (labetuzumab; anti-CEACAM5); hMN15 (anti-CEACAM6) hA19 (anti-CD19), hA20 (anti-CD22), hMu-9 (anti-CSAp), hL243 (anti-HLA-DR), hImmu-31 (anti-AFP), and antigen-binding fragments thereof. In some embodiments, the anti-PD-(L)1 antibody is pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, or zimberelimab. In some embodiments, the anti-PD-(L)1 antibody is zimberelimab. In some embodiments, the anti-PD-(L)1 antibody is durvalumab. In some embodiments, the anti-PD-(L)1 antibody is an Fe-silent antibody. In some embodiments, the anti-PD-(L)1 antibody is an Fc-enabled antibody. In some embodiments, the method further comprises administering domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer. In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or unresectable locally advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the treatment naïve subject has not received prior anti-cancer therapy for (i) advanced cancer or (ii) metastatic cancer. In some embodiments, the subject has received one or more anti-cancer therapies before administration of the combination of the ADC, anti-PD-(L)1 antibody, and optionally domvanalimab. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more prior anti-cancer therapies for (i) advanced cancer or (ii) metastatic cancer. In some embodiments, the subject has received one or more anti-cancer therapies before administration of the combination of the ADC, anti-PD-(L)1 antibody, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, anti-PD-(L)1 antibody, and optionally domvanalimab is administered in a neoadjuvant setting (e.g., in preparation for surgery or radiation therapy). In some embodiments, the combination of the ADC, anti-PD-(L)1 antibody, and optionally domvanalimab is administered in an adjuvant setting (e.g., following a primary treatment such as surgery or radiation therapy). In some embodiments, the combination of the ADC, anti-PD-(L)1 antibody, and optionally domvanalimab is administered in a therapeutic setting (e.g., as the primary therapy). In some embodiments, the combination of the ADC, anti-PD-(L)1 antibody, and optionally domvanalimab is administered in a maintenance setting. In some embodiments, the subject receives one or more doses of the ADC. In some embodiments, the subject receives one or more doses of the anti-PD-(L)1 antibody. In some embodiments, the subject receives one or more doses of domvanalimab. In some embodiments, the ADC and the anti-PD-(L)1 antibody are administered concurrently. In some embodiments, the ADC and the anti-PD-(L)1 antibody are administered sequentially. In some embodiments, the ADC and domvanalimab are administered concurrently. In some embodiments, the ADC and domvanalimab are administered sequentially. In some embodiments, the anti-PD-(L)1 antibody and domvanalimab are administered concurrently. In some embodiments, the anti-PD-(L)1 antibody and domvanalimab are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, the anti-PD-(L)1 antibody is administered at one or more doses in the range of 300 mg to 400 mg. In some embodiments, the anti-PD-(L)1 antibody is administered at dose of 360 mg. In some embodiments, the anti-PD-(L)1 antibody is administered intravenously. In some embodiments, the anti-PD-(L)1 antibody is administered on day 1 of a 21-day cycle. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ft13L, and SYM-027. In some embodiments, the FLT3 agonist is GS-3583.

In some embodiments, provided herein are methods of treating a tumor antigen positive (TA⁺) cancer comprising co-administering to a subject an effective amount of: (a) a tumor antigen-targeted antibody-drug conjugate (ADC) comprising a topoisomerase I inhibitor (TopI ADC); (b) an anti-PD-(L)1 antibody; and, optionally, (c) domvanalimab. In some embodiments, the topoisomerase I inhibitor is a camptothecin. In some embodiments, the camptothecin is selected from irinotecan, topotecan, belotecan, and exatecan derivative. In some embodiments, the exatecan derivative is selected from Dxd or SN38. In some embodiments, the topoisomerase I inhibitor is selected from irinotecan, topetecan, and SN-38. In some embodiments, the TopI ADC has a structural formula of mAb-CL2A-SN-38, with a structure represented by:

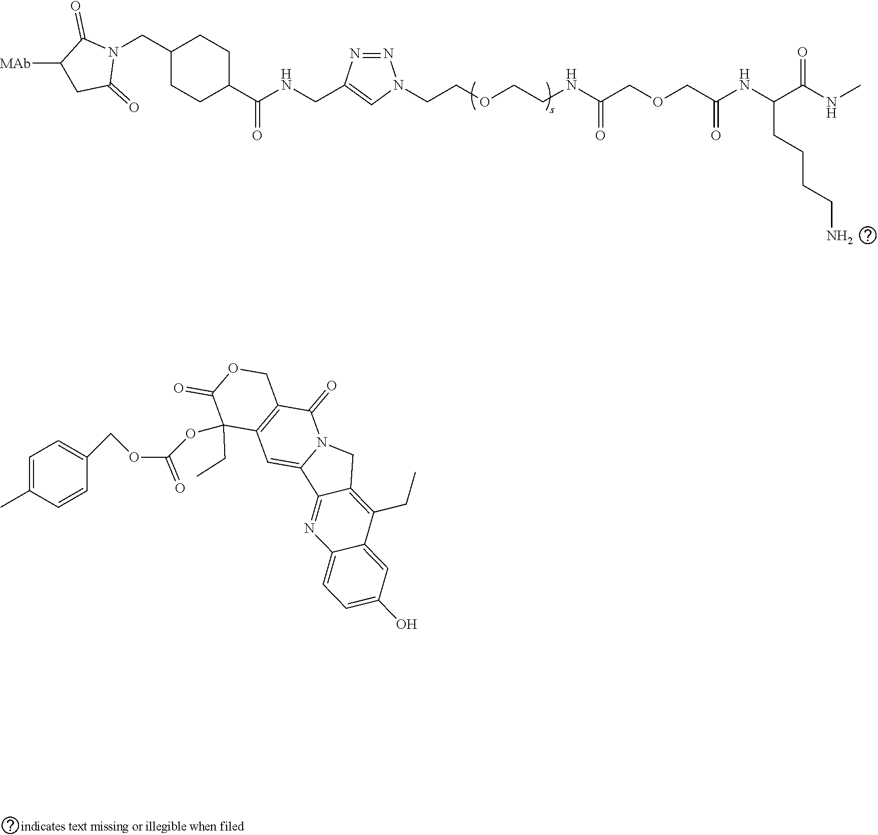

(described, e.g., in U.S. Pat. No. 7,999,083). In some embodiments, the TopI ADC comprises an antibody that binds a tumor antigen. In some embodiments, the tumor antigen is selected from carbonic anhydrase IX, B7, CCCL19, CCCL21, CSAp, HER-2/neu, BrE3, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD80, CD83, CD95, CD126, CD133, CD138, CD147, CD154, CEACAM5, CEACAM-6, alpha-fetoprotein (AFP), VEGF, ED-B fibronectin, EGP-1, EGP-2, EGF receptor (ErbB1), ErbB2, ErbB3, Factor H, FHL-1, Flt-3, folate receptor, Ga 733, GROB, HMGB-1, hypoxia inducible factor (HIF), HM1.24, insulin-like growth factor (ILGF), IFN-a, IFN-b, IL-g, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, HLA-DR, HLA-G, CD66a-d, MAGE, mCRP, MCP-1, MIP-1A, MIP-1B, macrophage migration-inhibitory factor (MIF), MUC1, MUC2, MUC3, MUC4, MUC5, placental growth factor (P1GF), PSA (prostate-specific antigen), PSMA, PSMA dimer, PAM4 antigen, NCA-95, NCA-90, A3, A33, Ep-CAM, KS-1, Le(y), mesothelin, S100, tenascin, TAC, Tn antigen, Thomas-Friedenreich antigens, tumor necrosis antigens, tumor angiogenesis antigens, TNF-a, TRAIL receptor (R1 and R2), VEGFR, RANTES, T101, cancer stem cell antigens, complement factors C3, C3a, C3b, C5a, C5, and an oncogene product. In some embodiments, the TopI ADC comprises an antibody selected from gemtuzumab, brentuximab, belantamab, camidanlumab, trastuzumab, inotuzumab, glembatumumab, anetumab, mirvetuximab, depatuxizumab, vadastuximab, labetuzumab, ladiratuzumab, loncastuximab, patritumab, lifastuzumab, indusatumab, polatuzumab, pinatuzumab, coltuximab, upifitamab, indatuximab, milatuzumab, rovalpituzumab, enfortumab, tisotumab, tusamitamab, disitamab, telisotuzumab, and antigen-binding fragments thereof. In some embodiments, the TopI ADC comprises an antibody selected from hLL1 (anti-CD74), hLL2 (anti-CD22), hRFB4 (anti-CD22), h PAM4 (anti-MUC5ac), hMN-3 (anti-NOTCH3), hMN-14 (labetuzumab; anti-CEACAM5); hMN15 (anti-CEACAM6) hA19 (anti-CD19), hA20 (anti-CD22), hMu-9 (anti-CSAp), hL243 (anti-HLA-DR), hImmu-31 (anti-AFP), and antigen-binding fragments thereof. In some embodiments, the anti-PD-(L)1 antibody is pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, or zimberelimab. In some embodiments, the anti-PD-(L)1 antibody is zimberelimab. In some embodiments, the anti-PD-(L)1 antibody is durvalumab. In some embodiments, the anti-PD-(L)1 antibody is an Fe-silent antibody. In some embodiments, the anti-PD-(L)1 antibody is an Fc-enabled antibody. In some embodiments, the method further comprises administering domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer. In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or unresectable locally advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the treatment naïve subject has not received prior anti-cancer therapy for (i) advanced cancer or (ii) metastatic cancer. In some embodiments, the subject has received one or more anti-cancer therapies before administration of the combination of the ADC, anti-PD-(L)1 antibody, and optionally domvanalimab. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more prior anti-cancer therapies for (i) advanced cancer or (ii) metastatic cancer. In some embodiments, the subject has received one or more anti-cancer therapies before administration of the combination of the ADC, anti-PD-(L)1 antibody, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, anti-PD-(L)1 antibody, and optionally domvanalimab is administered in a neoadjuvant setting (e.g., in preparation for surgery or radiation therapy). In some embodiments, the combination of the ADC, anti-PD-(L)1 antibody, and optionally domvanalimab is administered in an adjuvant setting (e.g., following a primary treatment such as surgery or radiation therapy). In some embodiments, the combination of the ADC, anti-PD-(L)1 antibody, and optionally domvanalimab is administered in a therapeutic setting (e.g., as the primary therapy). In some embodiments, the combination of the ADC, anti-PD-(L)1 antibody, and optionally domvanalimab is administered in a maintenance setting. In some embodiments, the subject receives one or more doses of the ADC. In some embodiments, the subject receives one or more doses of the anti-PD-(L)1 antibody. In some embodiments, the subject receives one or more doses of domvanalimab. In some embodiments, the ADC and the anti-PD-(L)1 antibody are administered concurrently. In some embodiments, the ADC and the anti-PD-(L)1 antibody are administered sequentially. In some embodiments, the ADC and domvanalimab are administered concurrently. In some embodiments, the ADC and domvanalimab are administered sequentially. In some embodiments, the anti-PD-(L)1 antibody and domvanalimab are administered concurrently. In some embodiments, the anti-PD-(L)1 antibody and domvanalimab are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, the anti-PD-(L)1 antibody is administered at one or more doses in the range of 300 mg to 400 mg. In some embodiments, the anti-PD-(L)1 antibody is administered at dose of 360 mg. In some embodiments, the anti-PD-(L)1 antibody is administered intravenously. In some embodiments, the anti-PD-(L)1 antibody is administered on day 1 of a 21-day cycle. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ft13L, and SYM-027. In some embodiments, the FLT3 agonist is GS-3583.

In some embodiments, provided herein are methods of treating, mitigating, reducing, preventing or delaying the recurrence or metastasis of urothelial cancer, comprising co-administering to a subject an effective amount of: (a) a tumor antigen-targeted antibody-drug conjugate (ADC) comprising a topoisomerase I inhibitor (TopI ADC); (b) zimberelimab; and, optionally, (c) an anti-TIGIT antibody. In some embodiments, the topoisomerase I inhibitor is a camptothecin. In some embodiments, the camptothecin is selected from irinotecan, topotecan, belotecan, and exatecan derivative. In some embodiments, the exatecan derivative is selected from Dxd or SN38. In some embodiments, the topoisomerase I inhibitor is selected from irinotecan, topetecan, and SN-38. In some embodiments, the TopI ADC has a structural formula of mAb-CL2A-SN-38, with a structure represented by:

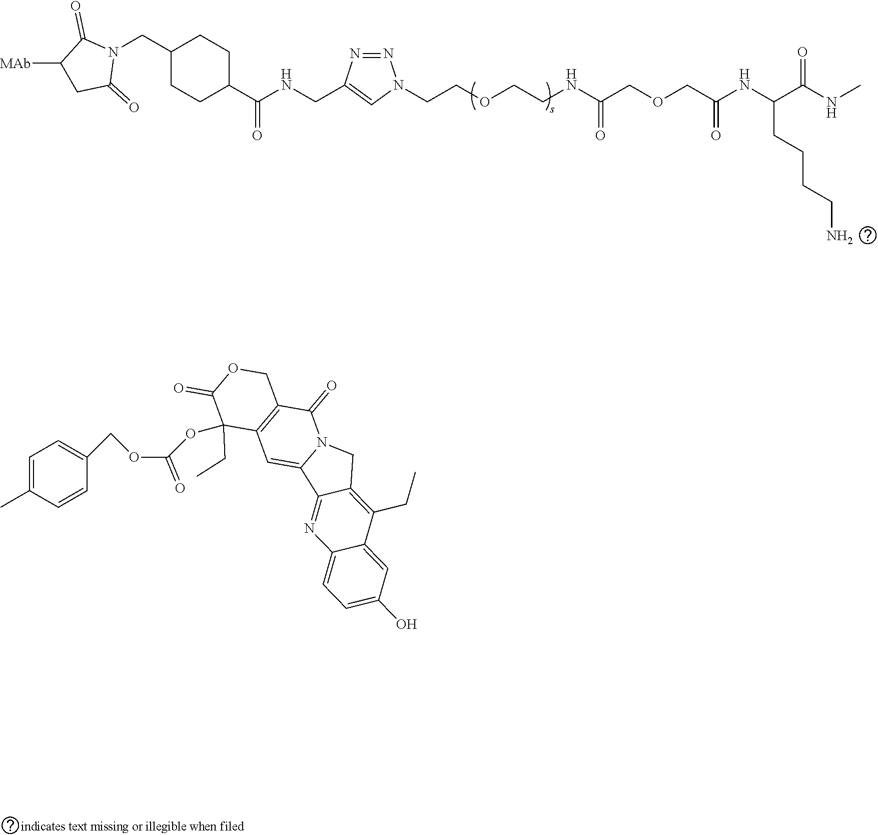

(described, e.g., in U.S. Pat. No. 7,999,083). In some embodiments, the TopI ADC comprises an antibody that binds a tumor antigen. In some embodiments, the tumor antigen is selected from carbonic anhydrase IX, B7, CCCL19, CCCL21, CSAp, HER-2/neu, BrE3, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD80, CD83, CD95, CD126, CD133, CD138, CD147, CD154, CEACAM5, CEACAM-6, alpha-fetoprotein (AFP), VEGF, ED-B fibronectin, EGP-1, EGP-2, EGF receptor (ErbB1), ErbB2, ErbB3, Factor H, FHL-1, Flt-3, folate receptor, Ga 733, GROB, HMGB-1, hypoxia inducible factor (HIF), HM1.24, insulin-like growth factor (ILGF), IFN-a, IFN-b, IL-g, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, HLA-DR, HLA-G, CD66a-d, MAGE, mCRP, MCP-1, MIP-1A, MIP-1B, macrophage migration-inhibitory factor (MIF), MUC1, MUC2, MUC3, MUC4, MUC5, placental growth factor (P1GF), PSA (prostate-specific antigen), PSMA, PSMA dimer, PAM4 antigen, NCA-95, NCA-90, A3, A33, Ep-CAM, KS-1, Le(y), mesothelin, S100, tenascin, TAC, Tn antigen, Thomas-Friedenreich antigens, tumor necrosis antigens, tumor angiogenesis antigens, TNF-a, TRAIL receptor (R1 and R2), VEGFR, RANTES, T101, cancer stem cell antigens, complement factors C3, C3a, C3b, C5a, C5, and an oncogene product. In some embodiments, the TopI ADC comprises an antibody selected from gemtuzumab, brentuximab, belantamab, camidanlumab, trastuzumab, inotuzumab, glembatumumab, anetumab, mirvetuximab, depatuxizumab, vadastuximab, labetuzumab, ladiratuzumab, loncastuximab, patritumab, lifastuzumab, indusatumab, polatuzumab, pinatuzumab, coltuximab, upifitamab, indatuximab, milatuzumab, rovalpituzumab, enfortumab, tisotumab, tusamitamab, disitamab, telisotuzumab, and antigen-binding fragments thereof. In some embodiments, the TopI ADC comprises an antibody selected from hLL1 (anti-CD74), hLL2 (anti-CD22), hRFB4 (anti-CD22), h PAM4 (anti-MUC5ac), hMN-3 (anti-NOTCH3), hMN-14 (labetuzumab; anti-CEACAM5); hMN15 (anti-CEACAM6) hA19 (anti-CD19), hA20 (anti-CD22), hMu-9 (anti-CSAp), hL243 (anti-HLA-DR), hImmu-31 (anti-AFP), and antigen-binding fragments thereof. In some embodiments, the method further comprises administering an anti-TIGIT antibody. In some embodiments, the anti-TIGIT antibody is AB308, AGEN-1307 (AGEN-1327), AGEN-1777, AK127, BMS-986207, domvanalimab, EOS-448, etigilimab, JS006, M6223, ociperlimab, ralzapastotug, SEA-TGT (SGN-TGT), tiragolumab, or vibostolimab. In some embodiments, the anti-TIGIT antibody is domvanalimab. In some embodiments, the anti-TIGIT antibody is M6223. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is (i) unresectable, locally advanced, (ii) metastatic, or (iii) muscle invasive. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally anti-TIGIT antibody. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally anti-TIGIT antibody. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, zimberelimab, and optionally anti-TIGIT antibody is administered in a neoadjuvant setting (e.g., in preparation for surgery or radiation therapy). In some embodiments, the combination of the ADC, zimberelimab, and optionally anti-TIGIT antibody is administered in an adjuvant setting (e.g., following a primary treatment such as surgery or radiation therapy). In some embodiments, the combination of the ADC, zimberelimab, and optionally anti-TIGIT antibody is administered in a therapeutic setting (e.g., as the primary therapy). In some embodiments, the combination of the ADC, zimberelimab, and optionally anti-TIGIT antibody is administered in a maintenance setting. In some embodiments, the subject receives one or more doses of the ADC. In some embodiments, the subject receives one or more doses of zimberelimab. In some embodiments, the subject receives one or more doses of the anti-TIGIT antibody. In some embodiments, the ADC and zimberelimab are administered concurrently. In some embodiments, the ADC and zimberelimab are administered sequentially. In some embodiments, the ADC and the anti-TIGIT antibody are administered concurrently. In some embodiments, the ADC and the anti-TIGIT antibody are administered sequentially. In some embodiments, zimberelimab and the anti-TIGIT antibody are administered concurrently. In some embodiments, zimberelimab and the anti-TIGIT antibody are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, the anti-TIGIT antibody is administered at one or more doses in the range of 800 mg to 1600 mg. In some embodiments, the anti-TIGIT antibody is administered at a dose of 1200 mg. In some embodiments, the anti-TIGIT antibody is administered intravenously. In some embodiments, the anti-TIGIT antibody is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ft13L, and SYM-027. In some embodiments, the FLT3 agonist is GS-3583.

In some embodiments, provided herein are methods of treating urothelial cancer comprising co-administering to a subject an effective amount of: (a) a tumor antigen-targeted antibody-drug conjugate (ADC) comprising a topoisomerase I inhibitor (TopI ADC); (b) zimberelimab; and, optionally, (c) an anti-TIGIT antibody. In some embodiments, the topoisomerase I inhibitor is a camptothecin. In some embodiments, the camptothecin is selected from irinotecan, topotecan, belotecan, and exatecan derivative. In some embodiments, the exatecan derivative is selected from Dxd or SN38. In some embodiments, the topoisomerase I inhibitor is selected from irinotecan, topetecan, and SN-38. In some embodiments, the TopI ADC has a structural formula of mAb-CL2A-SN-38, with a structure represented by:

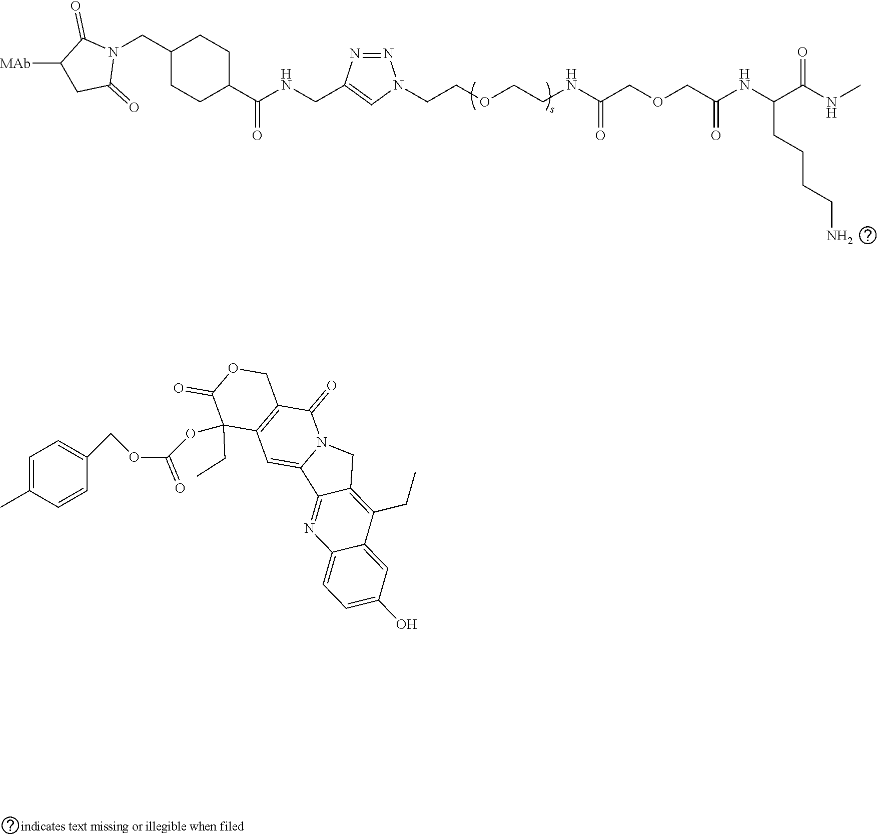

(described, e.g., in U.S. Pat. No. 7,999,083). In some embodiments, the TopI ADC comprises an antibody that binds a tumor antigen. In some embodiments, the tumor antigen is selected from carbonic anhydrase IX, B7, CCCL19, CCCL21, CSAp, HER-2/neu, BrE3, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD80, CD83, CD95, CD126, CD133, CD138, CD147, CD154, CEACAM5, CEACAM-6, alpha-fetoprotein (AFP), VEGF, ED-B fibronectin, EGP-1, EGP-2, EGF receptor (ErbB1), ErbB2, ErbB3, Factor H, FHL-1, Flt-3, folate receptor, Ga 733, GROB, HMGB-1, hypoxia inducible factor (HIF), HM1.24, insulin-like growth factor (ILGF), IFN-a, IFN-b, IL-g, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, HLA-DR, HLA-G, CD66a-d, MAGE, mCRP, MCP-1, MIP-1A, MIP-1B, macrophage migration-inhibitory factor (MIF), MUC1, MUC2, MUC3, MUC4, MUC5, placental growth factor (P1GF), PSA (prostate-specific antigen), PSMA, PSMA dimer, PAM4 antigen, NCA-95, NCA-90, A3, A33, Ep-CAM, KS-1, Le(y), mesothelin, S100, tenascin, TAC, Tn antigen, Thomas-Friedenreich antigens, tumor necrosis antigens, tumor angiogenesis antigens, TNF-a, TRAIL receptor (R1 and R2), VEGFR, RANTES, T101, cancer stem cell antigens, complement factors C3, C3a, C3b, C5a, C5, and an oncogene product. In some embodiments, the TopI ADC comprises an antibody selected from gemtuzumab, brentuximab, belantamab, camidanlumab, trastuzumab, inotuzumab, glembatumumab, anetumab, mirvetuximab, depatuxizumab, vadastuximab, labetuzumab, ladiratuzumab, loncastuximab, patritumab, lifastuzumab, indusatumab, polatuzumab, pinatuzumab, coltuximab, upifitamab, indatuximab, milatuzumab, rovalpituzumab, enfortumab, tisotumab, tusamitamab, disitamab, telisotuzumab, and antigen-binding fragments thereof. In some embodiments, the TopI ADC comprises an antibody selected from hLL1 (anti-CD74), hLL2 (anti-CD22), hRFB4 (anti-CD22), h PAM4 (anti-MUC5ac), hMN-3 (anti-NOTCH3), hMN-14 (labetuzumab; anti-CEACAM5); hMN15 (anti-CEACAM6) hA19 (anti-CD19), hA20 (anti-CD22), hMu-9 (anti-CSAp), hL243 (anti-HLA-DR), hImmu-31 (anti-AFP), and antigen-binding fragments thereof. In some embodiments, the method further comprises administering an anti-TIGIT antibody. In some embodiments, the anti-TIGIT antibody is AB308, AGEN-1307 (AGEN-1327), AGEN-1777, AK127, BMS-986207, domvanalimab, EOS-448, etigilimab, JS006, M6223, ociperlimab, ralzapastotug, SEA-TGT (SGN-TGT), tiragolumab, or vibostolimab. In some embodiments, the anti-TIGIT antibody is domvanalimab. In some embodiments, the anti-TIGIT antibody is M6223. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is (i) unresectable, locally advanced, (ii) metastatic, or (iii) muscle invasive. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally anti-TIGIT antibody. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally anti-TIGIT antibody. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, zimberelimab, and optionally anti-TIGIT antibody is administered in a neoadjuvant setting (e.g., in preparation for surgery or radiation therapy). In some embodiments, the combination of the ADC, zimberelimab, and optionally anti-TIGIT antibody is administered in an adjuvant setting (e.g., following a primary treatment such as surgery or radiation therapy). In some embodiments, the combination of the ADC, zimberelimab, and optionally anti-TIGIT antibody is administered in a therapeutic setting (e.g., as the primary therapy). In some embodiments, the combination of the ADC, zimberelimab, and optionally anti-TIGIT antibody is administered in a maintenance setting. In some embodiments, the subject receives one or more doses of the ADC. In some embodiments, the subject receives one or more doses of zimberelimab. In some embodiments, the subject receives one or more doses of the anti-TIGIT antibody. In some embodiments, the ADC and zimberelimab are administered concurrently. In some embodiments, the ADC and zimberelimab are administered sequentially. In some embodiments, the ADC and the anti-TIGIT antibody are administered concurrently. In some embodiments, the ADC and the anti-TIGIT antibody are administered sequentially. In some embodiments, zimberelimab and the anti-TIGIT antibody are administered concurrently. In some embodiments, zimberelimab and the anti-TIGIT antibody are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, the anti-TIGIT antibody is administered at one or more doses in the range of 800 mg to 1600 mg. In some embodiments, the anti-TIGIT antibody is administered at a dose of 1200 mg. In some embodiments, the anti-TIGIT antibody is administered intravenously. In some embodiments, the anti-TIGIT antibody is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201. IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, 5-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating, mitigating, reducing, preventing or delaying the recurrence or metastasis of urothelial cancer, co-administering to a subject an effective amount of: (a) a tumor antigen-targeted antibody-drug conjugate (ADC) comprising a topoisomerase I inhibitor (TopI ADC); (b) zimberelimab; and, optionally, (c) domvanalimab. In some embodiments, the topoisomerase I inhibitor is a camptothecin. In some embodiments, the camptothecin is selected from irinotecan, topotecan, belotecan, and exatecan derivative. In some embodiments, the exatecan derivative is selected from Dxd or SN38. In some embodiments, the topoisomerase I inhibitor is selected from irinotecan, topetecan, and SN-38. In some embodiments, the TopI ADC has a structural formula of mAb-CL2A-SN-38, with a structure represented by:

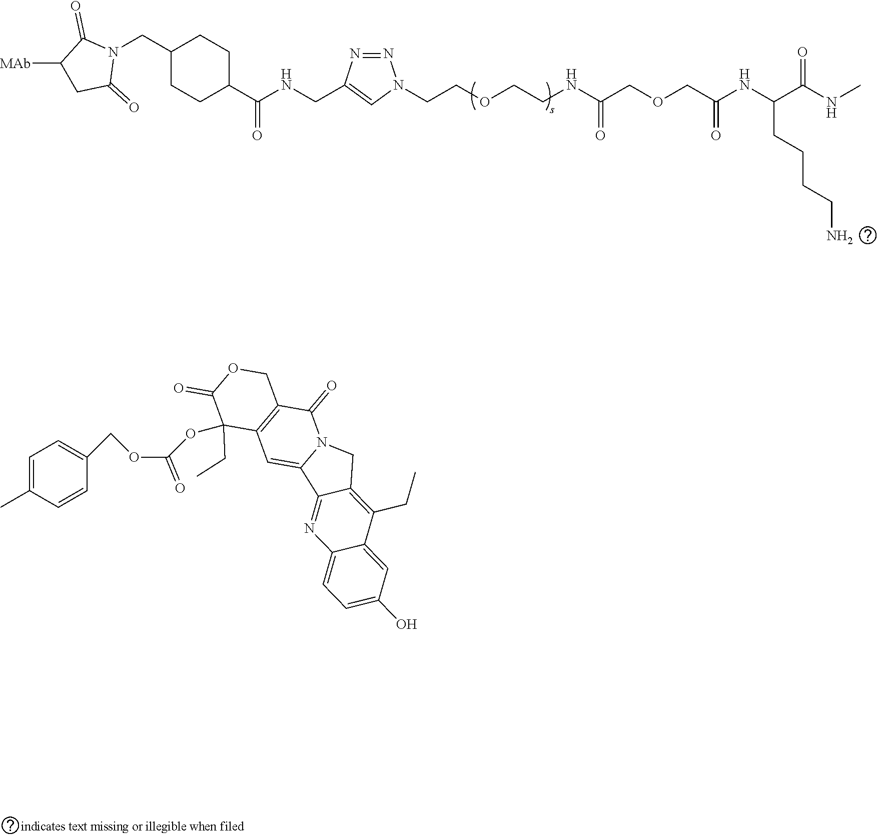

(described, e.g., in U.S. Pat. No. 7,999,083). In some embodiments, the TopI ADC comprises an antibody that binds a tumor antigen. In some embodiments, the tumor antigen is selected from carbonic anhydrase IX, B7, CCCL19, CCCL21, CSAp, HER-2/neu, BrE3, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD80, CD83, CD95, CD126, CD133, CD138, CD147, CD154, CEACAM5, CEACAM-6, alpha-fetoprotein (AFP), VEGF, ED-B fibronectin, EGP-1, EGP-2, EGF receptor (ErbB1), ErbB2, ErbB3, Factor H, FHL-1, Flt-3, folate receptor, Ga 733, GROB, HMGB-1, hypoxia inducible factor (HIF), HM1.24, insulin-like growth factor (ILGF), IFN-a, IFN-b, IL-g, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, HLA-DR, HLA-G, CD66a-d, MAGE, mCRP, MCP-1, MIP-1A, MIP-1B, macrophage migration-inhibitory factor (MIF), MUC1, MUC2, MUC3, MUC4, MUC5, placental growth factor (P1GF), PSA (prostate-specific antigen), PSMA, PSMA dimer, PAM4 antigen, NCA-95, NCA-90, A3, A33, Ep-CAM, KS-1, Le(y), mesothelin, S100, tenascin, TAC, Tn antigen, Thomas-Friedenreich antigens, tumor necrosis antigens, tumor angiogenesis antigens, TNF-a, TRAIL receptor (R1 and R2), VEGFR, RANTES, T101, cancer stem cell antigens, complement factors C3, C3a, C3b, C5a, C5, and an oncogene product. In some embodiments, the TopI ADC comprises an antibody selected from gemtuzumab, brentuximab, belantamab, camidanlumab, trastuzumab, inotuzumab, glembatumumab, anetumab, mirvetuximab, depatuxizumab, vadastuximab, labetuzumab, ladiratuzumab, loncastuximab, patritumab, lifastuzumab, indusatumab, polatuzumab, pinatuzumab, coltuximab, upifitamab, indatuximab, milatuzumab, rovalpituzumab, enfortumab, tisotumab, tusamitamab, disitamab, telisotuzumab, and antigen-binding fragments thereof. In some embodiments, the TopI ADC comprises an antibody selected from hLL1 (anti-CD74), hLL2 (anti-CD22), hRFB4 (anti-CD22), h PAM4 (anti-MUC5ac), hMN-3 (anti-NOTCH3), hMN-14 (labetuzumab; anti-CEACAM5); hMN15 (anti-CEACAM6) hA19 (anti-CD19), hA20 (anti-CD22), hMu-9 (anti-CSAp), hL243 (anti-HLA-DR), hImmu-31 (anti-AFP), and antigen-binding fragments thereof. In some embodiments, the method further comprises administering domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is (i) unresectable, locally advanced, (ii) metastatic, or (iii) muscle invasive. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally domvanalimab. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, zimberelimab, and optionally domvanalimab is administered in a neoadjuvant setting (e.g., in preparation for surgery or radiation therapy). In some embodiments, the combination of the ADC, zimberelimab, and optionally domvanalimab is administered in an adjuvant setting (e.g., following a primary treatment such as surgery or radiation therapy). In some embodiments, the combination of the ADC, zimberelimab, and optionally domvanalimab is administered in a therapeutic setting (e.g., as the primary therapy). In some embodiments, the combination of the ADC, zimberelimab, and optionally domvanalimab is administered in a maintenance setting. In some embodiments, the subject receives one or more doses of the ADC. In some embodiments, the subject receives one or more doses of zimberelimab. In some embodiments, the subject receives one or more doses of domvanalimab. In some embodiments, the ADC and zimberelimab are administered concurrently. In some embodiments, the ADC and zimberelimab are administered sequentially. In some embodiments, the ADC and domvanalimab are administered concurrently. In some embodiments, the ADC and domvanalimab are administered sequentially. In some embodiments, zimberelimab and domvanalimab are administered concurrently. In some embodiments, zimberelimab and domvanalimab are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating urothelial cancer comprising co-administering to a subject an effective amount of: (a) a tumor antigen-targeted antibody-drug conjugate (ADC) comprising a topoisomerase I inhibitor (TopI ADC); (b) zimberelimab; and, optionally, (c) domvanalimab. In some embodiments, the topoisomerase I inhibitor is a camptothecin. In some embodiments, the camptothecin is selected from irinotecan, topotecan, belotecan, and exatecan derivative. In some embodiments, the exatecan derivative is selected from Dxd or SN38. In some embodiments, the topoisomerase I inhibitor is selected from irinotecan, topetecan, and SN-38. In some embodiments, the TopI ADC has a structural formula of mAb-CL2A-SN-38, with a structure represented by:

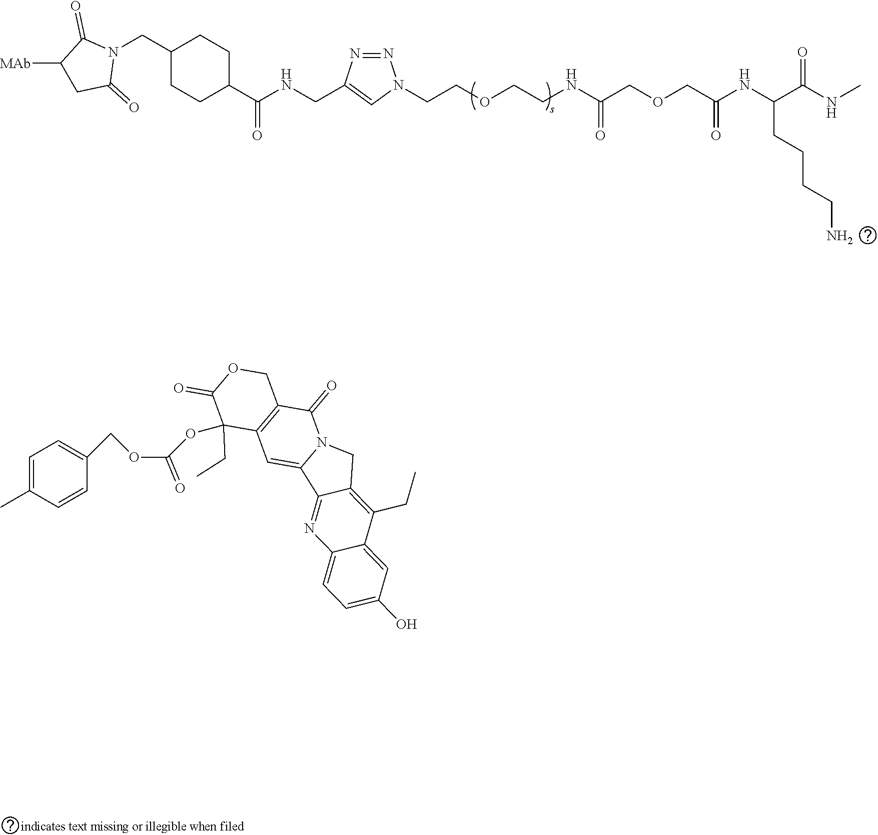

(described, e.g., in U.S. Pat. No. 7,999,083). In some embodiments, the TopI ADC comprises an antibody that binds a tumor antigen. In some embodiments, the tumor antigen is selected from carbonic anhydrase IX, B7, CCCL19, CCCL21, CSAp, HER-2/neu, BrE3, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD80, CD83, CD95, CD126, CD133, CD138, CD147, CD154, CEACAM5, CEACAM-6, alpha-fetoprotein (AFP), VEGF, ED-B fibronectin, EGP-1, EGP-2, EGF receptor (ErbB1), ErbB2, ErbB3, Factor H, FHL-1, Flt-3, folate receptor, Ga 733, GROB, HMGB-1, hypoxia inducible factor (HIF), HM1.24, insulin-like growth factor (ILGF), IFN-a, IFN-b, IL-g, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, HLA-DR, HLA-G, CD66a-d, MAGE, mCRP, MCP-1, MIP-1A, MIP-1B, macrophage migration-inhibitory factor (MIF), MUC1, MUC2, MUC3, MUC4, MUC5, placental growth factor (P1GF), PSA (prostate-specific antigen), PSMA, PSMA dimer, PAM4 antigen, NCA-95, NCA-90, A3, A33, Ep-CAM, KS-1, Le(y), mesothelin, S100, tenascin, TAC, Tn antigen, Thomas-Friedenreich antigens, tumor necrosis antigens, tumor angiogenesis antigens, TNF-a, TRAIL receptor (R1 and R2), VEGFR, RANTES, T101, cancer stem cell antigens, complement factors C3, C3a, C3b, C5a, C5, and an oncogene product. In some embodiments, the TopI ADC comprises an antibody selected from gemtuzumab, brentuximab, belantamab, camidanlumab, trastuzumab, inotuzumab, glembatumumab, anetumab, mirvetuximab, depatuxizumab, vadastuximab, labetuzumab, ladiratuzumab, loncastuximab, patritumab, lifastuzumab, indusatumab, polatuzumab, pinatuzumab, coltuximab, upifitamab, indatuximab, milatuzumab, rovalpituzumab, enfortumab, tisotumab, tusamitamab, disitamab, telisotuzumab, and antigen-binding fragments thereof. In some embodiments, the TopI ADC comprises an antibody selected from hLL1 (anti-CD74), hLL2 (anti-CD22), hRFB4 (anti-CD22), h PAM4 (anti-MUC5ac), hMN-3 (anti-NOTCH3), hMN-14 (labetuzumab; anti-CEACAM5); hMN15 (anti-CEACAM6) hA19 (anti-CD19), hA20 (anti-CD22), hMu-9 (anti-CSAp), hL243 (anti-HLA-DR), hImmu-31 (anti-AFP), and antigen-binding fragments thereof. In some embodiments, the method further comprises administering domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is (i) unresectable, locally advanced, (ii) metastatic, or (iii) muscle invasive. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally domvanalimab. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more anti-cancer therapies before administration of the combination of the ADC, zimberelimab, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, zimberelimab, and optionally domvanalimab is administered in a neoadjuvant setting (e.g., in preparation for surgery or radiation therapy). In some embodiments, the combination of the ADC, zimberelimab, and optionally domvanalimab is administered in an adjuvant setting (e.g., following a primary treatment such as surgery or radiation therapy). In some embodiments, the combination of the ADC, zimberelimab, and optionally domvanalimab is administered in a therapeutic setting (e.g., as the primary therapy). In some embodiments, the combination of the ADC, zimberelimab, and optionally domvanalimab is administered in a maintenance setting. In some embodiments, the subject receives one or more doses of the ADC. In some embodiments, the subject receives one or more doses of zimberelimab. In some embodiments, the subject receives one or more doses of domvanalimab. In some embodiments, the ADC and zimberelimab are administered concurrently. In some embodiments, the ADC and zimberelimab are administered sequentially. In some embodiments, the ADC and domvanalimab are administered concurrently. In some embodiments, the ADC and domvanalimab are administered sequentially. In some embodiments, zimberelimab and domvanalimab are administered concurrently. In some embodiments, zimberelimab and domvanalimab are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating, mitigating, reducing, preventing or delaying the recurrence or metastasis of urothelial cancer, comprising co-administering to a subject an effective amount of: (a) a tumor antigen-targeted antibody-drug conjugate (ADC) comprising a topoisomerase I inhibitor (TopI ADC); (b) an anti-PD-(L)1 antibody; and, optionally, (c) domvanalimab. In some embodiments, the topoisomerase I inhibitor is a camptothecin. In some embodiments, the camptothecin is selected from irinotecan, topotecan, belotecan, and exatecan derivative. In some embodiments, the exatecan derivative is selected from Dxd or SN38. In some embodiments, the topoisomerase I inhibitor is selected from irinotecan, topetecan, and SN-38. In some embodiments, the TopI ADC has a structural formula of mAb-CL2A-SN-38, with a structure represented by:

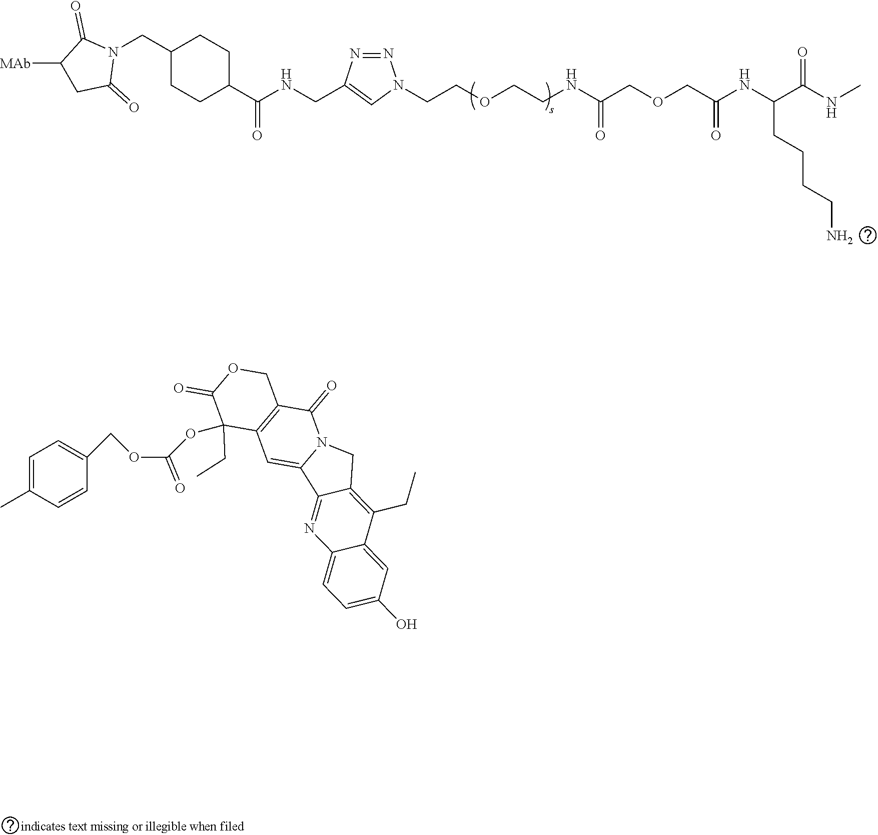

(described, e.g., in U.S. Pat. No. 7,999,083). In some embodiments, the TopI ADC comprises an antibody that binds a tumor antigen. In some embodiments, the tumor antigen is selected from carbonic anhydrase IX, B7, CCCL19, CCCL21, CSAp, HER-2/neu, BrE3, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD80, CD83, CD95, CD126, CD133, CD138, CD147, CD154, CEACAM5, CEACAM-6, alpha-fetoprotein (AFP), VEGF, ED-B fibronectin, EGP-1, EGP-2, EGF receptor (ErbB1), ErbB2, ErbB3, Factor H, FHL-1, Flt-3, folate receptor, Ga 733, GROB, HMGB-1, hypoxia inducible factor (HIF), HM1.24, insulin-like growth factor (ILGF), IFN-a, IFN-b, IL-g, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, 1a, HM1.24, gangliosides, HCG, HLA-DR, HLA-G, CD66a-d, MAGE, mCRP, MCP-1, MIP-1A, MIP-1B, macrophage migration-inhibitory factor (MIF), MUC1, MUC2, MUC3, MUC4, MUC5, placental growth factor (P1GF), PSA (prostate-specific antigen), PSMA, PSMA dimer, PAM4 antigen, NCA-95, NCA-90, A3, A33, Ep-CAM, KS-1, Le(y), mesothelin, S100, tenascin, TAC, Tn antigen, Thomas-Friedenreich antigens, tumor necrosis antigens, tumor angiogenesis antigens, TNF-a, TRAIL receptor (R1 and R2), VEGFR, RANTES, T101, cancer stem cell antigens, complement factors C3, C3a, C3b, C5a, C5, and an oncogene product. In some embodiments, the TopI ADC comprises an antibody selected from gemtuzumab, brentuximab, belantamab, camidanlumab, trastuzumab, inotuzumab, glembatumumab, anetumab, mirvetuximab, depatuxizumab, vadastuximab, labetuzumab, ladiratuzumab, loncastuximab, patritumab, lifastuzumab, indusatumab, polatuzumab, pinatuzumab, coltuximab, upifitamab, indatuximab, milatuzumab, rovalpituzumab, enfortumab, tisotumab, tusamitamab, disitamab, telisotuzumab, and antigen-binding fragments thereof. In some embodiments, the TopI ADC comprises an antibody selected from hLL1 (anti-CD74), hLL2 (anti-CD22), hRFB4 (anti-CD22), h PAM4 (anti-MUC5ac), hMN-3 (anti-NOTCH3), hMN-14 (labetuzumab; anti-CEACAM5); hMN15 (anti-CEACAM6) hA19 (anti-CD19), hA20 (anti-CD22), hMu-9 (anti-CSAp), hL243 (anti-HLA-DR), hImmu-31 (anti-AFP), and antigen-binding fragments thereof. In some embodiments, the anti-PD-(L)1 antibody is pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, or zimberelimab. In some embodiments, the anti-PD-(L)1 antibody is zimberelimab. In some embodiments, the anti-PD-(L)1 antibody is durvalumab. In some embodiments, the anti-PD-(L)1 antibody is an Fc-silent antibody. In some embodiments, the anti-PD-(L)1 antibody is an Fc-enabled antibody. In some embodiments, the method further comprises administering domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is (i) unresectable, locally advanced, (ii) metastatic, or (iii) muscle invasive. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of the ADC, anti-PD-(L)1 antibody, and optionally domvanalimab. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more anti-cancer therapies before administration of the combination of the ADC, anti-PD-(L)1 antibody, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, anti-PD-(L)1 antibody, and optionally domvanalimab is administered in a neoadjuvant setting (e.g., in preparation for surgery or radiation therapy). In some embodiments, the combination of the ADC, anti-PD-(L)1 antibody, and optionally domvanalimab is administered in an adjuvant setting (e.g., following a primary treatment such as surgery or radiation therapy). In some embodiments, the combination of the ADC, anti-PD-(L)1 antibody, and optionally domvanalimab is administered in a therapeutic setting (e.g., as the primary therapy). In some embodiments, the combination of the ADC, anti-PD-(L)1 antibody, and optionally domvanalimab is administered in a maintenance setting. In some embodiments, the subject receives one or more doses of the ADC. In some embodiments, the subject receives one or more doses of the anti-PD-(L)1 antibody. In some embodiments, the subject receives one or more doses of domvanalimab. In some embodiments, the ADC and the anti-PD-(L)1 antibody are administered concurrently. In some embodiments, the ADC and the anti-PD-(L)1 antibody are administered sequentially. In some embodiments, the ADC and domvanalimab are administered concurrently. In some embodiments, the ADC and domvanalimab are administered sequentially. In some embodiments, the anti-PD-(L)1 antibody and domvanalimab are administered concurrently. In some embodiments, the anti-PD-(L)1 antibody and domvanalimab are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, the anti-PD-(L)1 antibody is administered at one or more doses in the range of 300 mg to 400 mg. In some embodiments, the anti-PD-(L)1 antibody is administered at dose of 360 mg. In some embodiments, the anti-PD-(L)1 antibody is administered intravenously. In some embodiments, the anti-PD-(L)1 antibody is administered on day 1 of a 21-day cycle. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, 5-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating urothelial cancer comprising co-administering to a subject an effective amount of: (a) a tumor antigen-targeted antibody-drug conjugate (ADC) comprising a topoisomerase I inhibitor (TopI ADC); (b) an anti-PD-(L)1 antibody; and, optionally, (c) domvanalimab. In some embodiments, the topoisomerase I inhibitor is a camptothecin. In some embodiments, the camptothecin is selected from irinotecan, topotecan, belotecan, and exatecan derivative. In some embodiments, the exatecan derivative is selected from Dxd or SN38. In some embodiments, the topoisomerase I inhibitor is selected from irinotecan, topetecan, and SN-38. In some embodiments, the TopI ADC has a structural formula of mAb-CL2A-SN-38, with a structure represented by:

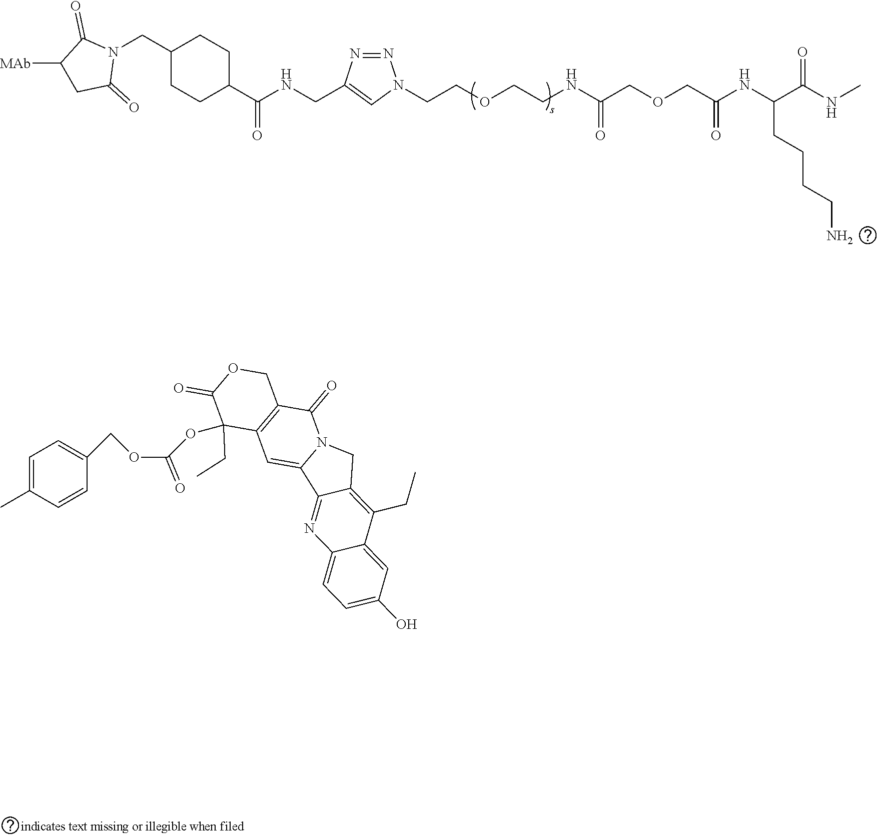

(described, e.g., in U.S. Pat. No. 7,999,083). In some embodiments, the TopI ADC comprises an antibody that binds a tumor antigen. In some embodiments, the tumor antigen is selected from carbonic anhydrase IX, B7, CCCL19, CCCL21, CSAp, HER-2/neu, BrE3, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD80, CD83, CD95, CD126, CD133, CD138, CD147, CD154, CEACAM5, CEACAM-6, alpha-fetoprotein (AFP), VEGF, ED-B fibronectin, EGP-1, EGP-2, EGF receptor (ErbB1), ErbB2, ErbB3, Factor H, FHL-1, Flt-3, folate receptor, Ga 733, GROB, HMGB-1, hypoxia inducible factor (HIF), HM1.24, insulin-like growth factor (ILGF), IFN-a, IFN-b, IL-g, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, HLA-DR, HLA-G, CD66a-d, MAGE, mCRP, MCP-1, MIP-1A, MIP-1B, macrophage migration-inhibitory factor (MIF), MUC1, MUC2, MUC3, MUC4, MUC5, placental growth factor (P1GF), PSA (prostate-specific antigen), PSMA, PSMA dimer, PAM4 antigen, NCA-95, NCA-90, A3, A33, Ep-CAM, KS-1, Le(y), mesothelin, S100, tenascin, TAC, Tn antigen, Thomas-Friedenreich antigens, tumor necrosis antigens, tumor angiogenesis antigens, TNF-a, TRAIL receptor (R1 and R2), VEGFR, RANTES, T101, cancer stem cell antigens, complement factors C3, C3a, C3b, C5a, C5, and an oncogene product. In some embodiments, the TopI ADC comprises an antibody selected from gemtuzumab, brentuximab, belantamab, camidanlumab, trastuzumab, inotuzumab, glembatumumab, anetumab, mirvetuximab, depatuxizumab, vadastuximab, labetuzumab, ladiratuzumab, loncastuximab, patritumab, lifastuzumab, indusatumab, polatuzumab, pinatuzumab, coltuximab, upifitamab, indatuximab, milatuzumab, rovalpituzumab, enfortumab, tisotumab, tusamitamab, disitamab, telisotuzumab, and antigen-binding fragments thereof. In some embodiments, the TopI ADC comprises an antibody selected from hLL1 (anti-CD74), hLL2 (anti-CD22), hRFB4 (anti-CD22), h PAM4 (anti-MUC5ac), hMN-3 (anti-NOTCH3), hMN-14 (labetuzumab; anti-CEACAM5); hMN15 (anti-CEACAM6) hA19 (anti-CD19), hA20 (anti-CD22), hMu-9 (anti-CSAp), hL243 (anti-HLA-DR), hImmu-31 (anti-AFP), and antigen-binding fragments thereof. In some embodiments, the anti-PD-(L)1 antibody is pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, or zimberelimab. In some embodiments, the anti-PD-(L)1 antibody is zimberelimab. In some embodiments, the anti-PD-(L)1 antibody is durvalumab. In some embodiments, the anti-PD-(L)1 antibody is an Fc-silent antibody. In some embodiments, the anti-PD-(L)1 antibody is an Fc-enabled antibody. In some embodiments, the method further comprises administering domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is (i) unresectable, locally advanced, (ii) metastatic, or (iii) muscle invasive. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of the ADC, anti-PD-(L)1 antibody, and optionally domvanalimab. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more anti-cancer therapies before administration of the combination of the ADC, anti-PD-(L)1 antibody, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of the ADC, anti-PD-(L)1 antibody, and optionally domvanalimab is administered in a neoadjuvant setting (e.g., in preparation for surgery or radiation therapy). In some embodiments, the combination of the ADC, anti-PD-(L)1 antibody, and optionally domvanalimab is administered in an adjuvant setting (e.g., following a primary treatment such as surgery or radiation therapy). In some embodiments, the combination of the ADC, anti-PD-(L)1 antibody, and optionally domvanalimab is administered in a therapeutic setting (e.g., as the primary therapy). In some embodiments, the combination of the ADC, anti-PD-(L)1 antibody, and optionally domvanalimab is administered in a maintenance setting. In some embodiments, the subject receives one or more doses of the ADC. In some embodiments, the subject receives one or more doses of the anti-PD-(L)1 antibody. In some embodiments, the subject receives one or more doses of domvanalimab. In some embodiments, the ADC and the anti-PD-(L)1 antibody are administered concurrently. In some embodiments, the ADC and the anti-PD-(L)1 antibody are administered sequentially. In some embodiments, the ADC and domvanalimab are administered concurrently. In some embodiments, the ADC and domvanalimab are administered sequentially. In some embodiments, the anti-PD-(L)1 antibody and domvanalimab are administered concurrently. In some embodiments, the anti-PD-(L)1 antibody and domvanalimab are administered sequentially. In some embodiments, the subject is human. In some embodiments, the subject is cisplatin-ineligible. In some embodiments, the ADC is administered at one or more doses in the range of 8 mg/kg to 10 mg/kg. In some embodiments, the ADC is administered at one or more doses of 8 mg/kg or 10 mg/kg. In some embodiments, the ADC is administered at one or more doses of 10 mg/kg. In some embodiments, the ADC is administered intravenously. In some embodiments, the ADC is administered on days 1 and 8 of a 21-day cycle. In some embodiments, the anti-PD-(L)1 antibody is administered at one or more doses in the range of 300 mg to 400 mg. In some embodiments, the anti-PD-(L)1 antibody is administered at dose of 360 mg. In some embodiments, the anti-PD-(L)1 antibody is administered intravenously. In some embodiments, the anti-PD-(L)1 antibody is administered on day 1 of a 21-day cycle. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ft13L, and SYM-027. In some embodiments, the FLT3 agonist is GS-3583.

In some embodiments, provided herein are methods of treating, mitigating, reducing, preventing or delaying the recurrence or metastasis of a Trop-2 positive cancer comprising co-administering to a human patient an effective amount of a) sacituzumab govitecan; b) zimberelimab; and, optionally, c) domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer. In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or unresectable locally advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the treatment naïve subject has not received prior anti-cancer therapy for (i) advanced cancer or (ii) metastatic cancer. In some embodiments, the subject has received one or more anti-cancer therapies before administration of the combination of sacituzumab govitecan, zimberelimab, and optionally domvanalimab. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more prior anti-cancer therapies for (i) advanced cancer or (ii) metastatic cancer. In some embodiments, the subject has received one or more anti-cancer therapies before administration of the combination of sacituzumab govitecan, zimberelimab, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of sacituzumab govitecan, zimberelimab, and optionally domvanalimab is administered in a neoadjuvant setting (e.g., in preparation for surgery or radiation therapy). In some embodiments, the combination of sacituzumab govitecan, zimberelimab, and optionally domvanalimab is administered in an adjuvant setting (e.g., following a primary treatment such as surgery or radiation therapy). In some embodiments, the combination of sacituzumab govitecan, zimberelimab, and optionally domvanalimab is administered in a therapeutic setting (e.g., as the primary therapy). In some embodiments, the combination of sacituzumab govitecan, zimberelimab, and optionally domvanalimab is administered in a maintenance setting. In some embodiments, the subject receives one or more doses of sacituzumab govitecan. In some embodiments, the subject receives one or more doses of the zimberelimab. In some embodiments, the subject receives one or more doses of the domvanalimab. In some embodiments, sacituzumab govitecan and the zimberelimab are administered concurrently. In some embodiments, sacituzumab govitecan and the zimberelimab are administered sequentially. In some embodiments, sacituzumab govitecan and the domvanalimab are administered concurrently. In some embodiments, sacituzumab govitecan and the domvanalimab are administered sequentially. In some embodiments, the zimberelimab and the domvanalimab are administered concurrently. In some embodiments, the zimberelimab and the domvanalimab are administered sequentially. In some embodiments, the subject is human. In some embodiments, the subject is cisplatin-ineligible. In some embodiments, sacituzumab govitecan is administered at one or more doses in the range of 8 mg/kg to 10 mg/kg. In some embodiments, sacituzumab govitecan is administered at one or more doses of 10 mg/kg. In some embodiments, sacituzumab govitecan is administered intravenously. In some embodiments, sacituzumab govitecan is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, the method further comprises administering domvanalimab. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle, and domvanalimab is administered at a dose of 1200 mg on day 1 of the 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating a Trop-2 positive cancer comprising co-administering to a human patient an effective amount of a) sacituzumab govitecan; b) zimberelimab; and, optionally, c) domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer. In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or unresectable locally advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the treatment naïve subject has not received prior anti-cancer therapy for (i) advanced cancer or (ii) metastatic cancer. In some embodiments, the subject has received one or more anti-cancer therapies before administration of the combination of sacituzumab govitecan, zimberelimab, and optionally domvanalimab. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more prior anti-cancer therapies for (i) advanced cancer or (ii) metastatic cancer. In some embodiments, the subject has received one or more anti-cancer therapies before administration of the combination of sacituzumab govitecan, zimberelimab, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of sacituzumab govitecan, zimberelimab, and optionally domvanalimab is administered in a neoadjuvant setting (e.g., in preparation for surgery or radiation therapy). In some embodiments, the combination of sacituzumab govitecan, zimberelimab, and optionally domvanalimab is administered in an adjuvant setting (e.g., following a primary treatment such as surgery or radiation therapy). In some embodiments, the combination of sacituzumab govitecan, zimberelimab, and optionally domvanalimab is administered in a therapeutic setting (e.g., as the primary therapy). In some embodiments, the combination of sacituzumab govitecan, zimberelimab, and optionally domvanalimab is administered in a maintenance setting. In some embodiments, the subject receives one or more doses of sacituzumab govitecan. In some embodiments, the subject receives one or more doses of the zimberelimab. In some embodiments, the subject receives one or more doses of the domvanalimab. In some embodiments, sacituzumab govitecan and the zimberelimab are administered concurrently. In some embodiments, sacituzumab govitecan and the zimberelimab are administered sequentially. In some embodiments, sacituzumab govitecan and the domvanalimab are administered concurrently. In some embodiments, sacituzumab govitecan and the domvanalimab are administered sequentially. In some embodiments, the zimberelimab and the domvanalimab are administered concurrently. In some embodiments, the zimberelimab and the domvanalimab are administered sequentially. In some embodiments, the subject is human. In some embodiments, the subject is cisplatin-ineligible. In some embodiments, sacituzumab govitecan is administered at one or more doses in the range of 8 mg/kg to 10 mg/kg. In some embodiments, sacituzumab govitecan is administered at one or more doses of 10 mg/kg. In some embodiments, sacituzumab govitecan is administered intravenously. In some embodiments, sacituzumab govitecan is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, the method further comprises administering domvanalimab. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle, and domvanalimab is administered at a dose of 1200 mg on day 1 of the 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating, mitigating, reducing, preventing or delaying the recurrence or metastasis of a tumor antigen positive (TA⁺) cancer comprising co-administering to a human patient an effective amount of a) sacituzumab govitecan; b) zimberelimab; and, optionally, c) domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer. In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or unresectable locally advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the treatment naïve subject has not received prior anti-cancer therapy for (i) advanced cancer or (ii) metastatic cancer. In some embodiments, the subject has received one or more anti-cancer therapies before administration of the combination of sacituzumab govitecan, zimberelimab, and optionally domvanalimab. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more prior anti-cancer therapies for (i) advanced cancer or (ii) metastatic cancer. In some embodiments, the subject has received one or more anti-cancer therapies before administration of the combination of sacituzumab govitecan, zimberelimab, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of sacituzumab govitecan, zimberelimab, and optionally domvanalimab is administered in a neoadjuvant setting (e.g., in preparation for surgery or radiation therapy). In some embodiments, the combination of sacituzumab govitecan, zimberelimab, and optionally domvanalimab is administered in an adjuvant setting (e.g., following a primary treatment such as surgery or radiation therapy). In some embodiments, the combination of sacituzumab govitecan, zimberelimab, and optionally domvanalimab is administered in a therapeutic setting (e.g., as the primary therapy). In some embodiments, the combination of sacituzumab govitecan, zimberelimab, and optionally domvanalimab is administered in a maintenance setting. In some embodiments, the subject receives one or more doses of sacituzumab govitecan. In some embodiments, the subject receives one or more doses of the zimberelimab. In some embodiments, the subject receives one or more doses of the domvanalimab. In some embodiments, sacituzumab govitecan and the zimberelimab are administered concurrently. In some embodiments, sacituzumab govitecan and the zimberelimab are administered sequentially. In some embodiments, sacituzumab govitecan and the domvanalimab are administered concurrently. In some embodiments, sacituzumab govitecan and the domvanalimab are administered sequentially. In some embodiments, the zimberelimab and the domvanalimab are administered concurrently. In some embodiments, the zimberelimab and the domvanalimab are administered sequentially. In some embodiments, the subject is human. In some embodiments, the subject is cisplatin-ineligible. In some embodiments, sacituzumab govitecan is administered at one or more doses in the range of 8 mg/kg to 10 mg/kg. In some embodiments, sacituzumab govitecan is administered at one or more doses of 10 mg/kg. In some embodiments, sacituzumab govitecan is administered intravenously. In some embodiments, sacituzumab govitecan is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, the method further comprises administering domvanalimab. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle, and domvanalimab is administered at a dose of 1200 mg on day 1 of the 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating a tumor antigen positive (TA⁺) cancer comprising co-administering to a human patient an effective amount of a) sacituzumab govitecan; b) zimberelimab; and, optionally, c) domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is a solid epithelial cancer. In some embodiments, the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer. In some embodiments, the bladder cancer is urothelial cancer (UC). In some embodiments, the bladder cancer is (i) unresectable, locally advanced bladder cancer, (ii) metastatic bladder cancer, or (iii) muscle-invasive bladder cancer. In some embodiments, the urothelial cancer is (i) unresectable, locally advanced urothelial cancer, (ii) metastatic urothelial cancer, or (iii) muscle-invasive urothelial cancer. In some embodiments, the lung cancer is non-small cell lung cancer (NSCLC). In some embodiments, the lung cancer is (i) advanced lung cancer or (ii) metastatic lung cancer. In some embodiments, the lung cancer is (i) advanced NSCLC or (ii) metastatic NSCLC. In some embodiments, the NSCLC is squamous NSCLC. In some embodiments, the NSCLC is non-squamous NSCLC. In some embodiments, the metastatic NSCLC is metastatic squamous NSCLC. In some embodiments, the metastatic NSCLC is metastatic non-squamous NSCLC. In some embodiments, the NSCLC is NSCLC without EGFR, ALK, or other actionable genomic alterations. In some embodiments, the cancer is (i) unresectable, locally advanced, (ii) metastatic, or (iii) muscle invasive. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the subject is treatment naïve in the metastatic or unresectable locally advanced setting (e.g., the subject has not received treatment for metastatic or advanced lung cancer or NSCLC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the treatment naïve subject has not received prior anti-cancer therapy for (i) advanced cancer or (ii) metastatic cancer. In some embodiments, the subject has received one or more anti-cancer therapies before administration of the combination of sacituzumab govitecan, zimberelimab, and optionally domvanalimab. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more prior anti-cancer therapies for (i) advanced cancer or (ii) metastatic cancer. In some embodiments, the subject has received one or more anti-cancer therapies before administration of the combination of sacituzumab govitecan, zimberelimab, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of sacituzumab govitecan, zimberelimab, and optionally domvanalimab is administered in a neoadjuvant setting (e.g., in preparation for surgery or radiation therapy). In some embodiments, the combination of sacituzumab govitecan, zimberelimab, and optionally domvanalimab is administered in an adjuvant setting (e.g., following a primary treatment such as surgery or radiation therapy). In some embodiments, the combination of sacituzumab govitecan, zimberelimab, and optionally domvanalimab is administered in a therapeutic setting (e.g., as the primary therapy). In some embodiments, the combination of sacituzumab govitecan, zimberelimab, and optionally domvanalimab is administered in a maintenance setting. In some embodiments, the subject receives one or more doses of sacituzumab govitecan. In some embodiments, the subject receives one or more doses of the zimberelimab. In some embodiments, the subject receives one or more doses of the domvanalimab. In some embodiments, sacituzumab govitecan and the zimberelimab are administered concurrently. In some embodiments, sacituzumab govitecan and the zimberelimab are administered sequentially. In some embodiments, sacituzumab govitecan and the domvanalimab are administered concurrently. In some embodiments, sacituzumab govitecan and the domvanalimab are administered sequentially. In some embodiments, the zimberelimab and the domvanalimab are administered concurrently. In some embodiments, the zimberelimab and the domvanalimab are administered sequentially. In some embodiments, the subject is human. In some embodiments, the subject is cisplatin-ineligible. In some embodiments, sacituzumab govitecan is administered at one or more doses in the range of 8 mg/kg to 10 mg/kg. In some embodiments, sacituzumab govitecan is administered at one or more doses of 10 mg/kg. In some embodiments, sacituzumab govitecan is administered intravenously. In some embodiments, sacituzumab govitecan is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, the method further comprises administering domvanalimab. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle, and domvanalimab is administered at a dose of 1200 mg on day 1 of the 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating, mitigating, reducing, preventing or delaying the recurrence or metastasis of urothelial cancer (UC) comprising co-administering to a human patient an effective amount of a) sacituzumab govitecan; b) zimberelimab; and, optionally, c) domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is (i) unresectable, locally advanced, (ii) metastatic, or (iii) muscle invasive. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of sacituzumab govitecan, zimberelimab, and optionally domvanalimab. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more anti-cancer therapies before administration of the combination of sacituzumab govitecan, zimberelimab, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of sacituzumab govitecan, zimberelimab, and optionally domvanalimab is administered in a neoadjuvant setting (e.g., in preparation for surgery or radiation therapy). In some embodiments, the combination of sacituzumab govitecan, zimberelimab, and optionally domvanalimab is administered in an adjuvant setting (e.g., following a primary treatment such as surgery or radiation therapy). In some embodiments, the combination of sacituzumab govitecan, zimberelimab, and optionally domvanalimab is administered in a therapeutic setting (e.g., as the primary therapy). In some embodiments, the combination of sacituzumab govitecan, zimberelimab, and optionally domvanalimab is administered in a maintenance setting. In some embodiments, the subject receives one or more doses of sacituzumab govitecan. In some embodiments, the subject receives one or more doses of the zimberelimab. In some embodiments, the subject receives one or more doses of the domvanalimab. In some embodiments, sacituzumab govitecan and the zimberelimab are administered concurrently. In some embodiments, sacituzumab govitecan and the zimberelimab are administered sequentially. In some embodiments, sacituzumab govitecan and the domvanalimab are administered concurrently. In some embodiments, sacituzumab govitecan and the domvanalimab are administered sequentially. In some embodiments, the zimberelimab and the domvanalimab are administered concurrently. In some embodiments, the zimberelimab and the domvanalimab are administered sequentially. In some embodiments, the subject is human. In some embodiments, the subject is cisplatin-ineligible. In some embodiments, sacituzumab govitecan is administered at one or more doses in the range of 8 mg/kg to 10 mg/kg. In some embodiments, sacituzumab govitecan is administered at one or more doses of 10 mg/kg. In some embodiments, sacituzumab govitecan is administered intravenously. In some embodiments, sacituzumab govitecan is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, the method further comprises administering domvanalimab. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle, and domvanalimab is administered at a dose of 1200 mg on day 1 of the 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating urothelial cancer (UC) comprising co-administering to a human patient an effective amount of a) sacituzumab govitecan; b) zimberelimab; and, optionally, c) domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is (i) unresectable, locally advanced, (ii) metastatic, or (iii) muscle invasive. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of sacituzumab govitecan, zimberelimab, and optionally domvanalimab. In some embodiments, the subject is not treatment naïve. In some embodiments, the subject has received one or more prior anti-cancer therapies for (i) unresectable, locally advanced cancer, (ii) metastatic cancer, or (iii) muscle invasive cancer. In some embodiments, the subject has received one or more anti-cancer therapies before administration of the combination of sacituzumab govitecan, zimberelimab, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of sacituzumab govitecan, zimberelimab, and optionally domvanalimab is administered in a neoadjuvant setting (e.g., in preparation for surgery or radiation therapy). In some embodiments, the combination of sacituzumab govitecan, zimberelimab, and optionally domvanalimab is administered in an adjuvant setting (e.g., following a primary treatment such as surgery or radiation therapy). In some embodiments, the combination of sacituzumab govitecan, zimberelimab, and optionally domvanalimab is administered in a therapeutic setting (e.g., as the primary therapy). In some embodiments, the combination of sacituzumab govitecan, zimberelimab, and optionally domvanalimab is administered in a maintenance setting. In some embodiments, the subject receives one or more doses of sacituzumab govitecan. In some embodiments, the subject receives one or more doses of the zimberelimab. In some embodiments, the subject receives one or more doses of the domvanalimab. In some embodiments, sacituzumab govitecan and the zimberelimab are administered concurrently. In some embodiments, sacituzumab govitecan and the zimberelimab are administered sequentially. In some embodiments, sacituzumab govitecan and the domvanalimab are administered concurrently. In some embodiments, sacituzumab govitecan and the domvanalimab are administered sequentially. In some embodiments, the zimberelimab and the domvanalimab are administered concurrently. In some embodiments, the zimberelimab and the domvanalimab are administered sequentially. In some embodiments, the subject is human. In some embodiments, the subject is cisplatin-ineligible. In some embodiments, sacituzumab govitecan is administered at one or more doses in the range of 8 mg/kg to 10 mg/kg. In some embodiments, sacituzumab govitecan is administered at one or more doses of 10 mg/kg. In some embodiments, sacituzumab govitecan is administered intravenously. In some embodiments, sacituzumab govitecan is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, the method further comprises administering domvanalimab. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle, and domvanalimab is administered at a dose of 1200 mg on day 1 of the 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ft13L, and SYM-027. In some embodiments, the FLT3 agonist is GS-3583.

In some embodiments, provided herein are methods of treating, mitigating, reducing, preventing or delaying the recurrence of metastatic urothelial cancer (mUC) comprising co-administering to a human patient an effective amount of a) sacituzumab govitecan; b) zimberelimab; and, optionally, c) domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is (i) unresectable, locally advanced, (ii) metastatic, or (iii) muscle invasive. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of sacituzumab govitecan, zimberelimab, and optionally domvanalimab. In some embodiments, the subject is not treatment naïve. In some embodiments, the subject has received one or more anti-cancer therapies before administration of the combination of sacituzumab govitecan, zimberelimab, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of sacituzumab govitecan, zimberelimab, and optionally domvanalimab is administered in a neoadjuvant setting (e.g., in preparation for surgery or radiation therapy). In some embodiments, the combination of sacituzumab govitecan, zimberelimab, and optionally domvanalimab is administered in an adjuvant setting (e.g., following a primary treatment such as surgery or radiation therapy). In some embodiments, the combination of sacituzumab govitecan, zimberelimab, and optionally domvanalimab is administered in a therapeutic setting (e.g., as the primary therapy). In some embodiments, the combination of sacituzumab govitecan, zimberelimab, and optionally domvanalimab is administered in a maintenance setting. In some embodiments, the subject receives one or more doses of sacituzumab govitecan. In some embodiments, the subject receives one or more doses of the zimberelimab. In some embodiments, the subject receives one or more doses of the domvanalimab. In some embodiments, sacituzumab govitecan and the zimberelimab are administered concurrently. In some embodiments, sacituzumab govitecan and the zimberelimab are administered sequentially. In some embodiments, sacituzumab govitecan and the domvanalimab are administered concurrently. In some embodiments, sacituzumab govitecan and the domvanalimab are administered sequentially. In some embodiments, the zimberelimab and the domvanalimab are administered concurrently. In some embodiments, the zimberelimab and the domvanalimab are administered sequentially. In some embodiments, the subject is human. In some embodiments, the subject is cisplatin-ineligible. In some embodiments, sacituzumab govitecan is administered at one or more doses in the range of 8 mg/kg to 10 mg/kg. In some embodiments, sacituzumab govitecan is administered at one or more doses of 10 mg/kg. In some embodiments, sacituzumab govitecan is administered intravenously. In some embodiments, sacituzumab govitecan is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, the method further comprises administering domvanalimab. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle, and domvanalimab is administered at a dose of 1200 mg on day 1 of the 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating metastatic urothelial cancer (mUC) comprising co-administering to a human patient an effective amount of a) sacituzumab govitecan; b) zimberelimab; and, optionally, c) domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is (i) unresectable, locally advanced, (ii) metastatic, or (iii) muscle invasive. In some embodiments, the cancer has progressed following at least one prior anti-cancer therapy. In some embodiments, the subject is treatment naïve. In some embodiments, the subject is treatment naïve in the metastatic or unresectable locally advanced setting (e.g., the subject has not received treatment for mUC or unresectable locally advanced UC). In some embodiments, the human patient is cisplatin ineligible and treatment naïve in the metastatic or unresectable locally advanced setting. In some embodiments, the treatment naïve subject has not received prior anti-cancer therapy for (i) unresectable, locally advanced cancer, (ii) metastatic cancer, or (iii) muscle invasive cancer. In some embodiments, the subject has received one or more anti-cancer therapies before administration of the combination of sacituzumab govitecan, zimberelimab, and optionally domvanalimab. In some embodiments, the subject is not treatment naïve. In some embodiments, the subject has received one or more anti-cancer therapies before administration of the combination of sacituzumab govitecan, zimberelimab, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of sacituzumab govitecan, zimberelimab, and optionally domvanalimab is administered in a neoadjuvant setting (e.g., in preparation for surgery or radiation therapy). In some embodiments, the combination of sacituzumab govitecan, zimberelimab, and optionally domvanalimab is administered in an adjuvant setting (e.g., following a primary treatment such as surgery or radiation therapy). In some embodiments, the combination of sacituzumab govitecan, zimberelimab, and optionally domvanalimab is administered in a therapeutic setting (e.g., as the primary therapy). In some embodiments, the combination of sacituzumab govitecan, zimberelimab, and optionally domvanalimab is administered in a maintenance setting. In some embodiments, the subject receives one or more doses of sacituzumab govitecan. In some embodiments, the subject receives one or more doses of the zimberelimab. In some embodiments, the subject receives one or more doses of the domvanalimab. In some embodiments, sacituzumab govitecan and the zimberelimab are administered concurrently. In some embodiments, sacituzumab govitecan and the zimberelimab are administered sequentially. In some embodiments, sacituzumab govitecan and the domvanalimab are administered concurrently. In some embodiments, sacituzumab govitecan and the domvanalimab are administered sequentially. In some embodiments, the zimberelimab and the domvanalimab are administered concurrently. In some embodiments, the zimberelimab and the domvanalimab are administered sequentially. In some embodiments, the subject is human. In some embodiments, the subject is cisplatin-ineligible. In some embodiments, sacituzumab govitecan is administered at one or more doses in the range of 8 mg/kg to 10 mg/kg. In some embodiments, sacituzumab govitecan is administered at one or more doses of 10 mg/kg. In some embodiments, sacituzumab govitecan is administered intravenously. In some embodiments, sacituzumab govitecan is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, the method further comprises administering domvanalimab. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle, and domvanalimab is administered at a dose of 1200 mg on day 1 of the 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ft13L, and SYM-027. In some embodiments, the FLT3 agonist is GS-3583.

In some embodiments, provided herein are methods of treating, mitigating, reducing, preventing or delaying the recurrence of non-small cell lung cancer (NSCLC) comprising co-administering to a human patient an effective amount of a) sacituzumab govitecan; b) zimberelimab; and c) domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or advanced setting (e.g., the subject has not received treatment for metastatic NSCLC or advanced NSCLC). In some embodiments, the treatment naïve subject has not received prior anti-cancer therapy for (i) advanced cancer or (ii) metastatic cancer. In some embodiments, the subject has received one or more anti-cancer therapies before administration of the combination of sacituzumab govitecan, zimberelimab, and domvanalimab. In some embodiments, the subject is not treatment naïve. In some embodiments, the subject has received one or more anti-cancer therapies before administration of the combination of sacituzumab govitecan, zimberelimab, and domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of sacituzumab govitecan, zimberelimab, and domvanalimab is administered in a neoadjuvant setting (e.g., in preparation for surgery or radiation therapy). In some embodiments, the combination of sacituzumab govitecan, zimberelimab, and domvanalimab is administered in an adjuvant setting (e.g., following aprimary treatment such as surgery or radiation therapy). In some embodiments, the combination of sacituzumab govitecan, zimberelimab, and domvanalimab is administered in a therapeutic setting (e.g., as the primary therapy). In some embodiments, the combination of sacituzumab govitecan, zimberelimab, and domvanalimab is administered in a maintenance setting. In some embodiments, the subject receives one or more doses of sacituzumab govitecan. In some embodiments, the subject receives one or more doses of the zimberelimab. In some embodiments, the subject receives one or more doses of the domvanalimab. In some embodiments, sacituzumab govitecan and the zimberelimab are administered concurrently. In some embodiments, sacituzumab govitecan and the zimberelimab are administered sequentially. In some embodiments, sacituzumab govitecan and the domvanalimab are administered concurrently. In some embodiments, sacituzumab govitecan and the domvanalimab are administered sequentially. In some embodiments, the zimberelimab and the domvanalimab are administered concurrently. In some embodiments, the zimberelimab and the domvanalimab are administered sequentially. In some embodiments, the subject is human. In some embodiments, sacituzumab govitecan is administered at one or more doses in the range of 8 mg/kg to 10 mg/kg. In some embodiments, sacituzumab govitecan is administered at one or more doses of 10 mg/kg. In some embodiments, sacituzumab govitecan is administered intravenously. In some embodiments, sacituzumab govitecan is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, the method further comprises administering domvanalimab. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle, and domvanalimab is administered at a dose of 1200 mg on day 1 of the 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating non-small cell lung cancer (NSCLC) comprising co-administering to a human patient an effective amount of a) sacituzumab govitecan; b) zimberelimab; and c) domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the cancer is (i) advanced or (ii) metastatic. In some embodiments, the cancer has progressed following at least one prior anti-cancer therapy. In some embodiments, the subject is treatment naïve. In some embodiments, the subject is treatment naïve in the metastatic or advanced setting (e.g., the subject has not received treatment for metastatic NSCLC or advanced NSCLC). In some embodiments, the treatment naïve subject has not received prior anti-cancer therapy for (i) advanced cancer or (ii) metastatic cancer. In some embodiments, the subject has received one or more anti-cancer therapies before administration of the combination of sacituzumab govitecan, zimberelimab, and domvanalimab. In some embodiments, the subject is not treatment naïve. In some embodiments, the subject has received one or more anti-cancer therapies before administration of the combination of sacituzumab govitecan, zimberelimab, and domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of sacituzumab govitecan, zimberelimab, and domvanalimab is administered in a neoadjuvant setting (e.g., in preparation for surgery or radiation therapy). In some embodiments, the combination of sacituzumab govitecan, zimberelimab, and domvanalimab is administered in an adjuvant setting (e.g., following a primary treatment such as surgery or radiation therapy). In some embodiments, the combination of sacituzumab govitecan, zimberelimab, and domvanalimab is administered in a therapeutic setting (e.g., as the primary therapy). In some embodiments, the combination of sacituzumab govitecan, zimberelimab, and domvanalimab is administered in a maintenance setting. In some embodiments, the subject receives one or more doses of sacituzumab govitecan. In some embodiments, the subject receives one or more doses of the zimberelimab. In some embodiments, the subject receives one or more doses of the domvanalimab. In some embodiments, sacituzumab govitecan and the zimberelimab are administered concurrently. In some embodiments, sacituzumab govitecan and the zimberelimab are administered sequentially. In some embodiments, sacituzumab govitecan and the domvanalimab are administered concurrently. In some embodiments, sacituzumab govitecan and the domvanalimab are administered sequentially. In some embodiments, the zimberelimab and the domvanalimab are administered concurrently. In some embodiments, the zimberelimab and the domvanalimab are administered sequentially. In some embodiments, the subject is human. In some embodiments, sacituzumab govitecan is administered at one or more doses in the range of 8 mg/kg to 10 mg/kg. In some embodiments, sacituzumab govitecan is administered at one or more doses of 10 mg/kg. In some embodiments, sacituzumab govitecan is administered intravenously. In some embodiments, sacituzumab govitecan is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, the method further comprises administering domvanalimab. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle, and domvanalimab is administered at a dose of 1200 mg on day 1 of the 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating, mitigating, reducing, preventing or delaying the recurrence of breast cancer comprising co-administering to a human patient an effective amount of a) sacituzumab govitecan; b) zimberelimab; and, optionally, c) domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the breast cancer is (i) ductal carcinoma in situ (DCIS) or (ii) invasive breast cancer (ILC or IDC). In some embodiments, the cancer has progressed following at least one prior anti-cancer therapy. In some embodiments, the invasive breast cancer is triple-negative breast cancer. In some embodiments, the invasive breast cancer is HR positive/HER-2 negative (HR⁺/HER-2^{low}) breast cancer. In some embodiments, the invasive breast cancer is HR positive/Her-2 low (HR⁺/Her2^{low}) breast cancer. In some embodiments, the invasive breast cancer is inflammatory breast cancer. In some embodiments, the breast cancer is Paget disease of the breast, angiosarcoma, or phyllodes tumor. In some embodiments, the subject is treatment naïve. In some embodiments, the treatment naïve subject has not received prior anti-cancer therapy for (i) ductal carcinoma in situ (DCIS), (ii) invasive breast cancer (ILC or IDC), (iii) Paget disease of the breast, (iv) angiosarcoma, or (v) phyllodes tumor. In some embodiments, the subject has received one or more anti-cancer therapies before administration of the combination of sacituzumab govitecan, zimberelimab, and optionally domvanalimab. In some embodiments, the subject is not treatment naïve. In some embodiments, the subject has received one or more anti-cancer therapies before administration of the combination of sacituzumab govitecan, zimberelimab, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of sacituzumab govitecan, zimberelimab, and optionally domvanalimab is administered in a neoadjuvant setting (e.g., in preparation for surgery or radiation therapy). In some embodiments, the combination of sacituzumab govitecan, zimberelimab, and optionally domvanalimab is administered in an adjuvant setting (e.g., following a primary treatment such as surgery or radiation therapy). In some embodiments, the combination of sacituzumab govitecan, zimberelimab, and optionally domvanalimab is administered in a therapeutic setting (e.g., as the primary therapy). In some embodiments, the combination of sacituzumab govitecan, zimberelimab, and optionally domvanalimab is administered in a maintenance setting. In some embodiments, the subject receives one or more doses of sacituzumab govitecan. In some embodiments, the subject receives one or more doses of the zimberelimab. In some embodiments, the subject receives one or more doses of the domvanalimab. In some embodiments, sacituzumab govitecan and the zimberelimab are administered concurrently. In some embodiments, sacituzumab govitecan and the zimberelimab are administered sequentially. In some embodiments, sacituzumab govitecan and the domvanalimab are administered concurrently. In some embodiments, sacituzumab govitecan and the domvanalimab are administered sequentially. In some embodiments, the zimberelimab and the domvanalimab are administered concurrently. In some embodiments, the zimberelimab and the domvanalimab are administered sequentially. In some embodiments, the subject is human. In some embodiments, the subject is cisplatin-ineligible. In some embodiments, sacituzumab govitecan is administered at one or more doses in the range of 8 mg/kg to 10 mg/kg. In some embodiments, sacituzumab govitecan is administered at one or more doses of 10 mg/kg. In some embodiments, sacituzumab govitecan is administered intravenously. In some embodiments, sacituzumab govitecan is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, the method further comprises administering domvanalimab. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle, and domvanalimab is administered at a dose of 1200 mg on day 1 of the 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, provided herein are methods of treating breast cancer comprising co-administering to a human patient an effective amount of a) sacituzumab govitecan; b) zimberelimab; and, optionally, c) domvanalimab. In some embodiments, the method further comprises co-administering an additional therapeutic agent or therapeutic modality. In some embodiments, the additional therapeutic agent or therapeutic modality comprises one, two, three, or four additional therapeutic agents and/or therapeutic modalities. In some embodiments, the breast cancer is (i) ductal carcinoma in situ (DCIS) or (ii) invasive breast cancer (ILC or IDC). In some embodiments, the cancer has progressed following at least one prior anti-cancer therapy. In some embodiments, the invasive breast cancer is triple-negative breast cancer. In some embodiments, the invasive breast cancer is HR positive/HER-2 negative (HR⁺/HER-2^{low}) breast cancer. In some embodiments, the invasive breast cancer is HR positive/Her-2 low (HR⁺/Her-21^{ow}) breast cancer. In some embodiments, the invasive breast cancer is inflammatory breast cancer. In some embodiments, the breast cancer is Paget disease of the breast, angiosarcoma, or phyllodes tumor. In some embodiments, the subject is treatment naïve. In some embodiments, the treatment naïve subject has not received prior anti-cancer therapy for (i) ductal carcinoma in situ (DCIS), (ii) invasive breast cancer (ILC or IDC), (iii) Paget disease of the breast, (iv) angiosarcoma, or (v) phyllodes tumor. In some embodiments, the subject has received one or more anti-cancer therapies before administration of the combination of sacituzumab govitecan, zimberelimab, and optionally domvanalimab. In some embodiments, the subject is not treatment naïve. In some embodiments, the subject has received one or more anti-cancer therapies before administration of the combination of sacituzumab govitecan, zimberelimab, and optionally domvanalimab. In some embodiments, the cancer is resistant or refractory to one or more anti-cancer therapies. In some embodiments, the anti-cancer therapy is selected from surgery, radiation therapy, chemotherapy, and checkpoint inhibitor therapy. In some embodiments, the combination of sacituzumab govitecan, zimberelimab, and optionally domvanalimab is administered in a neoadjuvant setting (e.g., in preparation for surgery or radiation therapy). In some embodiments, the combination of sacituzumab govitecan, zimberelimab, and optionally domvanalimab is administered in an adjuvant setting (e.g., following a primary treatment such as surgery or radiation therapy). In some embodiments, the combination of sacituzumab govitecan, zimberelimab, and optionally domvanalimab is administered in a therapeutic setting (e.g., as the primary therapy). In some embodiments, the combination of sacituzumab govitecan, zimberelimab, and optionally domvanalimab is administered in a maintenance setting. In some embodiments, the subject receives one or more doses of sacituzumab govitecan. In some embodiments, the subject receives one or more doses of the zimberelimab. In some embodiments, the subject receives one or more doses of the domvanalimab. In some embodiments, sacituzumab govitecan and the zimberelimab are administered concurrently. In some embodiments, sacituzumab govitecan and the zimberelimab are administered sequentially. In some embodiments, sacituzumab govitecan and the domvanalimab are administered concurrently. In some embodiments, sacituzumab govitecan and the domvanalimab are administered sequentially. In some embodiments, the zimberelimab and the domvanalimab are administered concurrently. In some embodiments, the zimberelimab and the domvanalimab are administered sequentially. In some embodiments, the subject is human. In some embodiments, the subject is cisplatin-ineligible. In some embodiments, sacituzumab govitecan is administered at one or more doses in the range of 8 mg/kg to 10 mg/kg. In some embodiments, sacituzumab govitecan is administered at one or more doses of 10 mg/kg. In some embodiments, sacituzumab govitecan is administered intravenously. In some embodiments, sacituzumab govitecan is administered on days 1 and 8 of a 21-day cycle. In some embodiments, zimberelimab is administered at one or more doses in the range of 300 mg to 400 mg. In some embodiments, zimberelimab is administered at dose of 360 mg. In some embodiments, zimberelimab is administered intravenously. In some embodiments, zimberelimab is administered on day 1 of a 21-day cycle. In some embodiments, the method further comprises administering domvanalimab. In some embodiments, domvanalimab is administered at one or more doses in the range of 800 mg to 1600 mg. In some embodiments, domvanalimab is administered at a dose of 1200 mg. In some embodiments, domvanalimab is administered intravenously. In some embodiments, domvanalimab is administered on day 1 of a 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle. In some embodiments, sacituzumab govitecan is administered at a dose of 10 mg/kg on days 1 and 8 of a 21-day cycle; zimberelimab is administered at a dose of 360 mg on day 1 of the 21-day cycle, and domvanalimab is administered at a dose of 1200 mg on day 1 of the 21-day cycle. In some embodiments of the methods provided herein, an anti-CD47 antibody is not co-administered to the subject or human patient. In some embodiments, the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643. In some embodiments, the anti-CD47 antibody is magrolimab. In some embodiments of the methods provided herein, an MCL1 inhibitor is not co-administered to the subject or human patient. In some embodiments, the MCL1 inhibitor is selected from GS-9716, AMG-176, AMG-397, S-64315, AZD-5991, 483-LM, A-1210477, UMI-77 and JKY-5-037. In some embodiments, the MCL1 inhibitor is GS-9716. In some embodiments of the methods provided herein, a FLT3 agonist is not administered to the subject or human patient. In some embodiments, the FLT3 agonist is selected from GS-3583, CDX-301, TAK-605, ONCR-177, Alb-Ftl3L, and SYM-027. In some embodiments, the FLT3 agonist is GS-3583.

In some embodiments, any of the methods provided herein demonstrate anticancer efficacy as determined by one or more efficacy endpoint selected from overall response rate (ORR), radiographic response, disease control rate (DCR), progression free survival (PFS), and overall survival (OS). In some embodiments ORR is defined as the composite proportion of participants with a PSA response or a radiographic complete response (CR) or partial response (PR). In some embodiments radiographic response is defined as the proportion of participants with a best overall response of CR or PR according to RECIST v1.1. In some embodiments DCR is defined as the proportion of participants with a best overall RECIST response of CR, PR, or stable disease (SD). In some embodiments the duration of time required for a patient with SD to be considered in disease control is defined for each treatment arm in a clinical study. In some embodiments PFS is defined as the time from treatment assignment until first documentation of progressive disease (e.g., radiographic progression, bone scan progression, or the like) or death, whichever occurs first. In some embodiments OS is defined as the time from treatment assignment until death due to any cause.

### EXAMPLES

The following examples are offered to illustrate, but not to limit the claimed invention.

### Example 1

### A Phase 2 Study of Sacituzumab Govitecan in Metastatic Urothelial Cancer

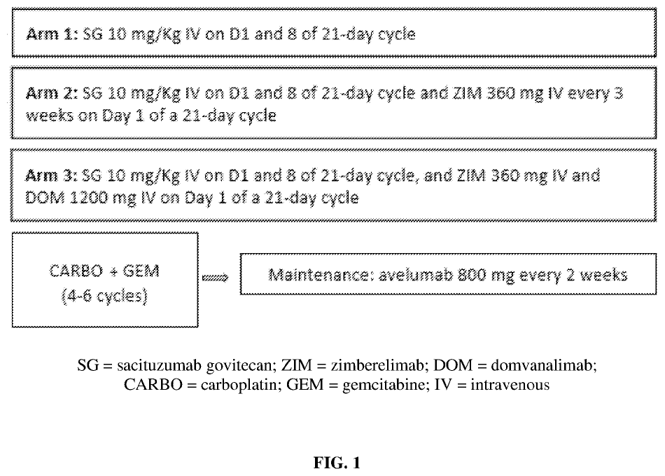

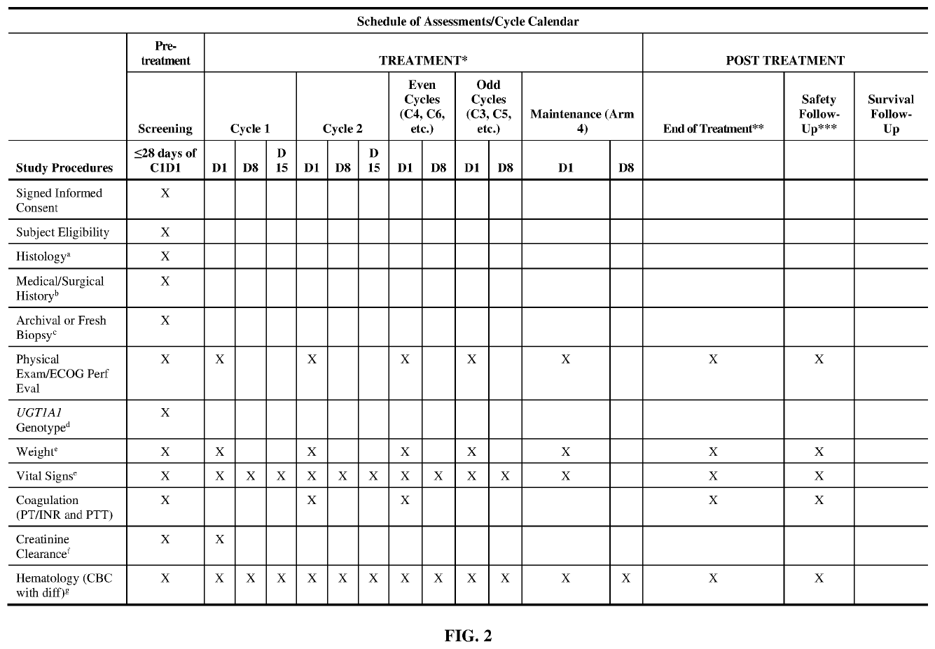

A Phase 1b/2 clinical study is conducted in human cisplatin-ineligible patients who have never received therapy in the metastatic setting or for unresectable locally advanced disease. This study evaluates the administration of an anti-TROP-2 antibody, an anti-PD-(L)1 antibody, and/or an anti-TIGIT antibody in human cisplatin-ineligible patients. A schematic of the treatment arms of the study is shown in FIG. 1.

In Arm 1 of the study, human cisplatin-ineligible patients who have never received therapy for unresectable locally advanced urothelial cancer or in the metastatic setting for urothelial cancer are treated with a sacituzumab govitecan monotherapy (administered at a dose of 10 mg/kg on days 1 and 8 of a 21-day cycle) until PD, unacceptable toxicity, or loss of clinical benefit.

In Arm 2 of the study, human cisplatin-ineligible patients who have never received therapy for unresectable locally advanced urothelial cancer or in the metastatic setting for urothelial cancer are treated with a combination of sacituzumab govitecan (administered at a dose of 10 mg/kg on days 1 and 8 of a 21-day cycle) and zimberelimab (administered at a dose of 360 mg on day 1 of the 21-day cycle) until PD, unacceptable toxicity, or loss of clinical benefit.

In Arm 3 of the study, human cisplatin-ineligible patients who have never received therapy for unresectable locally advanced urothelial cancer or in the metastatic setting for urothelial cancer are treated with a combination of sacituzumab govitecan (administered at a dose of 10 mg/kg on days 1 and 8 of a 21-day cycle), zimberelimab (administered at a dose of 360 mg on day 1 of the 21-day cycle), and domvanalimab (administered at a dose of 1200 mg on day 1 of the 21-day cycle) until PD, unacceptable toxicity, or loss of clinical benefit. Subjects who must discontinue 1 agent may continue the others until PD, unacceptable toxicity, or loss of clinical benefit.

In Arm 4 of the study, human cisplatin-ineligible patients who have never received therapy for unresectable locally advanced urothelial cancer or in the metastatic setting for urothelial cancer are treated with carboplatin (CARBO) AUC 4.5 or 5 by IV infusion on Day 1 of each 21-day cycle for the first 4 to 6 cycles. Gemcitabine (GEM) is administered at a dose of 1000 mg/n² by IV infusion on Days 1 and 8 of each 21-day cycle for the first 4 to 6 cycles. If premature termination of 1 agent occurs due to toxicity, the other agent may be continued to complete up to 6 cycles of therapy. After completion of up to 6 cycles of CARBO and GEM, maintenance therapy is permitted for subjects without disease progression after platinum-based chemotherapy. During maintenance, subjects receive 800 mg of avelumab Q2W until PD, unacceptable toxicity, or loss of clinical benefit.

After discontinuation of treatment with all study drugs, all subjects complete the end of treatment (EOT) visit at the time of study drug discontinuation and complete a safety follow-up visit 30 days after the last study drug dose. Subjects are then be followed up every 12 weeks for survival for a maximum of 2 years. The follow-up may be conducted via telephone or as a visit and includes documentation of any further active therapy administered for their urothelial cancer (UC).

Study Population: Female or male subjects, ≥18 years of age, must be able to give signed, written informed consent. Subjects must have measurable disease by computed tomography imaging (CT)/magnetic resonance imaging (MRI); UC of the renal pelvis, ureter, bladder, or urethra.

Sample Size: The study begins with a safety lead-in of approximately 6 to 8 subjects to assess the safety and tolerability of SG in combination with ZIM (SG+ZIM). If SG+ZIM SRT review of data is deemed safe, an additional 6 to 8 safety lead-in subjects are enrolled to receive SG in combination with ZIM and DOM (SG+ZIM+DOM). After the safety lead-in, about 210 subjects are randomized in a 1:2:2:2 ratio to the 4 arms (SG alone vs SG+ZIM vs SG+ZIM+DOM vs CARBO+GEM). A total of up to 226 subjects are enrolled in this study.

With 60 subjects each in SG+ZIM/SG+ZIM+DOM/CARBO+GEM combination Arms 2, 3, and Control Arm 4, a comparison of SG+ZIM (or SG+ZIM+DOM) versus control has about 88% power to demonstrate a significant improvement in ORR at a one-sided 0.05α with a null hypothesis of ORR=40% for both comparison arms and an alternative hypothesis of ORR=65% for the doublet or triplet arms.

Primary objectives may include determination of the objective response rate (ORR) based on central review by Response Evaluation Criteria in Solid Tumors (RESICST) 1.1 criteria.

Secondary objectives may include:
DOR, CBR, and PFS based on central review by RECIST 1.1 criteria
ORR, DOR, CBR, and PFS based on investigator review by RECIST 1.1 criteria
OS
Safety and tolerability of SG (Arm 1)
Safety and tolerability of SG in combination with ZIM (Arm 2)
Safety and tolerability of SG in combination with ZIM and domvanalimab (DOM) (Arm 3)

Exploratory objectives may include (1) correlation of clinical response (PFS, OS, DOR, and ORR) with Trop-2 expression; and/or (2) Correlation of clinical response (PFS, OS, DOR, and ORR) with programmed death ligand 1 (PD-L1) expression and other biomarkers.

Primary endpoints in the study can include composite overall response rate (ORR; PSA/RECIST Response) and safety.

Secondary endpoints can include ORR per RECIST 1.1, PSA response rate per PCWG3, disease control rate (DCR), or pharmacokinetics (PK).

Exploratory endpoints can include progression free survival, overall survival, or certain biomarkers.

Centralized Procedures: Imaging evidence of efficacy is based on CT/MRI scans with contrast using RECIST 1.1 criteria to classify tumor response, time to onset of ORR, DOR, and PFS. An independent centralized assessment is used to determine the responses.

### Data Analysis

Efficacy analyses are performed on All Treated Subjects, defined as all subjects who received at least 1 dose of study drug(s). The primary endpoint is ORR based on central review by RECIST 1.1 criteria. Secondary endpoints include DOR, CBR, and PFS based on central review by RECIST 1.1 criteria and ORR, DOR, CBR, and PFS based on investigator assessment by RECIST 1.1 criteria. Overall survival, safety, and tolerability is evaluated for this study.

The safety analysis population is all subjects who received at least 1 dose of study drug(s) that was tested. For dose combinations that are tested in the safety lead-in group but not further tested in Stage 2, their safety results may be summarized separately, if deemed necessary and sample sizes are sufficient. Safety and tolerability is evaluated from AEs, standard safety laboratories (CBC with differential and platelet count, serum chemistries, and urinalysis), physical examination, ECG, and vital signs and TSH/free T3/4.

Immunogenicity is determined from serum samples in subjects using an assay performed by the Sponsor's designee for the occurrence of ADAs against hRS7 and SN-38. The occurrences of any positive ADAs are characterized by descriptive statistics.

Pharmacokinetic (PK) analyses of serum SG, hRS7 immunoglobulin G (IgG), SN-38 and SN-38G is performed on serum samples obtained in all subjects treated with SG. ECGs are collected to document SG possible effects on corrected QT intervals. Descriptive statistics are used to summarize both peak and trough values and formal PK analysis based on a non-compartmental model is performed on subjects with adequate serum concentration data, if feasible.

An exploratory exposure-response analysis for the efficacy (PFS and OS) and safety (incidence of Grade 3 to 5 AEs and relationship to UGT1A1) is conducted in all subjects, if feasible. The impact of immunogenicity, if detected, is evaluated in relation to PK and clinical responses including safety/tolerability and efficacy of SG, if feasible. The safety and efficacy profile of SG is also analyzed in relation to renal and hepatic function and UGT1A1 status, if feasible. Correlation between Trop-2 tumor expression and PD-L1 status with efficacy and safety is investigated. Correlation of other biomarkers and any prior genomic testing with efficacy may also be investigated.

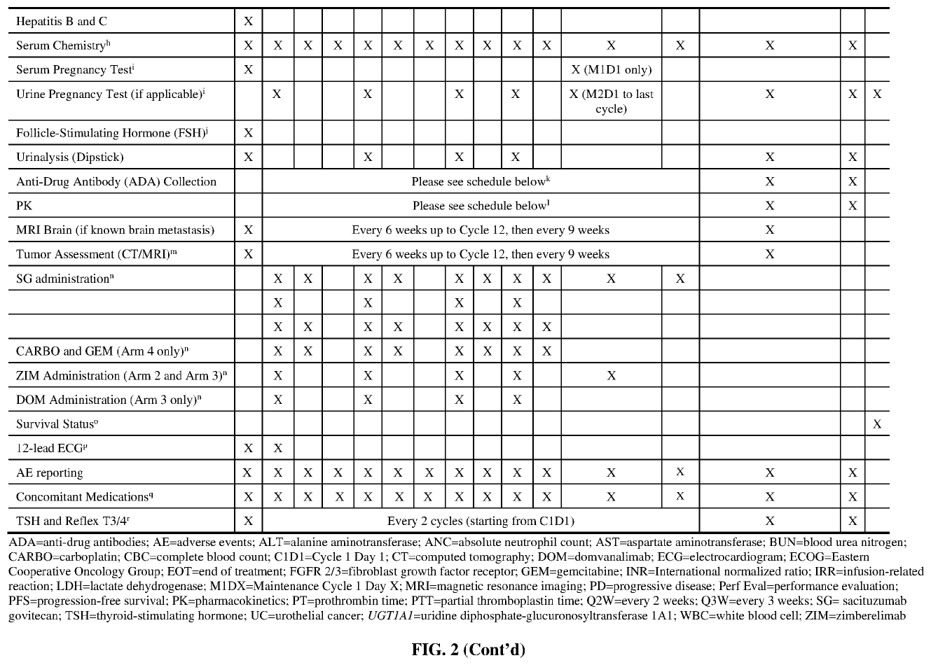

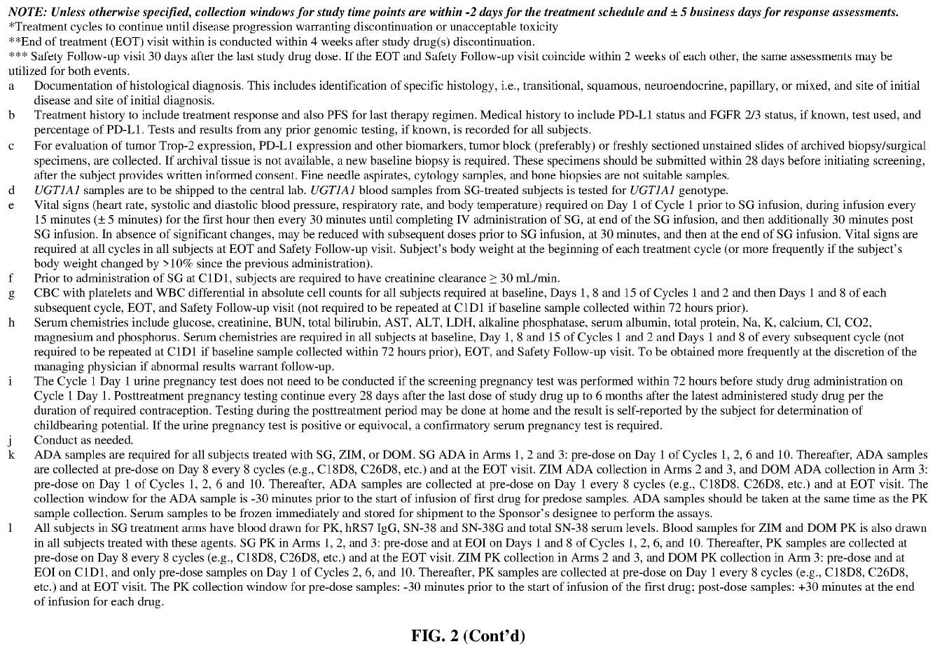

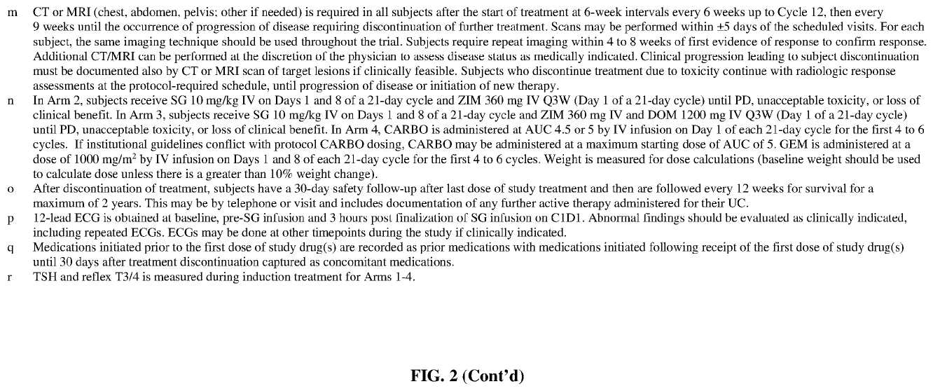

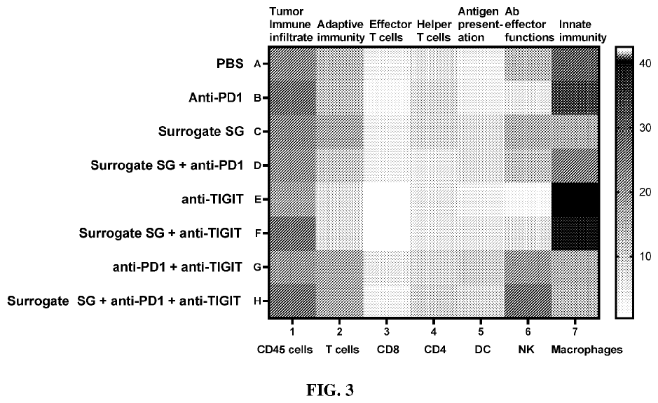

A schedule of assessments/study calendar is shown in FIG. 2.

### Description of Study Therapies

### Sacituzumab Govitecan (IMMU-132)

SN-38, a topoisomerase I inhibitor, is the active metabolite of irinotecan, which is approved as a single agent for colorectal cancer and included in treatment regimens such as FOLFIRI (Zanaan, et al., Second-line chemotherapy with fluorouracil, leucovorin, and irinotecan (FOLFIRI regimen) in patients with advanced small bowel adenocarcinoma after failure of first-line platinum-based chemotherapy: a multicenter AGEO study. Cancer 2011; 117 (7):1422-8). Only 5% of irinotecan is converted to the active SN-38 form by esterase activity residing primarily in the liver, albeit there are esterases in the tumor that can cleave irinotecan to SN-38 (Mathijssen, et al., Clinical pharmacokinetics and metabolism of irinotecan (CPT-11). Clin Cancer Res 2001; 7 (8):2182-94). Its catabolism in the liver results in its transport through the bile duct into the intestines, resulting in gastrointestinal (GI) toxicity.

To improve the bioavailability of SN-38, the Sponsor developed a procedure for coupling SN-38 to an antibody that retains the antibody molecular integrity and specific binding (Govindan, et al., CEACAM5-targeted therapy of human colonic and pancreatic cancer xenografts with potent labetuzumab-SN-38 immunoconjugates. Clin Cancer Res 2009; 15 (19):6052-61; Moon, et al., Cross-linker evaluation In the design of antibody .SN-38 conjugates for cancer therapy [Abstract 2439]. AACR 101st Annual Meeting 2010; 2010 17-21 Apr.; Washington, DC). In murine models bearing human tumor xenografts, 1 particular linker with an SN-38 release half-life of 1.0 to 1.5 days was found to have the best therapeutic response. Importantly, significant antitumor effects were possible at doses that were completely non-toxic to the mice.

Sacituzumab govitecan (SG; IMMU-132) is an antibody-drug conjugate (ADC) composed of the following 3 compounds:
The humanized monoclonal antibody (mAb), hRS7 IgG1κ, binds to Trop-2 (trophoblastic cell-surface antigen, also known as EGP-1, epithelial glycoprotein-1). It is a cell surface, transmembrane calcium signal transducer glycoprotein belonging to the TACSTD gene family that is highly expressed in many epithelial cancers, particularly metastatic sites (Alberti, et al, TROP2 is a major determinant of growth and metastatic spreading of human cancer. J Clin Oncol 2007; 25 (18_Suppl):10510), with much lower expression in normal tissues (Trerotola, et al., CD133, Trop-2 and alpha2beta1 integrin surface receptors as markers of putative human prostate cancer stem cells. American journal of translational research 2010; 2 (2):135-44). Trop-2 has been implicated in oncogenesis, often being found in more aggressive tumors (Fang, et al., Elevated expressions of MMP7, TROP2, and survivin are associated with survival, disease recurrence, and liver metastasis of colon cancer. International journal of colorectal disease 2009; 24 (8):875-84). The antibody was initially developed as a murine mAb by the immunization of mice with a fresh specimen of a human lung cancer. The properties of the parent murine antibody have been extensively described (Basu, et al., The epithelial/carcinoma antigen EGP-1, recognized by monoclonal antibody RS7-3G11, is phosphorylated on serine 303. Int J Cancer 1995; 62 (4):472-9; Shih, et al., In vitro and in vivo reactivity of an internalizing antibody, RS7, with human breast cancer. Cancer Res 1995; 55 (23 Suppl):5857s-63s; Stein, et al., Specificity and properties of MAb RS7-3G11 and the antigen defined by this pancarcinoma monoclonal antibody. Int J Cancer 1993; 55 (6):938-46; Stein, et al., Characterization of cluster 13: the epithelial/carcinoma antigen recognized by MAb RS7. Int J Cancer Suppl 1994; 8:98-102; Stein, et al., Effects of radiolabeling monoclonal antibodies with a residualizing iodine radiolabel on the accretion of radioisotope in tumors. Cancer Res 1995; 55 (14):3132-9; Stein, et al., Advantage of a residualizing iodine radiolabel for radioimmunotherapy of xenografts of human non-small-cell carcinoma of the lung. J Nucl Med 1997; 38 (3):391-5).
The camptothecin-derived agent, SN-38, a topoisomerase 1 inhibitor.
A linker, with the company designation, CL2A, which binds SN-38 to the antibody.

SG is prepared at a ratio of 7 to 8 molecules of SN-38 per molecule of the anti-TROP-2 antibody, hRS7 (Goldenberg, et al., Trop-2 is a novel target for solid cancer therapy with sacituzumab govitecan (IMMU-132), an antibody-drug conjugate (ADC). Oncotarget 2015; 6 (26):22496-512). For clinical use, 10 mg/mL are formulated in 22 mM 2-(N-morpholino)ethane sulfonic acid buffer, pH 6.5, together with the other excipients (25 mM trehalose, 0.01% polysorbate 80). SG is supplied in 50-mL clear glass vials to be stored under refrigerated conditions (2 to 8° C.) until used. Since the formulated drug product contains no preservative, vials should be used only once and within 4 hours of reconstitution.

Each vial of SG is reconstituted with 20 mL of saline and the resulting 10 mg/mL solution is further diluted with saline for intravenous (IV) administration so that the final concentration of SG is in the 1.1 to 3.4 mg/mL range.

### Carboplatin

Carboplatin (CARBO; Paraplatin®) is an anticancer platinum coordination compound that produces intrastrand DNA crosslinks. CARBO is indicated for the treatment of solid tumors, including locally advanced or metastatic bladder cancer (National Comprehensive Cancer Network. NCCN Clinical Practice Guidelines in Oncology (NCCN Guidelines) Bladder Cancer. Version 4.2021-Jul. 27, 2021.2021). CARBO has been used as a treatment for neoplasia for an extended period of time. CARBO avoids several of the AEs associated with cisplatin, including renal toxicity, ototoxicity, and peripheral neuropathy.

### Gemcitabine

GEM is a nucleoside metabolic inhibitor indicated in combination with CARBO, for the treatment of locally advanced or metastatic bladder cancer in cisplatin-ineligible patients (National Comprehensive Cancer Network. NCCN Clinical Practice Guidelines in Oncology (NCCN Guidelines) Bladder Cancer. Version 4.2021-Jul. 27, 2021. 2021). Refer to GEM package insert (GEMCITABINE, Accord Healthcare Limited. GEMCITABINE injection, for intravenous use. U. S. Prescribing Information. Durham, NC. Revised June. 2019) or SmPC for more information on GEM mechanism of action.

### Avelumab

Avelumab is a PD-L1 blocking antibody and belongs to the group of immune checkpoint inhibitor (CPI) cancer therapies. It selectively blocks the interaction between PD-1 and B7.1 PD-L1 receptors, while still allowing interaction between PD-L2 and PD-1 (Kaufman, et al., Avelumab in patients with chemotherapy-refractory metastatic Merkel cell carcinoma: a multicentre, single-group, open-label, phase 2 trial. Lancet Oncol 2016; 17 (10):1374-85). This interaction then allows T-cell receptor activation and cell lysis.

Avelumab is used for the treatment of subjects with locally advanced or metastatic UC who have disease progression during or following platinum-containing chemotherapy or have disease progression within 12 months of neoadjuvant or adjuvant treatment with platinum-containing chemotherapy. Avelumab was approved in June 2020 as maintenance treatment for subjects with locally advanced or metastatic UC that has not progressed with first-line platinum-containing chemotherapy. Refer to avelumab package insert (BAVENCIO, EMD Serono Inc. BAVENCIO® (avelumab) injection, for intravenous use. U.S. Prescribing Information. Rockland, MA. Revised June. 2020) or SmPC for more information on avelumab mechanism of action.

### Zimberelimab (ZIM)

ZIM is a fully human IgG4 monoclonal antibody targeting human PD-1. PD-1 is a type 1 transmembrane protein that is part of the immunoglobulin gene superfamily and the CD28 family of cell surface receptors. The structure of PD-1 consists of 1 immunoglobulin variable-like extracellular domain and 1 cytoplasmic domain containing an immunoreceptor tyrosine-based inhibitory motif and an immunoreceptor tyrosine-based switch motif. PD-1 has 2 known ligands, PD-L1 (B7 HI and CD274) and programmed cell death ligand 2 (PD-L2; B7 DC and CD73), which are members of the B7 family and are expressed on the plasma membrane of cancer cells and tumor-infiltrating leukocytes. Both PD-L1 and PD-L2 are B7 homologs that bind to PD-1, but they do not bind to other CD28 family members.

PD-1 is an inhibitory immune checkpoint protein that is expressed on activated B cells, T cells, and myeloid cells, and it plays a key role in limiting the activity of effector T cells. It also provides a major resistance mechanism by which tumor cells can escape immune surveillance. When activated by its ligands, PD-1 induces a state of anergy or unresponsiveness in T cells, and the cells are unable to produce optimal levels of effector cytokines or carry out other effector T cell functions. PD-1 may also induce apoptosis in T cells via its ability to inhibit survival signals. Under normal circumstances, PD-1 is important for limiting the extent of T cell-mediated immune responses. PD-1-deficient animals develop various autoimmune phenotypes, including autoimmune cardiomyopathy and a lupus-like syndrome with arthritis and nephritis.

The interaction of PD-1 expressed on activated T cells and PD-L1 expressed on tumor cells negatively regulates immune response and dampens antitumor immunity. PD-L1 is abundantly expressed on a variety of human tumors, and its expression correlates with reduced subject survival in esophageal, pancreatic, and other types of cancers. Therefore, the PD-(L)1 pathway is an important target for tumor immunotherapy. Activation of the PD-(L)1 signaling pathway results in a decrease in tumor-infiltrating lymphocytes, a decrease in T cell proliferation, and an increase in immune evasion by cancerous cells. Immune suppression can be reversed by inhibiting the local interaction of PD-1 with PD-L1, and the effect is additive when the interaction of PD-1 with PD-L2 is also blocked.

### Domvanalimab (DOM)

DOM is a humanized immunoglobulin G1 (IgG1) monoclonal antibody that targets T-cell immunoreceptor with immunoglobulin and immunoreceptor tyrosine-based inhibitory motif domains (TIGIT), which functions as an immune checkpoint. DOM comprises 2 heavy chains of the IgG1 isotype and 2 light chains of the kappa subclass.

### Study Drug Treatment

### SG Treatment

Note: This section specifically provides guidance for SG administration and toxicity management, including guidelines for infusion reactions, dose delay, dose reduction and treatment discontinuation.

### Concomitant Medications and Procedures

No anticancer therapies, aside from the study drug(s) are permitted during this study. However, palliative and/or supportive medications, such as bone-modifying medications (bisphosphonates or denosumab), and/or procedures such as radiation and surgery are allowed at the investigator's discretion. If a subject requires palliative radiotherapy, SG should be interrupted 1 week before the procedure and reinstated 2 weeks after the procedure, provided that palliative radiotherapy is not indicated for tumor progression, in which case the subject has to be discontinued from study drug(s) unless there is evidence of clinical benefit supporting continuation of therapy. If a subject requires surgery, SG should be interrupted 1 week before the procedure if clinically feasible and dosing should be held for 2 weeks after the procedure. Dosing may resume thereafter if the subject is clinically stable. Extensive surgical procedures such as abdominal, cranial surgeries for example, may require suspension of dosing for 4 weeks before dosing may resume to allow for an adequate period for healing.
Cytokines, growth factors or blood product transfusions should be administered as clinically necessary.
Other supportive and palliative care is allowed as medically warranted.
Routine vaccinations including for COVID-19 are permitted but should be initiated after Cycle 2 if clinically feasible. For details on the risk and mitigation strategy for concurrent administration of the COVID-19 vaccine.
Live attenuated vaccines are not allowed within 30 days prior to first dose of study drug and while participating in the study. It is also recommended that subjects do not receive live attenuated vaccines 90 days after last dose of study drug.

### Drug Interactions

No formal drug-drug interaction studies with SG have been conducted. Concomitant administration of inhibitors or inducers of UGT1A1, with SG, should be avoided due to the potential to either increase (inhibitors) or decrease (inducers) the exposure to SN-38, unless there are no therapeutic alternatives.

### UGT1A1 Inhibitors

Coadministration of SG with inhibitors of UGT1A1 may increase systemic exposure to the active metabolite, SN-38. Do not administer UGT1A1 inhibitors with SG unless there are no therapeutic alternatives. Examples of UGT1A1 inhibitors include amitriptyline, atazanavir, dacomitinib, dasabuvir, deferasirox, eltrombopag, enasidenib, erlotinib, flunitrazepam, flurbiprofen, fostamatinib, gemfibrozil, glecaprevir, indinavir, indomethacin, ketoconazole, nilotinib, ombitasvir, paritaprevir, pazopanib, pexidartinib, pibrentasvir, probenecid, propofol, regorafenib, rucaparib, silibinin, sorafenib, and valproic acid.

### UGT1A1 Inducers

Exposure to SN-38 may be substantially reduced in subjects concomitantly receiving UGT1A1 enzyme inducers. Do not administer UGT1A1 inducers with SG unless there are no therapeutic alternatives. Examples of UGT1A1 inducers include carbamazepine, efavirenz, ethinylestradiol, lamotrigine, phenobarbital, phenytoin, primidone, rifampicin, ritonavir, and tipranavir.

### Intravenous Administration of SG

The recommended dose of SG is 10 mg/kg administered as an IV infusion once weekly on Days 1 and 8 of continuous 21-day cycles. Continue treatment until disease progression or unacceptable toxicity. Do not administer SG at doses greater than 10 mg/kg. Administer SG as an IV infusion only. Do not administer as an IV push or bolus.
SG is a cytotoxic drug. Follow applicable special handling and disposal procedures.
Administer IMP as an IV infusion.
Administer the first infusion over 3 hours. Monitor the subject during, and for at least 30 minutes after infusion. Subsequent infusions may be administered over 1 to 2 hours if previous infusions were well tolerated.
Protect the infusion bag from light.
An infusion pump may be used.
Compatibility with polypropylene, polyvinyl chloride, and ethylene/propylene copolymer infusion bags have been demonstrated and are acceptable for use with SG.
Do not mix IMP, or administer as an infusion, with other medicinal products.
Upon completion of the infusion, flush the IV line with 20 mL 0.9% Sodium Chloride Injection, United States Pharmacopeia.

### Intravenous Administration of Carboplatin

CARBO is administered by IV infusion at a dose of AUC 4.5 or AUC 5 on Day 1 of each 21-day cycle for the first 4 to 6 cycles. If institutional guidelines conflict with protocol CARBO dosing, CARBO may be administered at a maximum starting dose of AUC of 5. The dose of CARBO is based on the subject's baseline weight (measured ≤14 days prior to initiation of study treatment) and remains the same throughout the study unless the subject's weight changes by >10% relative to the weight recorded at the time of the previous dose calculation.

### Intravenous Administration of Gemcitabine

GEM is administered by IV infusion at a dose of 1000 mg/m² on Days 1 and 8 of each 21-day cycle for the first 4 to 6 cycles. The dose of GEM is based on the subject's baseline weight (measured ≤14 days prior to initiation of study treatment) and remains the same throughout the study unless the subject's weight changes by >10% relative to the weight recorded at the time of the previous dose calculation.

### Intravenous Administration of Avelumab

If a subject experiences IRR during SG infusion, avelumab should not be given until IRR is considered resolved by investigator.

### Intravenous Administration of ZIM

For Arm 2 and Arm 3 ZIM doses are administered by IV infusion), followed by an observation period, on Day 1 of each 21-day cycle.

During the maintenance period (dose expansion phase) of Arm 2 and Arm 3, SG is administered first followed by ZIM.

If a subject experiences IRR during SG infusion, ZIM should not be given until IRR is considered resolved by investigator.

### Intravenous Administration of DOM

DOM 1200 mg is administered by IV infusion followed by an observation period, on Day 1 of each 21-day cycle.

During Arm 3, SG is administered first followed by ZIM and DOM.

### Study Rationale

This study is designed to study SG as both monotherapy and combination treatment in different lines of therapy for the treatment of subjects with UC. Arm 1 provides information on the activity of monotherapy SG in treatment-naïve cisplatin-ineligible subjects with metastatic or unresectable locally advanced UC. Arm 2 provides information on the activity of SG in combination with ZIM in treatment-naïve cisplatin-ineligible subjects with metastatic or unresectable locally advanced UC. Arm 3 provides information on the activity of SG in combination with ZIM and DOM in treatment-naïve cisplatin-ineligible subjects with metastatic or unresectable locally advanced UC. Arm 4 provides information on the activity of CARBO+GEM in cisplatin-ineligible subjects with metastatic or unresectable locally advanced UC.

Arm 1 assesses the safety, tolerability, and clinical activity of SG in cisplatin-ineligible UC subjects who are treatment naïve in the metastatic or unresectable locally advanced setting.

Arm 2 assesses the safety, tolerability, and clinical activity of SG in combination with ZIM in cisplatin-ineligible UC subjects who are treatment naïve in the metastatic or unresectable locally advanced setting. SG and ZIM are being combined with the expectation that the combination potentially provides a greater clinical benefit and durability of response. Safety data support the combination of SG and ZIM. To ensure thorough surveillance of any early and unexpected safety signals with the combination, before randomization, a Safety Review Team (SRT) performs a review of safety data in approximately 6 to 8 subjects in Arm 2 (safety lead-in) who have received at least 1 study dose and completed 1 full cycle; if safe, enrollment continues.

Arm 3 assesses the safety, tolerability, and clinical activity of SG in combination with ZIM and DOM in cisplatin-ineligible UC subjects who are treatment naïve in the metastatic or unresectable locally advanced setting. SG, ZIM, and DOM are being combined with the belief that the addition of DOM provides a greater clinical benefit, such as durability of response of SG. To ensure thorough surveillance of any early and unexpected safety signals with the combinations, before randomization, an SRT performs a review of safety data in approximately 6 to 8 subjects in Arm 3 (safety lead-in) who completed 1 full cycle. Arm 3 (safety lead-in) starts after Arm 2 (safety lead-in) is completed; if safe, enrollment continues.

Arm 4 assesses the safety, tolerability, and clinical activity of CARBO in combination with GEM in cisplatin-ineligible UC subjects who are treatment naïve in the metastatic or unresectable locally advanced setting followed by avelumab maintenance for subjects who do not progress following chemotherapy.

Given the tolerable safety profile and the known clinical activity of SG in mUC, this study assesses SG's clinical activity as monotherapy in the first-line cisplatin-ineligible subject population. Data from Arm 1 also complements Arm 2 and Arm 3 data and helps in determining the contribution of SG in the SG+ZIM combination (Arm 2) or SG+ZIM+DOM combination (Arm 3) in this subject population. Arm 4 is being added since the standard of care for cisplatin-ineligible patients with mUC is CARBO+GEM followed by avelumab maintenance for patients who do not progress following chemotherapy.

Subjects are randomized 1:2:2:2 to Arm 1, Arm 2, Arm 3, or Arm 4 using the IRT system after the safety lead-in in Arms 2 and 3 are completed

### Rationale for Dose Regimen

Subjects in Arm 1 receive the standard approved dose of SG 10 mg/kg IV on Days 1 and 8 of a 21-day cycle until PD, unacceptable toxicity, or loss of clinical benefit. SG at 10 mg/kg has an established clinical activity in mUC based on the recent accelerated FDA approval.

Subjects in Arm 2 receive SG 10 mg/kg IV on Days 1 and 8 of a 21-day cycle and ZIM 360 mg IV Q3W (Day 1 of a 21-day cycle) until PD, unacceptable toxicity, or loss of clinical benefit.

The full dose (10 mg/kg) of SG is tested in Arm 2. Data from 41 subjects treated with a full dose of SG in combination with pembrolizumab show that the combination had a manageable safety profile with no new safety signals and demonstrated encouraging antitumor efficacy. To ensure thorough surveillance of any early and unexpected safety signals with the combination, before randomization, an SRT performs a review of safety data in approximately 6 to 8 subjects in Arm 2 (safety lead-in) who completed 1 full cycle.

Subjects who must discontinue 1 agent may continue the other until PD, unacceptable toxicity, or loss of clinical benefit is documented. Discontinuation or skipping of 1 agent does not require discontinuation of the other agent. Skipped doses may not be made up.

The selected dose of DOM in Arm 3 is 1200 mg administered IV Q3W.

Subjects in Arm 4 receive CARBO at AUC 4.5 or 5 by IV infusion on Day 1 of each 21-day cycle for the first 4 to 6 cycles. GEM is administered at a dose of 1000 mg/m² by IV infusion on Days 1 and 8 of each 21-day cycle for the first 4 to 6 cycles.

A delay of CARBO or GEM requires delay of the other agent. If subjects become ineligible for additional CARBO administration prior to completion of 6 cycles of therapy, CARBO must be discontinued, and the induction phase completed with only GEM. Substitution of CARBO is not permitted. Discontinuation or skipping of 1 induction agent does not require discontinuation of the other agent. Skipped doses may not be made up.

After completion of up to 6 cycles of CARBO and GEM, maintenance therapy is permitted to continue with 800 mg of avelumab every 2 weeks.

### Study Design

Arm 1 (up to 30 subjects) evaluates SG monotherapy in treatment-naïve cisplatin-ineligible subjects with metastatic or unresectable locally advanced UC. Treatment may be discontinued at any time, but subjects continue to be followed for tumor response until progression is documented, or alternate therapy is initiated.

Arm 2 (up to 60 subjects) evaluates SG in combination with ZIM in treatment naïve cisplatin-ineligible subjects with metastatic or unresectable locally advanced UC. The standard approved dose of SG is tested with ZIM in Arm 2, since other studies have established that SG can be safely administered with full-dose CPI therapy. If a subject must discontinue or skip 1 agent, then the other agent may be continued. Skipped doses may not be made up. Treatment may be discontinued at any time, but subjects continue to be followed for tumor response until progression is documented, or alternate therapy is initiated. If subjects discontinue therapy before evidence of radiologic progression, imaging should continue until radiologic progression is documented, if feasible.

To ensure thorough surveillance of any early and unexpected safety signals with the combination, before randomization, an SRT performs a review of safety data in approximately 6 to 8 subjects in Arm 2 (safety lead-in) who have received at least 1 study dose and completed 1 full cycle.

Arm 3 (up to 60 subjects) evaluates SG in combination with ZIM and DOM in treatment naïve cisplatin-ineligible subjects with metastatic or unresectable locally advanced UC.

Arm 3 starts enrollment after Arm 2 safety lead-in evaluation is completed. To ensure thorough surveillance of any early and unexpected safety signals with the combinations, before randomization, an SRT performs a review of safety data in approximately 6 to 8 subjects in Arm 3 (safety lead-in) who have received at least 1 study dose and completed 1 full cycle.

Arm 4 (up to 60 subjects) evaluates CARBO in combination with GEM in treatment naïve cisplatin-ineligible subjects with metastatic or unresectable locally advanced UC.

### Inclusion Criteria

Subjects meeting all the following inclusion criteria at screening/day-1 of treatment are eligible for participation in the study.
1) Female or male subjects, ≥18 years of age, able to understand and give written informed consent.
2) Subjects with histologically documented locally advanced (tumor [T]4b, any node [N]; or any T, N2-3) or metastatic (M1, Stage IV) UC. Upper and lower tract tumors are permitted and mixed histologies are permitted if UC is the predominant histology.
3) Archival tumor tissue comprising muscle-invasive or metastatic urothelial carcinoma, or a biopsy of metastatic urothelial carcinoma must be provided for biomarker testing including PD-L1 and Trop-2.
4) Eastern Cooperative Oncology Group (ECOG) Performance status score of 0 or 1.
   a) Received a first-line platinum-containing regimen in the metastatic setting or for inoperable locally advanced disease;
   b) Or received neo/adjuvant platinum-containing therapy for localized muscle-invasive UC, with recurrence/progression ≤12 months following completion of therapy.
5) Subject with no prior therapy for metastatic disease or for unresectable locally advanced disease with the following exceptions:
   a) Subjects who received neoadjuvant chemotherapy with curative intent with recurrence >12 months from completion of therapy are permitted.
   b) Subjects who received adjuvant chemotherapy following cystectomy with curative intent with recurrence >12 months from completion of therapy are permitted.
6) Cisplatin-ineligible based on criteria below:
   a) Creatinine clearance <60 mL/min
   b) Grade ≥2 audiometric hearing loss
   c) Grade ≥2 peripheral neuropathy
   d) New York Heart Association Class III heart failure
7) Checkpoint inhibitor therapy naïve or >12 months from completion of for adjuvant therapy are permitted.
8) Adequate hematology without transfusion support or growth factor support within 2 weeks of study drug initiation (total WBC count must be ≥4000 per mm³, ANC ≥1500 per mm³, platelets ≥100,000 per mcL, and hemoglobin ≥9 g/dL).
9) Adequate hepatic function (Bilirubin ≤1.5 ULN, AST and ALT ≤2.5xULN or ≤5xULN if known liver metastases and serum albumin ≥3 g/dL).
10) Creatinine clearance ≥30 mL/min as calculated by the Cockcroft-Gault formula unless otherwise specified.
11) Subjects must have a 3-month life expectancy.
12) Adequate coagulation (Prothrombin Time [PT]) or International Normalized Ratio [INR] and Activated Partial Thromboplastin Time [aPTT])≤1.5xULN unless subject is receiving anticoagulant therapy as long as PT or PTT is within therapeutic range of intended use of anticoagulants.
13) Have measurable disease by CT or MRI as per RECIST 1.1 criteria. Tumor lesions situated in a previously irradiated area are considered measurable if progression has been demonstrated in such lesions.
14) Male subjects and female subjects of childbearing potential who engage in heterosexual intercourse must agree to use protocol-specified method(s) of contraception.
15) Subject must be willing and able to comply with all protocol requirements.

### Exclusion Criteria

Subjects meeting any of the following exclusion criteria at Screening/day −1 of treatment are not be enrolled in the study
1) Women who are pregnant or lactating.
2) Is currently participating in or has participated in a study of an investigational agent or using an investigational device within 4 weeks prior to the first dose of trial treatment.
3) Has a diagnosis of immunodeficiency.
4) Has had a prior anticancer mAb within 4 weeks prior to study Day 1 or who has not recovered (i.e., ≤Grade 1) from AEs due to agents administered more than 4 weeks earlier.
5) Has had prior chemotherapy, targeted small molecule therapy, or radiation therapy within 2 weeks prior to study Day 1 or who has not recovered (i.e., ≤Grade 1 from AEs due to a previously administered agent).
   Note: Subjects with ≤Grade 2 neuropathy or ≤Grade 2 alopecia are an exception to this criterion and may qualify for the study.
   Note: If subject received major surgery, they must have recovered adequately from the toxicity and/or complications from the intervention prior to starting therapy.
6) Requires concomitant medications that significantly interfere with ABCA1 transporter or UGT1A1 with no alternate option available.
7) Subjects with Gilbert's disease.
8) Subjects who previously received irinotecan.
9) Has an active second malignancy.
   Note: Subjects with a history of malignancy that has been completely treated, with no evidence of active cancer for 3 years prior to enrollment, or subjects with surgically cured tumors with low risk of recurrence are allowed to enroll.
10) Has known active CNS metastases and/or carcinomatous meningitis. Subjects with previously treated brain metastases may participate provided they have stable CNS disease for at least 4 weeks prior to the first dose of study drug and all neurologic symptoms have returned to baseline), have no evidence of new or enlarging brain metastases, and are not using steroids greater than 20 mg of prednisone daily for brain metastases (or the equivalent) for at least 7 days prior to trial treatment. All subjects with carcinomatous meningitis are excluded regardless of clinical stability. 11) Has active cardiac disease, defined as:
   a) Myocardial infarction or unstable angina pectoris within 6 months of the first date of study therapy.
   b) History of serious ventricular arrhythmia (i.e., ventricular tachycardia or ventricular fibrillation), high-grade atrioventricular block, or other cardiac arrhythmias requiring antiarrhythmic medications (except for atrial fibrillation that is well controlled with antiarrhythmic medication); history of QT interval prolongation.
   c) New York Heart Association Class III or greater congestive heart failure or left ventricular ejection fraction of <40%.
12) Has active chronic inflammatory bowel disease (ulcerative colitis, Crohn's disease) and subjects with a history of bowel obstruction.
13) Has prior history of clinically significant bleeding, intestinal obstruction, or GI perforation within 6 months of enrollment.
14) Must be at least 2 weeks beyond high dose systemic corticosteroids (however, low dose corticosteroids ≤20 mg prednisone or equivalent daily are permitted for reasons outside of CNS disease provided the dose is stable for 4 weeks).
15) Has an active infection requiring systemic therapy.
16) Have known history of HIV-1 or 2 with uncontrolled viral load (i.e., ≥200 copies/mL or CD4+ T-cell count <350 cells/μL) or taking medications that may interfere with metabolism of study drugs. No HIV testing is required unless mandated by local health authority.
17) Has active hepatitis B virus or hepatitis C virus defined as those with a detectable viral load.
18) Has other concurrent medical or psychiatric conditions that, in the investigator's opinion, may be likely to confound study interpretation or prevent completion of study procedures and follow-up examinations.
19) Use of live attenuated vaccine(s) 30 days before start of study drug.
20) Has received a live vaccine including COVID-19 vaccines within 30 days prior to the first dose of study drug(s). Seasonal flu and COVID-19 vaccines that do not contain live virus are permitted.
21) Has received immunosuppressive therapy within 3 years prior to C1D1.
22) Has history or evidence of interstitial lung disease or non-infectious pneumonitis.
23) Hypersensitivity or anaphylaxis to cisplatin, CARBO, or GEM.
24) Have inability to tolerate or are allergic to SG, or CPIs, or are unable or unwilling to receive the doses specified in the protocol.

### Study Procedures

Unless otherwise specified, collection windows for study timepoints are within −2 days for the treatment schedule and ±5 business days for response assessments. Collection windows for PK samples are +5 minutes.

Blood samples obtained for ADAs (hRS7 and SN-38), PK, TSH and reflex T3/4, and UGT1A1 levels. Tumor blocks (preferably) or freshly sectioned unstained slides of archived biopsy/surgical specimens for Trop-2 expression, PD-L1 expression, and other biomarkers are collected. The results from any prior genomic testing, PD-L1 status, and FGFR 2/3 status, if known is recorded for all subjects at Screening.

### Pre-Study/Baseline Evaluations

Baseline and screening evaluations conducted to establish eligibility is performed within 28 days prior to first study drug (see FIG. 2):
Signed informed consent
Serum beta human chorionic gonadotropin, in women of childbearing potential during screening, and a urine pregnancy test at baseline. The urine pregnancy test at baseline does not need to be conducted if the serum pregnancy test at screening was performed within 72 hours before study treatment administration on C1D1.
Follicle-stimulating hormone (FSH) testing is conducted as needed for determination of childbearing potential.
Documentation of histological diagnosis of UC
Medical/surgical history review with treatment history to include treatment response and time until progression for last therapy regimen
ECOG Performance Status
Prior/concomitant medications review
Physical examination with vital signs defined as heart rate, blood pressure, respiratory rate, body weight, and body temperature
Standard 12-lead ECG
CBC including platelet count, with WBC differential in absolute cell counts, to be repeated within 72 hours of Cycle 1, Day 1 to confirm eligibility
Routine serum chemistries (glucose, creatinine, blood urea nitrogen [BUN], total bilirubin, AST, ALT, lactate dehydrogenase (LDH), alkaline phosphatase, serum albumin, total protein, sodium (Na), potassium (K), calcium, chlorine (Cl), carbon dioxide (CO₂), magnesium and phosphorus)
Hepatitis B surface antigen test, hepatitis C antibody test.
TSH and reflex T3/4 for all subjects
Tests and results from any prior genomic testing, PD-L1 status, and FGFR 2/3 status, if known is recorded for all subjects
Creatinine clearance (may be calculated using Cockcroft-Gault equation)
Baseline serum sample prior to receiving SG for ADA (hRS7 and SN-38) response (frozen, to be shipped to Sponsor's designee for analysis)
Tumor blocks (preferably) or freshly sectioned unstained slides of archived biopsy/surgical specimens are collected for biomarker testing including PD-L1 and Trop-2. If archival tissue is not available, a new baseline biopsy is required.
A single whole-blood sample to be collected prior to receiving SG, for determination of UGT1A1 genotype
Urinalysis
CT or MRI with contrast (chest, abdomen, pelvis, other regions of known/suspected involvement)
Brain MRI in subjects with known or suspicion of CNS disease

### Procedures During Treatment

Dosing (SG dosing is based on subject's body weight on Day 1 of each cycle, or more frequently if the subject's body weight changed by >10% since the previous administration) (see FIG. 2):
Vital signs (heart rate, systolic and diastolic blood pressure, respiratory rate, and body temperature) (prior to first SG infusion and every 15 minutes for the first hour then every 30 minutes until completing SG IV administration, at completion, and 30 minutes post SG infusion). In absence of significant changes, may be reduced with subsequent doses to prior to SG infusion, at 30 minutes, and then at completion of SG IV administration). Time points can be ±5 min.
Blood samples prior to SG infusion and Day 15 for Cycles 1 and 2 only:
   CBC (including platelet count, with WBC differential in absolute cell counts)
   Serum chemistry panel (glucose, creatinine, BUN, total bilirubin, AST, ALT, LDH, alkaline phosphatase, serum albumin, total protein, Na, K, calcium, C1, CO₂, magnesium and phosphorus)
Coagulation: INR or PT and aPTT (Day 1 on even cycles)
TSH and reflex T3/4 (every 2 cycles starting from CID1)
Urinalysis, pregnancy test (if applicable at 72 hours prior to first dose of study drug, and on Day 1 of all cycles starting from Cycle 2)
Physical examination. (Day 1 of all cycles)
Record AEs (at every visit)
Record concomitant medications; continued, changed (at every visit)
12-lead ECG is obtained at baseline, pre-SG infusion and 3 hours post finalization of SG infusion on C1D1. Abnormal findings should be evaluated as clinically indicated, including repeated ECGs. ECGs may be done at other timepoints during the study if clinically indicated.
PK samples
ADA samples
CT or MRI examinations. Starting with the initial dose of study drug, CT or MRI scans are to be obtained in all subjects after the first treatment dose every 6 weeks. Scans may be performed within ±5 days of the scheduled visits. After 12 cycles of therapy, scan can be done every 9 weeks until the occurrence of progression of disease requiring discontinuation of further treatment. CT or MRI imaging includes chest, abdomen, pelvis, other areas of known/suspected involvement, with contrast; scans that lack contrast may only be permitted after discussion and agreement with the medical monitor. Subjects with known brain metastasis must have a brain MRI. Target and non-target lesions must be determined by the clinical site at baseline. The same imaging technique should be used throughout for tumor assessment. Clinical progression leading to subject discontinuation should be documented also by CT or MRI scan of target lesions if possible. Subjects who discontinue treatment due to toxicity continue with radiologic response assessments at the protocol-required schedule, until progression of disease or initiation of new therapy. Additional CT or MRI studies may be performed at the discretion of the physician to assess disease status as medically indicated. Subjects require repeat imaging within 4 to 6 weeks of first evidence of response to confirm response.

### Pharmacokinetic Evaluations

For SG PK in Arms 1, 2, and 3: pre-dose and at EOI on C1D1 and C1D8, and pre-dose on Days 1 and 8 of Cycles 2, 6, and 10. Thereafter, PK samples are collected at pre-dose on Day 8 every 8 cycles (e.g., C18D8, C26D8, etc.) and at the EOT visit. ZIM PK collection in Arms 2 and 3, and DOM PK collection in Arm 3: pre-dose and at EOI on C1D1, and only pre-dose samples on Day 1 of Cycles 2, 6, and 10. Thereafter, PK samples are collected at pre-dose on Day 1 every 8 cycles (e.g., C18D8, C26D8, etc.) and at EOT visit. The PK collection window for pre-dose samples: −30 minutes prior to the start of infusion of the first drug; post-dose samples: +30 minutes at the end of infusion for each drug.

PK is evaluated from serum samples using an assay performed by the Sponsor's designee.

The immunogenicity assessment is conducted to detect and measure ADAs against moieties of SG. SG ADA in Arms 1, 2 and 3: pre-dose on Day 1 of Cycles 1, 2, 6 and 10. Thereafter, ADA samples are collected at pre-dose on Day 8 every 8 cycles (e.g., C18D8, C26D8, etc.) and at the EOT visit. ZIM ADA collection in Anns 2 and 3, and DOM ADA collection in Arm 3: pre-dose on Day 1 of Cycles 1, 2, 6 and 10. Thereafter, ADA samples are collected at pre-dose on Day 1 every 8 cycles (e.g., C18D8, C26D8, etc.) and at EOT visit. The collection window for the ADA sample is −30 minutes prior to the start of infusion of first drug for predose samples.

### Biomarker Testing

### Biomarker Samples to Address the Study Objectives

Tumor samples are collected from all subjects who have provided consent to biomarker analyses and evaluate the association of tissue-based biomarkers with treatment response (including efficacy and/or AEs) and dosage selection, and to better understand the biological pathways, biology of UC, and/or the validation of a potential companion diagnostic for SG. These include Trop-2 expression across all arms and PD-L1 expression and other biomarkers. A baseline biopsy is required if archival tissue is not available.

Because biomarker science is a rapidly evolving area of investigation, and AEs in particular are difficult to predict, it may not be possible to prospectively specify all tests that may be performed on the specimens provided. The specific analyses include but may not be limited to the biomarkers outlined above and assays listed below. The testing outlined below is based upon the current state of scientific knowledge. It may be modified during or after the end of the study to remove tests no longer indicated and/or to add new tests based upon new state-of-the-art knowledge.

Biomarkers may include, but are not limited to protein expression, genetic testing, analyses of specific immune and tumor signatures (RNA), as well as tumor mutational burden and specific tumor mutations (DNA). Examples may include Trop-2 expression, PD-L1 expression, mutations/gene expression related to the DNA damage repair pathways, tumor mutational burden, oncogenic mutations, composition of immune subsets in the tumor microenvironment, pathological features of the tumor, and spatial heterogeneity of Trop-2 expression. These biomarkers are used to evaluate response and resistance to SG and other study treatments and to better understand molecular attributes predictive of SG response in UC. In addition, these biomarkers may also be used to evaluate the response and resistance to treatment that is combined with SG in some arms of the study. The specimen may be used to develop biomarker and/or diagnostic assays and establish the performance characteristics of these assays. Samples collected for biomarker analyses are destroyed no later than 15 years after the end of the study or per country requirements.

### End of Treatment

All subjects must complete an EOT visit after discontinuation from all study treatments, and complete assessments and procedures specified. If the EOT and Safety Follow-up visit coincide within 2 weeks of each other, the same assessments may be utilized for both events. Subjects who discontinue treatment due to toxicity continue with radiologic response assessments at the protocol-required schedule, until radiologic progression of disease or initiation of new therapy with capture of radiologic evidence of progression if clinically feasible. The reason for study discontinuation is documented and any treatment-related AE or clinically significant abnormal laboratory values at that time are followed until resolution or stabilization.

To be conducted within 4 weeks after the last dose of study drug(s) in the event of premature study termination:
Physical examination
Weight
Pregnancy test, if applicable
Vital signs defined as heart rate, systolic and diastolic blood pressure, respiratory rate, body weight, and body temperature.
CT or MRI with contrast (chest, abdomen, pelvis, other regions of known/suspected involvement)
MRI Brain (if known brain metastasis)
Blood samples
   CBC (with differential and platelet count).
   Serum chemistry panel
   Coagulation: INR or PT and aPTT
   Serum sample for ADAs (frozen immediately, to be shipped to Sponsor's designee for analysis)
   TSH and reflex T3/4 for all subjects
Urinalysis
PK and immunogenicity samples
Concomitant medications (continued, changed)
AE reporting
   Criteria for Removal from Protocol Treatment

Subjects are removed from protocol treatment under the following conditions:
First documentation of PD or symptomatic deterioration indicating treatment failure unless the investigator assesses that the subject is receiving clinical benefit from continued therapy.

Treatment beyond PD is permitted if there is evidence of clinical benefit.
Unacceptable toxicity
Treatment delay for any reason >5 weeks
Pregnancy
Non-compliance of the subject
Withdrawal of consent to be treated at any time for any reason; all subjects continue to be followed for survival unless they expressly withdraw consent for this follow up as well.
Death
Lost to follow-up

### Safety Follow-Up

A Safety Follow-up visit occurs after the EOT visit, with the time window dependent on the study drug administered. Subjects who discontinue treatment due to reasons other than radiologic PD continue with radiologic response assessments at the protocol-required schedule, until progression of disease or initiation of new therapy if clinically feasible.

The following assessments are performed:
Physical examination
Weight
Pregnancy test, if applicable
Vital signs defined as heart rate, systolic and diastolic blood pressure, respiratory rate, body weight, and body temperature.
Blood samples
   CBC (with differential and platelet count). Tests that report clinically significant abnormal values should be repeated within 48 hours.
   Serum chemistry panel (tests that report clinically significant abnormal values should be repeated within 48 hours).
   Coagulation: INR or PT and aPTT
   Serum sample for ADA (frozen immediately, to be shipped to Sponsor's designee for analysis)
   Serum sample for PK analysis (frozen immediately, to be shipped to Sponsor's designee for analysis)
   TSH and reflex T3/4 for all subjects
Urinalysis
Concomitant medications (continued, changed)
AE reporting

### Survival Follow-Up

All subjects are followed every 12 weeks for survival for a maximum of 2 years, after Safety Follow-up visit. This may be by telephone or visit and includes documentation of any further anticancer therapy administered for their UC. Survival status may also be documented from public databases (e.g., Social Security database), as allowed by local regulations.

### End of Study

Subjects end the study under any of the following conditions:
Death
Withdrawal of consent from the study at any time for any reason
Loss to follow-up
Sponsor decision
Completion of the 2 years follow-up period

The end of the study is the date of the last visit/contact/survival follow-up in the study.

### Example 2

### A Phase 2 Study of Sacituzumab Govitecan as a Maintenance Treatment in Participants With Bladder Cancer

The purpose of this study is to assess the safety and efficacy of sacituzumab govitecan in combination with other anti-tumor agents as a maintenance treatment in participants with bladder cancer.

In Arm 1 of the study, human patients with bladder cancer are treated with a sacituzumab govitecan monotherapy (administered at a dose of 10 mg/kg on days 1 and 8 of a 21-day cycle) until PD, unacceptable toxicity, or loss of clinical benefit.

In Arm 2 of the study, human patients with bladder cancer are treated with a combination of sacituzumab govitecan (administered at a dose of 10 mg/kg on days 1 and 8 of a 21-day cycle) and zimberelimab (administered at a dose of 360 mg on day 1 of the 21-day cycle) until PD, unacceptable toxicity, or loss of clinical benefit.

In Arm 3 of the study, human patients with bladder cancer are treated with a combination of sacituzumab govitecan (administered at a dose of 10 mg/kg on days 1 and 8 of a 21-day cycle), zimberelimab (administered at a dose of 360 mg on day 1 of the 21-day cycle), and domvanalimab (administered at a dose of 1200 mg on day 1 of the 21-day cycle) until PD, unacceptable toxicity, or loss of clinical benefit. Subjects who must discontinue 1 agent may continue the others until PD, unacceptable toxicity, or loss of clinical benefit.

Primary outcome measures include: (1) Progression-Free Survival (PFS) according to Response Evaluation Criteria in Solid Tumors (RECIST Version 1.1) Assessed by Investigator [Time Frame: Time from randomization of study drug until first documentation of progressive disease (PD) or death, assessed approximately up to 51 months]; and (2) Number of Participants with Treatment Emergent Adverse Events (TEAEs), Treatment-Related Adverse Events, and AEs of Special Interest (AESIs) as per Qualitative Toxicity Scale [National Cancer Institute-Common Terminology Criteria for Adverse Events 5.0][Time Frame: Randomization up to the last safety follow-up visit at approximately up to 51 months].

Secondary outcome measures include:
Overall Survival (OS) [Time Frame: Time from randomization of study drug until death, assessed approximately up to 51 months]
Objective Response (OR) According to Response Evaluation Criteria in Solid Tumor (RECIST) v1.1 Assessed by Investigator [Time Frame: Time from randomization of study drug up to 51 months]
Duration of Response (DoR) According to Response Evaluation Criteria in Solid Tumor (RECIST) v1.1 Assessed by Investigator [Time Frame: Time from first documented objective response to PD or death due to any cause, assessed approximately up to 51 months]
Pharmacokinetic Serum Concentration of sacituzumab govitecan, zimberelimab, and domvanalimab [Time Frame: Pre-dose up to safety follow up, assessed approximately up to maximum 51 months]
Number of Participants with Positive Anti-Drug Antibody (ADA) of sacituzumab govitecan, zimberelimab, and domvanalimab [Time Frame: Baseline up to 51 months]
Change From Baseline in National Comprehensive Cancer Network-Functional Assessment of Cancer Therapy (NCCN-FACT) Bladder Symptom Index-18 (FB1SI-18) Disease Related Symptoms-Physical Subscale (DRS-P) Scores [Time Frame: Baseline, Month 51]

### Inclusion Criteria

Participants with histologically confirmed, unresectable locally advanced or metastatic urothelial carcinoma. Both transitional cell and mixed transitional/non-transitional cell histologies are allowed, but transitional cell carcinoma must be the predominant histology
Participants has documented Stage IIIA/IIIB with N1-N3, or Stage IV disease (per American Joint Committee on Cancer/International Union for Cancer Control Tumor Node Metastasis system, 8th edition) at the start of first line chemotherapy.
The last dose of first line chemotherapy must have been received no less than 4 weeks, and no more than 10 weeks, prior to randomization in the present study
Estimated life expectancy of at least 3 months
Participants without progressive disease as per RECIST v1.1 guidelines following completion of 4 to 6 cycles of 1 L chemotherapy. Eligibility based on this criterion will be determined by Investigator review of pre chemotherapy and post chemotherapy radiological assessments (CT/MRI scans).
Eastern Cooperative Oncology Group (ECOG) performance status (PS) 0 or 1
Adequate hematological, hepatic, and renal function as defined in the protocol Other protocol defined inclusion criteria could apply

### Exclusion Criteria:

Participants with prior immunotherapy with Interleukin-2 (IL-2), IL-15, interferon alfa (IFN-α), or an anti-programmed death receptor-1 (PD-1), anti-programmed death-ligand 1 (PD-L1), anti PD-L2, anti CD137, or cytotoxic T cell lymphocyte-4 (CTLA-4) antibody (including ipilimumab), anti TROP2, any other antibody or drug specifically targeting T cell costimulation or immune checkpoint pathways, or any of the investigational drugs used in combination with avelumab.
Participants with active infection 48 hours before randomization requiring systemic therapy
Participants with known prior or suspected hypersensitivity to study drugs or any component in their formulations
Participants with prior adjuvant or neoadjuvant systemic therapy within 12 months of randomization
Participants with vaccination within 4 weeks of the first dose of study treatment and while on trial is prohibited except for administration of inactivated vaccines (for example, inactivated influenza vaccines) and replication-deficient coronavirus vaccines approved or authorized by local Health Authorities
Other protocol defined exclusion criteria could apply

Additional analyses, including, but not limited to, safety analyses, efficacy analyses, pharmacokinetic analyses, exploratory analyses, and/or biomarker analyses, may be performed as described in Example 1.

### Example 3

A Phase 2 Platform Study Evaluating the Safety and Efficacy of Novel Treatment Combinations in Patients with Lung Cancer

The purpose of this study is to assess the safety and efficacy of sacituzumab govitecan in combination with other anti-tumor agents for the treatment of non-small cell lung cancer (NSCLC).

In this study, human patients with NSCLC are treated with a combination of sacituzumab govitecan (administered at a dose of 10 mg/kg on days 1 and 8 of a 21-day cycle) zimberelimab (administered at a dose of 360 mg on day 1 of the 21-day cycle), and domvanalimab (administered at a dose of 1200 mg on day 1 of the 21-day cycle) until PD, unacceptable toxicity, or loss of clinical benefit.

Primary outcome measure includes objective response rate (ORR), defined as the proportion of participants achieving a complete response (CR) or partial response (PR). CR and PR require confirmation at least 4 weeks after the first detection of response and as assessed by the investigator according to RECIST version 1.1.

Secondary outcome measures include: (i) progression-free survival (PFS), defined as the time from the date of randomization until disease progression (PD), as assessed by the investigator according to RECIST version 1.1 or death, whichever comes first; (ii) duration of response (DOR), defined as the time from the first response (CR or PR) until the first documented PD, as assessed by the investigator according to RECIST version 1.1 or death, whichever comes first; (iii) overall survival (OS), defined as the time from the date of randomization until death from any cause; and (iv) the incidence of treatment-emergent adverse events (TEAEs), treatment-related adverse events and laboratory abnormalities.

### Inclusion Criteria:

Confirmed NSCLC with evidence of stage IV disease.
ECOG performance status score of 0 or 1 (ECOG: Eastern Cooperative Oncology Group; ECOG performance status: A tool that measures how cancer affects a patient's ability to carry out tasks related to daily living).
Measurable disease as per RECIST 1.1 criteria (RECIST: Response Evaluation Criteria in Solid Tumors. RECIST criteria: A standard way to measure how well a cancer patient responds to treatment. It is based on whether tumors shrink, stay the same, or get bigger).
Adequate function of internal organs.
Agree to use birth control methods per study instructions while participating in this study.
No epidermal growth factor receptor (EGFR) or anaplastic lymphoma kinase (ALK) gene alteration.
No known actionable genomic alterations for which therapies are already available in the market.
No prior treatment was received for metastatic NSCLC.

### Exclusion Criteria:

Confirmed mixed small-cell lung cancer and NSCLC.
Participants with active cancer spread from the original tumor to the brain and/or meninges.
Received previous anticancer therapy within 4 weeks before enrollment.
Active second malignancy within 3 years before enrollment.
Active autoimmune disease.
History of or current non-infectious pneumonitis/interstitial lung disease.
Active serious infection within 4 weeks before study treatment.

Additional analyses, including, but not limited to, safety analyses, efficacy analyses, pharmacokinetic analyses, exploratory analyses, and/or biomarker analyses, may be performed as described in Example 1.

### Example 4

### Development of Immunocompetent Mouse Models Bearing Solid Tumors Expressing Murine or Human Trop-2

The purpose of this study was to develop immunocompetent mouse models bearing solid tumors expressing mouse Trop-2 or human Trop-2.

### Cell Culture Conditions and Lentiviral Transduction

Murine breast cancer EMT6 (ATCC CRL-2755) were grown in basal cell culture medium comprised of DMEM+GlutaMAX (Thermo Fisher) and E0771 cells (ATCC CRL-3461) n Waymouth's media plus Glutamax, 10% FBS, and P/S. Media were supplemented with 10% FBS, 100 U/mL penicillin, and 0.1 mg/mL streptomycin.

To generate mouse or human Trop-2 overexpressing cells, 2.5×10⁵ of cells were seeded in a 6-well plate and incubate at 37° C., 5% CO₂ incubator overnight to allow cell attachment. Next day, cells were washed with PBS twice and treated with 2 mL fresh completed culture medium containing 8 μg/mL of polybrene following by infected with 5 MOIs of lentivirus (VectorBuilder) carried mouse TACSTD2 [nM_020047.3] or human TACSTD2 [nM_002353.3] gene overnight. On day 3, the supernatants were replaced with 2 mL fresh medium containing 3 μg/mL puromycin for selection every three days.

To confirm cell transduction with lentivirus, flow analysis for surface Trop-2 was performed. Briefly, cells were trypsinized, pelleted by centrifugation, and washed twice with stain buffer (BD Bioscience). Then cells were stained with 0.2 mg/mL of an anti-mouse Trop-2 (biotinylated BAF1122, 10 μg/ml) or anti-human Trop-2 (hRS7, 1:200 dilution) for 30 min on ice, and washed with PBS. After washing twice with stain buffer, cells were stained with Alexa Fluor-647 Streptavidin (Biolegend #405237, 1/500) or Alexa Fluor-647 goat anti-human IgG Fc (Jackson ImmunoResearch, 1:500 dilution) for another 30 mins on ice, then wash twice with stain buffer. SYTOX™ blue (Invitrogen) staining was used as live/dead staining. The signal intensity of Alexa Fluor-647 was measured by flow cytometry using a LSRFortessa cell analyzer (BD Bioscience).

Transduced cells with high expression level of surface Trop-2 were further sorted out twice via a FACS Aria Fusion sorter (BD Bioscience).

### Generation of Mouse Models Bearing Solid Tumors Expressing Mouse or Human Trop-2

Female Balb/c mice were implanted subcutaneously with murine Trop-2 transduced EMT6 triple negative breast cancer cells (Jin Y, An X, Mao B, et al. Different syngeneic tumors show distinctive intrinsic tumor-immunity and mechanisms of actions (MOA) of anti-PD-1 treatment. Sci Rep 2022; 12(1): 3278), thereby producing an immunocompetent mouse model expressing mouse Trop-2.

Female hTrop-2 transgenic mice (C57bl/c background) were orthotopically implanted in the mammary fad pad with human Trop-2 transduced E0771 luminal B subtype breast cancer cells (Le Naour A, Rossary A, Vasson M P, Cancer Med 2020; 9(21): 8074-85), thereby producing an immunocompetent mouse model expressing human Trop-2.

### Example 5

### Development of Murinized Sacituzumab Govitecan (SG) ADC, Surrogate SG ADC and Control ADC

The purpose of this study was to develop murinized sacituzumab govitecan (SG) ADC, surrogate SG ADC and control ADC.

To engineer an anti-human Trop-2 ADC tolerated in an immunocompetent mouse model, we used the parental mouse antibody RS7 variable regions and fused it to a mouse IgG2a backbone. The resulting murinized RS7 was coupled to CL2A linker/SN-38 payload at a high DAR (˜8-9).

To engineer an anti-mouse Trop-2 ADC binding to mouse Trop-2 with proprieties similar to SG, we selected a commercially available anti-mouse Trop-2 rabbit antibody (Sino Biological, #50922-R064) and murinized it to obtain an anti-mouse Trop-2 IgG2a (Rab64/mG2a/mKap). Rab64 mAb was then coupled to CL2A linker/SN-38 payload at a high DAR (˜8-9). The binding of hRS7 mAb, SG, Rab64 Ab and surrogate SG to Trop-2 was characterized by surface plasmon resonance (SPR, Table 6) and demonstrated that surrogate SG bound to both mouse and human Trop-2 in the low nanomolar range (1.37 nM to mTrop-2 and 1.1 nM to hTrop-2).

TABLE 6
Constants of association (Kₐ), constants of dissociation
(K_{d}) and affinity constant (K_{D}) measured by SPR

| Human Trop−2 | kₐ (M⁻¹s⁻¹) | k_{d} (s⁻¹) | K_{D} (nM) |
|---|---|---|---|
| hRS7-hIgG1k | 4.397(1)e5 | 1.440(1)e−4 | 0.3276 |
| SG | 4.2340(9)e5 | 1.530(1)e−4 | 0.3615 |
| Rab64-mIgG2a | 8.299(8)e4 | 5.44(1)e−5 | 0.655 |
| Surrogate SG | 8.46(2)e4 | 9.68(3)e−5 | 1.145 |
| Mouse Trop-2 | kₐ (M⁻¹s⁻¹) | k_{d} (s⁻¹) | K_{D} (nM) |
| hRS7-hIgG1k | No binding detected | | |
| SG | No binding detected | | |
| Rab64-mIgG2a | 5.812(1)e5 | 8.107(2)e−4 | 1.3950 |
| Surrogate SG | 5.776(2)e5 | 7.949(3)e−4 | 1.3760 |

A mouse isotype antibody control (mMAB 1129/mG2a/mKap) was used as control ADC and coupled to CL2A linker/SN-38 payload in the same condition that Rab64 mAb. Table 7 summarizes the ADC constructs.

TABLE 7
Summary of ADC Constructs

| Name | Biologics type | DAR |
|---|---|---|
| Sacituzumab Govitecan (SG, | hRS7 hIgG1-CL2A-SN38 ADC | 7.5 |
| Trodelvy ®) | | |
| Murinized SG | mRS7 mIgG2a-CL2A-SN-38 ADC | 7.7 |
| Surrogate SG | Rab64 mIgG2a-CL2A-SN-38 ADC | 8.1 |
| Control surrogate and | mMAB1129 mIgG2a-CL2A-SN38 | 7.8 |
| mouse SG | ADC | |

### Example 6

Pharmacodynamic (PD) Study of Surrogate SG Combined to Anti-Mouse PD1 and/or Anti-Mouse TIGIT Antibodies in EMT6-mTrop-2 Syngeneic Mouse Model

Female BALB/c mice (6-8 weeks old) were obtained from Charles River Laboratories. Murine Trop-2-EMT6 cells (3×10⁵ cells in 0.1 mL) were resuspended in Waymouth's media and injected subcutaneously in the flank. When tumor volume reached a mean volume of 100 mm³, mice were randomized into 8 groups (5 mice per group) and treated intraperitoneally (IP) twice in one week as follow:
A. PBS
B. 200 μg/mouse of anti-mouse PD1 antibody (RMP1.14 mIgG1 D265A/mKap)
C. 100 μg/mouse of surrogate SG
D. 100 μg/mouse of surrogate SG+200 μg/mouse of anti-mouse PD1 antibody
E. 200 μg/mouse of anti-mouse TIGIT antibody (10A7 mIgG2a LALAPG)
F. 100 μg/mouse of surrogate SG+200 μg/mouse of anti-mouse TIGIT antibody
G. 200 μg/mouse of anti-mouse PD1 antibody+200 μg/mouse anti-mouse TIGIT antibody
H. 100 μg/mouse of surrogate SG+200 μg/mouse of anti-mouse PD1 antibody+anti-mouse TIGIT antibody.

Surrogate SG was produced as described in Example 5. The anti-mouse PD1 antibody RMP1.14 mIgG1 D265A is a commercially available antibody that can be purchased, for example, from Invivogen (cat. code mpd1-mab15-1). The anti-mouse TIGIT antibody 10A7 is a commercially available antibody that can be purchased, for example, from Invivogen (cat. code mtigit-mab10). The 10A7 antibody was further modified to contain LALAPG mutations.

Mice were sacrificed one week after the initiation of the treatment and harvested tumors were analyzed by flow cytometry using an immunophenotyping panel (Chou C-CK, J; Yang, B; Orf, J; Dave, R; Lai, Y; Lee, CV; Papalia, GA; Boyd, K; Diehl, L; Scholler, N. Development of triple-negative breast cancer (TNBC) syngeneic models and TROP2-directed antibody-drug conjugate (ADC) surrogate to model therapeutic combinations In: Research AAfC, editor. 2022 San Antonio Breast Cancer Symposium; Cancer Research). The percentage of tumor infiltrating immune cell types (per live cells for CD45+ cells or per CD45+ cells for T cells, CD8 T cells, CD4 T cells, DC, NK and macrophages) was plotted per treatment type as a heatmap (FIG. 3).

### Results

Flow cytometry analysis of mTrop-2-EMT6 tumors harvested suggests a trend of increase of T cells and decrease of macrophages in the in the tumor microenvironment (TME) after one week of monotherapy with surrogate SG (group C) to compare to control PBS (group A) (FIG. 3, 2^{nd} and 7^{th} columns). The immune composition was further modified by the combination of anti-TIGIT and anti-PD1 to surrogate SG.

The percentages of natural killer (NK) cells trended to increase in animals treated by surrogate SG in combination with anti-PD1 and anti-TIGIT (group H) to compare to all the other groups (FIG. 3, 6^{th} column).

The percentage of macrophage infiltration, often immunosuppressive in tumor microenvironment (Noy R, Pollard J W. Tumor-associated macrophages: from mechanisms to therapy. Immunity 2014; 41(1): 49-61, Nielsen S R, Schmid M C. Macrophages as Key Drivers of Cancer Progression and Metastasis. Mediators Inflamm 2017; 2017: 9624760), was decreased in animals treated with anti-TIGIT and anti-PD1 (group G) to compare with anti-TIGIT in monotherapy (group E) (p=0.019) and also trended to decrease in animals treated with SG in combination with anti-TIGIT and anti-PD1 antibodies (group H) (FIG. 3, 7^{th} column, light grey).

### Example 7

### In Vivo Efficacy of Murinized SG in an Immunocompetent Mouse Model of Orthotopic Breast Cancer

This study evaluates the efficacy of murinized SG in an immunocompetent mouse model of orthotopic breast cancer.

C57BL/6N-Tacstd2^{tml(TACSTD2}/Bcgen mice were acquired from Biocytogen. Human TROP2 mRNA was detectable only in homozygous B-hTROP2 mice (H/H) in skin and kidney; human Trop-2 protein was detectable in lung. The introduction of hTrop-2 in place of its mouse counterpart does not change the overall development, differentiation, or distribution of immune cells (www.biocytogen.jp/models/humanized/other/B-hTROP2-mice.html). Mice were treated with PBS (Group 1); 200 μg/mouse control ADC (Group 3); 500 μg/mouse control ADC (Group 2); 200 μg/mouse murinized SG (Group 5); or 500 μg/mouse murinized SG (Group 4).

FIG. 4 shows the survival curves of female huTROP2 KI C57BL/6 mice (n=10/group) bearing an orthotopic breast tumor expressing hTrop-2 (hTrop-2 E0771) treated with PBS; 200 μg/mouse control ADC; 500 μg/mouse control ADC; 200 μg/mouse murinized SG; or 500 μg/mouse murinized SG. Murinized SG demonstrated efficacy in this model system.

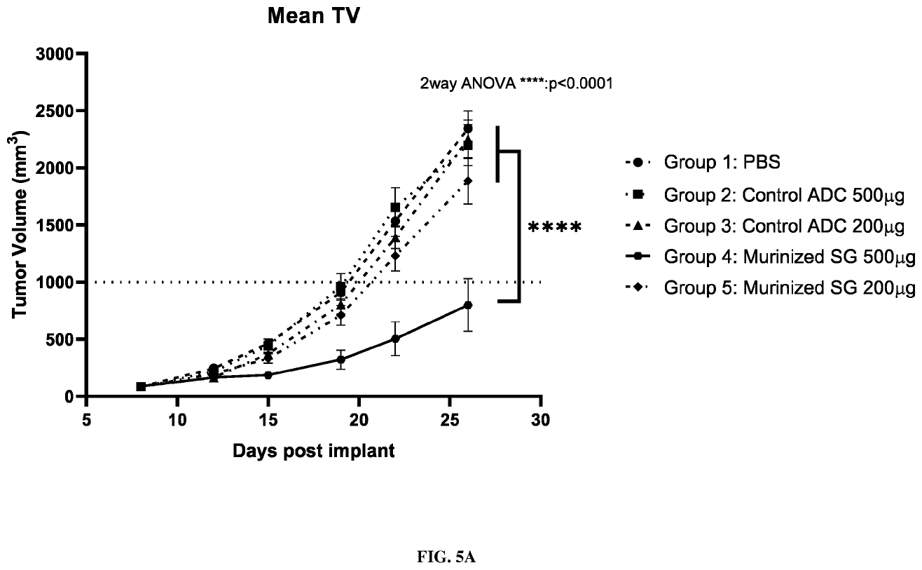

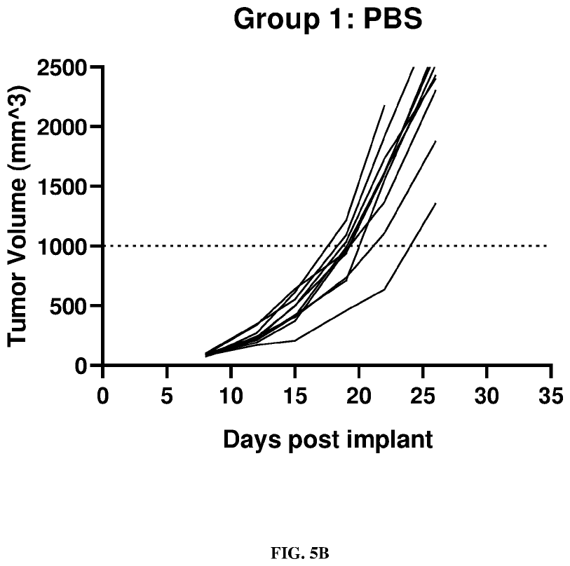

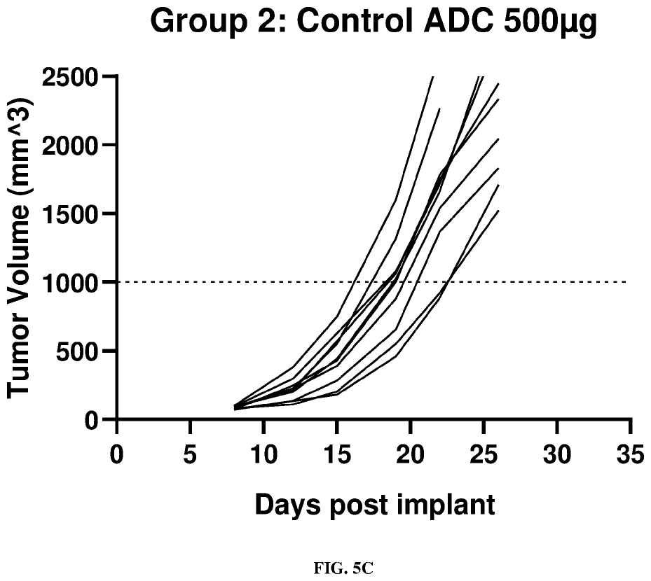

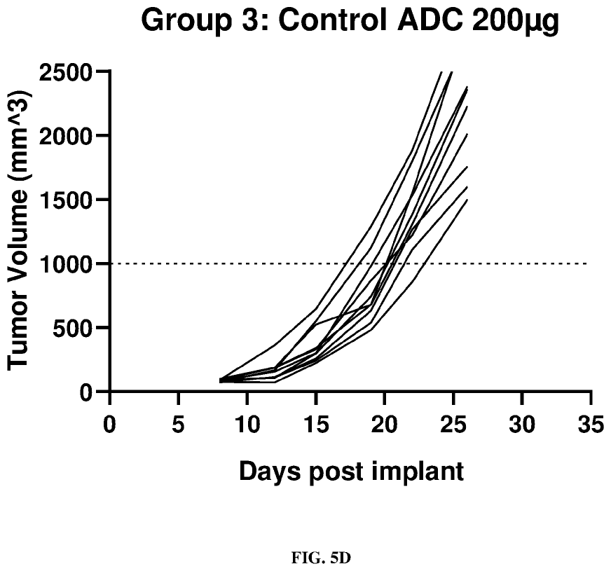

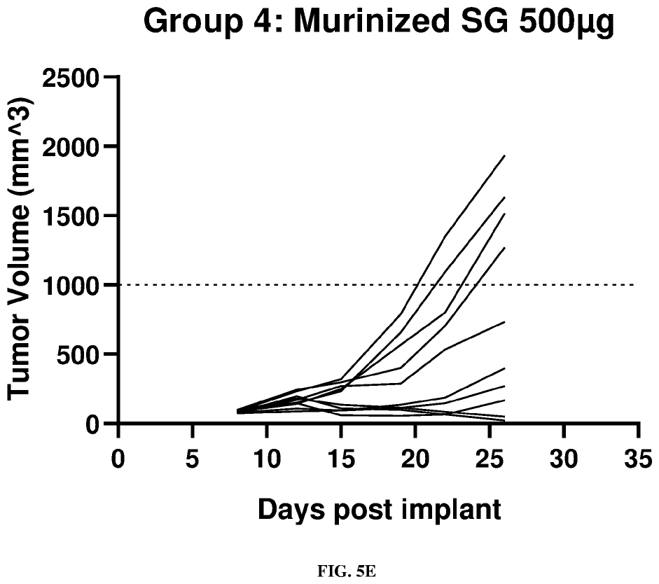

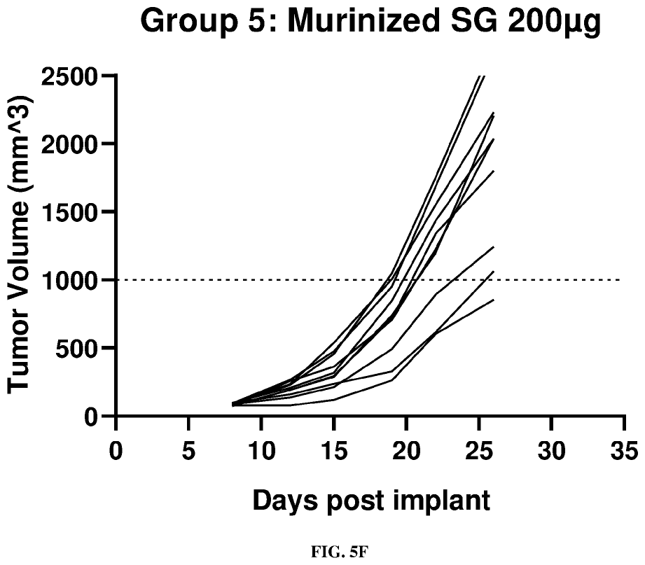

FIGS. 5A-F show growth curves of E0771 hTrop-2 in hTrop-2 KI mice, 26 days after tumor implant. FIG. 5A shows the mean tumor volume in mice treated with PBS (Group 1), 500 μg of control ADC (Group 2), 200 μg of control ADC (Group 3), 500 μg of murinized SG (Group 4), or 200 μg murinized SG (Group 5). FIG. 5B shows growth curves of E0771 hTrop-2 in hTrop-2 KI mice treated with PBS (Group 1). FIG. 5C shows growth curves of E0771 hTrop-2 in hTrop-2 KI mice treated with 500 μg of control ADC (Group 2). FIG. 5D shows growth curves of E0771 hTrop-2 in hTrop-2 KI mice treated with 200 μg of control ADC (Group 3). FIG. 5E shows growth curves of E0771 hTrop-2 in hTrop-2 KI mice treated with 500 μg of murinized SG (Group 4). FIG. 5F shows growth curves of curves E0771 hTrop-2 in hTrop-2 KI mice treated with 200 μg of murinized SG (Group 5).

### Example 8

In Vivo Efficacy of Murinized SG Combined to Anti-Mouse PD1 and/or Anti-Mouse TIGIT Antibodies in an Immunocompetent Mouse Model of Orthotopic Breast Cancer Expressing Human Trop-2

The purpose of this study is to determine the in vivo efficacy of murinized SG combined to anti-mouse PD1 and/or anti-mouse TIGIT antibodies in an immunocompetent mouse model of orthotopic breast cancer expressing human Trop-2.

C57BL/6N-Tacstd2^{tml(TACSTD2}/Bcgen mice were acquired from Biocytogen. Female C57BL/6N-Tacstd2^{tml(TACSTD2}/Bcgen (5-7 weeks old) are injected into the left fourth mammary fat pad with huTrop-2 transduced E0771 tumor cells. When the tumor volume reaches a mean volume of 100 mm³, mice are randomized into 12 groups (10 mice per group) and treated for four weeks with
1. PBS Biweekly (BIW)×4 intraperitoneally (IP)
2. 200 μg/mouse of anti-mouse PD1 antibody (RMP1.14 mIgG1 D265A/mKap) BIW×2 IP
3. 200 μg/mouse of murinized SG BIW×4 IP
4. 200 μg/mouse of murinized SG BIW×4 IP+200 μg/mouse of anti-mouse PD1 antibody BIW×2 IP
5. 200 μg/mouse of anti-mouse TIGIT antibody (10A7 mIgG2a LALAPG) BIW×3 IP
6. 200 μg/mouse of murinized SG BIW×4 IP+200 μg/mouse of anti-mouse TIGIT antibody BIW×3 IP
7. 200 μg/mouse of anti-mouse PD1 antibody BIW×2 IP+200 μg/mouse anti-mouse TIGIT antibody BIW×3 IP
8. 200 μg/mouse of murinized SG BIW×4 IP+200 μg/mouse of anti-mouse PD1 antibody BIW×2 IP+anti-mouse TIGIT antibody BIW×3 IP.

Murinized SG ADC was produced as described in Example 5. The anti-mouse PD1 antibody RMP1.14 mIgG1 D265A is a commercially available antibody that can be purchased, for example, from Invivogen (cat. code mpd1-mab15-1). The anti-mouse TIGIT antibody 10A7 is a commercially available antibody that can be purchased, for example, from Invivogen (cat. code mtigit-mab10). The 10A7 antibody was further modified to contain LALAPG mutations.

Palpable tumors are measured with calipers. Mice are sacrificed when tumor sizes reached ˜2000 mm³. It is anticipated that the group treated with triple combinations (group 8) will survive the longest due to the combination of the anti-tumor effect of murinized SG to the activation of the tumor infiltrate via IO drugs.

### EXEMPLARY EMBODIMENTS

1. A method of treating, mitigating, reducing, preventing or delaying the recurrence or metastasis of a Trop-2 positive cancer comprising co-administering to a subject an effective amount of:
   a) a TROP-2-targeted antibody-drug conjugate (ADC) comprising an anti-TROP-2 antibody (anti-TROP-2 ADC); and
   b) an anti-PD-(L)1 antibody.
2. A kit for use as a medicament, wherein the kit comprises
   a) a TROP-2-targeted antibody-drug conjugate (ADC) comprising an anti-TROP-2 antibody (anti-TROP-2 ADC); and
   b) an anti-PD-(L)1 antibody.
3. A kit for use in the treatment, mitigation, reduction, prevention, or delay of the recurrence or metastasis of a Trop-2 positive cancer, wherein the kit comprises
   a) a TROP-2-targeted antibody-drug conjugate (ADC) comprising an anti-TROP-2 antibody (anti-TROP-2 ADC); and
   b) an anti-PD-(L)1 antibody.
4. The method of embodiment 1 or kit of embodiment 2 or 3, wherein the anti-TROP-2 ADC comprises an anticancer agent payload.
5. The method or kit of embodiment 4, wherein the anticancer agent payload is selected from a microtubule inhibitor, DNA cleavage agent, and topoisomerase I inhibitor.
6. The method of embodiment 1 or kit of embodiment 2 or 3, wherein the anti-TROP-2 ADC comprises a topoisomerase I inhibitor.
7. The method or kit of embodiment 5 or 6, wherein the topoisomerase I inhibitor is selected from irinotecan, topetecan, and SN-38.
8. The method or kit of any one of embodiments 1 to 7, wherein the anti-TROP-2 ADC has a structural formula of mAb-CL2A-SN-38, with a structure represented by:

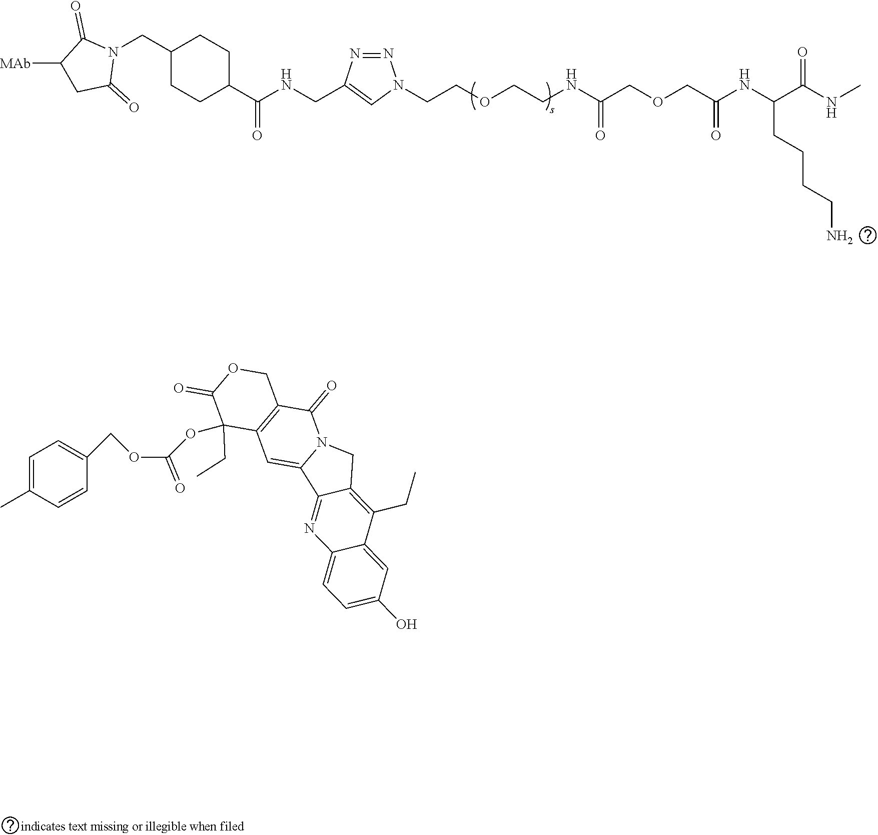

(described, e.g., in U.S. Pat. No. 7,999,083).
9. The method or kit of any one of embodiments 1 to 8 wherein the anti-TROP-2 ADC comprises sacituzumab (hRS7; described, e.g., in WO2003074566, FIGS. 3 and 4).
10. The method or kit of any one of embodiments 1 to 9, wherein the anti-TROP-2 ADC is selected from sacituzumab govitecan, datopotamab deruxtecan (DS-1062), ESG-401, SKB-264, DAC-02 and BAT-8003.
11. The method or kit of any one of embodiments 1 to 10, wherein the anti-TROP-2 ADC is sacituzumab govitecan.
12. The method or kit of embodiment 5, wherein the microtubule inhibitor is selected from is an auristatin, a taxane, a vinca alkaloid, an epothilone, and maytansinoid.
13. The method or kit of embodiment 12, wherein the auristatin is selected from monomethyl auristatin E (MMAE) and monomethyl auristatin F (MMAF).
14. The method or kit of embodiment 12, wherein the maytansinoid is selected from mertansine (DM1) and ravtansine (DM4).
15. The method or kit of embodiment 4, wherein the anticancer agent payload is selected from anthracyline (e.g., doxorubicin, daunorubicin, epirubicin, idarubicin), pyrrolobenzodiazepine (PBD), or dimer thereof, DNA cross-linking agent SC-DR002 (D6.5), duocarmycin, a duocarmycin (A, B1, B2, C1, C2, D, SA, CC-1065), tubulysin B and analogs thereof.
16. A method of treating, mitigating, reducing, preventing or delaying the recurrence or metastasis of a tumor antigen positive (TA⁺) cancer comprising co-administering to a subject an effective amount of:
   a) a tumor antigen-targeted antibody-drug conjugate (ADC) comprising a topoisomerase 1 inhibitor (TopI ADC); and
   b) an anti-PD-(L)1 antibody.
17. A kit for use as a medicament, wherein the kit comprises
   a) a tumor antigen-targeted antibody-drug conjugate (ADC) comprising a topoisomerase I inhibitor (TopI ADC); and
   b) an anti-PD-(L)1 antibody.
18. A kit for use in the treatment, mitigation, reduction, prevention, or delay of the recurrence or metastasis of a tumor antigen positive (TA⁺) cancer, wherein the kit comprises
   a) a tumor antigen-targeted antibody-drug conjugate (ADC) comprising a topoisomerase I inhibitor (TopI ADC); and
   b) an anti-PD-(L)1 antibody.
19. The method of embodiment 16 or kit of embodiment 17 or 18, wherein the topoisomerase I inhibitor is a camptothecin.
20. The method or kit of embodiment 19, wherein the camptothecin is selected from irinotecan, topotecan, belotecan, and exatecan derivative.
21. The method or kit of embodiment 20, wherein the exatecan derivative is selected from Dxd or SN38.
22. The method or kit of any one of embodiments 16 to 21, wherein the topoisomerase I inhibitor is selected from irinotecan, topetecan, and SN-38.
23. The method or kit of any one of embodiments 16 to 22, wherein the TopI ADC has a structural formula of mAb-CL2A-SN-38, with a structure represented by:

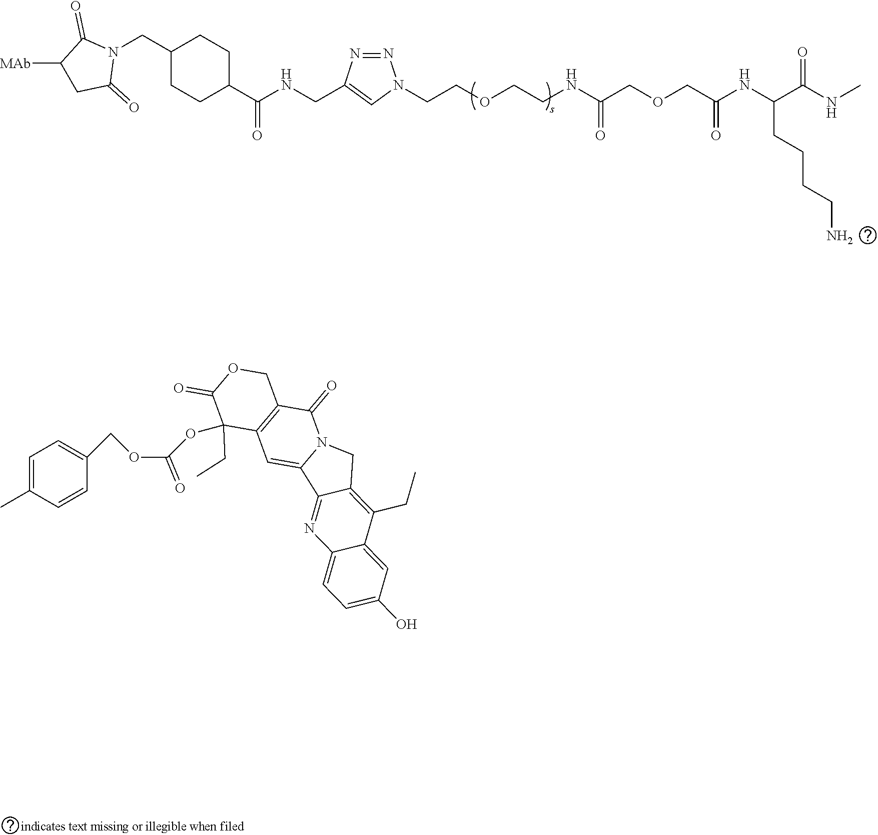

(described, e.g., in U.S. Pat. No. 7,999,083).
24. The method or kit of any one of embodiments 16 to 23, wherein the TopI ADC comprises an antibody that binds a tumor antigen.
25. The method or kit of embodiment 24, wherein the tumor antigen is selected from carbonic anhydrase IX, B7, CCCL19, CCCL21, CSAp, HER-2/neu, BrE3, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD80, CD83, CD95, CD126, CD133, CD138, CD147, CD154, CEACAM5, CEACAM-6, alpha-fetoprotein (AFP), VEGF, ED-B fibronectin, EGP-1, EGP-2, EGF receptor (ErbB1), ErbB2, ErbB3, Factor H, FHL-1, Flt-3, folate receptor, Ga 733, GROB, HMGB-1, hypoxia inducible factor (HIF), HM1.24, insulin-like growth factor (ILGF), IFN-a, IFN-b, IL-g, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, HLA-DR, HLA-G, CD66a-d, MAGE, mCRP, MCP-1, MIP-1A, MIP-1B, macrophage migration-inhibitory factor (MIF), MUC1, MUC2, MUC3, MUC4, MUC5, placental growth factor (P1GF), PSA (prostate-specific antigen), PSMA, PSMA dimer, PAM4 antigen, NCA-95, NCA-90, A3, A33, Ep-CAM, KS-1, Le(y), mesothelin, S100, tenascin, TAC, Tn antigen, Thomas-Friedenreich antigens, tumor necrosis antigens, tumor angiogenesis antigens, TNF-a, TRAIL receptor (R1 and R2), VEGFR, RANTES, T101, cancer stem cell antigens, complement factors C3, C3a, C3b, C5a, C5, and an oncogene product.
26. The method or kit of any one of embodiments 16 to 23, wherein the TopI ADC comprises an antibody selected from gemtuzumab, brentuximab, belantamab, camidanlumab, trastuzumab, inotuzumab, glembatumumab, anetumab, mirvetuximab, depatuxizumab, vadastuximab, labetuzumab, ladiratuzumab, loncastuximab, patritumab, lifastuzumab, indusatumab, polatuzumab, pinatuzumab, coltuximab, upifitamab, indatuximab, milatuzumab, rovalpituzumab, enfortumab, tisotumab, tusamitamab, disitamab, telisotuzumab, and antigen-binding fragments thereof.
27. The method or kit of any one of embodiments 16 to 23, wherein the TopI ADC comprises an antibody selected from hLL1 (anti-CD74), hLL2 (anti-CD22), hRFB4 (anti-CD22), h PAM4 (anti-MUC5ac), hMN-3 (anti-NOTCH3), hMN-14 (labetuzumab; anti-CEACAM5); hMN15 (anti-CEACAM6) hA19 (anti-CD19), hA20 (anti-CD22), hMu-9 (anti-CSAp), hL243 (anti-HLA-DR), hImmu-31 (anti-AFP), and antigen-binding fragments thereof.
28. The method or kit of any one of embodiments 1 to 27, wherein the anti-PD-(L)1 antibody is selected from pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolinizumab, prolgolimab, lodapolimab, camrelizumab, budigalimab, avelumab, dostarlimab, envafolimab, sintilimab, or zimberelimab.
29. The method or kit of embodiment 28, wherein the anti-PD-(L)1 antibody is zimberelimab.
30. The method or kit of any one of embodiments 1 to 28, wherein the anti-PD-(L)1 antibody is an Fc-silent antibody.
31. The method or kit of any one of embodiments 1 to 28, wherein the anti-PD-(L)1 antibody is an Fc-enabled antibody.
32. The method or kit of any one of embodiments 1 to 31, further comprising administering an anti-TIGIT antibody.
33. The method or kit of embodiment 32, wherein the anti-TIGIT antibody is AB308, AGEN-1307 (AGEN-1327), AGEN-1777, AK127, BMS-986207, domvanalimab, EOS-448, etigilimab, JS006, M6223, ociperlimab, ralzapastotug, SEA-TGT (SGN-TGT), tiragolumab, or vibostolimab.
34. The method or kit of embodiment 32 or 33, wherein the anti-TIGIT antibody is an Fc-silent antibody.
35. The method or kit of embodiment 32 or 33, wherein the anti-TIGIT antibody is an Fc-enabled antibody.
36. The method or kit of any one of embodiments 32 to 34, wherein the anti-TIGIT antibody is domvanalimab.
37. The method or kit of any one of embodiments 32, 33, or 35, wherein the anti-TIGIT antibody is AB308.
38. The method or kit of any one of embodiments 32, 33, or 35, wherein the anti-TIGIT antibody is ralzapastotug.
39. The method or kit of any one of embodiments 32, 33, or 35, wherein the anti-TIGIT antibody is AGEN-1372, ociperlimab, or tiragolumab.
40. The method or kit of embodiment 33, wherein the anti-TIGIT antibody is M6223.
41. The method or kit of embodiment 32 or 33, wherein the anti-PD-(L)1 antibody and anti-TIGIT antibody are a) zimberelimab and domvanalimab, b) zimberelimab and AB308, c) atezolizumab and tiragolumab, d) pembrolizumab and vibostolimab, e) pembrolizumab and domvanalimab, f) pembrolizumab and AB308, g) MK-7684A (pembrolizumab/vibostolimab coformulation), h) durvalumab and domvanalimab, i) zimberelimab and ralzapastotug, or j) pembrolizumab and ralzapastotug.
42. The method or kit of embodiment 41, wherein the anti-PD-(L)1 antibody and anti-TIGIT antibody are zimberelimab and domvanalimab.
43. The method or kit of any one of embodiments 1 to 42, wherein the cancer is a solid epithelial cancer.
44. The method or kit of embodiment 43, wherein the solid epithelial cancer is selected from breast cancer (e.g., triple negative breast cancer (TNBC), HR⁺/Her2⁻ breast cancer, or HR⁺/Her2^{low} breast cancer), colorectal cancer, lung cancer, stomach cancer, urinary tract cancer, urothelial cancer, bladder cancer, renal cancer, pancreatic cancer, ovarian cancer, uterine cancer, esophageal cancer and prostatic cancer.
45. The method or kit of embodiment 44, wherein the cancer is bladder cancer.
46. The method or kit of embodiment 44 or 45, wherein the bladder cancer is urothelial cancer.
47. The method or kit of any one of embodiments 44 to 46, wherein the bladder cancer is (i) unresectable, locally advanced bladder cancer, (ii) metastatic bladder cancer, or (iii) muscle-invasive bladder cancer.
48. The method or kit of embodiment 46, wherein the urothelial cancer is (i) unresectable, locally advanced urothelial cancer, (ii) metastatic urothelial cancer, or (iii) muscle-invasive urothelial cancer.
49. The method or kit of embodiment 44, wherein the cancer is lung cancer.
50. The method or kit of embodiment 44 or 49, wherein the lung cancer is non-small cell lung cancer (NSCLC).
51. The method or kit of embodiment 50, wherein the NSCLC is (i) squamous NSCLC or (ii) non-squamous NSCLC.
52. The method or kit of any one of embodiments 44 and 49 to 51, wherein the lung cancer is without EGFR, ALK, or other actionable genomic alterations.
53. The method or kit of any one of embodiments 43 and 48 to 51, wherein the cancer is (i) advanced NSCLC; or (ii) metastatic NSCLC.
54. The method or kit of embodiment 44, wherein the cancer is breast cancer.
55. The method or kit of embodiment 44 or 54, wherein the breast cancer is triple negative breast cancer, HR⁺/HER2⁻ breast cancer, or HR⁺/HER2^{low} breast cancer.
56. The method or kit of any one of embodiments 1 to 55, wherein the cancer is (i) unresectable, locally advanced, (ii) advanced, (iii) metastatic, (iv) muscle-invasive, or (v) resistant or refractory to one or more anti-cancer therapies.
57. The method or kit of embodiment 56, wherein the cancer is resistant or refractory to one or more anti-cancer therapies selected from radiation therapy, chemotherapy (including NHA therapy), and checkpoint inhibitor therapy (e.g., anti-PD(L)1 antibody).
58. The method or kit of any one of embodiments 1 to 57, wherein the cancer has progressed following at least one prior anti-cancer therapy.
59. The method or kit of any one of embodiments 1 to 58, wherein the subject is treatment naïve.
60. The method or kit of embodiment 59, wherein the treatment naïve subject has not received prior anti-cancer therapy for (i) unresectable, locally advanced cancer, (ii) advanced cancer, (iii) metastatic cancer, or (iv) muscle invasive cancer.
61. The method or kit of any one of embodiments 1 to 60, wherein the subject has received one or more anti-cancer therapies before administration of the combination of the ADC, anti-PD-(L)1 antibody, and optionally anti-TIGIT antibody.
62. The method or kit of any one of embodiments 1 to 58, wherein the subject is not treatment naïve.
63. The method or kit of any one of embodiments 1 to 58, wherein the subject has received one or more prior anti-cancer therapies for (i) unresectable, locally advanced cancer, (ii) advanced cancer, (iii) metastatic cancer, (iv) muscle invasive cancer, or (v) resistant or refractory to one or more anti-cancer therapies.
64. The method or kit of any one of embodiments 1 to 58, wherein the subject has received one or more anti-cancer therapies before administration of the combination of the ADC, anti-PD-(L)1 antibody, and optionally anti-TIGIT antibody.
65. The method or kit of any one of embodiments 1 to 64, wherein the cancer is resistant or refractory to one or more anti-cancer therapies.
66. The method or kit of any one of embodiments 58 to 65, wherein the anti-cancer therapy is selected from surgery, radiation therapy, chemotherapy, and checkpoint inhibitor therapy.
67. The method or kit of any one of embodiments 1 to 66, wherein the combination of the ADC, anti-PD-(L)1 antibody, and optionally anti-TIGIT antibody is administered in a neoadjuvant setting.
68. The method or kit of any one of embodiments 1 to 66, wherein the combination of the ADC, anti-PD-(L)1 antibody, and optionally anti-TIGIT antibody is administered in a adjuvant setting.
69. The method or kit of any one of embodiments 1 to 66, wherein the combination of the ADC, anti-PD-(L)1 antibody, and optionally anti-TIGIT antibody is administered in a therapeutic setting.
70. The method or kit of any one of embodiments 1 to 66, wherein the combination of the ADC, anti-PD-(L)1 antibody, and optionally anti-TIGIT antibody is administered in a maintenance setting.
71. The method or kit of any one of embodiments 1 to 70, wherein the subject receives one or more doses of the ADC.
72. The method or kit of any one of embodiments 1 to 71, wherein the subject receives one or more doses of the anti-PD-(L)1 antibody.
73. The method or kit of any one of embodiments 33 to 72, wherein the subject receives one or more doses of the anti-TIGIT antibody.
74. The method or kit of any one of embodiments 1 to 73, wherein the ADC and the anti-PD-(L)1 antibody are administered concurrently.
75. The method or kit of any one of embodiments 1 to 73, wherein the ADC and the anti-PD-(L)1 antibody are administered sequentially.
76. The method or kit of any one of embodiments 33 to 75, wherein the ADC and the anti-TIGIT antibody are administered concurrently.
77. The method or kit of any one of embodiments 33 to 75, wherein the ADC and the anti-TIGIT antibody are administered sequentially.
78. The method or kit of any one of embodiments 33 to 75, wherein the anti-PD-(L)1 antibody and the anti-TIGIT antibody are administered concurrently.
79. The method or kit of any one of embodiments 33 to 75, wherein the anti-PD-(L)1 antibody and the anti-TIGIT antibody are administered sequentially.
80. The method or kit of any one of embodiments 1 to 79, wherein the subject is human.
81. The method or kit of any one of embodiments 1 to 80, wherein the subject is cisplatin-ineligible.
82. The method or kit of any one of embodiments 1 to 81, wherein the anti-TROP-2 ADC is administered at one or more doses in the range of 8 mg/kg to 10 mg/kg.
83. The method or kit of embodiment 82, wherein the anti-TROP-2 ADC is administered at one or more doses of 10 mg/kg.
84. The method or kit of any one of embodiments 1 to 83, wherein the anti-TROP-2 ADC is administered intravenously.
85. The method or kit of any one of embodiments 1 to 84, wherein the anti-TROP-2 ADC is administered on days 1 and 8 of a 21-day cycle.
86. The method or kit of any one of embodiments 1 to 85, wherein the anti-PD-(L)1 antibody is administered at one or more doses in the range of 300 mg to 400 mg.
87. The method or kit of embodiment 86, wherein the anti-PD-(L)1 antibody is administered at dose of 360 mg.
88. The method or kit of any one of embodiments 1 to 87, wherein the anti-PD-(L)1 antibody is administered intravenously.
89. The method or kit of any one of embodiments 1 to 88, wherein the anti-PD-(L)1 antibody is administered on day 1 of a 21-day cycle.
90. The method or kit of embodiment 89, wherein the anti-TIGIT antibody is administered at one or more doses in the range of 800 mg to 1600 mg.
91. The method or kit of any one of embodiments 33 to 90, wherein the anti-TIGIT antibody is administered at a dose of 1200 mg.
92. The method or kit of any one of embodiments 33 to 91, wherein the anti-TIGIT antibody is administered intravenously.
93. The method or kit of any one of embodiments 33 to 92, wherein the anti-TIGIT antibody is administered on day 1 of a 21-day cycle.
94. A method of treating, mitigating, reducing, preventing or delaying the recurrence or metastasis of urothelial cancer comprising co-administering to a human cisplatin-ineligible patient an effective amount of:
   a) sacituzumab govitecan; and
   b) zimberelimab.
95. A kit for use as a medicament, wherein the kit comprises
   a) sacituzumab govitecan; and
   b) zimberelimab.
96. A kit for use in the treatment, mitigation, reduction, prevention, or delay of the recurrence or metastasis of urothelial cancer, wherein the kit comprises
   a) sacituzumab govitecan; and
   b) zimberelimab.
97. The method of embodiment 94 or kit of embodiment 95 or 96, wherein sacituzumab govitecan is administered at one or more doses in the range of 8 mg/kg to 10 mg/kg.
98. The method or kit of embodiment 97, wherein sacituzumab govitecan is administered at one or more doses of 10 mg/kg.
99. The method or kit of any one of embodiments 94 to 98, wherein sacituzumab govitecan is administered intravenously.
100. The method or kit of any one of embodiments 94 to 99, wherein sacituzumab govitecan is administered on days 1 and 8 of a 21-day cycle.
101. The method or kit of any one of embodiments 94 to 100, wherein zimberelimab is administered at one or more doses in the range of 300 mg to 400 mg.
102. The method or kit of embodiment 101, wherein zimberelimab is administered at dose of 360 mg.
103. The method or kit of any one of embodiments 94 to 102, wherein zimberelimab is administered intravenously.
104. The method or kit of any one of embodiments 94 to 103, wherein zimberelimab is administered on day 1 of a 21-day cycle.
105. The method or kit of any one of embodiments 94 to 104, wherein sacituzumab govitecan is administered at a dose of 10 mg/kg on days 1 and 8 of a 21-day cycle; zimberelimab is administered at a dose of 360 mg on day 1 of the 21-day cycle.
106. A method of treating, mitigating, reducing, preventing or delaying the recurrence or metastasis of urothelial cancer comprising co-administering to a human cisplatin-ineligible patient an effective amount of:
   a) sacituzumab govitecan;
   b) zimberelimab; and
   c) domvanalimab.
107. A kit for use as a medicament, wherein the kit comprises
   a) sacituzumab govitecan;
   b) zimberelimab; and
   c) domvanalimab.
108. A kit for use in the treatment, mitigation, reduction, prevention, or delay of the recurrence or metastasis of urothelial cancer, wherein the kit comprises
   a) sacituzumab govitecan;
   b) zimberelimab; and
   c) domvanalimab.
109. The method of embodiment 106 or kit of embodiment 107 or 108, wherein sacituzumab govitecan is administered at one or more doses in the range of 8 mg/kg to 10 mg/kg.
110. The method or kit of embodiment 109, wherein sacituzumab govitecan is administered at one or more doses of 10 mg/kg.
111. The method or kit of any one of embodiments 106 to 110, wherein sacituzumab govitecan is administered intravenously.
112. The method or kit of any one of embodiments 106 to 111, wherein sacituzumab govitecan is administered on days 1 and 8 of a 21-day cycle.
113. The method or kit of any one of embodiments 106 to 112, wherein zimberelimab is administered at one or more doses in the range of 300 mg to 400 mg.
114. The method or kit of embodiment 113, wherein zimberelimab is administered at dose of 360 mg.
115. The method or kit of any one of embodiments 106 to 114, wherein zimberelimab is administered intravenously.
116. The method or kit of any one of embodiments 106 to 115, wherein zimberelimab is administered on day 1 of a 21-day cycle.
117. The method or kit of any one of embodiments 106 to 116, wherein domvanalimab is administered at one or more doses in the range of 800 mg to 1600 mg.
118. The method or kit of embodiment 117, wherein domvanalimab is administered at a dose of 1200 mg.
119. The method or kit of any one of embodiments 106 to 118, wherein domvanalimab is administered intravenously.
120. The method or kit of any one of embodiments 106 to 119, wherein domvanalimab is administered on day 1 of a 21-day cycle.
121. The method or kit of any one of embodiments 106 to 120, wherein sacituzumab govitecan is administered at a dose of 10 mg/kg on days 1 and 8 of a 21-day cycle; zimberelimab is administered at a dose of 360 mg on day 1 of the 21-day cycle, and domvanalimab is administered at a dose of 1200 mg on day 1 of the 21-day cycle.
122. The method or kit of any one of embodiments 1 to 121, wherein an anti-CD47 antibody is not administered to the subject or human patient.
123. The method or kit of embodiment 122, wherein the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643.
124. The method or kit of embodiment 122, wherein the anti-CD47 antibody is magrolimab.
125. A method of treating, mitigating, reducing, preventing or delaying the recurrence or metastasis of non-small cell lung cancer comprising co-administering to a human patient an effective amount of:
   a) sacituzumab govitecan;
   b) zimberelimab; and
   c) domvanalimab.
126. A kit for use in the treatment, mitigation, reduction, prevention, or delay of the recurrence or metastasis of non-small cell lung cancer, wherein the kit comprises
   a) sacituzumab govitecan;
   b) zimberelimab; and
   c) domvanalimab.
127. The method of embodiment 125 or kit of embodiment 126, wherein sacituzumab govitecan is administered at one or more doses in the range of 8 mg/kg to 10 mg/kg.
128. The method or kit of embodiment 127, wherein sacituzumab govitecan is administered at one or more doses of 10 mg/kg.
129. The method or kit of any one of embodiments 125 to 128 wherein sacituzumab govitecan is administered intravenously.
130. The method or kit of any one of embodiments 125 to 129, wherein sacituzumab govitecan is administered on days 1 and 8 of a 21-day cycle.
131. The method or kit of any one of embodiments 125 to 130, wherein zimberelimab is administered at one or more doses in the range of 300 mg to 400 mg.
132. The method or kit of embodiment 131, wherein zimberelimab is administered at dose of 360 mg.
133. The method or kit of any one of embodiments 125 to 132, wherein zimberelimab is administered intravenously.
134. The method or kit of any one of embodiments 125 to 133, wherein zimberelimab is administered on day 1 of a 21-day cycle.
135. The method or kit of any one of embodiments 125 to 134, wherein domvanalimab is administered at one or more doses in the range of 800 mg to 1600 mg.
136. The method or kit of embodiment 135, wherein domvanalimab is administered at a dose of 1200 mg.
137. The method or kit of any one of embodiments 125 to 136, wherein domvanalimab is administered intravenously.
138. The method or kit of any one of embodiments 125 to 137, wherein domvanalimab is administered on day 1 of a 21-day cycle.
139. The method or kit of any one of embodiments 125 to 138, wherein sacituzumab govitecan is administered at a dose of 10 mg/kg on days 1 and 8 of a 21-day cycle; zimberelimab is administered at a dose of 360 mg on day 1 of the 21-day cycle, and domvanalimab is administered at a dose of 1200 mg on day 1 of the 21-day cycle.
140. The method or kit of any one of embodiments 125 to 139, wherein an anti-CD47 antibody is not administered to the subject or human patient.
141. The method or kit of embodiment 140, wherein the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643.
142. The method or kit of embodiment 141, wherein the anti-CD47 antibody is magrolimab.
143. The method or kit of any one of embodiments 125 to 142, wherein the NSCLC is (i) squamous NSCLC or (ii) non-squamous NSCLC.
144. The method or kit of any one of embodiments 125 to 143, wherein the NSCLC is without EGFR, ALK, or other actionable genomic alterations.
145. The method or kit of any one of embodiments 125 to 144, wherein the NSCLC is (i) metastatic or (ii) advanced.
146. A method of treating, mitigating, reducing, preventing or delaying the recurrence or metastasis of breast cancer comprising co-administering to a human cisplatin-ineligible patient an effective amount of:
   a) sacituzumab govitecan; and
   b) zimberelimab.
147. A kit for use in the treatment, mitigation, reduction, prevention, or delay of the recurrence or metastasis of breast cancer, wherein the kit comprises
   a) sacituzumab govitecan; and
   b) zimberelimab.
148. The method of embodiment 146 or kit of embodiment 147, wherein sacituzumab govitecan is administered at one or more doses in the range of 8 mg/kg to 10 mg/kg.
149. The method or kit of embodiment 148, wherein sacituzumab govitecan is administered at one or more doses of 10 mg/kg.
150. The method or kit of any one of embodiments 146 to 149, wherein sacituzumab govitecan is administered intravenously.
151. The method or kit of any one of embodiments 146 to 150, wherein sacituzumab govitecan is administered on days 1 and 8 of a 21-day cycle.
152. The method or kit of any one of embodiments 146 to 151, wherein zimberelimab is administered at one or more doses in the range of 300 mg to 400 mg.
153. The method or kit of embodiment 152, wherein zimberelimab is administered at dose of 360 mg.
154. The method or kit of any one of embodiments 146 to 153, wherein zimberelimab is administered intravenously.
155. The method or kit of any one of embodiments 146 to 154, wherein zimberelimab is administered on day 1 of a 21-day cycle.
156. The method or kit of any one of embodiments 146 to 155, wherein sacituzumab govitecan is administered at a dose of 10 mg/kg on days 1 and 8 of a 21-day cycle; zimberelimab is administered at a dose of 360 mg on day 1 of the 21-day cycle.
157. A method of treating, mitigating, reducing, preventing or delaying the recurrence or metastasis of breast cancer comprising co-administering to a human cisplatin-ineligible patient an effective amount of:
   a) sacituzumab govitecan;
   b) zimberelimab; and
   c) domvanalimab.
158. A kit for use in the treatment, mitigation, reduction, prevention, or delay of the recurrence or metastasis of breast cancer, wherein the kit comprises
   a) sacituzumab govitecan;
   b) zimberelimab; and
   c) domvanalimab.
159. The method of embodiment 157 or kit of embodiment 158, wherein sacituzumab govitecan is administered at one or more doses in the range of 8 mg/kg to 10 mg/kg.
160. The method or kit of embodiment 159, wherein sacituzumab govitecan is administered at one or more doses of 10 mg/kg.
161. The method or kit of any one of embodiments 157 to 160, wherein sacituzumab govitecan is administered intravenously.
162. The method or kit of any one of embodiments 157 to 161, wherein sacituzumab govitecan is administered on days 1 and 8 of a 21-day cycle.
163. The method or kit of any one of embodiments 157 to 162, wherein zimberelimab is administered at one or more doses in the range of 300 mg to 400 mg.
164. The method or kit of embodiment 163, wherein zimberelimab is administered at dose of 360 mg.
165. The method or kit of any one of embodiments 157 to 164, wherein zimberelimab is administered intravenously.
166. The method or kit of any one of embodiments 157 to 165, wherein zimberelimab is administered on day 1 of a 21-day cycle.
167. The method or kit of any one of embodiments 157 to 166, wherein domvanalimab is administered at one or more doses in the range of 800 mg to 1600 mg.
168. The method or kit of embodiment 167, wherein domvanalimab is administered at a dose of 1200 mg.
169. The method or kit of any one of embodiments 157 to 168, wherein domvanalimab is administered intravenously.
170. The method or kit of any one of embodiments 157 to 169, wherein domvanalimab is administered on day 1 of a 21-day cycle.
171. The method or kit of any one of embodiments 157 to 170, wherein sacituzumab govitecan is administered at a dose of 10 mg/kg on days 1 and 8 of a 21-day cycle; zimberelimab is administered at a dose of 360 mg on day 1 of the 21-day cycle, and domvanalimab is administered at a dose of 1200 mg on day 1 of the 21-day cycle.
172. The method or kit of any one of embodiments 157 to 171, wherein an anti-CD47 antibody is not administered to the subject or human patient.
173. The method or kit of embodiment 172, wherein the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643.
174. The method or kit of embodiment 172, wherein the anti-CD47 antibody is magrolimab.
175. The method or kit of any one of embodiments 1 to 174, wherein an MCL1 inhibitor is not administered to the subject or human patient.
176. The method or kit of embodiment 175, wherein the MCL1 inhibitor is selected from GS-9716, AMG-176, AMG-397, S-64315, AZD-5991, 483-LM, A-1210477, UMI-77 and JKY-5-037.
177. The method or kit of embodiment 175, wherein the MCL1 inhibitor is GS-9716.
178. The method or kit of any one of embodiments 1 to 177, wherein a FLT3 agonist is not administered to the subject or human patient.
179. The method or kit of embodiment 178, wherein the FLT3 agonist is selected from GS-3583, CDX-301, TAK-605, ONCR-177, Alb-Ft13L, and SYM-027.
180. The method or kit of embodiment 178, wherein the FLT3 agonist is GS-3583.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of treating, mitigating, reducing, preventing or delaying the recurrence or metastasis of urothelial cancer comprising co-administering to a human cisplatin-ineligible patient an effective amount of:
a) sacituzumab govitecan; and
b) zimberelimab.

2. A kit for use as a medicament, wherein the kit comprises
a) sacituzumab govitecan; and
b) zimberelimab.

3. A kit for use in the treatment, mitigation, reduction, prevention, or delay of the recurrence or metastasis of urothelial cancer, wherein the kit comprises
a) sacituzumab govitecan; and
b) zimberelimab.

4. The method of claim 1 or kit of claim 2 or 3, wherein sacituzumab govitecan is administered at one or more doses in the range of 8 mg/kg to 10 mg/kg.

5. The method or kit of claim 4, wherein sacituzumab govitecan is administered at one or more doses of 10 mg/kg.

6. The method or kit of any one of claims 1 to 5, wherein sacituzumab govitecan is administered intravenously.

7. The method or kit of any one of claims 1 to 6, wherein sacituzumab govitecan is administered on days 1 and 8 of a 21-day cycle.

8. The method or kit of any one of claims 1 to 7, wherein zimberelimab is administered at one or more doses in the range of 300 mg to 400 mg.

9. The method or kit of claim 8, wherein zimberelimab is administered at dose of 360 mg.

10. The method or kit of any one of claims 1 to 9, wherein zimberelimab is administered intravenously.

11. The method or kit of any one of claims 1 to 10, wherein zimberelimab is administered on day 1 of a 21-day cycle.

12. The method or kit of any one of claims 1 to 11, wherein sacituzumab govitecan is administered at a dose of 10 mg/kg on days 1 and 8 of a 21-day cycle; zimberelimab is administered at a dose of 360 mg on day 1 of the 21-day cycle.

13. A method of treating, mitigating, reducing, preventing or delaying the recurrence or metastasis of urothelial cancer comprising co-administering to a human cisplatin-ineligible patient an effective amount of:
a) sacituzumab govitecan;
b) zimberelimab; and
c) domvanalimab.

14. A kit for use as a medicament, wherein the kit comprises
a) sacituzumab govitecan;
b) zimberelimab; and
c) domvanalimab.

15. A kit for use in the treatment, mitigation, reduction, prevention, or delay of the recurrence or metastasis of urothelial cancer, wherein the kit comprises
a) sacituzumab govitecan;
b) zimberelimab; and
c) domvanalimab.

16. The method of claim 13 or kit of claim 14 or 15, wherein sacituzumab govitecan is administered at one or more doses in the range of 8 mg/kg to 10 mg/kg.

17. The method or kit of claim 16, wherein sacituzumab govitecan is administered at one or more doses of 10 mg/kg.

18. The method or kit of any one of claims 13 to 17, wherein sacituzumab govitecan is administered intravenously.

19. The method or kit of any one of claims 13 to 18, wherein sacituzumab govitecan is administered on days 1 and 8 of a 21-day cycle.

20. The method or kit of any one of claims 13 to 19, wherein zimberelimab is administered at one or more doses in the range of 300 mg to 400 mg.

21. The method or kit of claim 20, wherein zimberelimab is administered at dose of 360 mg.

22. The method or kit of any one of claims 13 to 21, wherein zimberelimab is administered intravenously.

23. The method or kit of any one of claims 13 to 22, wherein zimberelimab is administered on day 1 of a 21-day cycle.

24. The method or kit of any one of claims 13 to 23, wherein domvanalimab is administered at one or more doses in the range of 800 mg to 1600 mg.

25. The method or kit of claim 24, wherein domvanalimab is administered at a dose of 1200 mg.

26. The method or kit of any one of claims 13 to 25, wherein domvanalimab is administered intravenously.

27. The method or kit of any one of claims 13 to 26, wherein domvanalimab is administered on day 1 of a 21-day cycle.

28. The method or kit of any one of claims 13 to 27, wherein sacituzumab govitecan is administered at a dose of 10 mg/kg on days 1 and 8 of a 21-day cycle; zimberelimab is administered at a dose of 360 mg on day 1 of the 21-day cycle, and domvanalimab is administered at a dose of 1200 mg on day 1 of the 21-day cycle.

29. The method or kit of any one of claims 1 to 28, wherein an anti-CD47 antibody is not administered to the subject or human patient.

30. The method or kit of claim 29, wherein the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643.

31. The method or kit of claim 29, wherein the anti-CD47 antibody is magrolimab.

32. A method of treating, mitigating, reducing, preventing or delaying the recurrence or metastasis of non-small cell lung cancer comprising co-administering to a human patient an effective amount of:
a) sacituzumab govitecan;
b) zimberelimab; and
c) domvanalimab.

33. A kit for use in the treatment, mitigation, reduction, prevention, or delay of the recurrence or metastasis of non-small cell lung cancer, wherein the kit comprises
a) sacituzumab govitecan;
b) zimberelimab; and
c) domvanalimab.

34. The method of claim 32 or kit of claim 33, wherein sacituzumab govitecan is administered at one or more doses in the range of 8 mg/kg to 10 mg/kg.

35. The method or kit of claim 34, wherein sacituzumab govitecan is administered at one or more doses of 10 mg/kg.

36. The method or kit of any one of claims 32 to 34, wherein sacituzumab govitecan is administered intravenously.

37. The method or kit of any one of claims 32 to 36, wherein sacituzumab govitecan is administered on days 1 and 8 of a 21-day cycle.

38. The method or kit of any one of claims 32 to 37, wherein zimberelimab is administered at one or more doses in the range of 300 mg to 400 mg.

39. The method or kit of claim 38, wherein zimberelimab is administered at dose of 360 mg.

40. The method or kit of any one of claims 32 to 39, wherein zimberelimab is administered intravenously.

41. The method or kit of any one of claims 32 to 40, wherein zimberelimab is administered on day 1 of a 21-day cycle.

42. The method or kit of any one of claims 32 to 41, wherein domvanalimab is administered at one or more doses in the range of 800 mg to 1600 mg.

43. The method or kit of claim 42, wherein domvanalimab is administered at a dose of 1200 mg.

44. The method or kit of any one of claims 32 to 43, wherein domvanalimab is administered intravenously.

45. The method or kit of any one of claims 32 to 44, wherein domvanalimab is administered on day 1 of a 21-day cycle.

46. The method or kit of any one of claims 32 to 45, wherein sacituzumab govitecan is administered at a dose of 10 mg/kg on days 1 and 8 of a 21-day cycle;
zimberelimab is administered at a dose of 360 mg on day 1 of the 21-day cycle, and domvanalimab is administered at a dose of 1200 mg on day 1 of the 21-day cycle.

47. The method or kit of any one of claims 32 to 46, wherein an anti-CD47 antibody is not administered to the subject or human patient.

48. The method or kit of claim 47, wherein the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643.

49. The method or kit of claim 48, wherein the anti-CD47 antibody is magrolimab.

50. The method or kit of any one of claims 32 to 49, wherein the NSCLC is (i) squamous NSCLC or (ii) non-squamous NSCLC.

51. The method or kit of any one of claims 32 to 50, wherein the NSCLC is without EGFR, ALK, or other actionable genomic alterations.

52. The method or kit of any one of claims 32 to 51, wherein the NSCLC is (i) metastatic or (ii) advanced.

53. A method of treating, mitigating, reducing, preventing or delaying the recurrence or metastasis of breast cancer comprising co-administering to a human cisplatin-ineligible patient an effective amount of:
a) sacituzumab govitecan; and
b) zimberelimab.

54. A kit for use in the treatment, mitigation, reduction, prevention, or delay of the recurrence or metastasis of breast cancer, wherein the kit comprises
a) sacituzumab govitecan; and
b) zimberelimab.

55. The method of claim 53 or kit of claim 54, wherein
sacituzumab govitecan is administered at one or more doses in the range of 8 mg/kg to 10 mg/kg.

56. The method or kit of claim 55, wherein sacituzumab govitecan is administered at one or more doses of 10 mg/kg.

57. The method or kit of any one of claims 53 to 56 wherein sacituzumab govitecan is administered intravenously.

58. The method or kit of any one of claims 53 to 57, wherein sacituzumab govitecan is administered on days 1 and 8 of a 21-day cycle.

59. The method or kit of any one of claims 53 to 58, wherein zimberelimab is administered at one or more doses in the range of 300 mg to 400 mg.

60. The method or kit of claim 59, wherein zimberelimab is administered at dose of 360 mg.

61. The method or kit of any one of claims 53 to 60 wherein zimberelimab is administered intravenously.

62. The method or kit of any one of claims 53 to 61, wherein zimberelimab is administered on day 1 of a 21-day cycle.

63. The method or kit of any one of claims 53 to 62, wherein sacituzumab govitecan is administered at a dose of 10 mg/kg on days 1 and 8 of a 21-day cycle; zimberelimab is administered at a dose of 360 mg on day 1 of the 21-day cycle.

64. A method of treating, mitigating, reducing, preventing or delaying the recurrence or metastasis of breast cancer comprising co-administering to a human cisplatin-ineligible patient an effective amount of:
a) sacituzumab govitecan;
b) zimberelimab; and
c) domvanalimab.

65. A kit for use in the treatment, mitigation, reduction, prevention, or delay of the recurrence or metastasis of breast cancer, wherein the kit comprises
a) sacituzumab govitecan;
b) zimberelimab; and
c) domvanalimab.

66. The method of claim 64 or kit of claim 65, wherein sacituzumab govitecan is administered at one or more doses in the range of 8 mg/kg to 10 mg/kg.

67. The method or kit of claim 66, wherein sacituzumab govitecan is administered at one or more doses of 10 mg/kg.

68. The method or kit of any one of claims 64 to 67, wherein sacituzumab govitecan is administered intravenously.

69. The method or kit of any one of claims 64 to 68, wherein sacituzumab govitecan is administered on days 1 and 8 of a 21-day cycle.

70. The method or kit of any one of claims 64 to 69, wherein zimberelimab is administered at one or more doses in the range of 300 mg to 400 mg.

71. The method or kit of claim 70, wherein zimberelimab is administered at dose of 360 mg.

72. The method or kit of any one of claims 64 to 71, wherein zimberelimab is administered intravenously.

73. The method or kit of any one of claims 64 to 72, wherein zimberelimab is administered on day 1 of a 21-day cycle.

74. The method or kit of any one of claims 64 to 73,
wherein domvanalimab is administered at one or more doses in the range of 800 mg to 1600 mg.

75. The method or kit of claim 74, wherein domvanalimab is administered at a dose of 1200 mg.

76. The method or kit of any one of claims 64 to 75, wherein domvanalimab is administered intravenously.

77. The method or kit of any one of claims 64 to 76, wherein domvanalimab is administered on day 1 of a 21-day cycle.

78. The method or kit of any one of claims 64 to 77, wherein sacituzumab govitecan is administered at a dose of 10 mg/kg on days 1 and 8 of a 21-day cycle; zimberelimab is administered at a dose of 360 mg on day 1 of the 21-day cycle, and domvanalimab is administered at a dose of 1200 mg on day 1 of the 21-day cycle.

79. The method or kit of any one of claims 64 to 78, wherein an anti-CD47 antibody is not administered to the subject or human patient.

80. The method or kit of claim 79, wherein the anti-CD47 antibody is selected from magrolimab, lemzoparlimab, letaplimab, ligufalimab, AO-176, IBI-322, ZL-1201, IMC-002, SRF-231, CC-90002 (a.k.a., INBRX-103), NI-1701 (a.k.a., TG-1801) and STI-6643.

81. The method or kit of claim 79, wherein the anti-CD47 antibody is magrolimab.

82. The method or kit of any one of claims 53 to 81, wherein the breast cancer is triple negative breast cancer, HR⁺/HER2⁻ breast cancer, or HR⁺/HER2^{low} breast cancer.

83. The method or kit of any one of claims 53 to 82, wherein the breast cancer is metastatic.

84. The method or kit of any one of claims 53 to 83, wherein the breast cancer is resistant or refractory to one or more anti-cancer therapies.

85. The method or kit of any one of claims 1 to 84, wherein an MCL1 inhibitor is not administered to the subject or human patient.

86. The method or kit of claim 85, wherein the MCL1 inhibitor is selected from GS-9716, AMG-176, AMG-397, S-64315, AZD-5991, 483-LM, A-1210477, UMI-77 and JKY-5-037.

87. The method or kit of claim 85, wherein the MCL1 inhibitor is GS-9716.

88. The method or kit of any one of claims 1 to 87, wherein a FLT3 agonist is not administered to the subject or human patient.

89. The method or kit of claim 88, wherein the FLT3 agonist is selected from GS-3583, CDX-301, TAK-605, ONCR-177, Alb-Ftl3L, and SYM-027.

90. The method or kit of claim 88, wherein the FLT3 agonist is GS-3583.
